(19) United States
(12) Patent Application Publication
(10) Pub. No.: US 2018/0226601 A1
(43) Pub. Date: Aug. 9, 2018

(54) ORGANIC ELECTROLUMINESCENT ELEMENT, ILLUMINATION DEVICE, DISPLAY DEVICE, AND MIXED MATERIAL

(71) Applicant: IDEMITSU KOSAN CO., LTD., Tokyo (JP)

(72) Inventor: Takeshi IKEDA, Sodegaura-shi, Chiba (JP)

(73) Assignee: IDEMITSU KOSAN CO., LTD., Tokyo (JP)

(21) Appl. No.: 15/749,406

(22) PCT Filed: Sep. 9, 2016
(86) PCT No.: PCT/JP2016/076696

§ 371 Date: Jan. 31, 2018

(30) Foreign Application Priority Data
Sep. 11, 2015 (JP) 2015-180020

(51) Int. Cl. H01L 51/50; H01L 51/52; H01L 51/00

(52) U.S. Cl. CPC H01L 51/5024; H01L 51/5056; H01L 51/5072; H01L 51/5092; C07D 209/82; H01L 51/5221; H01L 51/0072; H01L 51/0061; H01L 51/5206

(57) ABSTRACT

An organic electroluminescence device including a light emitting layer containing a compound represented by the following formula (1) and a compound represented by the formula (11) between a cathode and an anode. A lighting device or a display device including the organic electroluminescence device. A mixed material including a compound represented by the following formula (1) and a compound represented by the formula (11).

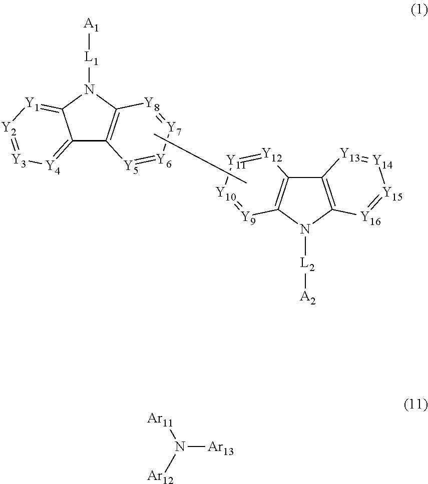

### TECHNICAL FIELD

The invention relates an organic electroluminescence device, a lighting device, a display device and mixed material.

### BACKGROUND ART

When a voltage is applied to an organic electroluminescence device (hereinafter referred to as an organic EL device), holes from anode and electrons from cathode are relatively injected into a light-emitting layer. In the light-emitting layer, the injected holes and electrons recombine to form an exciton.

An organic EL device comprises a light-emitting layer between an anode and a cathode. In addition, there are cases in which it has a laminated structure including an organic layer such as a hole injection layer, a hole transporting layer, an electron injection layer, and an electron transporting layer.

In Patent Documents 1 to 10, technologies in which two types of host materials are used for a light-emitting layer are disclosed. Patent Document 11 discloses an organic EL device using a biscarbazole derivative substituted with a fluoranthenyl group as a host material.

### RELATED ART DOCUMENTS

### Patent Document

Patent Document 1: JP-A-2000-164360
Patent Document 2: JP-A-2002-43063
Patent Document 3: JP-A-2004-515895
Patent Document 4: JP-A-2008-524848
Patent Document 5: JP-A-2004-221063
Patent Document 6: JP-A-2009-535812
Patent Document 7: WO 2006/112265
Patent Document 8: JP-A-2008-294404
Patent Document 9: WO 2012/018120
Patent Document 10: JP-A-2009-16693
Patent Document 11: WO 2012/108388

### SUMMARY OF INVENTION

An object of the present invention is to provide an organic EL device having high luminous efficiency and long lifetime.

A technology using two types of host materials for a light-emitting layer is reaching practical use levels as performance, but further higher performance has been required. Patent Document 11 discloses experimental examples using a biscarbazole derivative substituted with a fluoranthenyl group as a single host, but as a result of diligent research, the inventors of the present invention have found that the above-mentioned problems can be solved by using specific two types of host materials, and have completed the present invention.

One aspect of the invention provides the following organic EL device.

An organic electroluminescence device comprising a cathode, an anode, and a light-emitting layer therebetween, and the light-emitting layer comprising a compound represented by the following formula (1) and a compound represented by the following formula (11),

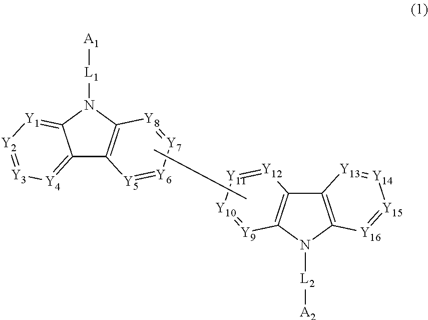

wherein A₁ is a substituted or unsubstituted aryl group including 6 to 18 carbon atoms that form a ring (hereinafter referred to as “ring carbon atoms”),

A₂ is a substituted or unsubstituted fluoranthenyl group or a substituted or unsubstituted azafluoranthenyl group,

Y₁ to Y₁₆ are independently C(R), R is independently a hydrogen atom, a substituted or unsubstituted aryl group including 5 to 30 ring carbon atoms, a substituted or unsubstituted alkyl group including 1 to 10 carbon atoms, a substituted or unsubstituted alkoxy group including 1 to 10 carbon atoms, a substituted or unsubstituted aralkyl group including 6 to 32 carbon atoms, a substituted or unsubstituted aryloxy group including 5 to 30 ring carbon atoms, a substituted or unsubstituted arylthio group including 5 to 30 ring carbon atoms, or a substituted or unsubstituted alkoxycarbonyl group including 2 to 11 carbon atoms, provided that R in one of Y₅ to Y₈ and R in one of Y₉ to Y₁₂ are a single bond which bonds with each other,

adjacent Rs may be bonded with each other to form a saturated or unsaturated, 5-membered or 6-membered ring structure which may have a substituent, and

L₁ and L₂ are independently a single bond, a substituted or unsubstituted arylene group including 6 to 18 ring carbon atoms or a substituted or unsubstituted heteroarylene group including 3 to 20 atoms that form a ring (hereinafter referred to as “ring atoms”),

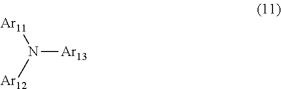

wherein Ar₁₁ to Ar₁₃ are independently a substituted or unsubstituted aryl group including 5 to 50 ring carbon atoms, a substituted or unsubstituted dibenzofuranyl group, a substituted or unsubstituted dibenzothiophenyl group, a substituted or unsubstituted alkyl group including 1 to 50 carbon atoms, a substituted or unsubstituted alkoxy group including 1 to 50 carbon atoms, a substituted or unsubstituted aralkyl group including 6 to 50 carbon atoms, a substituted or unsubstituted aryloxy group including 5 to 50 ring carbon atoms, a substituted or unsubstituted arylthio group including 5 to 50 ring carbon atoms, a substituted or unsubstituted alkoxycarbonyl group including 2 to 50 carbon atoms, an amino group substituted with a substituted or unsubstituted aryl group including 5 to 50 ring carbon atoms, an amino group substituted with a substituted or unsubstituted dibenzofuranyl group or a substituted or unsubstituted dibenzothiophenyl group, a halogen atom, a cyano group, a nitro group, a hydroxyl group, or a carboxyl group.

According to another aspect of the present invention, a lighting device or a display device including an organic EL device is provided.

According to another aspect of the present invention, a mixing material comprising a compound represented by the above formula (1) and a compound represented by the above formula (11) is provided.

According to the present invention, an organic EL device having high luminous efficiency and long lifetime can be provided.

### BRIEF EXPLANATION OF THE DRAWING

FIG. 1 is a diagram showing a schematic configuration of one embodiment of an organic EL device of the present invention.

### DESCRIPTION OF THE EMBODIMENTS

In the present specification, a hydrogen atom includes isomers differing in number of neutrons, i.e. protium, deuterium and tritium.

In the present specification, the number of “ring carbon atoms” means the number of carbon atoms among atoms constituting a ring of a compound in which atoms are bonded in the form of a ring (for example, a monocyclic compound, a fused ring compound, a cross-linked compound, a carbocyclic compound or a heterocyclic compound). When the ring is substituted with a substituent, the carbon contained in the substituent is not included in the number of ring carbon atoms. The same is applied to the “ring carbon atoms” mentioned below, unless otherwise indicated. For example, a benzene ring includes 6 ring carbon atoms, a naphthalene ring includes 10 ring carbon atoms, a pyridinyl group includes 5 ring carbon atoms, and a furanyl group includes 4 ring carbon atoms. When a benzene ring or a naphthalene ring is substituted with an alkyl group as a substituent, for example, the number of carbon atoms of the alkyl group is not included in the number of ring carbon atoms. When a fluorene ring is bonded with a fluorene ring as a substituent (including a spirofluorene ring), for example, the number of carbon atoms of the fluorene ring as the substituent is not included in the number of ring carbon atoms.

In the present specification, the number of “ring atoms” means the number of atoms constituting a ring of a compound having a structure in which atoms are bonded in the form of a ring (for example, monocycle, fused ring, ring assembly) (for example, a monocyclic compound, a fused ring compound, a cross-linked compound, a carbocyclic compound or a heterocyclic compound). It does not include atoms which do not form a ring or atoms contained in a substituent when the ring is substituted with the substituent. The same is applied to the “ring atoms” mentioned below, unless otherwise indicated. For example, a pyridine ring includes 6 ring carbon atoms, a quinazoline ring includes 10 ring atoms, and a furan ring includes 5 ring atoms. Hydrogen atoms respectively bonded with a carbon atom of a pyridine ring or a quinazoline ring or atoms constituting a substituent are not included in the number of ring atoms. When a fluorene ring is bonded with a fluorene ring as a substituent (including a spirofluorene ring), for example, the number of atoms of the fluorene ring as a substituent is not included in the number of ring atoms.

In the present specification, the “XX to YY carbon atoms” in the “substituted or unsubstituted ZZ group including XX to YY carbon atoms” means the number of carbon atoms when the ZZ group is unsubstituted. The number of carbon atoms of a substituent when the group is substituted is not included. Note that “YY” is larger than “XX”, and “XX” and “YY” are independently an integer equal to or larger than 1.

The expression “XX to YY atoms” used in connection with the expression “substituted or unsubstituted ZZ group including XX to YY atoms” refers to the number of atoms when the ZZ group is unsubstituted, and excludes the number of atoms included in a substituent when the ZZ group is substituted. Note that “YY” is larger than “XX”, and “XX” and “YY” are independently an integer equal to or larger than 1.

In the present specification, the “unsubstituted” in the “substituted or unsubstituted” means bonding of a hydrogen atom, not substitution by the substituent mentioned above.

An aspect of the organic EL device according to the invention comprises a light-emitting layer comprising a compound represented by the following formula (1) and a compound represented by the following formula (11).

An aspect of the mixing material according to the invention comprises a compound represented by the following formula (1) and a compound represented by the following formula (11). It is preferable that an aspect of the mixing material according to the invention is a material for an organic EL device. The mixing material may also be referred to as a composition, a premix, a premix material or the like.

The content of the compound represented by the formula (11) is preferably 5 to 30 wt %, more preferably 5 to 20 wt %, particularly preferably 5 to 15 wt %, based on 100 wt % of the mixing material.

The mixing material of the present invention may consist essentially of a compound represented by the formula (1), a compound represented by the formula (11), and optionally a metal complex, a heterocyclic compound, a fused aromatic compound, and an aromatic amine compound, and may contain inevitable impurities in a range that does not impair the effects of the present invention.

Since a compound represented by the formula (1) tends to have a higher ionization potential than the adjacent hole transporting material (a material for a hole transporting layer (described later)), the energy barrier of hole injection from the hole transporting material to the host material is relatively large. Therefore, when used alone as a host material, it is conceivable that injection of holes into the light-emitting layer is not sufficient with compared to injection of electrons into the light-emitting layer. In the case where hole injection into the light-emitting layer is not sufficient, lower efficiency may be caused by reduction in recombination probability. In addition, since holes are not sufficiently injected into the light-emitting layer with compared to injection of electrons into the light-emitting layer, electrons invade the hole transporting material and recombine even on the hole transporting material, which causes deterioration of the device, and the lifetime of the device may be shortened.

By combining the compound represented by the formula (1) and the compound represented by the formula (11), holes are appropriately injected from the hole transporting material into the light-emitting layer, and thus high efficiency and long lifetime can be achieved.

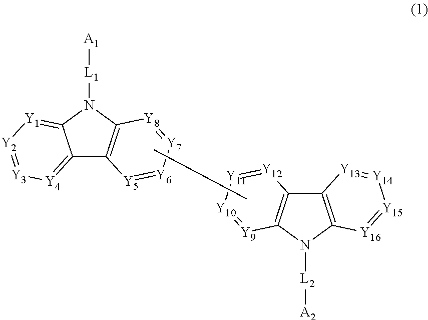

wherein A₁ is a substituted or unsubstituted aryl group including 6 to 18 ring carbon atoms,

A₂ is a substituted or unsubstituted fluoranthenyl group or a substituted or unsubstituted azafluoranthenyl group,

Y₁ to Y₁₆ are independently C(R), R is independently a hydrogen atom, a substituted or unsubstituted aryl group including 5 to 30 (preferably 6 to 30, more preferably 5 to 10, and particularly preferably 6 to 10) ring carbon atoms, a substituted or unsubstituted alkyl group including 1 to 10 carbon atoms, a substituted or unsubstituted alkoxy group including 1 to 10 carbon atoms, a substituted or unsubstituted aralkyl group including 6 to 32 (preferably 7 to 32, more preferably 6 to 12, and particularly preferably 7 to 12) carbon atoms, a substituted or unsubstituted aryloxy group including 5 to 30 (preferably 6 to 30, more preferably 5 to 10, and particularly preferably 6 to 10) ring carbon atoms, a substituted or unsubstituted arylthio group including 5 to 30 (preferably 6 to 30, more preferably 5 to 10, and particularly preferably 6 to 10) ring carbon atoms, a substituted or unsubstituted alkoxycarbonyl group including 2 to 11 carbon atoms, or a single bond bonding to the carbazole skeleton,

adjacent Rs may be bonded with each other to form a saturated or unsaturated, 5-membered or 6-membered ring structure which may have a substituent, and

it is preferable that R in any of Y₅ to Y₅ and R in any of Y₉ to Y₁₂ are a single bond which bonds to the carbazole skeleton. That is, it is preferable that R in one of Y₅ to Y₈ and R in one of Y₉ to Y₁₂ are a single bond which bonds with each other. That is, it is preferable that a carbon atom in one of Y₅ to Y₅ and a carbon atom in one of Y₉ to Y₁₂ are bonded by a single bond.

L₁ and L₂ are independently a single bond, a substituted or unsubstituted arylene group including 6 to 18 ring carbon atoms or a substituted or unsubstituted heteroarylene group including 3 to 20 (preferably 5 to 20, more preferably 3 to 15, and particularly preferably 5 to 15) ring atoms.

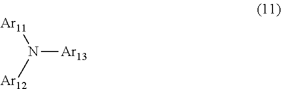

wherein Ar₁₁ to Ar₁₃ are independently a substituted or unsubstituted aryl group including 5 to 50 (preferably 6 to 50) ring carbon atoms, a substituted or unsubstituted dibenzofuranyl group, a substituted or unsubstituted dibenzothiophenyl group, a substituted or unsubstituted alkyl group including 1 to 50 carbon atoms, a substituted or unsubstituted alkoxy group including 1 to 50 carbon atoms, a substituted or unsubstituted aralkyl group including 6 to 50 (preferably 7 to 50) carbon atoms, a substituted or unsubstituted aryloxy group including 5 to 50 (preferably 6 to 50) ring carbon atoms, a substituted or unsubstituted arylthio group including 5 to 50 (preferably 6 to 50) ring carbon atoms, a substituted or unsubstituted alkoxycarbonyl group including 2 to 50 carbon atoms, an amino group substituted with a substituted or unsubstituted aryl group including 5 to 50 (preferably 6 to 50) ring carbon atoms, an amino group substituted with a substituted or unsubstituted dibenzofuranyl group or a substituted or unsubstituted dibenzothiophenyl group, a halogen atom, a cyano group, a nitro group, a hydroxyl group, or a carboxyl group.

The compound represented by the formula (11) preferably contains neither a carbazolyl group nor an azacarbazolyl group.

The compound represented by the formula (1) is preferably a compound represented by the following formula (2), (3) or (4).

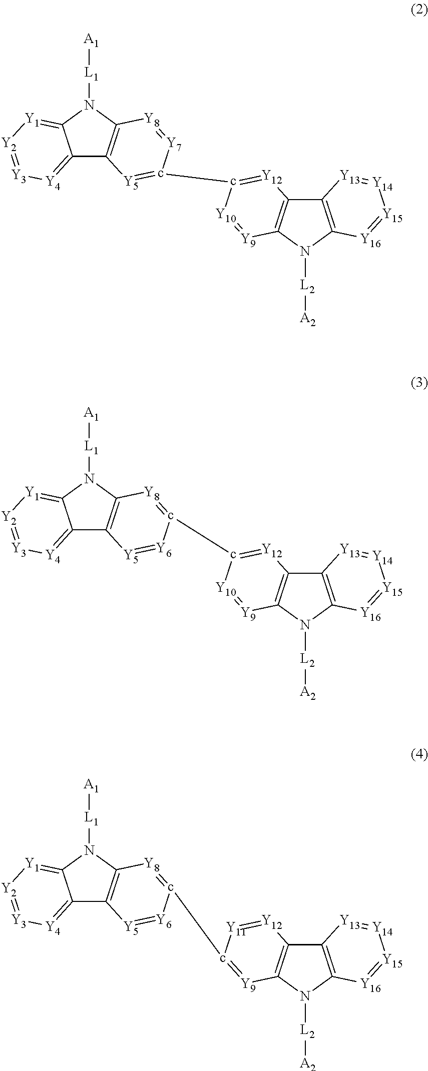

wherein A₁, A₂, Y₁ to Y₁₆, L₁ and L₂ are the same as defined for the formula (1), respectively.

Here, formula (2) corresponds to the case where R in Y₆ and Y₁₁ in formula (1) is a single bond which are bonded to each other. Formula (3) corresponds to the case where R in Y₇ and Y₁₁ in formula (1) is a single bond which are bonded to each other. Formula (4) corresponds to the case where R in Y₇ and Y₁₀ in formula (1) is a single bond which are bonded to each other.

R in Y₁ to Y₁₆ is preferably independently a hydrogen atom, a substituted or unsubstituted alkyl group including 1 to 10 carbon atoms, a substituted or unsubstituted alkoxy group including 1 to 10 carbon atoms, or a substituted or unsubstituted alkoxycarbonyl group including 2 to 10 carbon atoms, more preferably independently a hydrogen atom, or a substituted or unsubstituted alkyl group including 1 to 10 carbon atoms, and particularly preferably a hydrogen atom.

In formula (2), R in Y₁ to Y₅, Y₇ to Y₁₀ and Y₁₂ to Y₁₆ is preferably independently a hydrogen atom, a substituted or unsubstituted alkyl group including 1 to 10 carbon atoms, a substituted or unsubstituted alkoxy group including 1 to 10 carbon atoms, or a substituted or unsubstituted alkoxycarbonyl group including 2 to 10 carbon atoms, more preferably independently a hydrogen atom, or a substituted or unsubstituted alkyl group including 1 to 10 carbon atoms, and particularly preferably a hydrogen atom.

In formula (3), R in Y₁ to Y₆, Y₈ to Y₁₀ and Y₁₂ to Y₁₆ is preferably independently a hydrogen atom, a substituted or unsubstituted alkyl group including 1 to 10 carbon atoms, a substituted or unsubstituted alkoxy group including 1 to 10 carbon atoms, or a substituted or unsubstituted alkoxycarbonyl group including 2 to 10 carbon atoms, more preferably independently a hydrogen atom, or a substituted or unsubstituted alkyl group including 1 to 10 carbon atoms, and particularly preferably a hydrogen atom.

In formula (4), R in Y₁ to Y₆, Y₈ to Y₉ and Y₁₁ to Y₁₆ is preferably independently a hydrogen atom, a substituted or unsubstituted alkyl group including 1 to 10 carbon atoms, a substituted or unsubstituted alkoxy group including 1 to 10 carbon atoms, or a substituted or unsubstituted alkoxycarbonyl group including 2 to 10 carbon atoms, more preferably independently a hydrogen atom, or a substituted or unsubstituted alkyl group including 1 to 10 carbon atoms, and particularly preferably a hydrogen atom.

At least one of L₁ and L₂ is preferably a single bond, more preferably L₁ is a single bond and L₂ is not a single bond, and particularly preferably both L₁ and L₂ are a single bond.

L₂ is preferably a single bond or a phenylene group, and more preferably a single bond or m-phenylene group.

A₁ is preferably a phenyl group or a naphthyl group, and more preferably a phenyl group.

A₂ is preferably a group represented by following formula (7).

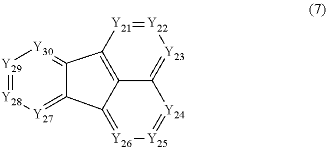

wherein Y₂₁ to Y₃₀ are independently C(R₄) or N,

R₄ is independently a hydrogen atom, a substituted or unsubstituted aryl group including 6 to 30 (preferably 6 to 10) ring carbon atoms, a substituted or unsubstituted alkyl group including 1 to 10 carbon atoms, a substituted or unsubstituted alkoxy group including 1 to 10 carbon atoms, a substituted or unsubstituted aralkyl group including 7 to 32 (preferably 7 to 12) carbon atoms, a substituted or unsubstituted aryloxy group including 6 to 30 (preferably 6 to 10) ring carbon atoms, a substituted or unsubstituted arylthio group including 6 to 30 (preferably 6 to 10) ring carbon atoms, or a substituted or unsubstituted alkoxycarbonyl group including 2 to 11 carbon atoms, provided that one of R₄ is a single bond which bonds to L₂.

At least one of Y₂₁ to Y₃₀ is preferably N.

Y₂₃ is preferably N.

It is preferable that Y₂₂ is C(R₄) and R₄ of Y₂₂ is a single bond which bonds to L₂.

It is preferable that Y₂₈ is C(R₄) and R₄ of Y₂₅ is a single bond which bonds to L₂.

The compound represented by the formula (1) is preferably a compound represented by the following formula (8). In formula (8), a nitrogen atom is bonded to any position of Y₂₁ to Y₃₀.

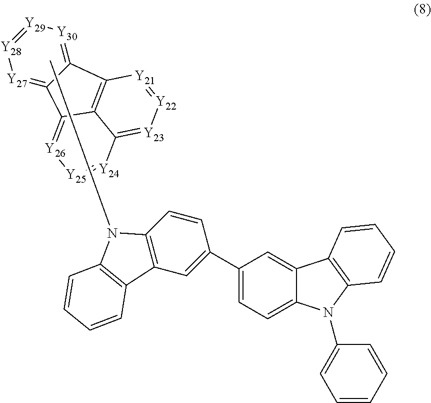

wherein Y₂₁ to Y₃₀ are the same as defined for the formula (7).

A₂ is preferably a group represented by the following formula (5),

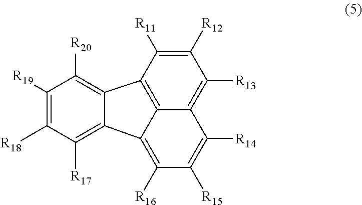

wherein R₁₁ to R₂₀ are independently a hydrogen atom, a substituted or unsubstituted aryl group including 5 to 30 (preferably 6 to 30, more preferably 5 to 10, and particularly preferably 6 to 10) ring carbon atoms, a substituted or unsubstituted alkyl group including 1 to 10 carbon atoms, a substituted or unsubstituted alkoxy group including 1 to 10 carbon atoms, a substituted or unsubstituted aralkyl group including 6 to 32 (preferably 7 to 32, more preferably 6 to 12, and particularly preferably 7 to 12) carbon atoms, a substituted or unsubstituted aryloxy group including 5 to 30 (preferably 6 to 30, more preferably 5 to 10, and particularly preferably 6 to 10) ring carbon atoms, a substituted or unsubstituted arylthio group including 5 to 30 (preferably 6 to 30, more preferably 5 to 10, and particularly preferably 6 to 10) ring carbon atoms, or a substituted or unsubstituted alkoxycarbonyl group including 2 to 11 carbon atoms, provided that one of R₁₁ to R₂₀ is a single bond which bonds to L₂,

R₁₁ to R₂₀ are the same as the above R,

It is preferable that R₁₄ bonds to L₂, and

It is preferable that R₁₈ bonds to L₂.

The compound represented by the formula (1) is preferably a compound represented by the following formula (6). In formula (6), a nitrogen atom is bonded to any position of fluoranthene.

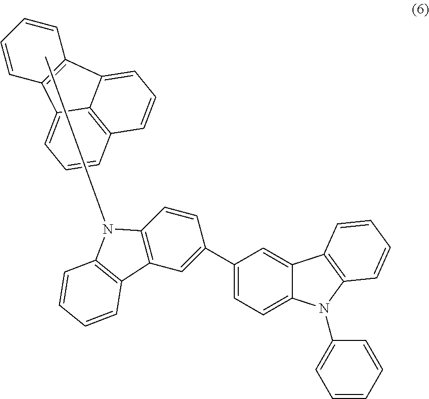

Examples of the aryl group including 6 to 18 (preferably 6 to 12) in A₁ include a non-fused aryl group and a fused aryl group, more specifically, a phenyl group, a naphthyl group, a phenanthryl group, a biphenyl group, a terphenyl group, a fluorenyl group, a benzo[c]phenanthrenyl group, a chrysenyl group, a triphenylenyl group, fluoranthenyl group, and the like.

Examples of the aryl group including 5 to 30 (preferably 6 to 30, more preferably 5 to 10, and particularly preferably 6 to 10) in R include a phenyl group, a naphthyl group, a tolyl group, a xylyl group, a phenanthryl group, a pyrenyl group, a chrysenyl group, a benzo[c]phenanthryl group, a benzo[g]chrysenyl group, a benzoanthryl group, a triphenylenyl group, a fluorenyl group, a 9,9-dimethylfluorenyl group, a benzofluorenyl group, a dibenzofluorenyl group, a biphenyl group, a terphenyl group, a quarterphenyl group, a fluoranthenyl group and the like, preferably, a phenyl group, a biphenyl group, a terphenyl group, a quarterphenyl group, a naphthyl group, a triphenylenyl group, and a fluorenyl group. Examples of the aryl group including 5 to 10 ring carbon atoms in R include a phenyl group, a naphthyl group, and the like. Examples of the aryl group including 6 to 10 ring carbon atoms in R include a phenyl group, a naphthyl group, and the like.

The alkyl group including 1 to 10 carbon atoms in R may be linear, branched or cyclic. Examples include a methyl group, an ethyl group, a propyl group, an isopropyl group, a n-butyl, a s-butyl group, an isobutyl group, a t-butyl group, a n-pentyl group, a n-hexyl group, a n-heptyl group, a n-octyl group, a n-nonyl group, a n-decyl group, a neopentyl group, 1-methylpentyl group, 2-methylpentyl group, 1-butylpentyl group, 3-methylpentyl group, cyclopentyl group, a cyclohexyl group, a cyclooctyl group, a 3,5-tetramethylcyclohexyl group and the like.

Examples of the alkoxy group including 1 to 10 carbon atoms in R include a group in which oxygen atom is bonded to the above alkyl group including 1 to 10 carbon atoms.

Examples of the aralkyl group including 6 to 32 carbon atoms (preferably 7 to 32, more preferably 6 to 12, particularly preferably 7 to 12) in R include a benzyl group, a 1-phenylethyl group, a 2-phenylethyl group, a 1-phenylisopropyl group, a 2-phenylisopropyl group, a phenyl-t-butyl group, an α-naphthylmethyl group, a 1-α-naphthylethyl group, a 2-α-naphthylethyl group, a β-naphthylmethyl group, a 1-β-naphthylethyl group, a 2-β-naphthylethyl group and the like. Examples of the aralkyl group including 6 to 12 carbon atoms in R include a benzyl group, a 1-phenylethyl group, a 2-phenylethyl group, a 1-phenylisopropyl group, a 2-phenylisopropyl group, a phenyl-t-butyl group, an α-naphthylmethyl group, a 1-α-naphthylethyl group, a 2-α-naphthylethyl group, a β-naphthylmethyl group, a 1-β-naphthylethyl group and a 2-β-naphthylethyl group and the like.

Examples of the aryloxy group including 5 to 30 (preferably 6 to 30, more preferably 5 to 10, particularly preferably 6 to 10) ring carbon atoms in R includes a group in which oxygen is bonded to the above aryl group including 5 to 30 (preferably 6 to 30, more preferably 5 to 10, particularly preferably 6 to 10) ring carbon atoms. Examples of the aryloxy group including 5 to 10 ring carbon atoms in R include a group in which oxygen is bonded to the above-mentioned aryl group including 5 to 10 ring carbon atoms.

Examples of the substituted or unsubstituted arylthio group including 5 to 30 (preferably 6 to 30, more preferably 5 to 10, particularly preferably 6 to 10) ring carbon atoms in R includes a group in which sulfer is bonded to the above aryl group including 5 to 30 (preferably 6 to 30, more preferably 5 to 10, particularly preferably 6 to 10) ring carbon atoms. Examples of the arylthio group including 5 to 10 ring carbon atoms in R include a group in which oxygen is bonded to the above-mentioned aryl group including 5 to 10 ring carbon atoms.

Examples of the alkoxycarbonyl group including 2 to 11 carbon atoms in R include a group in which oxygen atom of a oxycarbonyl is bonded to the above alkyl group including 1 to 10 carbon atoms.

Examples of the arylene group including 6 to 18 ring carbon atoms in L₁ and L₂ include a phenylene group (e.g. m-phenylene group), a naphthylene group, a biphenylene group, an anthranylene group, and a pyrenylene group.

Examples of the heteroarylene group including 3 to 20 (preferably 5 to 20, more preferably 3 to 15, particularly preferably 5 to 15) ring atoms in L₁ and L₂ includes a non-fused heteroarylene group and a fused heteroarylene group, more specifically, a divalent group derived from a pyrrolyl group, a pyrazinyl group, a pyridinyl group, an indolyl group, an isoindolyl group, a furyl group, a benzofuranyl group, an isobenzofuranyl group, a dibenzofuranyl group, a dibenzothiophenyl group, a quinolyl group, an isoquinolyl group, a quinoxalinyl group, a carbazolyl group, a phenanthridinyl group, an acridinyl group, a phenanthrolinyl group, and a thienyl group, and a divalent group formed from a pyridine ring, a pyrazine ring, a pyrimidine ring, a pyridazine ring, a triazine ring, an indole ring, a quinoline ring, an acridine ring, a pyrrolidine ring, an dioxane ring, a piperidine ring, a morpholine ring, a piperazine ring, a carbazole ring, a furan ring, a thiophene ring, oxazole ring, an oxadiazole ring, a benzoxazole ring, a thiazole ring, a thiadiazole ring, a benzothiazole ring, a triazole ring, an imidazole ring, a benzimidazole ring, a pyran ring, a dibenzofuran ring, and a benzo[c]dibenzofuran ring.

Examples of the heteroarylene group including 3 to 15 ring atoms in L₁ and L₂ includes a non-fused heteroarylene group and a fused heteroarylene group, more specifically, a divalent group derived from a pyrrolyl group, a pyrazinyl group, a pyridinyl group, an indolyl group, an isoindolyl group, a furyl group, a benzofuranyl group, an isobenzofuranyl group, a dibenzofuranyl group, a dibenzothiophenyl group, a quinolyl group, an isoquinolyl group, a quinoxalinyl group, a carbazolyl group, a phenanthridinyl group, an acridinyl group, a phenanthrolinyl group, and a thienyl group, and a divalent group formed from a pyridine ring, a pyrazine ring, a pyrimidine ring, a pyridazine ring, a triazine ring, an indole ring, a quinoline ring, an acridine ring, a pyrrolidine ring, an dioxane ring, a piperidine ring, a morpholine ring, a piperazine ring, a carbazole ring, a furan ring, a thiophene ring, oxazole ring, an oxadiazole ring, a benzoxazole ring, a thiazole ring, a thiadiazole ring, a benzothiazole ring, a triazole ring, an imidazole ring, a benzimidazole ring, a pyran ring, a dibenzofuran ring, and a benzo[c]dibenzofuran ring.

In the present specification, the heteroarylene group including 3 to 20 (preferably 5 to 20, more preferably 3 to 15, particularly preferably 5 to 15) ring atoms also include a divalent group derived from the following structure,

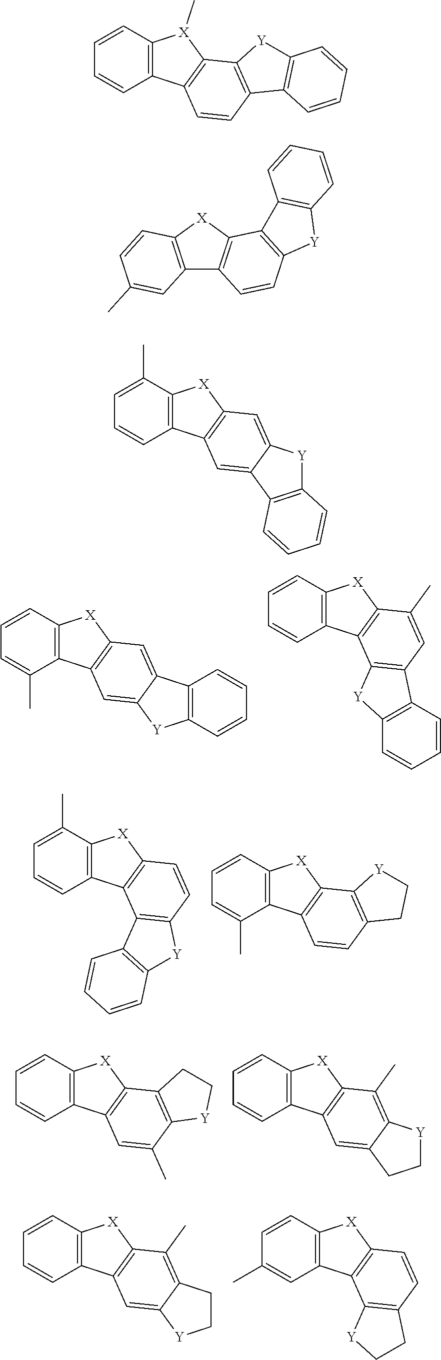

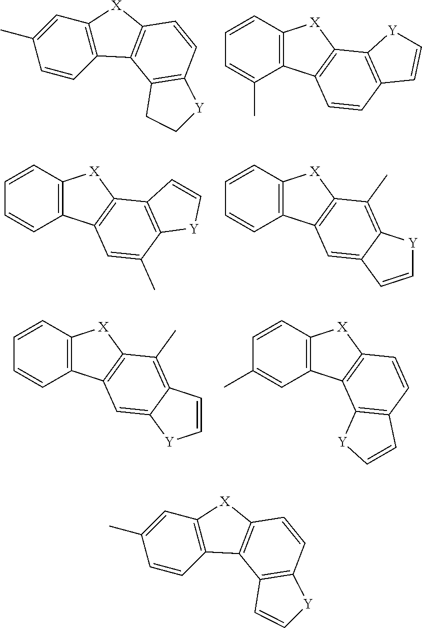

wherein X and Y are each an oxygen atom, a sulfur atom, a nitrogen atom or a —NH— group.

In in the compounds represented by the formulas (1) to (5), (7) and (8), examples of the substituent, in the case of “a substituted or unsubstituted” includes, for R, an unsubstituted aryl group including 5 to 30 (preferably 6 to 30, more preferably 5 to 10, particularly preferably 6 to 10) ring carbon atoms, an unsubstituted alkyl group including 1 to 10 carbon atoms, an unsubstituted alkoxy group including 1 to 10 carbon atoms, an unsubstituted aralkyl group including 6 to 32 (preferably 7 to 32, more preferably 6 to 12, particularly preferably 7 to 12) carbon atoms, an unsubstituted aryloxy group including 5 to 30 (preferably 6 to 30, more preferably 5 to 10, particularly preferably 6 to 10) ring carbon atoms, an unsubstituted arylthio group including 5 to 30 (preferably 6 to 30, more preferably 5 to 10, particularly preferably 6 to 10) ring carbon atoms, an unsubstituted alkoxycarbonyl group including 2 to 11 carbon atoms, and the like.

Specific examples of each of these substituents are the same as those described above as the substituent of R, respectively.

Further, in the compounds represented by the formulas (1) to (5), (7) and (8), examples of the substituent, in the case of “a substituted or unsubstituted” includes, an unsubstituted silyl group (—SiH₃), a silyl group substituted with an alkyl group including 1 to 10 carbon atoms, a halogen atom, a halogenated alkyl group including 1 to 10 carbon atoms, a cyano group, and the like,

Examples of the silyl group substituted with an alkyl group including 1 to 10 carbon atoms include trimethylsilyl group and the like.

Examples of the halogen atom include fluorine atom and the like.

Examples of the halogenated alkyl group including 1 to 10 carbon atoms include a trifluoromethyl group and the like.

Examples of the compounds represented by formulas (1) to (6) and (7) to (8) include the following compounds. In the following structural formula, D represents deuterium.

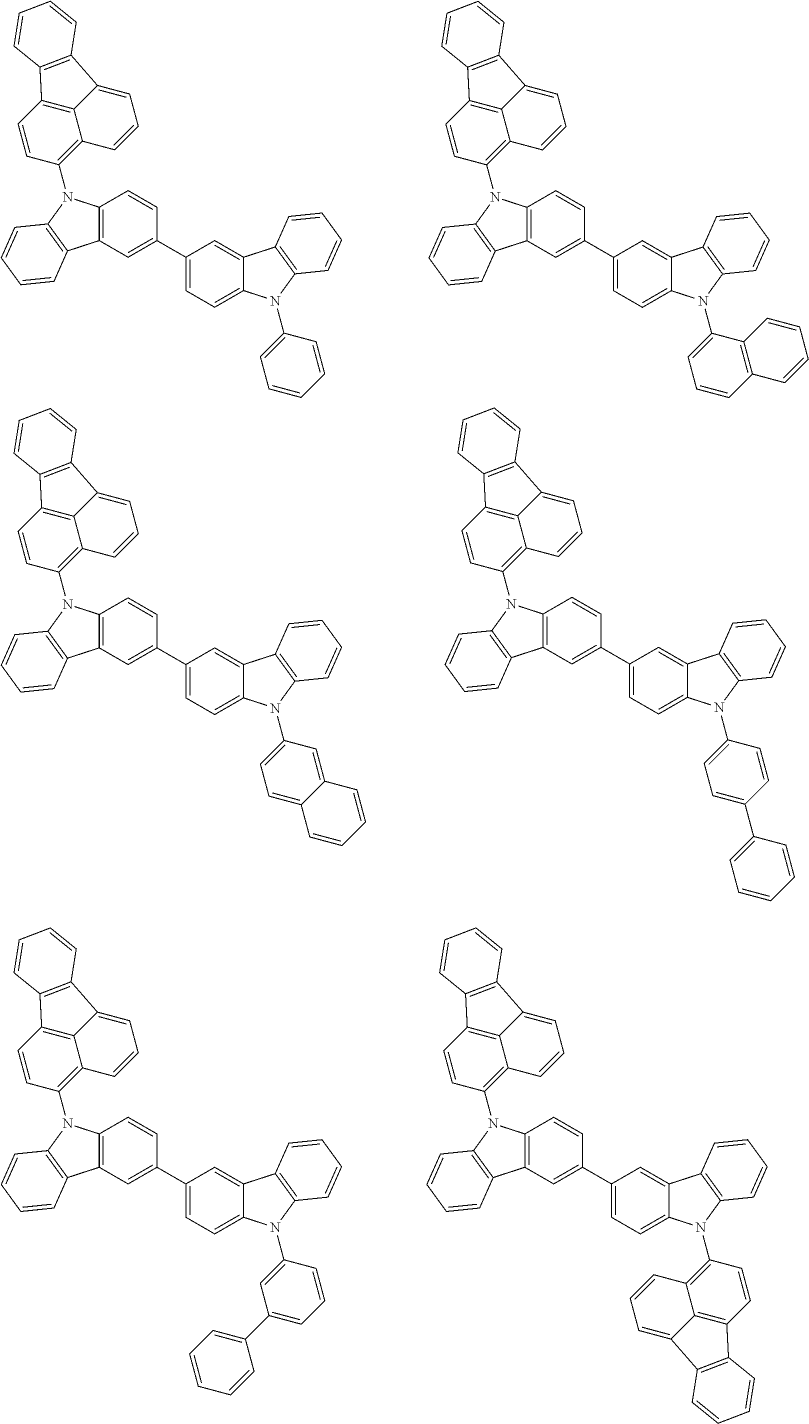

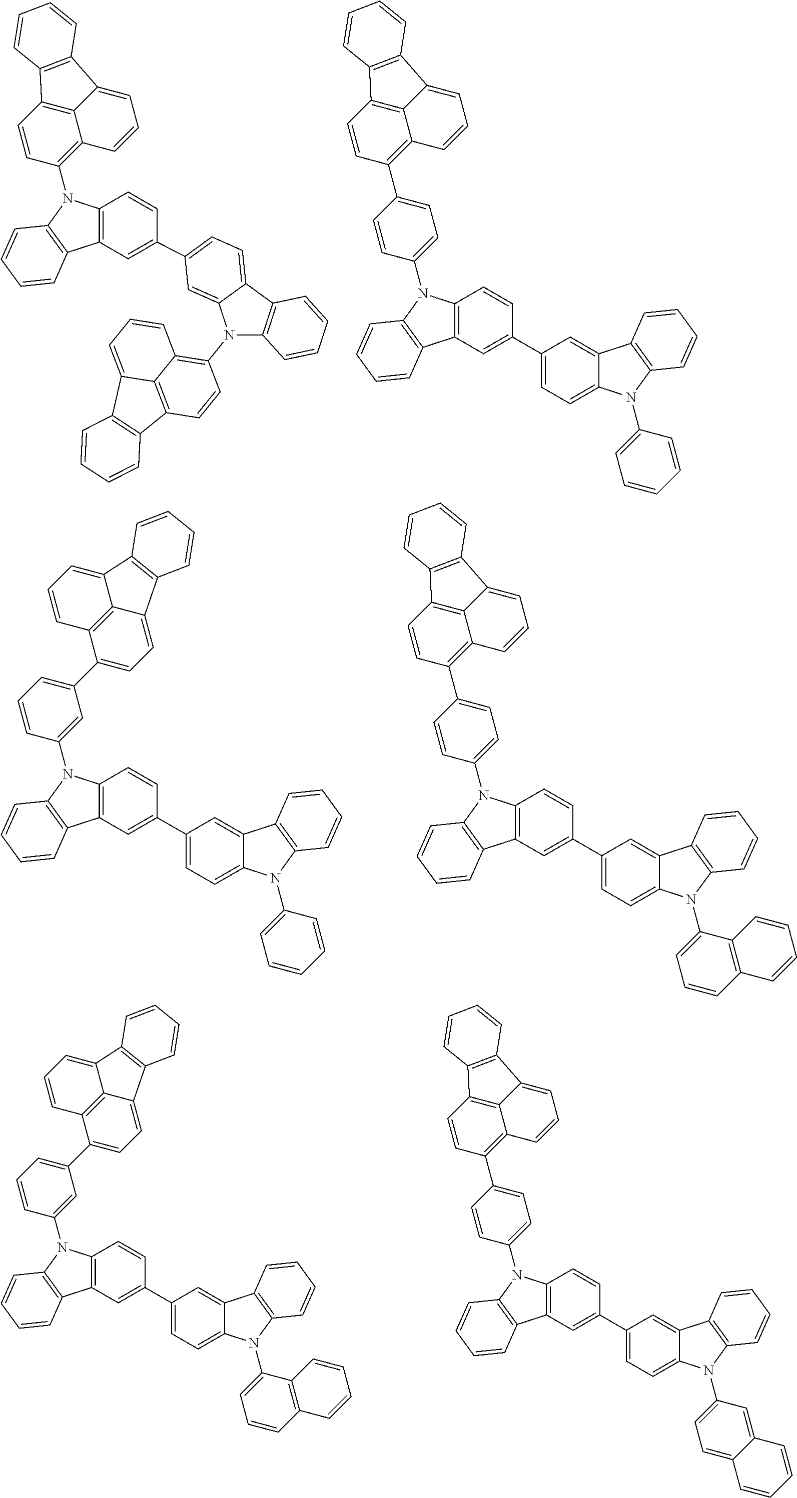

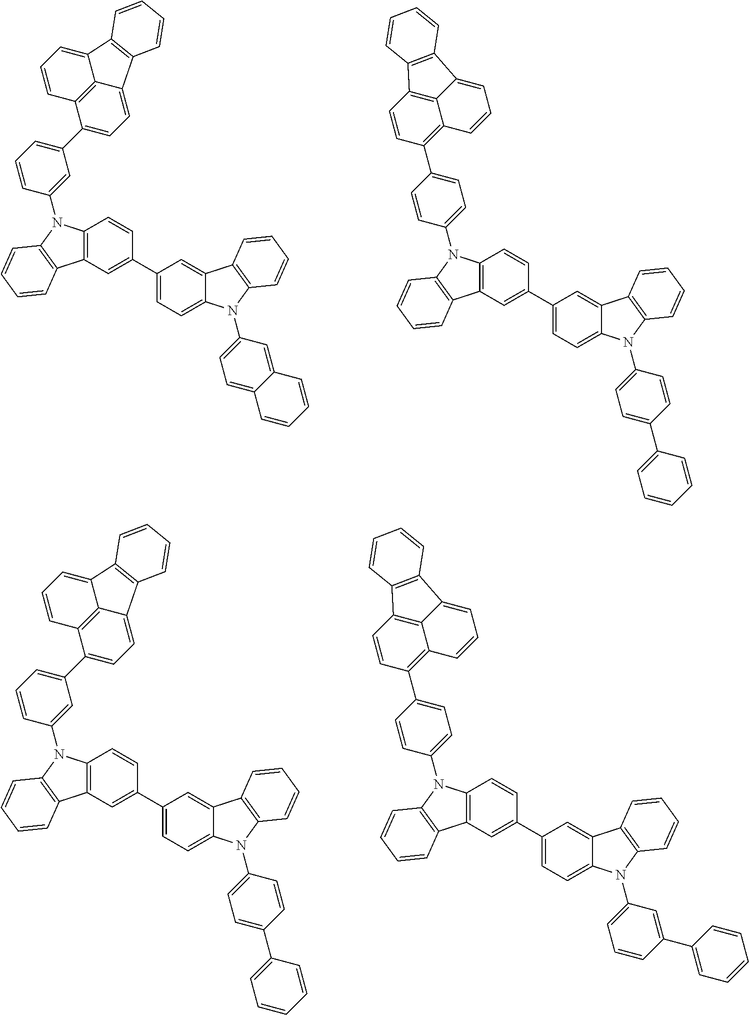 [SMILES: c1cc(-c2ccc3c4c(cccc24)-c2ccccc2-3)cc(-n2c3ccccc3c3cc(-c4ccc5c(c4)c4ccccc4n5-c4ccc5ccccc5c4)ccc32)c1 ; c1ccc(-c2ccc(-n3c4ccccc4c4cc(-c5ccc6c(c5)c5ccccc5n6-c5ccc(-c6ccc7c8c(cccc68)-c6ccccc6-7)cc5)ccc43)cc2)cc1 ; c1ccc(-c2ccc(-n3c4ccccc4c4cc(-c5ccc6c(c5)c5ccccc5n6-c5cccc(-c6ccc7c8c(cccc68)-c6ccccc6-7)c5)ccc43)cc2)cc1 ; c1ccc(-c2cccc(-n3c4ccccc4c4cc(-c5ccc6c(c5)c5ccccc5n6-c5ccc(-c6ccc7c8c(cccc68)-c6ccccc6-7)cc5)ccc43)c2)cc1]

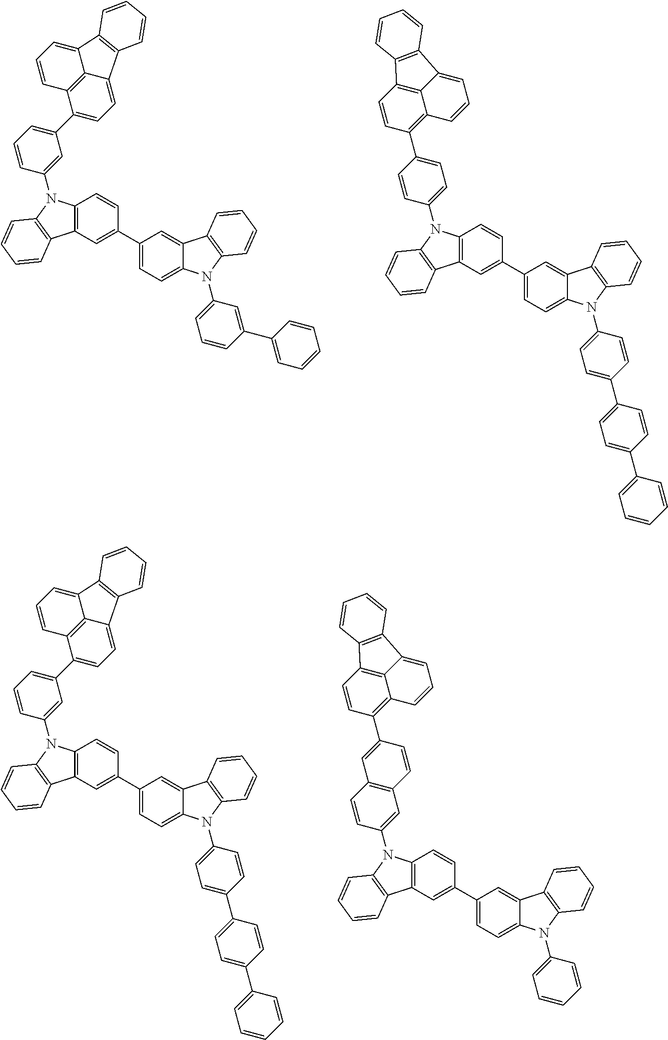 [SMILES: c1ccc(-c2cccc(-n3c4ccccc4c4cc(-c5ccc6c(c5)c5ccccc5n6-c5cccc(-c6ccc7c8c(cccc68)-c6ccccc6-7)c5)ccc43)c2)cc1 ; c1ccc(-c2ccc(-c3ccc(-n4c5ccccc5c5cc(-c6ccc7c(c6)c6ccccc6n7-c6ccc(-c7ccc8c9c(cccc79)-c7ccccc7-8)cc6)ccc54)cc3)cc2)cc1 ; c1ccc(-c2ccc(-c3ccc(-n4c5ccccc5c5cc(-c6ccc7c(c6)c6ccccc6n7-c6cccc(-c7ccc8c9c(cccc79)-c7ccccc7-8)c6)ccc54)cc3)cc2)cc1 ; c1ccc(-n2c3ccccc3c3cc(-c4ccc5c(c4)c4ccccc4n5-c4ccc5cc(-c6ccc7c8c(cccc68)-c6ccccc6-7)ccc5c4)ccc32)cc1]

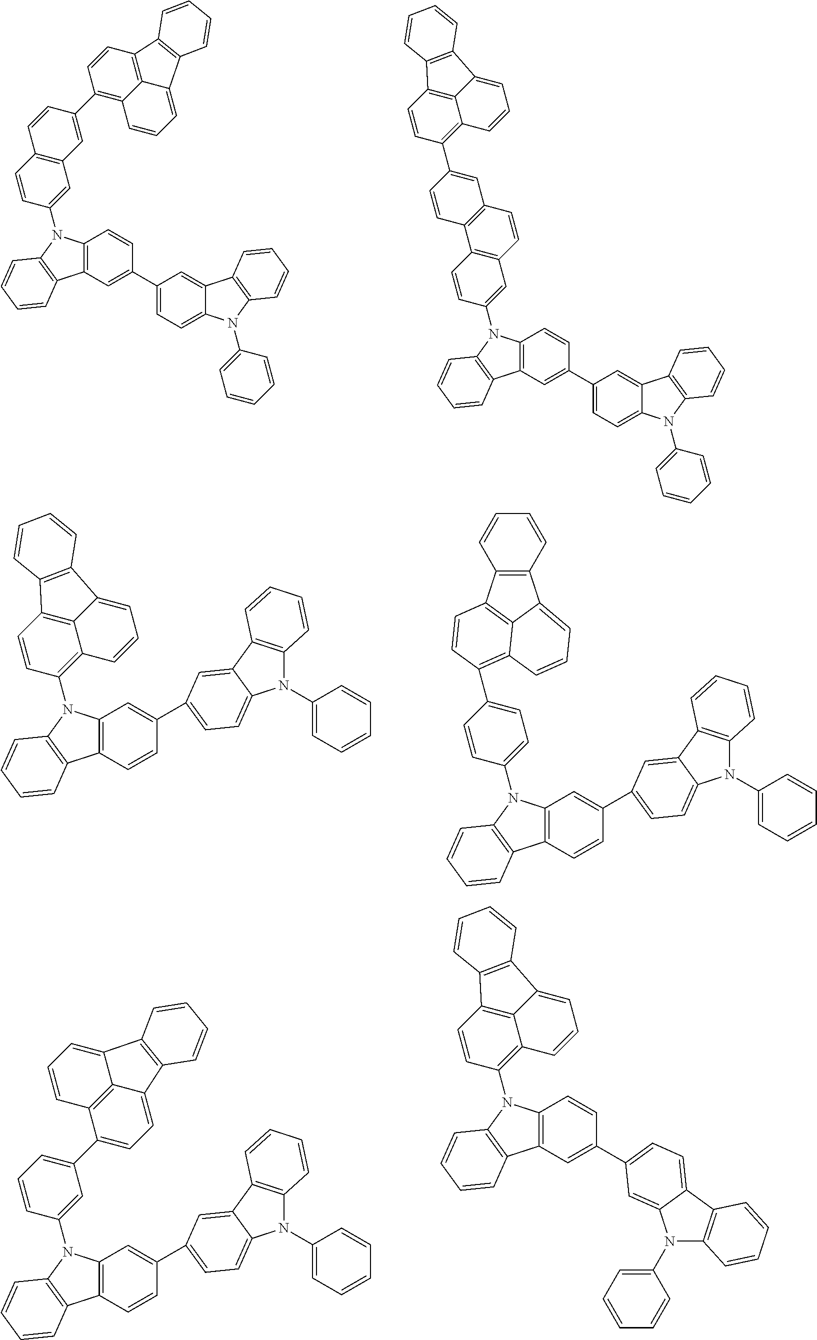

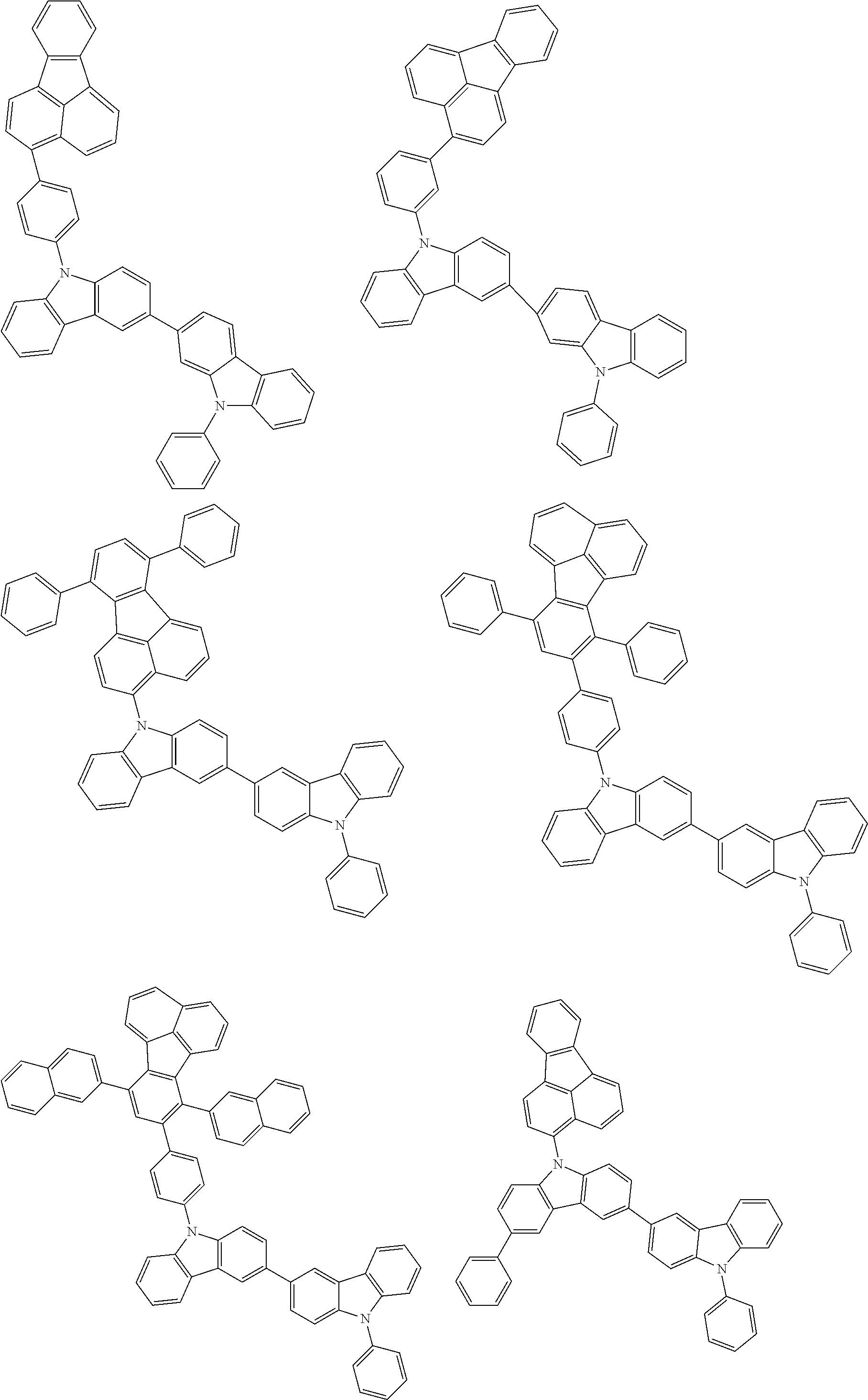

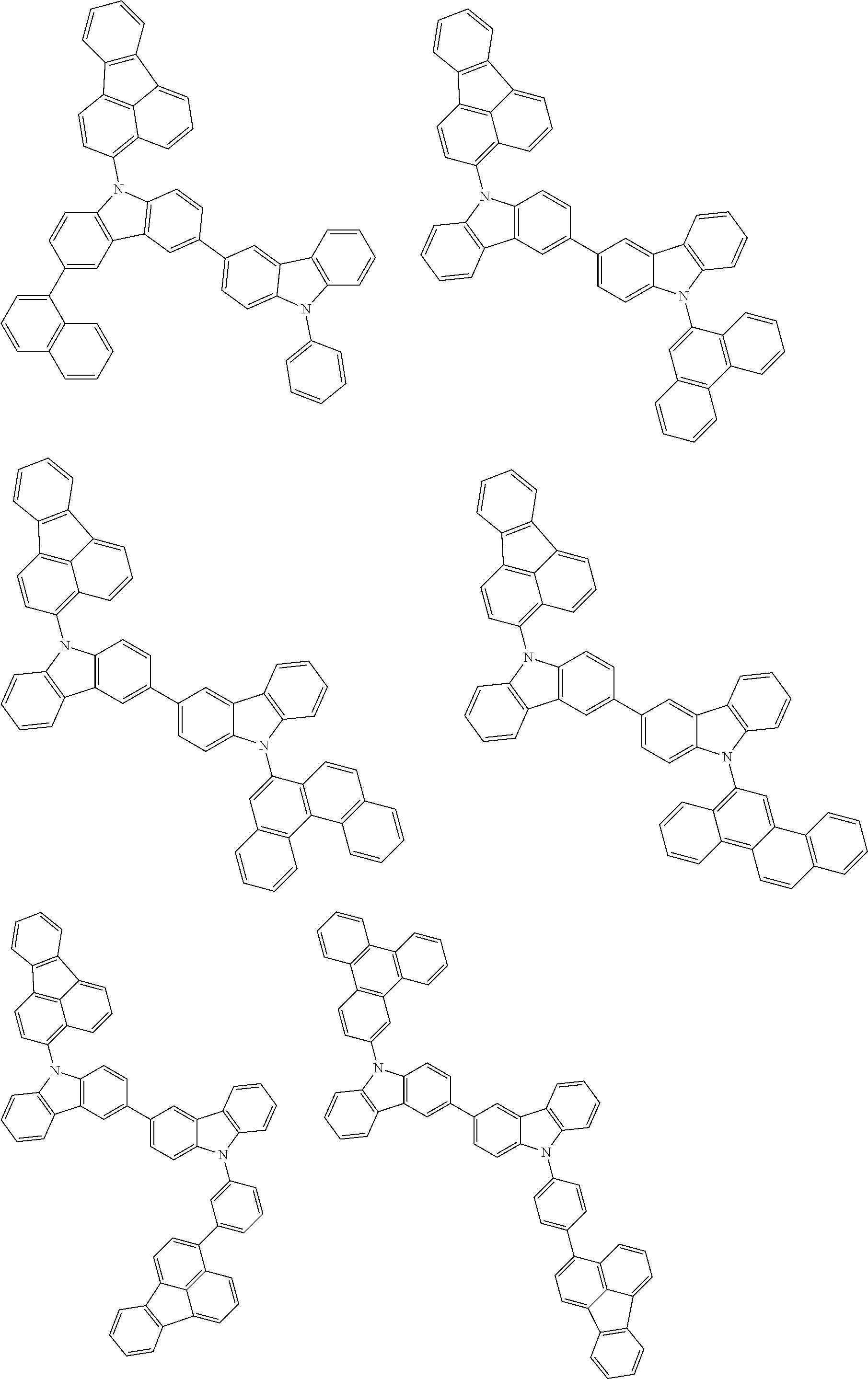

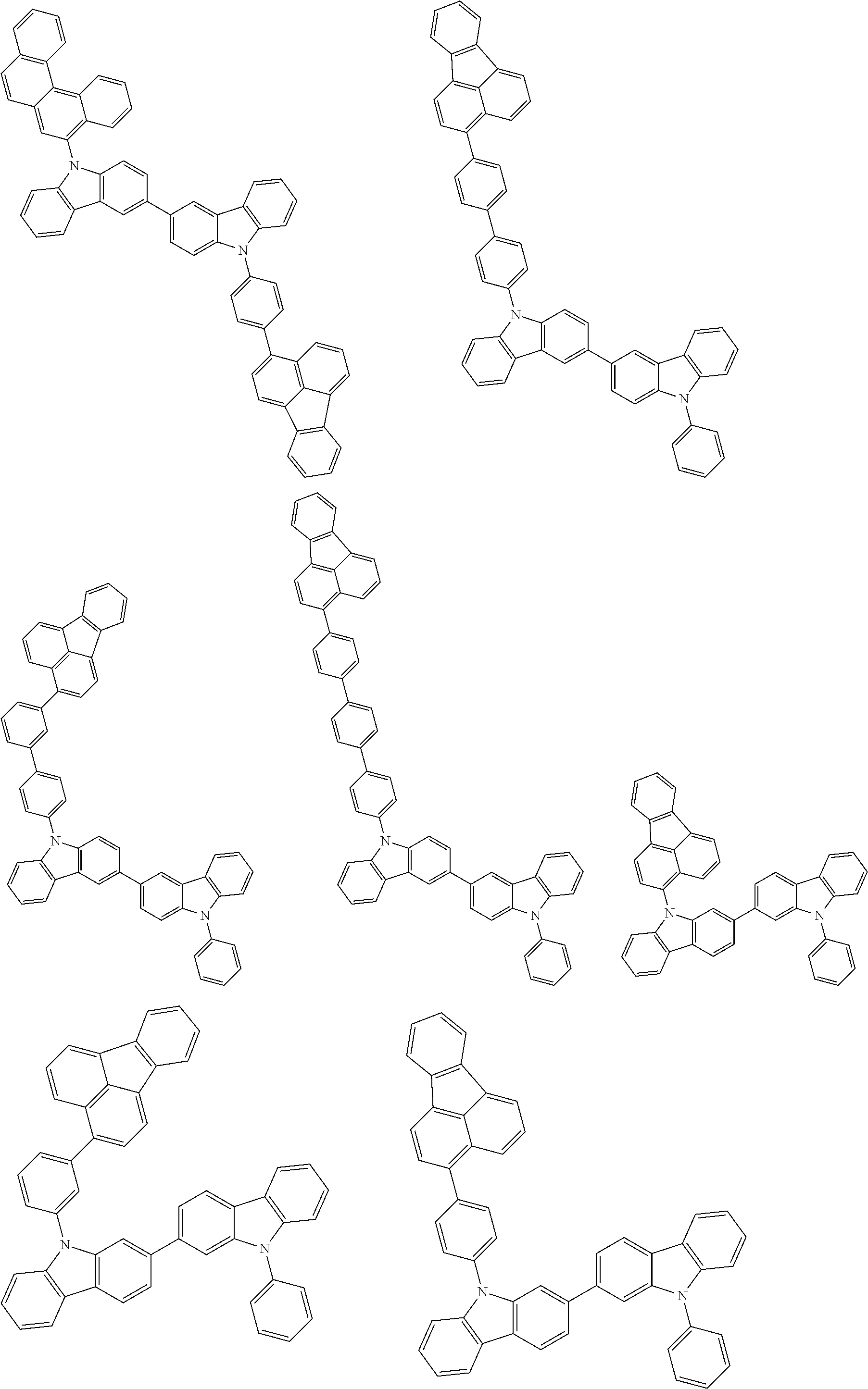

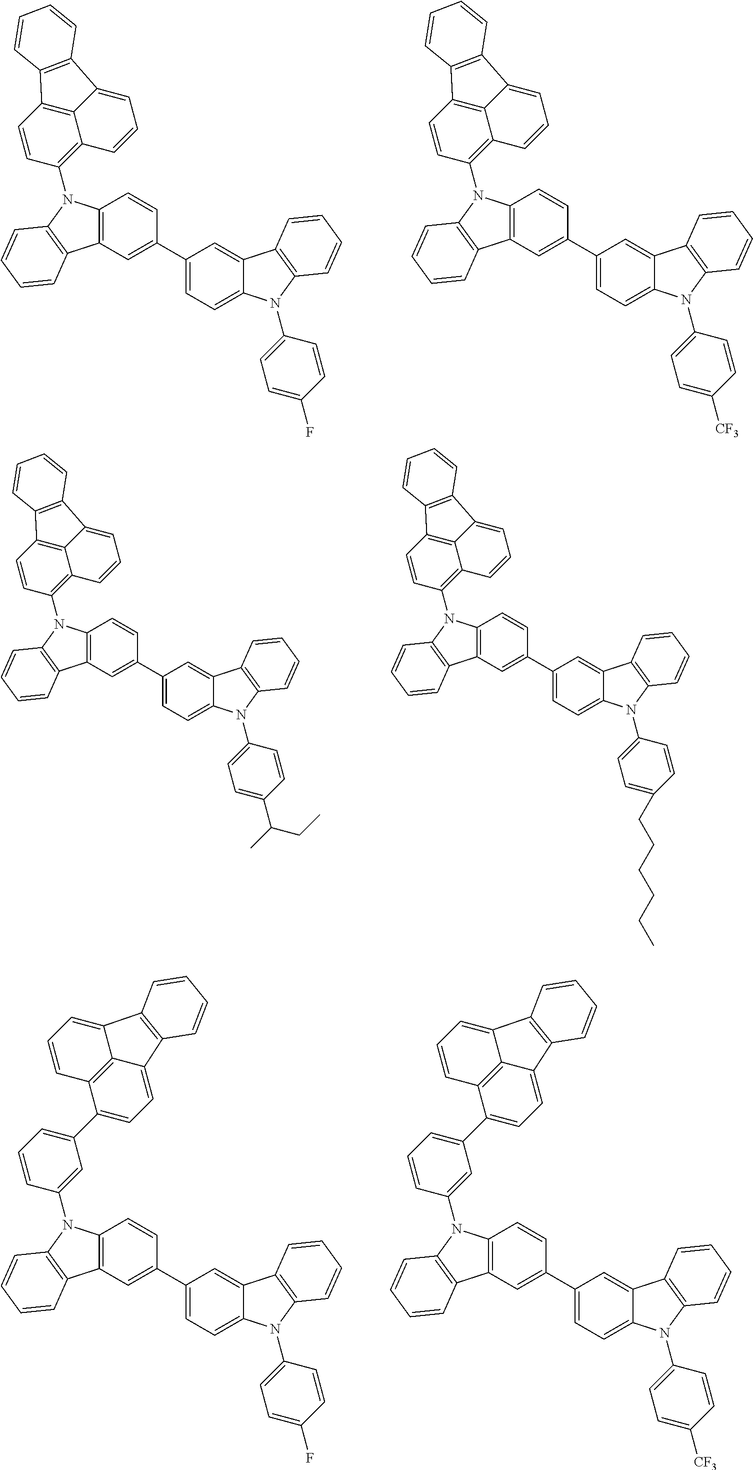

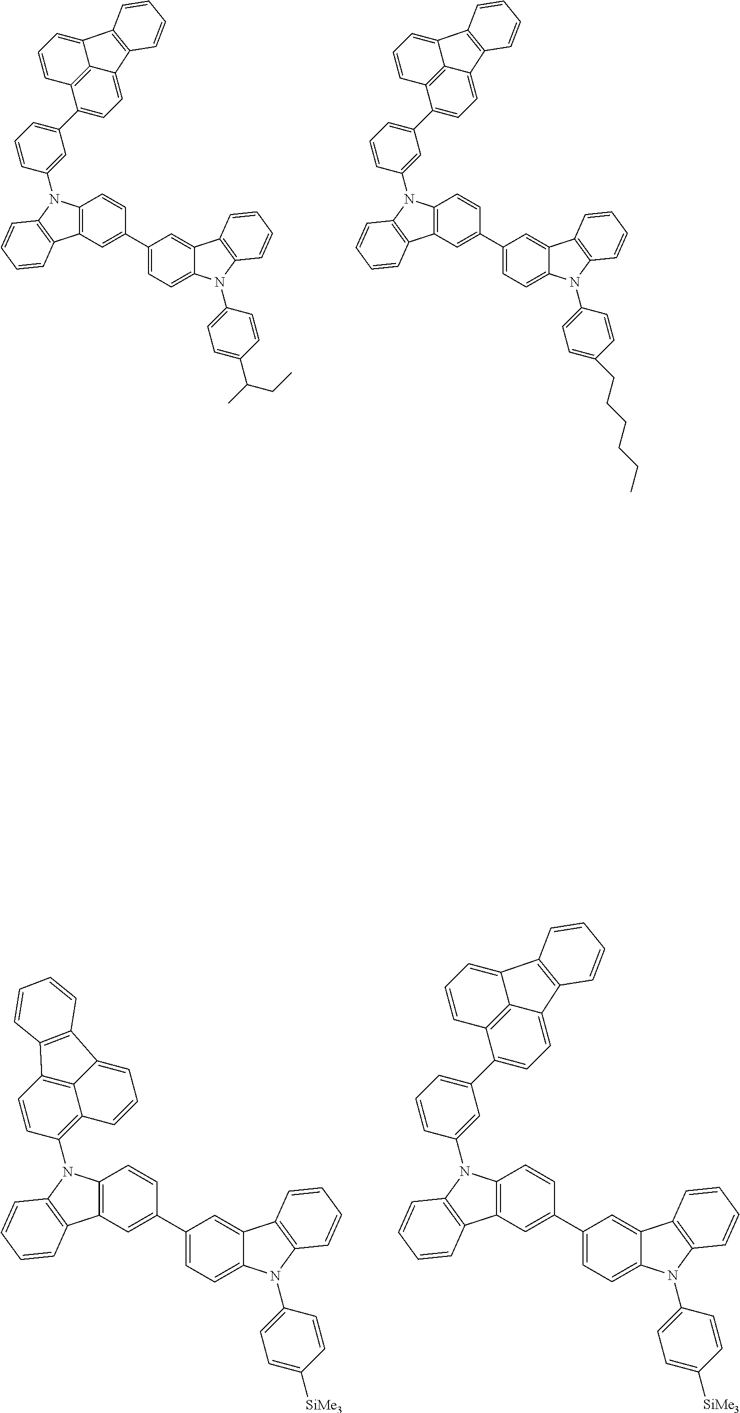 [SMILES: CCC(C)c1ccc(-n2c3ccccc3c3cc(-c4ccc5c(c4)c4ccccc4n5-c4cccc(-c5ccc6c7c(cccc57)-c5ccccc5-6)c4)ccc32)cc1 ; CCCCCCc1ccc(-n2c3ccccc3c3cc(-c4ccc5c(c4)c4ccccc4n5-c4cccc(-c5ccc6c7c(cccc57)-c5ccccc5-6)c4)ccc32)cc1 ; C[Si](C)(C)c1ccc(-n2c3ccccc3c3cc(-c4ccc5c(c4)c4ccccc4n5-c4ccc5c6c(cccc46)-c4ccccc4-5)ccc32)cc1 ; C[Si](C)(C)c1ccc(-n2c3ccccc3c3cc(-c4ccc5c(c4)c4ccccc4n5-c4cccc(-c5ccc6c7c(cccc57)-c5ccccc5-6)c4)ccc32)cc1]

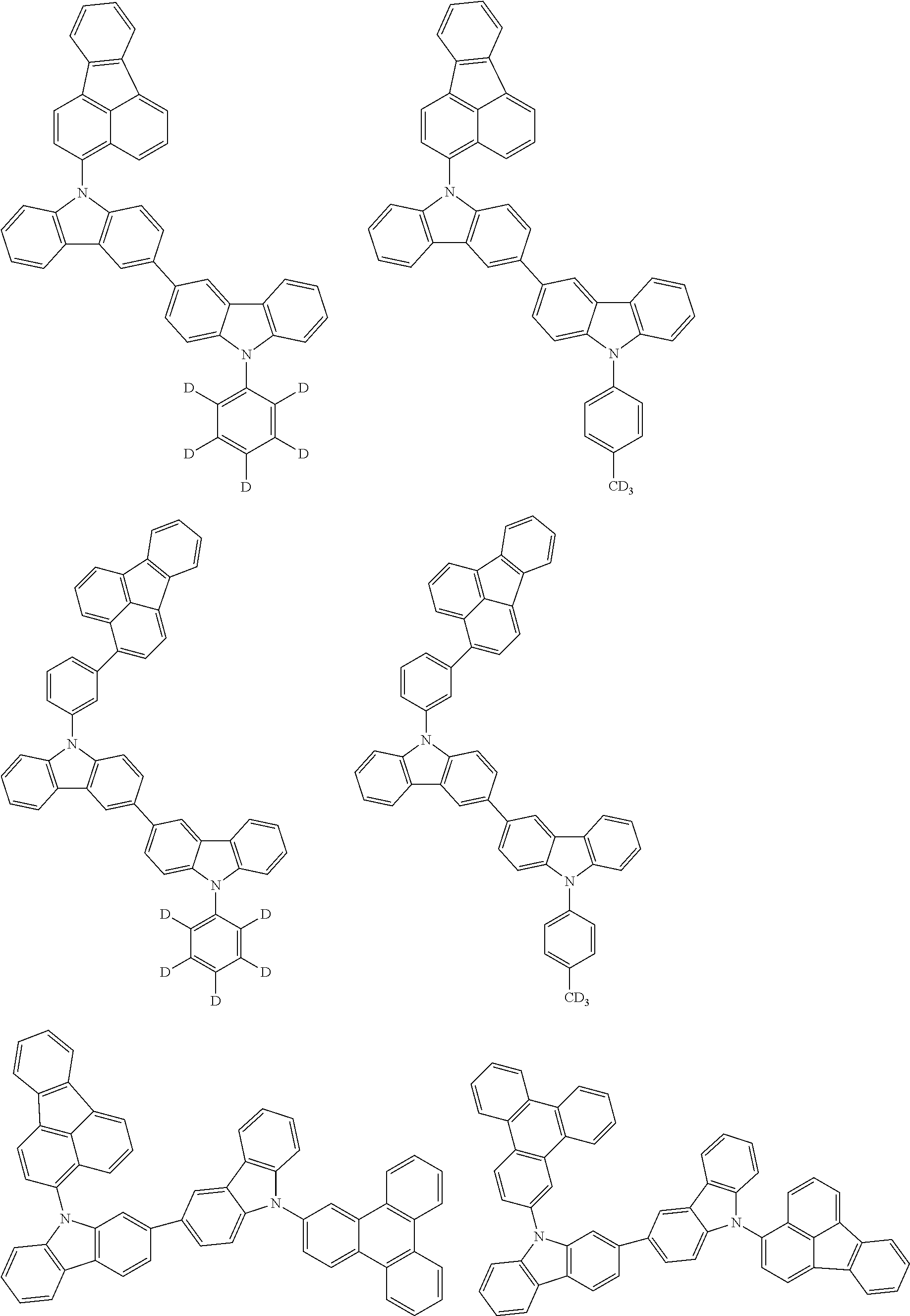

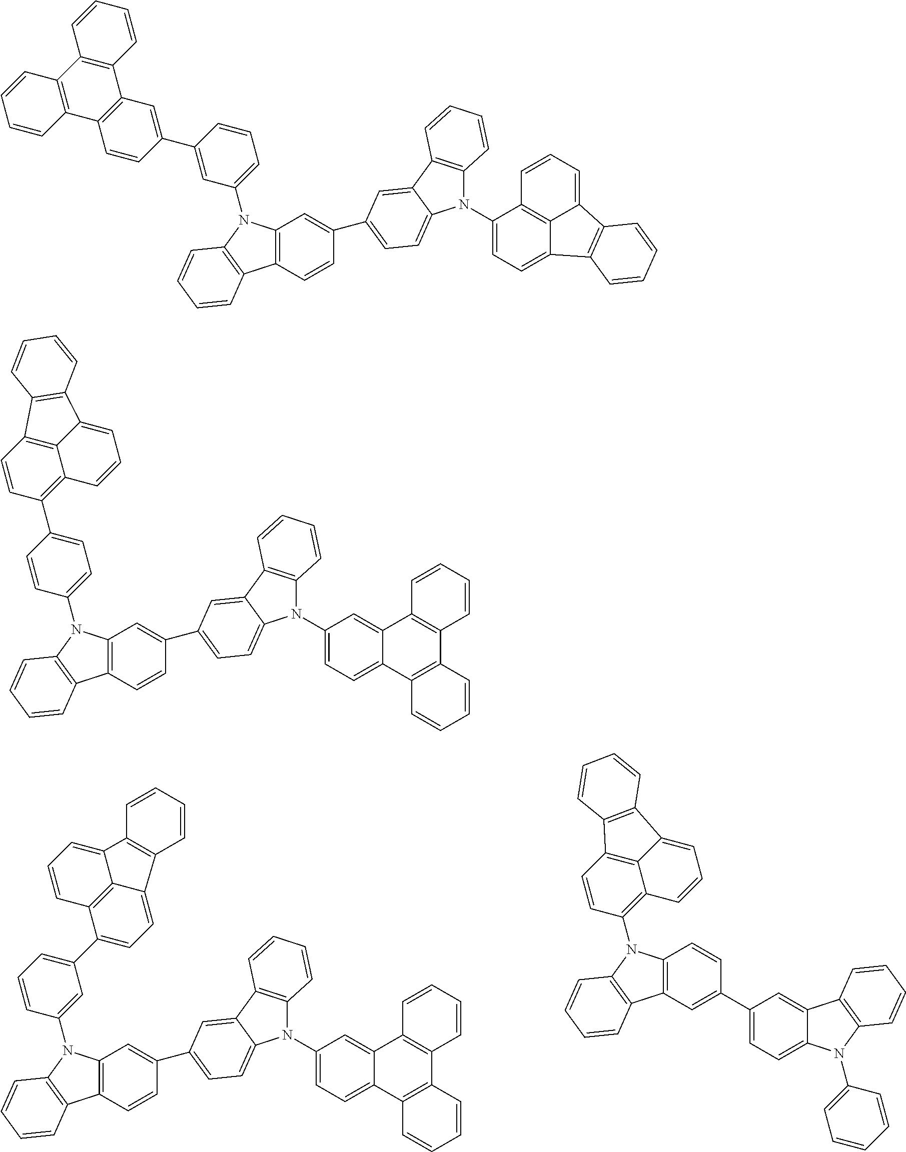 [SMILES: c1cc(-c2ccc3c4ccccc4c4ccccc4c3c2)cc(-n2c3ccccc3c3ccc(-c4ccc5c(c4)c4ccccc4n5-c4ccc5c6c(cccc46)-c4ccccc4-5)cc32)c1 ; c1ccc2c(c1)-c1cccc3c(-c4ccc(-n5c6ccccc6c6ccc(-c7ccc8c(c7)c7ccccc7n8-c7ccc8c9ccccc9c9ccccc9c8c7)cc65)cc4)ccc-2c13 ; c1cc(-c2ccc3c4c(cccc24)-c2ccccc2-3)cc(-n2c3ccccc3c3ccc(-c4ccc5c(c4)c4ccccc4n5-c4ccc5c6ccccc6c6ccccc6c5c4)cc32)c1 ; c1ccc(-n2c3ccccc3c3cc(-c4ccc5c(c4)c4ccccc4n5-c4ccc5c6c(cccc46)-c4ccccc4-5)ccc32)cc1]

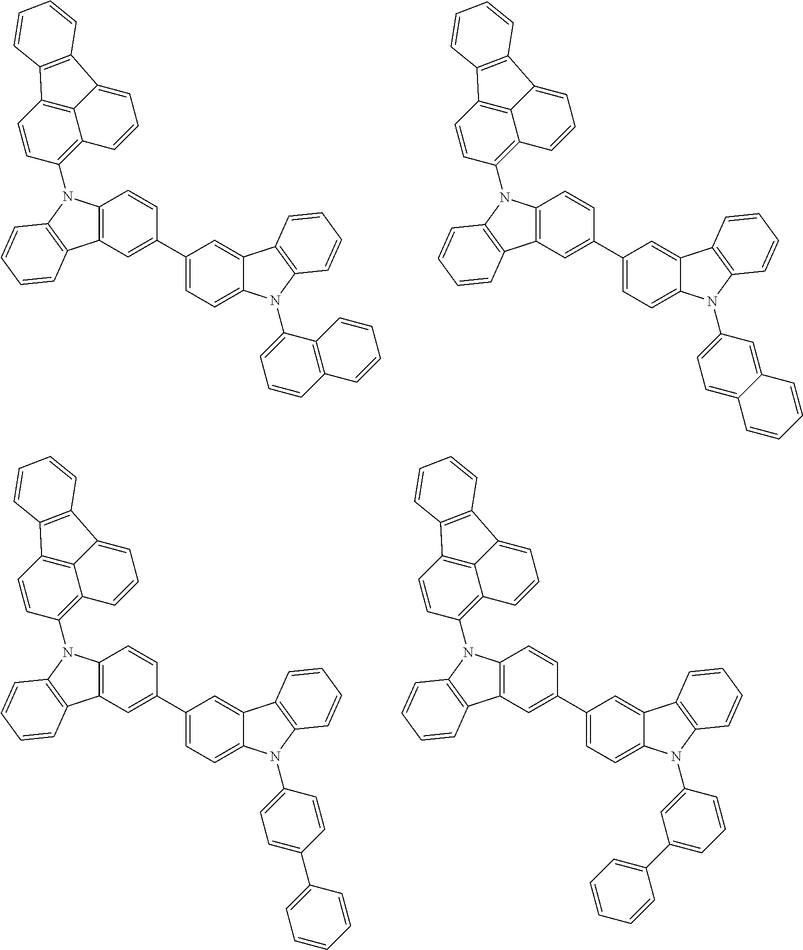 [SMILES: c1ccc2c(c1)-c1cccc3c(-n4c5ccccc5c5cc(-c6ccc7c(c6)c6ccccc6n7-c6cccc7ccccc67)ccc54)ccc-2c13 ; c1ccc2c(c1)-c1cccc3c(-n4c5ccccc5c5cc(-c6ccc7c(c6)c6ccccc6n7-c6ccc7ccccc7c6)ccc54)ccc-2c13 ; c1ccc(-c2ccc(-n3c4ccccc4c4cc(-c5ccc6c(c5)c5ccccc5n6-c5ccc6c7c(cccc57)-c5ccccc5-6)ccc43)cc2)cc1 ; c1ccc(-c2cccc(-n3c4ccccc4c4cc(-c5ccc6c(c5)c5ccccc5n6-c5ccc6c7c(cccc57)-c5ccccc5-6)ccc43)c2)cc1]

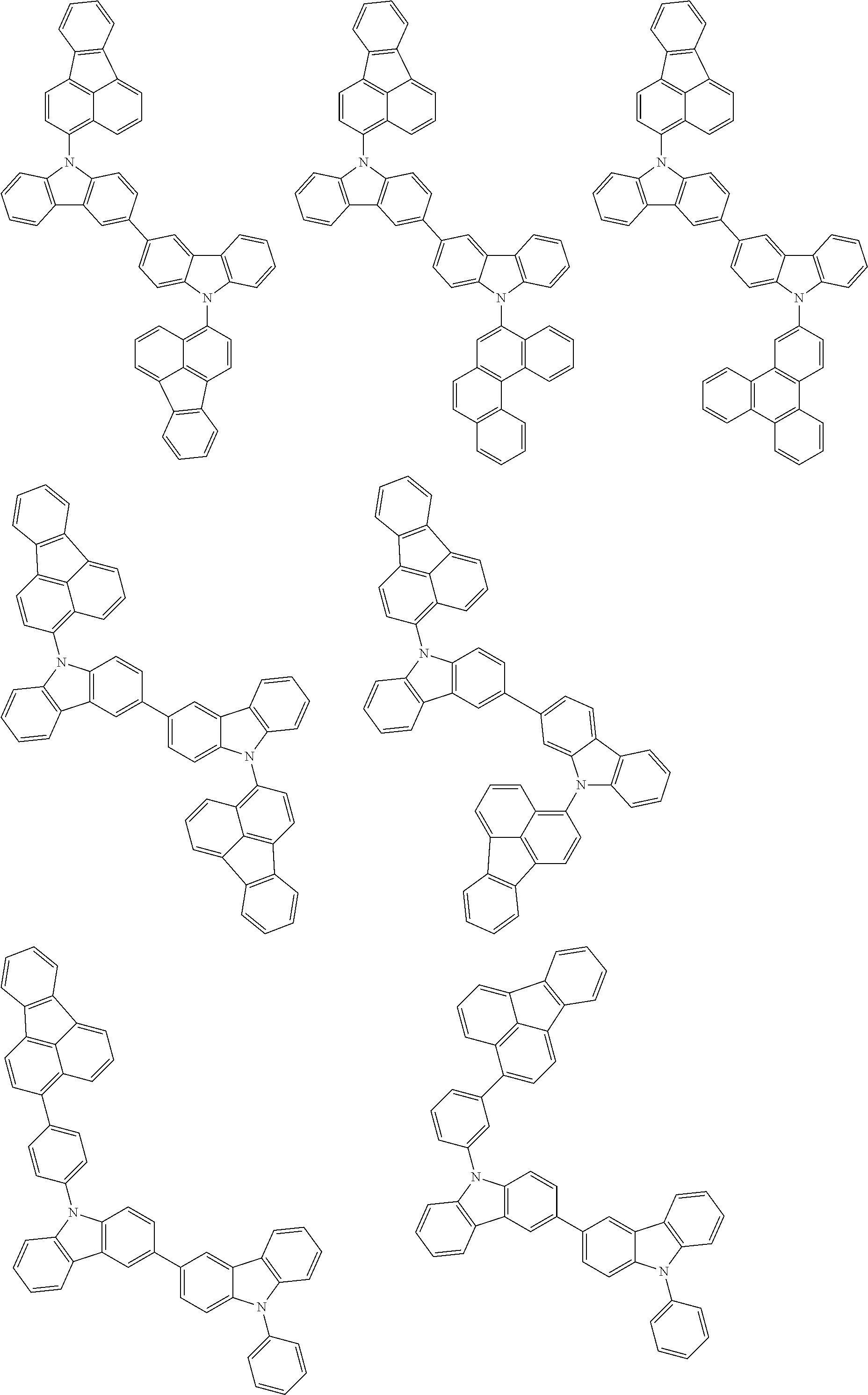

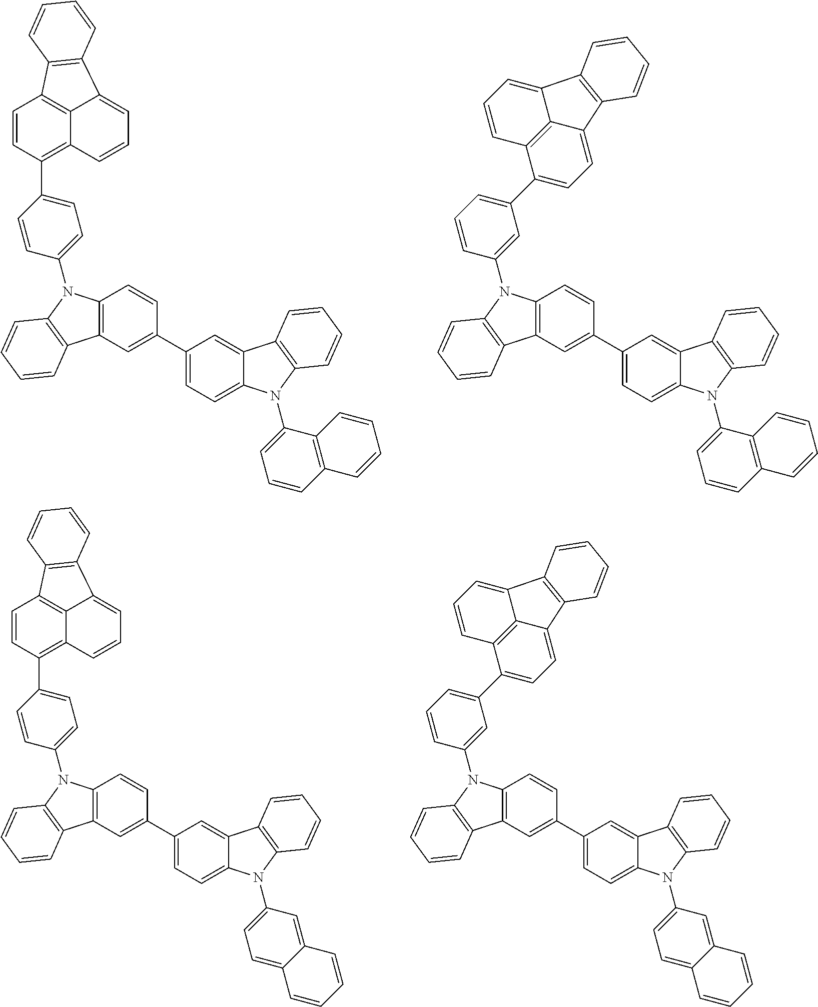 [SMILES: c1ccc2c(c1)-c1cccc3c(-c4ccc(-n5c6ccccc6c6cc(-c7ccc8c(c7)c7ccccc7n8-c7cccc8ccccc78)ccc65)cc4)ccc-2c13 ; c1cc(-c2ccc3c4c(cccc24)-c2ccccc2-3)cc(-n2c3ccccc3c3cc(-c4ccc5c(c4)c4ccccc4n5-c4cccc5ccccc45)ccc32)c1 ; c1ccc2c(c1)-c1cccc3c(-c4ccc(-n5c6ccccc6c6cc(-c7ccc8c(c7)c7ccccc7n8-c7ccc8ccccc8c7)ccc65)cc4)ccc-2c13 ; c1cc(-c2ccc3c4c(cccc24)-c2ccccc2-3)cc(-n2c3ccccc3c3cc(-c4ccc5c(c4)c4ccccc4n5-c4ccc5ccccc5c4)ccc32)c1]

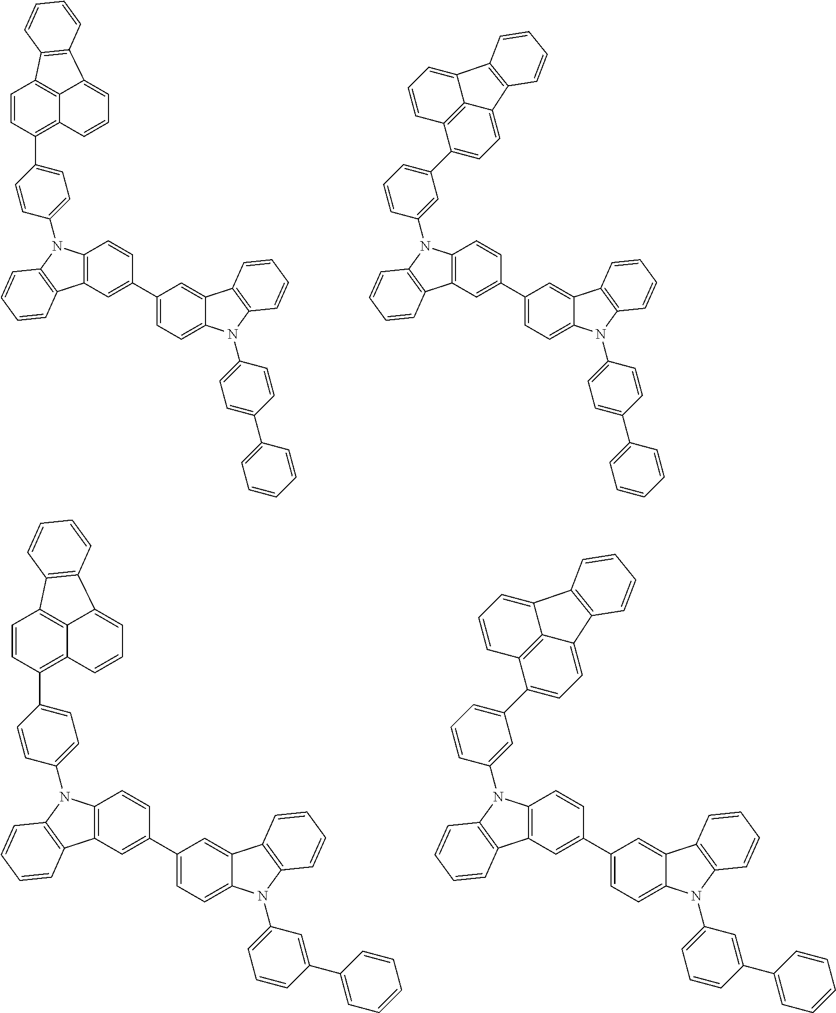 [SMILES: c1ccc(-c2ccc(-n3c4ccccc4c4cc(-c5ccc6c(c5)c5ccccc5n6-c5ccc(-c6ccc7c8c(cccc68)-c6ccccc6-7)cc5)ccc43)cc2)cc1 ; c1ccc(-c2ccc(-n3c4ccccc4c4cc(-c5ccc6c(c5)c5ccccc5n6-c5cccc(-c6ccc7c8c(cccc68)-c6ccccc6-7)c5)ccc43)cc2)cc1 ; c1ccc(-c2cccc(-n3c4ccccc4c4cc(-c5ccc6c(c5)c5ccccc5n6-c5ccc(-c6ccc7c8c(cccc68)-c6ccccc6-7)cc5)ccc43)c2)cc1 ; c1ccc(-c2cccc(-n3c4ccccc4c4cc(-c5ccc6c(c5)c5ccccc5n6-c5cccc(-c6ccc7c8c(cccc68)-c6ccccc6-7)c5)ccc43)c2)cc1]

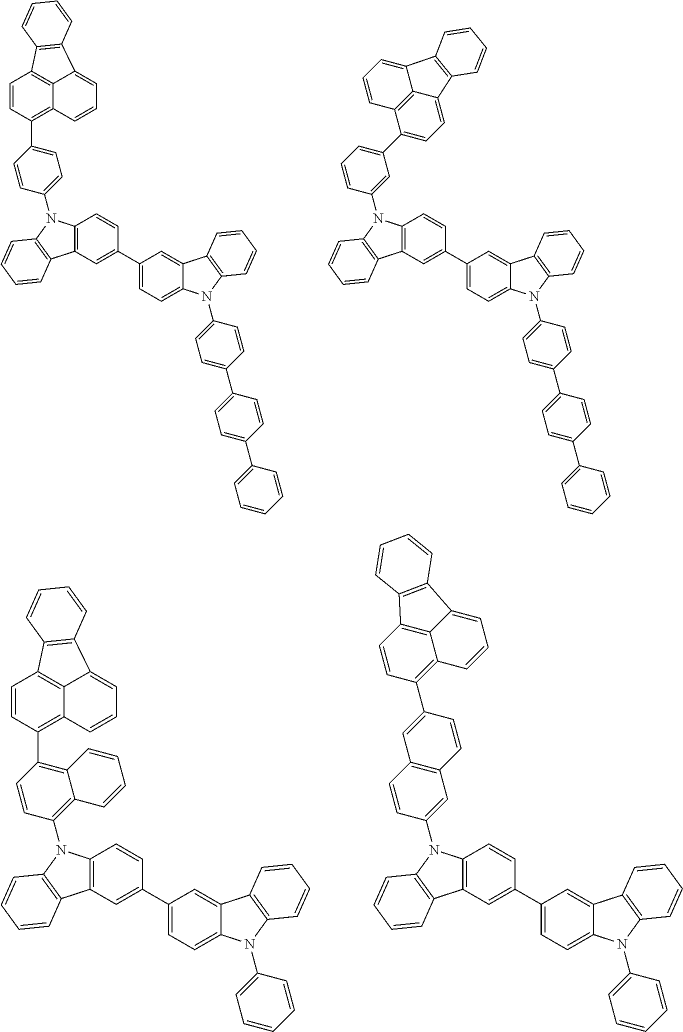 [SMILES: c1ccc(-c2ccc(-c3ccc(-n4c5ccccc5c5cc(-c6ccc7c(c6)c6ccccc6n7-c6ccc(-c7ccc8c9c(cccc79)-c7ccccc7-8)cc6)ccc54)cc3)cc2)cc1 ; c1ccc(-c2ccc(-c3ccc(-n4c5ccccc5c5cc(-c6ccc7c(c6)c6ccccc6n7-c6cccc(-c7ccc8c9c(cccc79)-c7ccccc7-8)c6)ccc54)cc3)cc2)cc1 ; c1ccc(-n2c3ccccc3c3cc(-c4ccc5c(c4)c4ccccc4n5-c4ccc(-c5ccc6c7c(cccc57)-c5ccccc5-6)c5ccccc45)ccc32)cc1 ; c1ccc(-n2c3ccccc3c3cc(-c4ccc5c(c4)c4ccccc4n5-c4ccc5cc(-c6ccc7c8c(cccc68)-c6ccccc6-7)ccc5c4)ccc32)cc1]

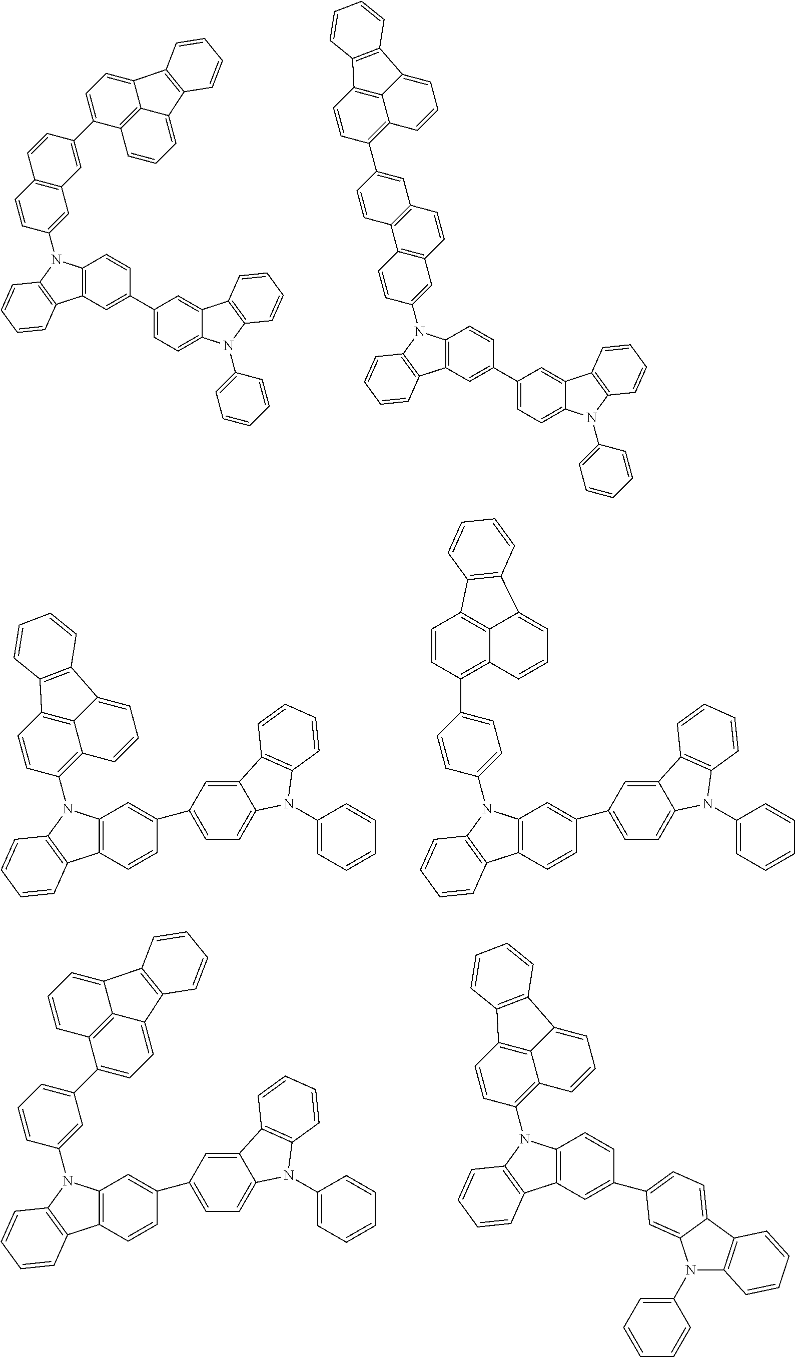

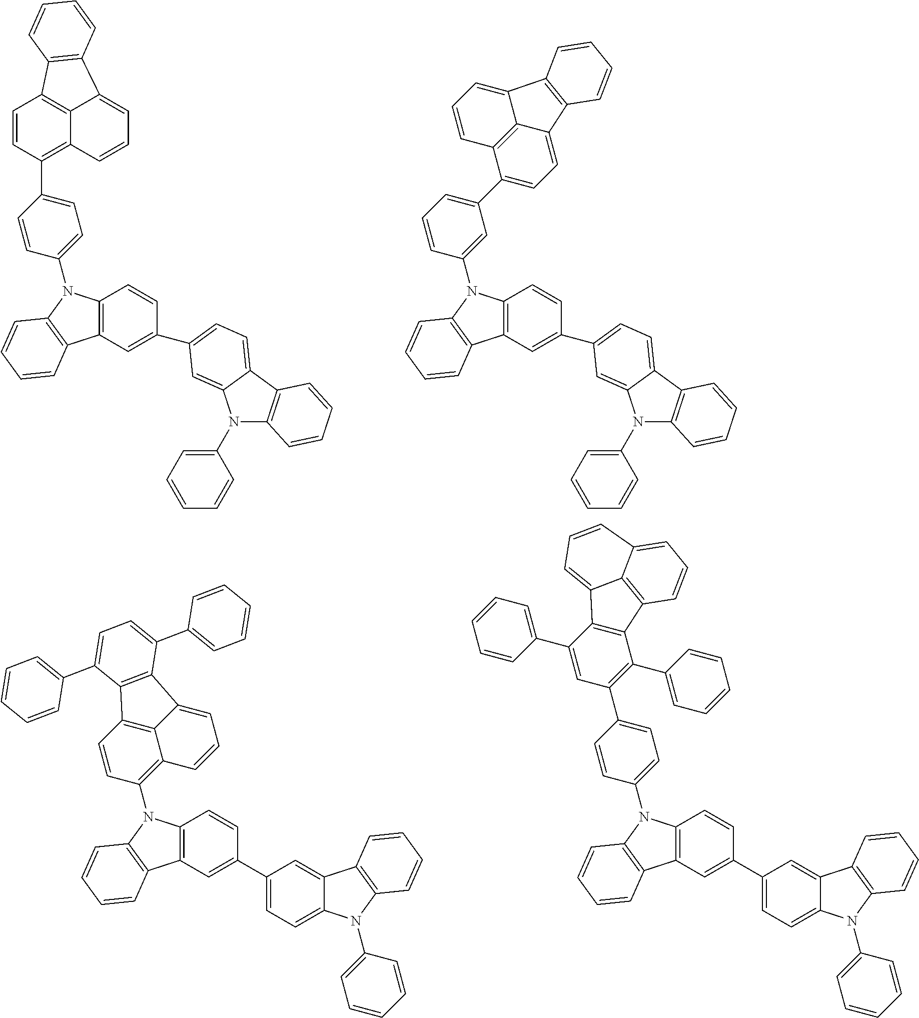

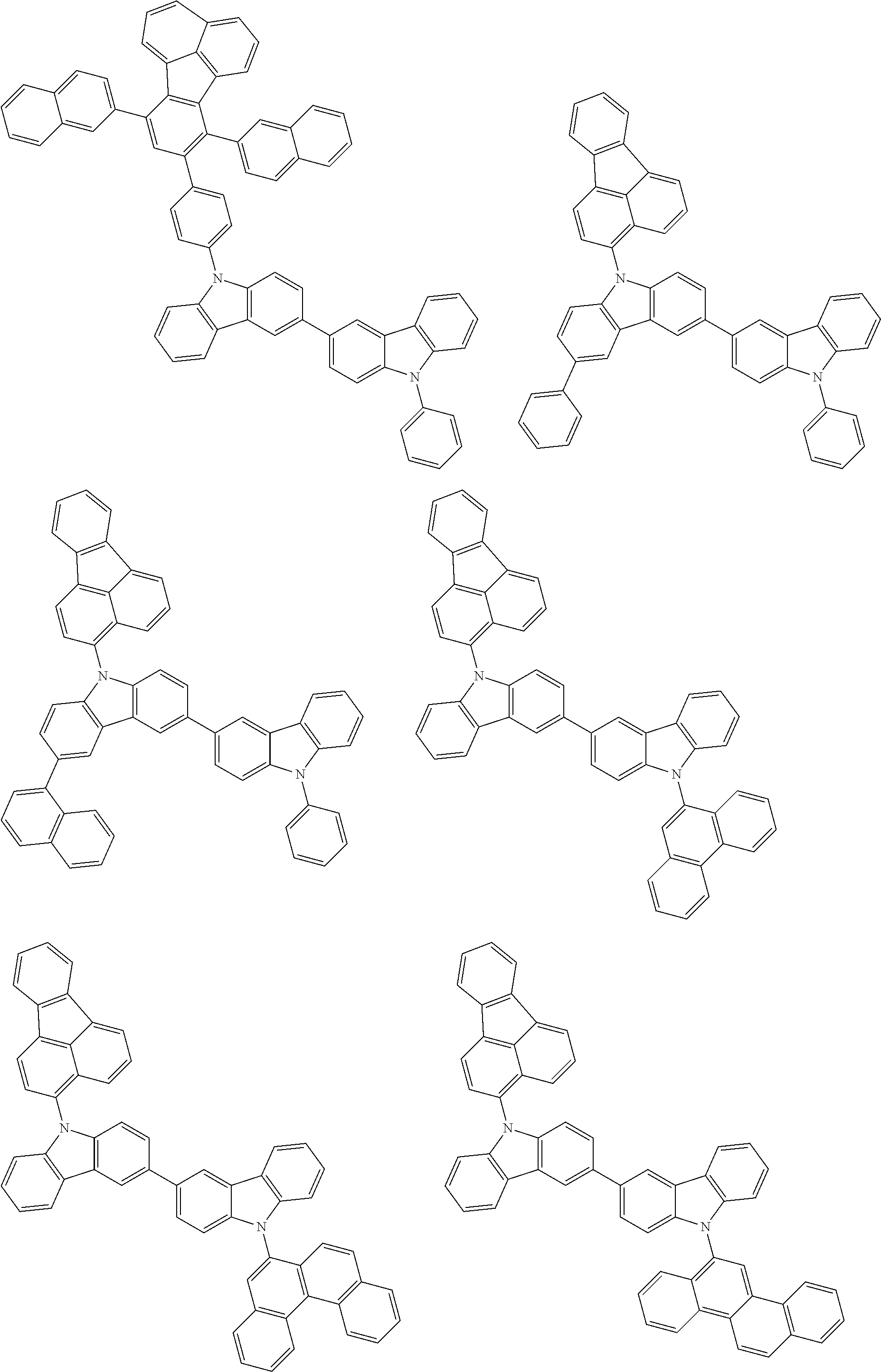

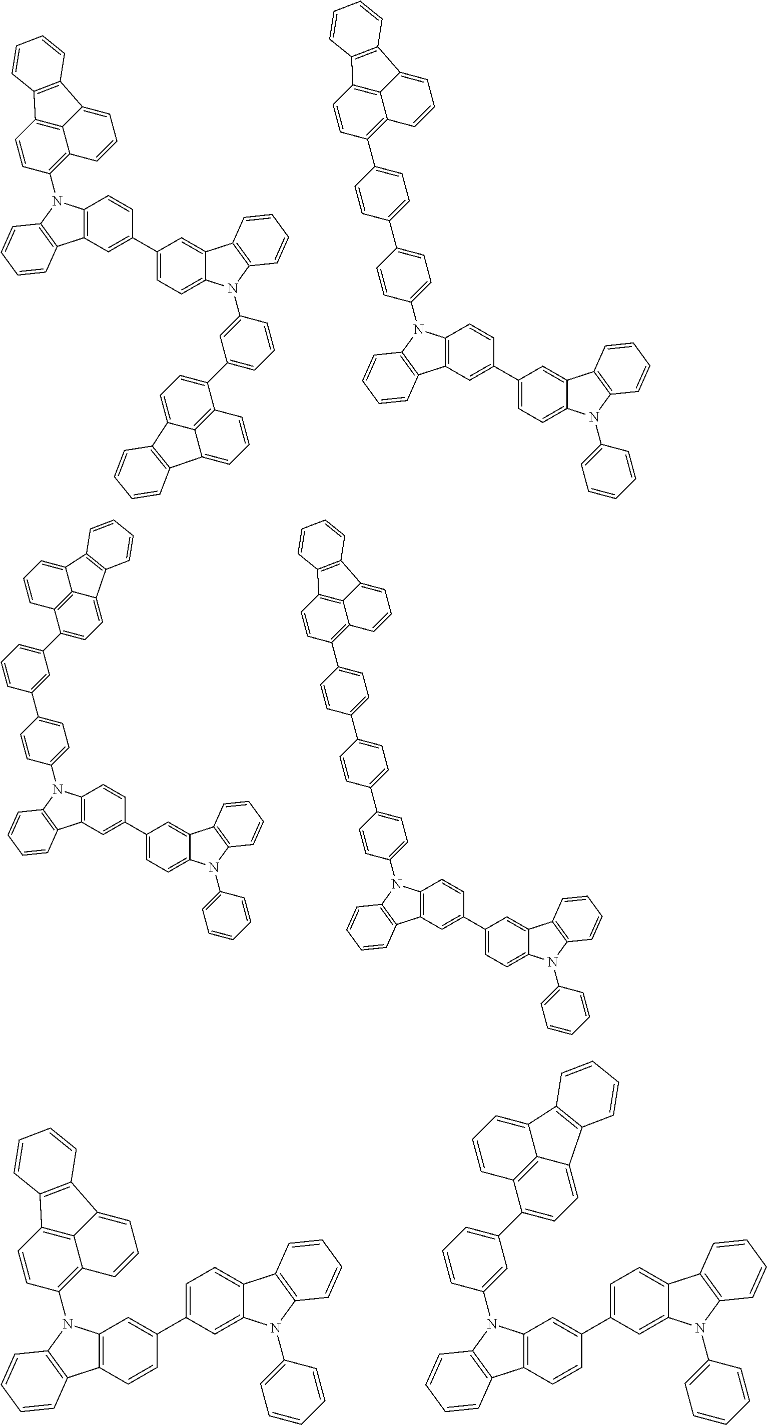

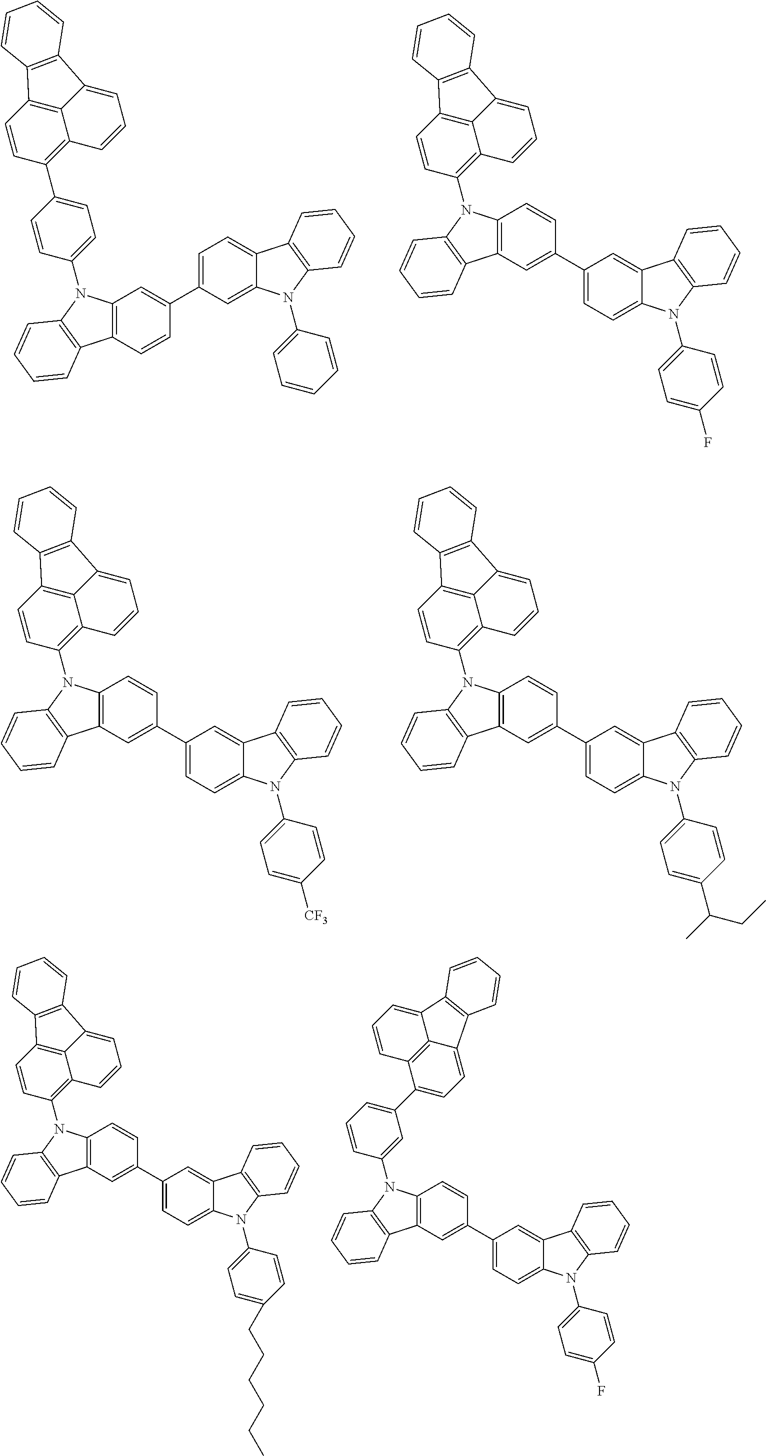

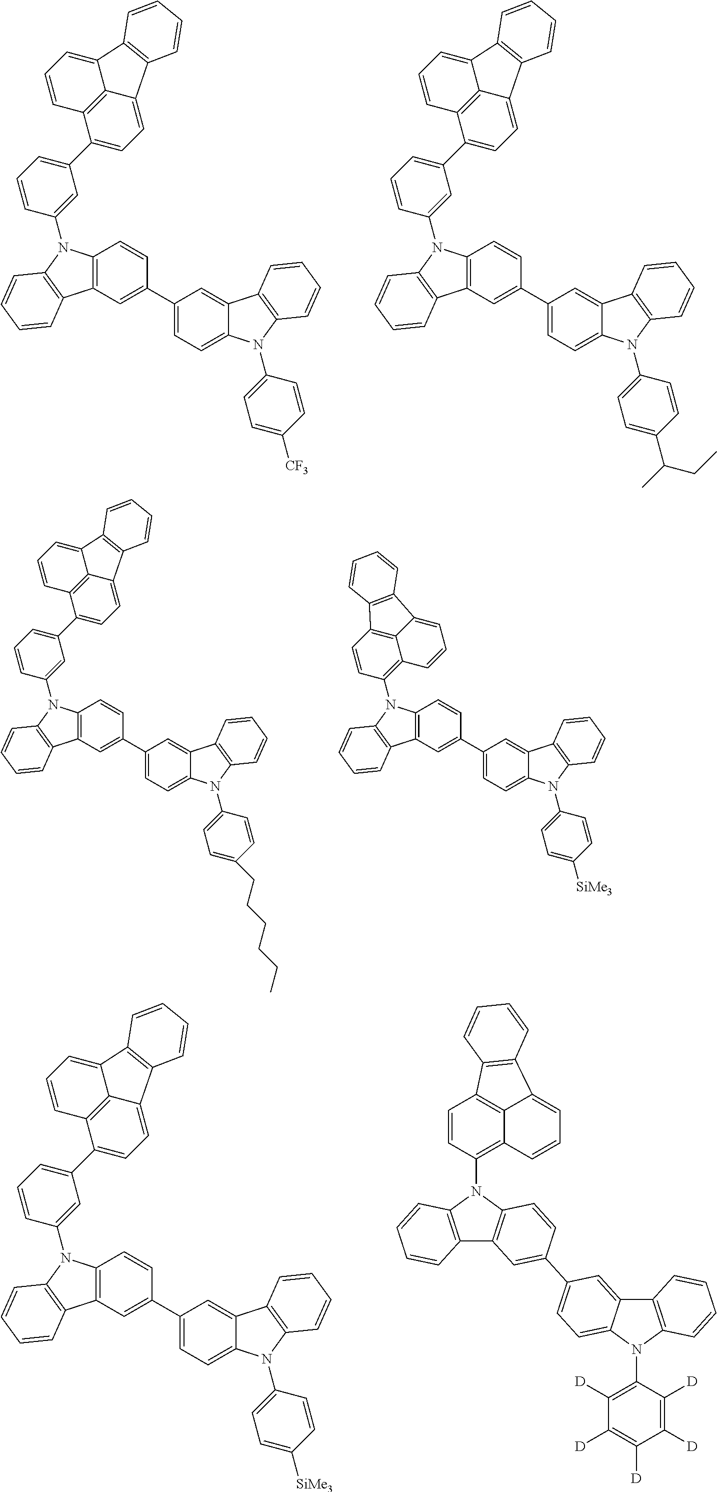

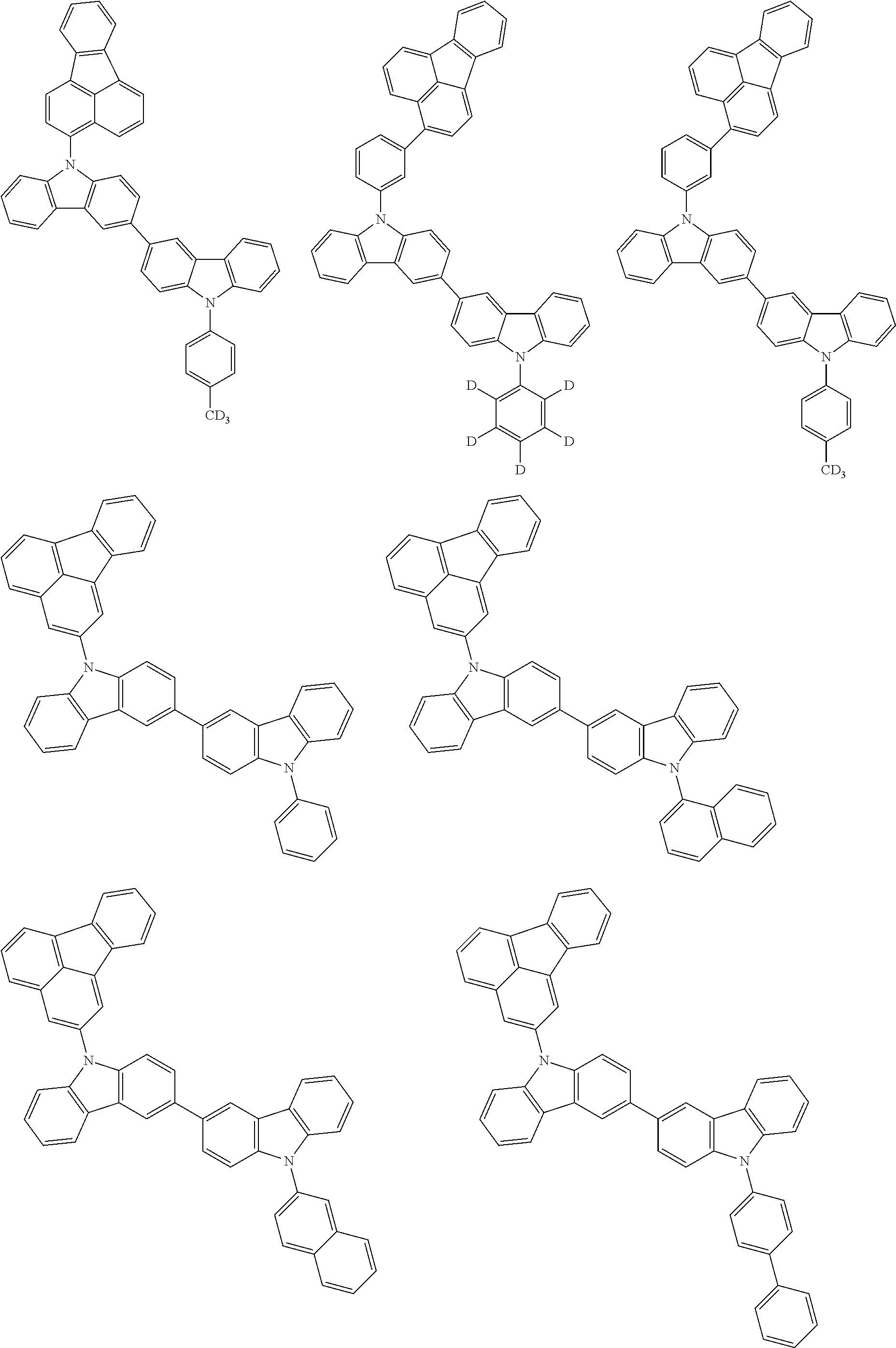

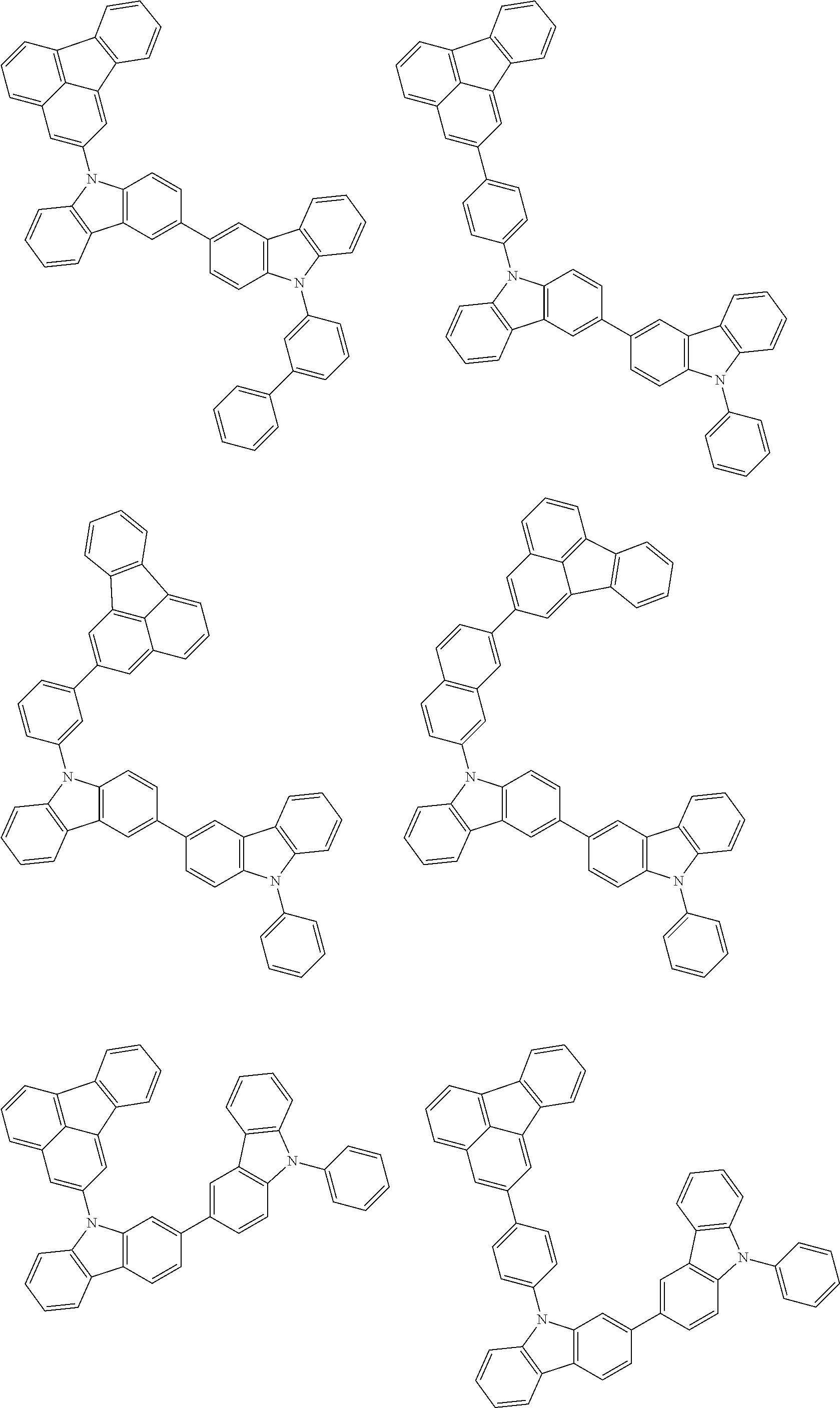

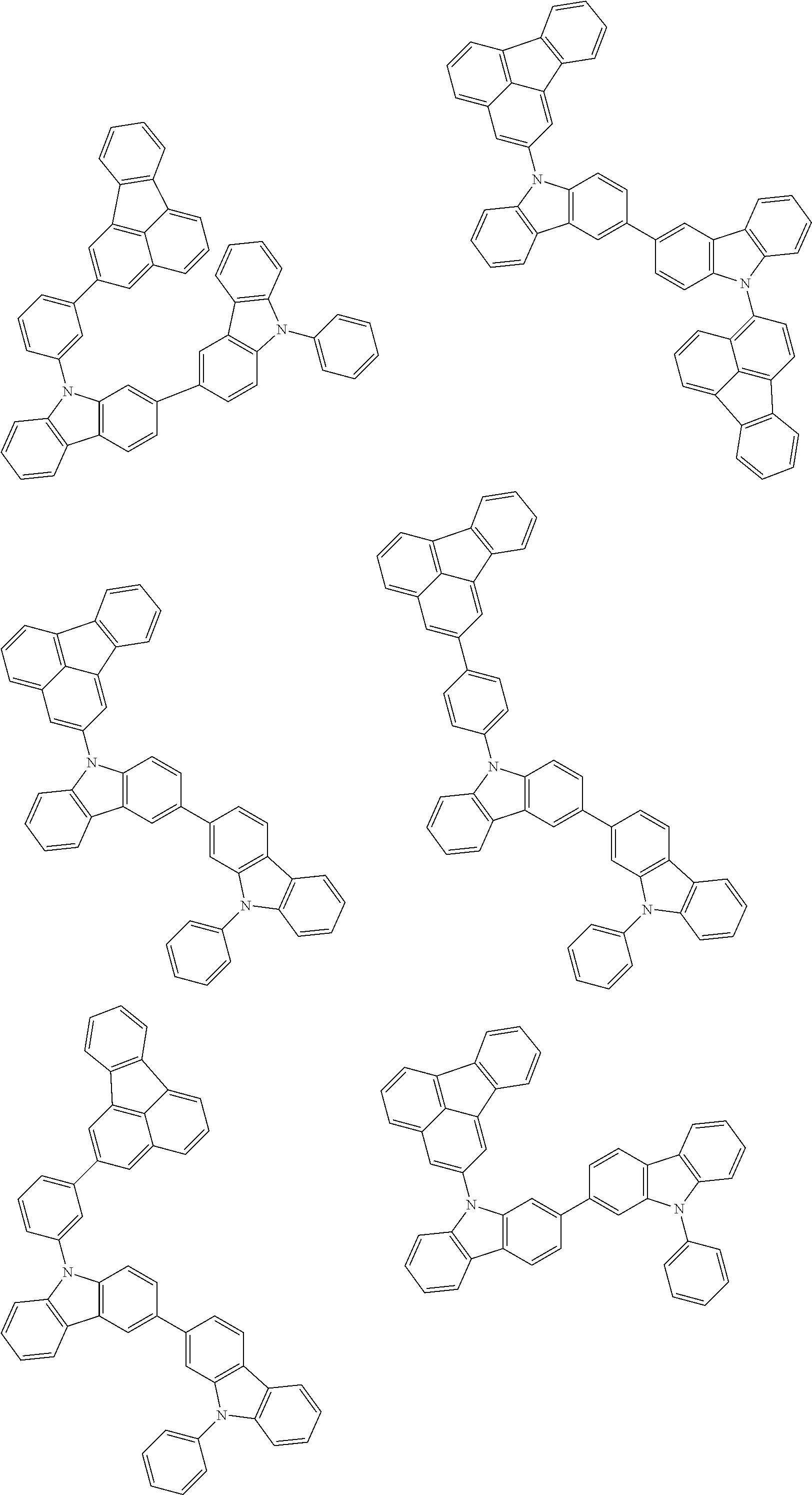

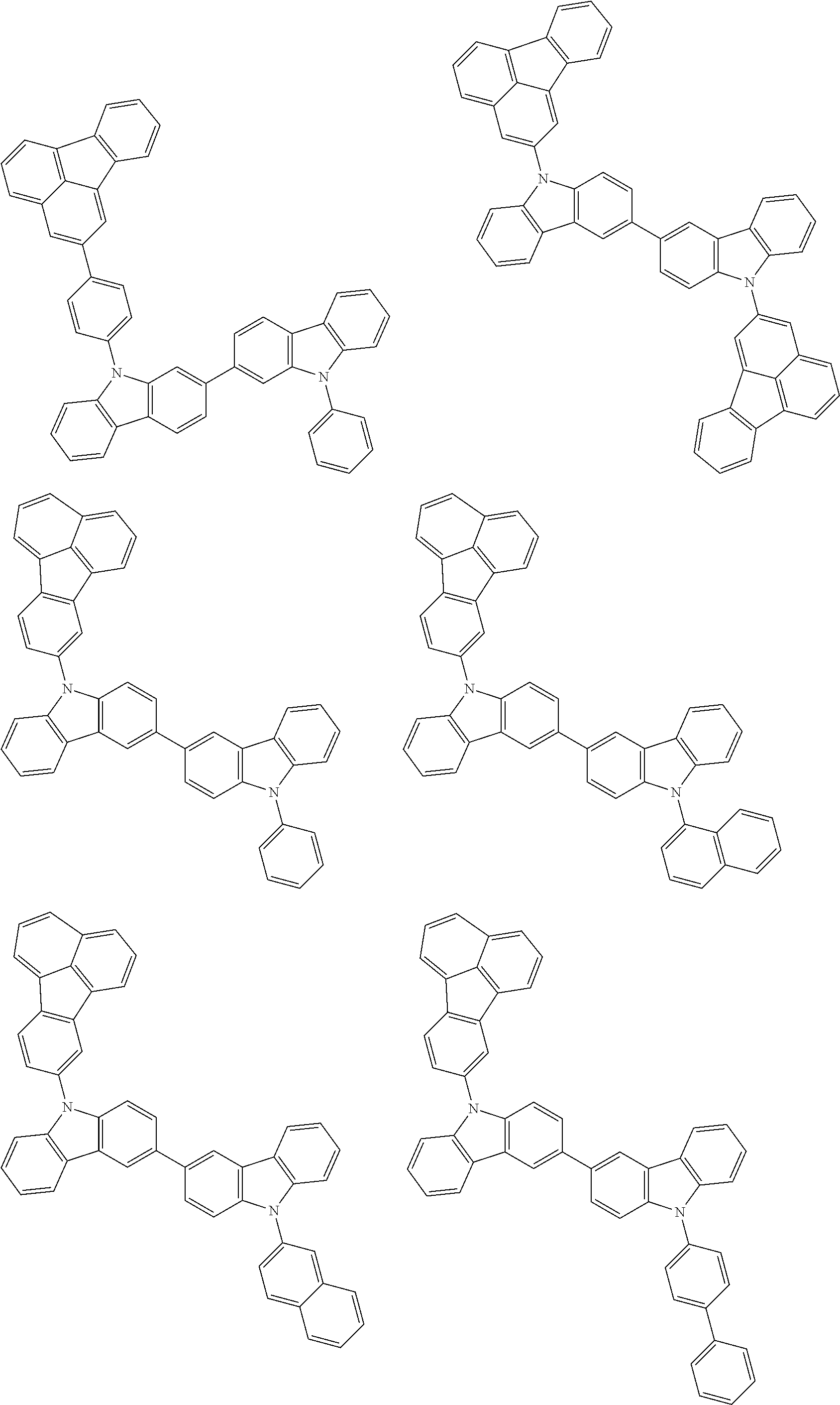

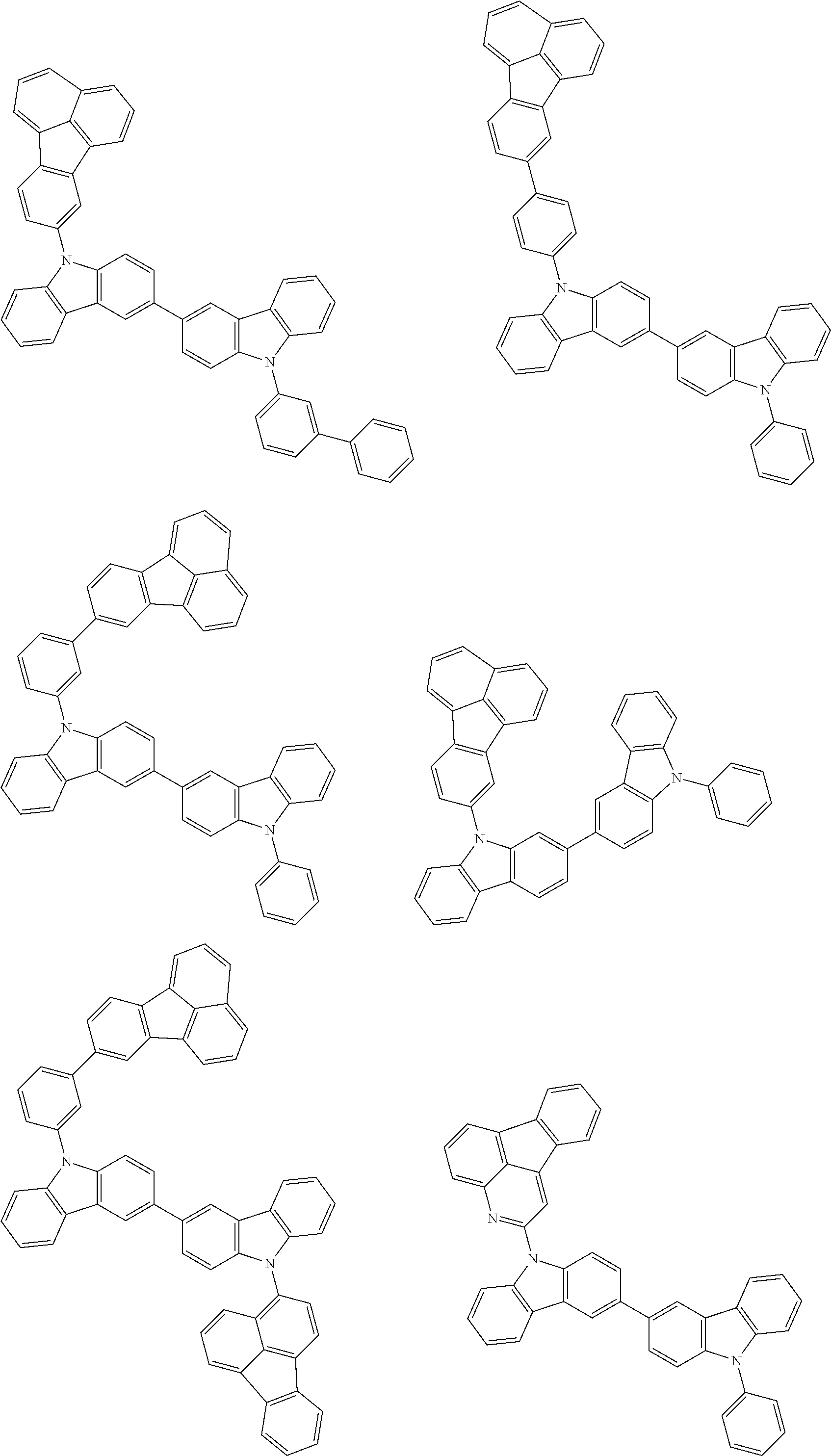

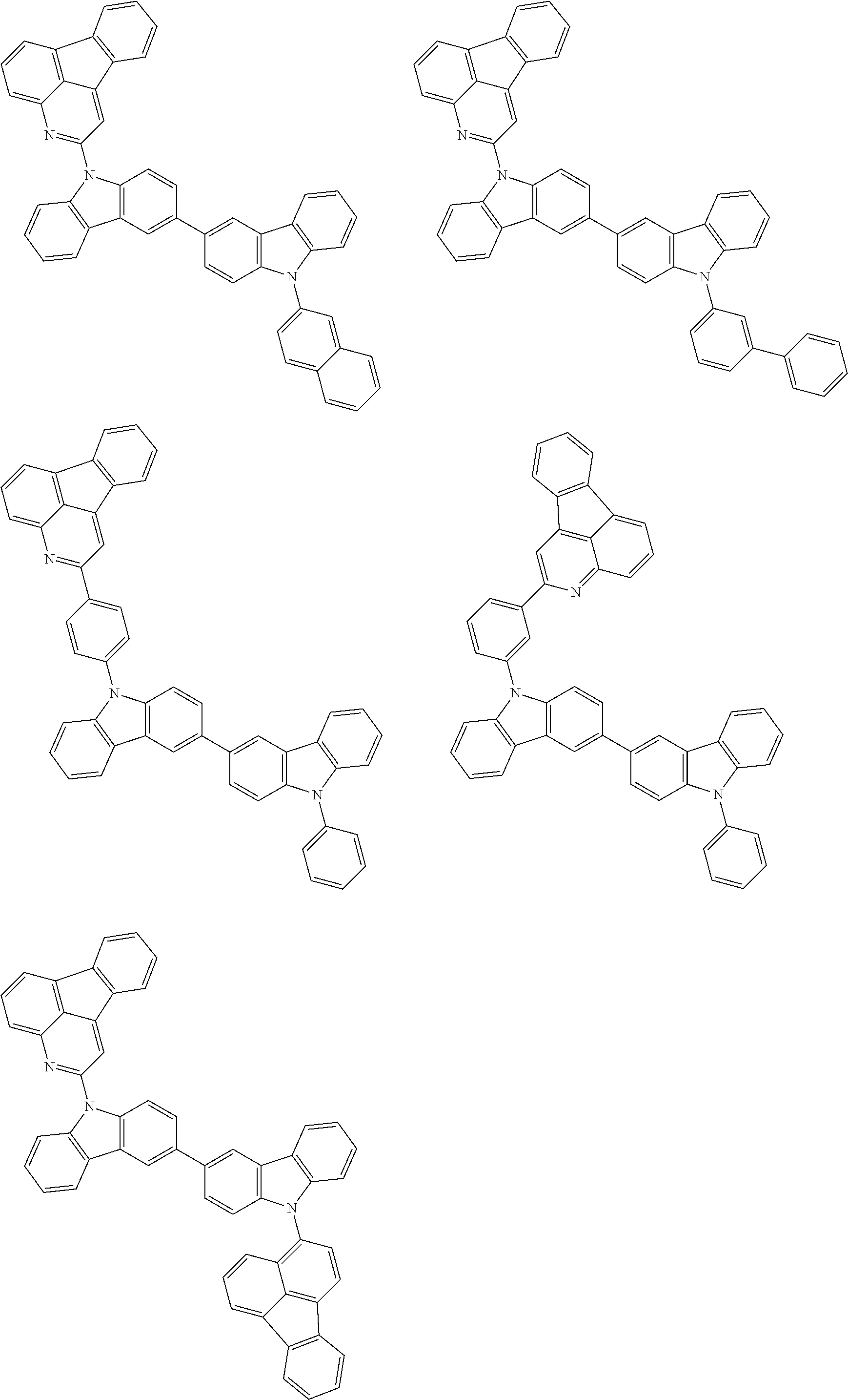 [SMILES: c1ccc2c(c1)-c1cccc3nc(-n4c5ccccc5c5cc(-c6ccc7c(c6)c6ccccc6n7-c6ccc7ccccc7c6)ccc54)cc-2c13 ; c1ccc(-c2cccc(-n3c4ccccc4c4cc(-c5ccc6c(c5)c5ccccc5n6-c5cc6c7c(cccc7n5)-c5ccccc5-6)ccc43)c2)cc1 ; c1ccc(-n2c3ccccc3c3cc(-c4ccc5c(c4)c4ccccc4n5-c4ccc(-c5cc6c7c(cccc7n5)-c5ccccc5-6)cc4)ccc32)cc1 ; c1ccc(-n2c3ccccc3c3cc(-c4ccc5c(c4)c4ccccc4n5-c4cccc(-c5cc6c7c(cccc7n5)-c5ccccc5-6)c4)ccc32)cc1 ; c1ccc2c(c1)-c1cccc3nc(-n4c5ccccc5c5cc(-c6ccc7c(c6)c6ccccc6n7-c6ccc7c8c(cccc68)-c6ccccc6-7)ccc54)cc-2c13]

Ar₁₁ to Ar₁₃ in formula (11) are preferably a substituted or unsubstituted aryl group including 5 to 50 ring carbon atoms. Ar₁₁ to Ar₁₃ in formula (11) are more preferably a substituted or unsubstituted aryl group including 6 to 50 ring carbon atoms.

It is preferable that at least one of Ar₁₁ to Ar₁₃ in formula (11) is a substituted aryl group including 5 to 50 ring carbon atoms, and at least one of the remaining is an aryl group including 5 to 50 ring carbon atoms substituted with a substituted or unsubstituted dibenzofuranyl group or a substituted or unsubstituted dibenzothiophenyl group. It is more preferable that at least one of Ar₁₁ to Ar₁₃ in formula (11) is a substituted aryl group including 6 to 50 ring carbon atoms, and at least one of the remaining is an aryl group including 6 to 50 ring carbon atoms substituted with a substituted or unsubstituted dibenzofuranyl group or a substituted or unsubstituted dibenzothiophenyl group.

The compound represented by the formula (11) is preferably a compound represented by the following formula (12) or (13),

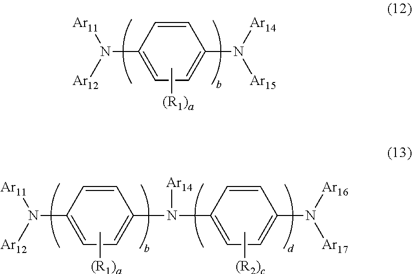

wherein Ar₁₁, Ar₁₂ and Ar₁₄ to Ar₁₇ are independently the same as those described above as Ar₁₁ to Ar₁₃, respectively.

R₁ and R₂ are independently a hydrogen atom, a substituted or unsubstituted aryl group including 5 to 50 (preferably 6 to 50) ring carbon atoms, a substituted or unsubstituted dibenzofuranyl group, a substituted or unsubstituted dibenzothiophenyl group, a substituted or unsubstituted alkyl group including 1 to 50 carbon atoms, a substituted or unsubstituted alkoxy group including 1 to 50 carbon atoms, a substituted or unsubstituted aralkyl group including 6 to 50 (preferably 7 to 50) carbon atoms, a substituted or unsubstituted aryloxy group including 5 to 50 (preferably 6 to 50) ring carbon atoms, a substituted or unsubstituted arylthio group including 5 to 50 (preferably 6 to 50) ring carbon atoms, a substituted or unsubstituted alkoxycarbonyl group including 2 to 50 carbon atoms, an amino group substituted with a substituted or unsubstituted aryl group including 5 to 50 (preferably 6 to 50) ring carbon atoms, an amino group substituted with a substituted or unsubstituted dibenzofuranyl group or a substituted or unsubstituted dibenzothiophenyl group, a halogen atom, a cyano group, a nitro group, a hydroxyl group, or a carboxyl group.

a and c are independently an integer of 0 to 4 (preferably 0 or 1, more preferably 0), and b and d are independently an integer of 1 to 3 (preferably 1 or 2),

plural R₁s may be bonded with each other to form a saturated or unsaturated, 5-membered or 6-membered ring structure which may have a substituent, and

plural R₂s may be bonded with each other to form a saturated or unsaturated, 5-membered or 6-membered ring structure which may have a substituent.

The compound represented by the formula (11) may be a compound represented by the following formula (14).

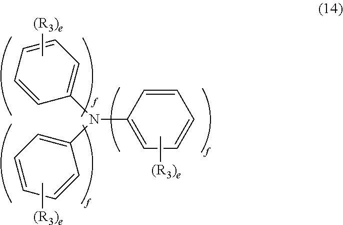

In the formula (14), R₃ is independently the same as those described above as R₁ and R₂, respectively.

e is independently an integer of 0 to 4 (preferably 0, 1 or 2, more preferably 0 or 1), and f is independently an integer of 1 to 3.

Adjacent Rₐs may be bonded with each other to form a saturated or unsaturated, 5-membered or 6-membered ring structure which may have a substituent.

Examples of the aryl group including 5 to 50 (preferably 6 to 50, more preferably 5 to 18, particularly preferably 6 to 18) ring carbon atoms in Ar₁₁ to Ar₁₇ and R₁ to R₃ include a phenyl group, a 1-naphthyl group, a 2-naphthyl group, a 1-anthryl group, a 2-anthryl group, a 9-anthryl group, a 1-phenanthryl group, a 2-phenanthryl group, a 3-phenanthryl group, a 4-phenanthryl group, a 9-phenanthryl group, a 1-naphthacenyl group, a 2-naphthacenyl group, a 9-naphthacenyl group, a 1-pyrenyl group, a 2-pyrenyl group, a 4-pyrenyl group, a 2-biphenylyl group, a 3-biphenylyl group, a 4-biphenylyl group, a p-terphenyl-4-yl group, a p-terphenyl-3-yl group, a p-terphenyl-2-yl group, a m-terphenyl-4-yl group, a m-terphenyl-3-yl group, a m-terphenyl-2-yl group, an o-tolyl group, a m-tolyl group, a p-tolyl group, a p-t-butylphenyl group, a p-(2-phenylpropyl) phenyl group, a 3-methyl-2-naphthyl group, a 4-methyl-1-naphthyl group, a 4-methyl-1-anthryl group, a 4′-methylbiphenylylgroup, a 4″-t-butyl-p-terphenyl-4-yl group, a chrysenyl group, a fluoranthenyl group, fluorenyl group, and the like.

Among these, a phenyl group, a naphthyl group, a biphenyl group, a terphenyl group, a phenanthryl group and a fluorenyl group are preferable, and a phenyl group, a naphthyl group, a biphenyl group, a terphenyl group and a fluorenyl group are more preferable.

In the present specification, a dibenzofuranyl group and a dibenzothiophenyl group also include the following structures,

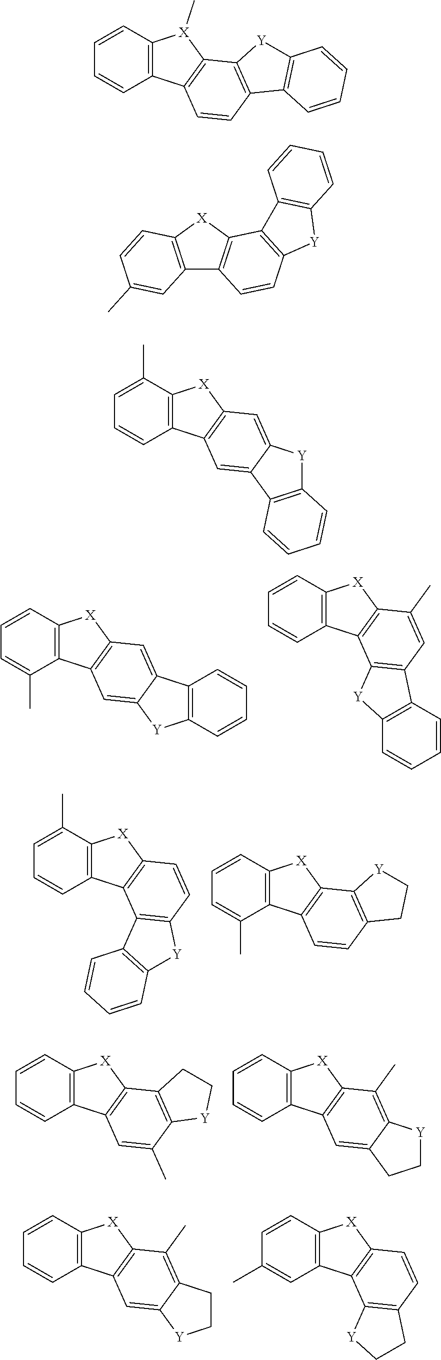

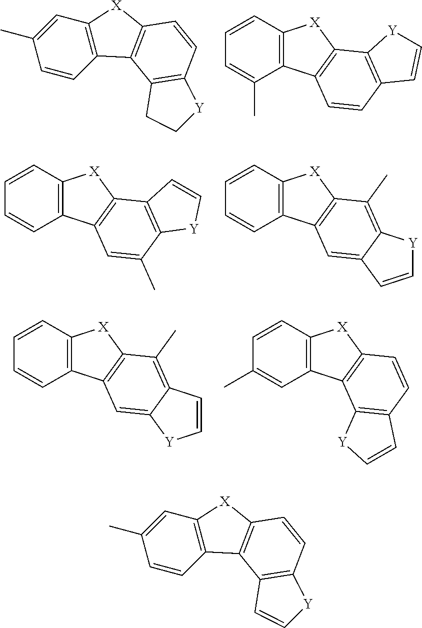

wherein X and Y are independently an oxygen atom or a sulfur atom.

Examples of the alkyl group including 1 to 50 (preferably 1 to 18) carbon atoms in Ar₁₁ to Ar₁₇ and R₁ to R₃ include a methyl group, an ethyl group, a propyl group, an isopropyl group, a n-butyl group, a s-butyl group, an isobutyl group, a t-butyl group, a n-pentyl group, a n-hexyl group, a n-heptyl group, a n-octyl group, a hydroxymethyl group, a 1-hydroxyethyl group, a 2-hydroxyethyl group, a 2-hydroxyisobutyl group, a 1,2-dihydroxyethyl group, a 1,3-dihydroxyisopropyl group, a 2,3-dihydroxy-t-butyl group, a 1,2,3-trihydroxypropyl group, a chloromethyl group, a 1-chloroethyl group, a 2-chloroethyl group, a 2-chloroisobutyl group, a 1,2-dichloroethyl group, a 1,3-dichloroisopropyl group, a 2,3-dichloro-t-butyl group, a 1,2,3-trichloropropyl group, a bromomethyl group, a 1-bromoethyl group, a 2-bromoethyl group, a 2-bromoisobutyl group, a 1,2-dibromoethyl group, a 1,3-dibromoisopropyl group, a 2,3-dibromo-t-butyl group, a 1,2,3-tribromopropyl group, an iodomethyl group, a 1-iodoethyl group, a 2-iodoethyl group, a 2-iodoisobutyl group, a 1,2-diiodoethyl group, a 1,3-diiodoisopropyl group, a 2,3-diiodo-t-butyl group, a 1,2,3-triiodopropyl group, an aminomethyl group, a 1-aminoethyl group, a 2-aminoethyl group, a 2-aminoisobutyl group, a 1,2-diaminoethyl group, a 1,3-diaminoisopropyl group, a 2,3-diamino-t-butyl group, a 1,2,3-triaminopropyl group, a cyanomethyl group, a 1-cyanoethyl group, a 2-cyanoethyl group, a 2-cyanoisobutyl group, a 1,2-dicyanoethyl group, a 1,3-dicyanoisopropyl group, a 2,3-dicyano-t-butyl group, a 1,2,3-tricyanopropyl group, a nitromethyl group, a 1-nitroethyl group, a 2-nitroethyl group, a 2-nitroisobutyl group, a 1,2-dinitroethyl group, a 1,3-dinitroisopropyl group, a 2,3-dinitro-t-butyl group, a 1,2,3-trinitropropyl group, a cyclopropyl group, a cyclobutyl group, a cyclopentyl group, a cyclohexyl group, a 4-methylcyclohexyl group, a 1-adamantyl group, a 2-adamantyl group, a 1-norbornyl group, a 2-norbornyl group and the like.

Examples of the alkoxy group including 1 to 50 (preferably 1 to 18) carbon atoms in Ar₁₁ to Ar₁₇ and R₁ to R₃ include a group in which oxygen atom is bonded to the above alkyl group including 1 to 50 carbon atoms.

Examples of the aralkyl group including 6 to 50 (preferably 7 to 50, more preferably 6 to 18, particularly preferably 7 to 18) carbon atoms in Ar₁₁ to Ar₁₇ and R₁ to R₃ include a benzyl group, a 1-phenylethyl group, a 2-phenylethyl group, a 1-phenylisopropyl group, a 2-phenylisopropyl group, a phenyl-t-butyl group, an α-naphthylmethyl group, a 1-α-naphthylethyl group, a 2-α-naphthylethyl group, a 1-α-naphthylisopropyl group, a 2-α-naphthylisopropyl group, a β-naphthylmethyl group, a 1-β-naphthylethyl group, a 2-β-naphthylethyl group, a 1-β-naphthylisopropyl group, a 2-β-naphthylisopropyl group, a 1-pyrrolylmethyl group, a 2-(1-pyrrolyl)ethyl group, a p-methylbenzyl group, a m-methylbenzyl group, an o-methylbenzyl group, a p-chlorobenzyl group, a m-chlorobenzyl group, an o-chlorobenzyl group, a p-bromobenzyl group, a m-bromobenzyl group, an o-bromobenzyl group, a p-iodobenzyl group, a m-iodobenzyl group, a o-iodobenzyl group, a p-hydroxybenzyl group, a m-hydroxybenzyl group, an o-hydroxybenzyl group, a p-aminobenzyl group, a m-aminobenzyl group, an o-aminobenzyl group, a p-nitrobenzyl group, a m-nitrobenzyl group, an o-nitrobenzyl group, a p-cyanobenzyl group, a m-cyanobenzyl group, an o-cyanobenzyl group, a 1-hydroxy-2-phenylisopropyl group, a 1-chloro-2-phenylisopropyl group, and the like.

Examples of the aryloxy group including 5 to 50 (preferably 6 to 50, more preferably 5 to 18, particularly preferably 6 to 18) ring carbon atoms in Ar₁₁ to Ar₁₇ and R₁ to R₃ include a group in which oxygen atom is bonded to the above aryl group including 5 to 50 (preferably 6 to 50, more preferably 5 to 18, particularly preferably 6 to 18) ring carbon atoms.

Examples of the arylthio group including 5 to 50 (preferably 6 to 50, more preferably 5 to 18, particularly preferably 6 to 18) ring carbon atoms in Ar₁₁ to Ar₁₇ and R₁ to R₃ include a group in which sulfur atom is bonded to the above aryl group including 5 to 50 (preferably 6 to 50, more preferably 5 to 18, particularly preferably 6 to 18) ring carbon atoms.

Examples of the alkoxycarbonyl group including 2 to 50 (preferably 2 to 18) carbon atoms in Ar₁₁ to Ar₁₇ and R₁ to R₃ include a group in which oxygen atom of oxycarbonyl is bonded to the above alkyl group including 1 to 50 carbon atoms.

Examples of the halogen atom in Ar₁₁ to Ar₁₇ and R₁ to R₃ include a fluorine atom, a chlorine atom, a bromine atom, an iodine atom and the like.

a, c and e are independently preferably 0 or 1.

b, d and f are independently preferably 1 or 2.

Specific examples of the compounds represented by the formulas (11) to (14) include the following compounds.

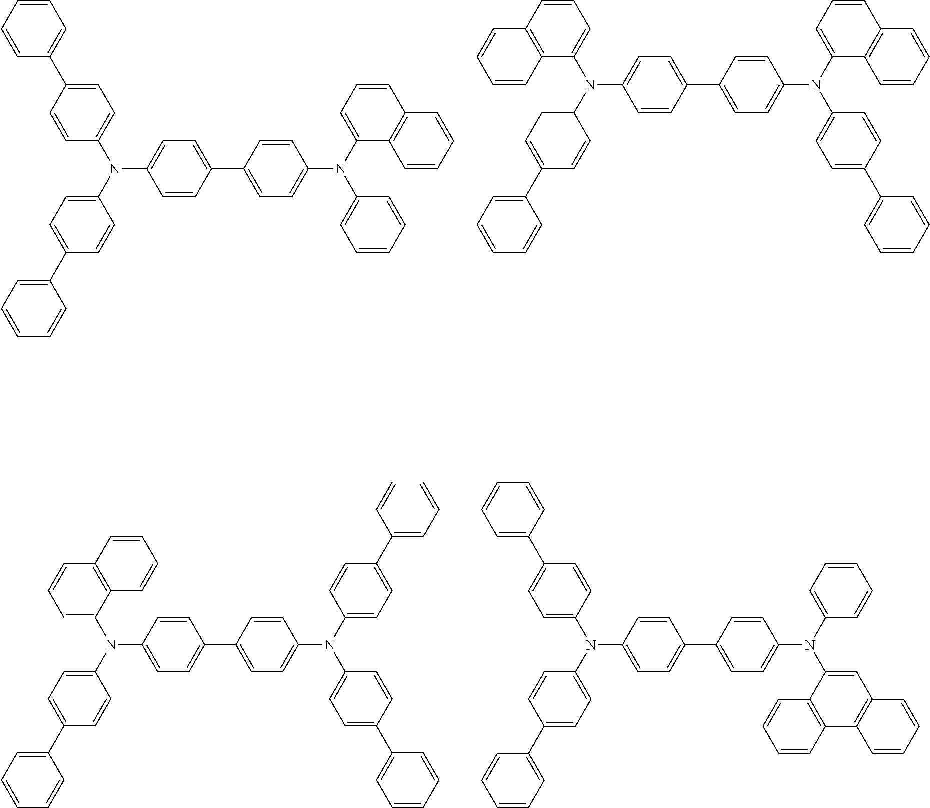

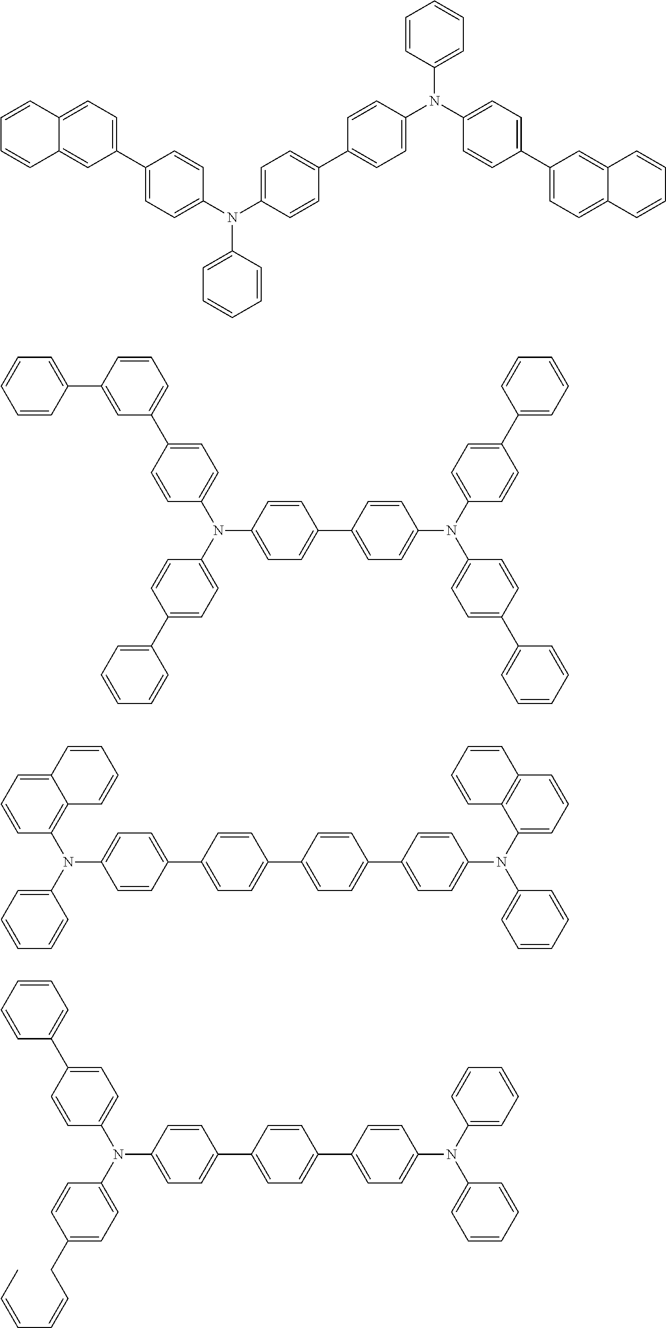

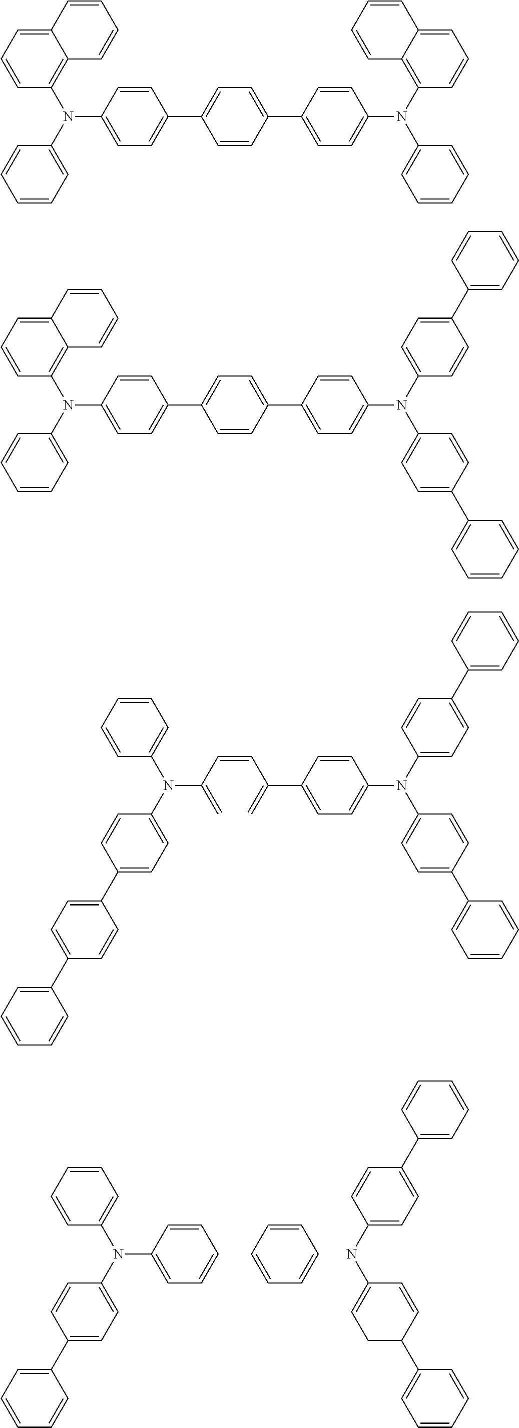

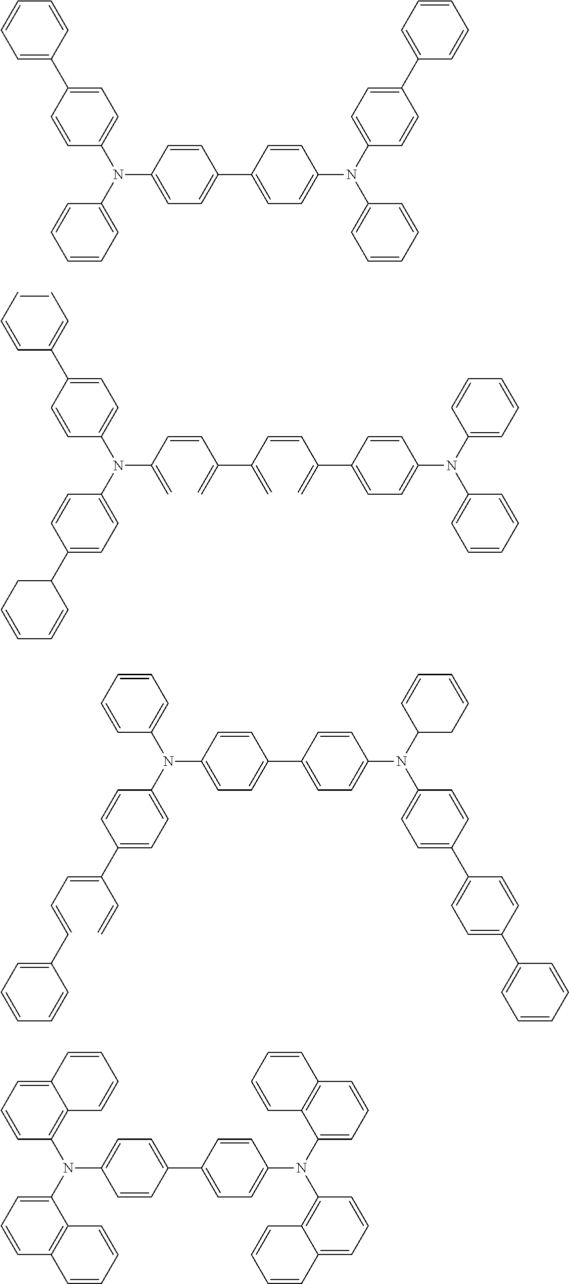

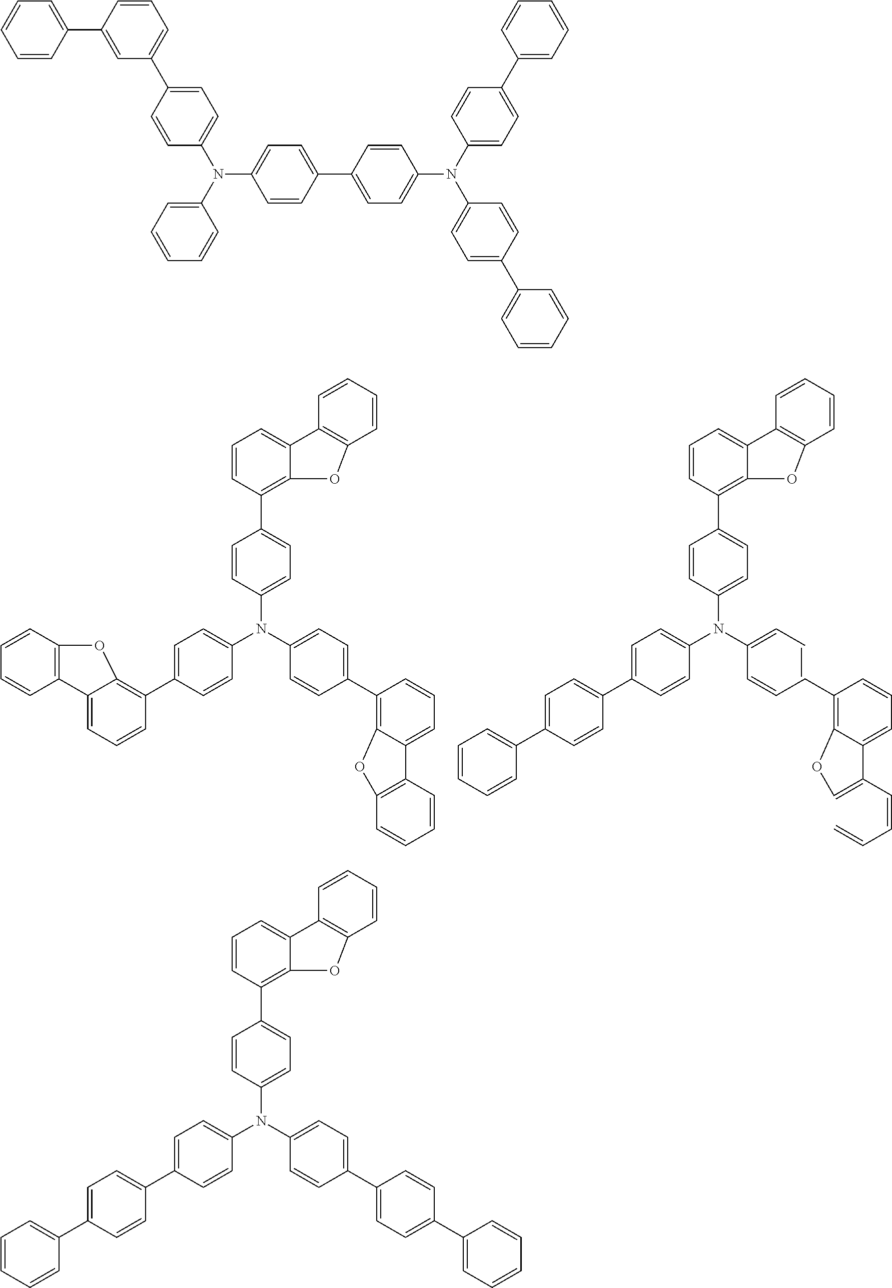

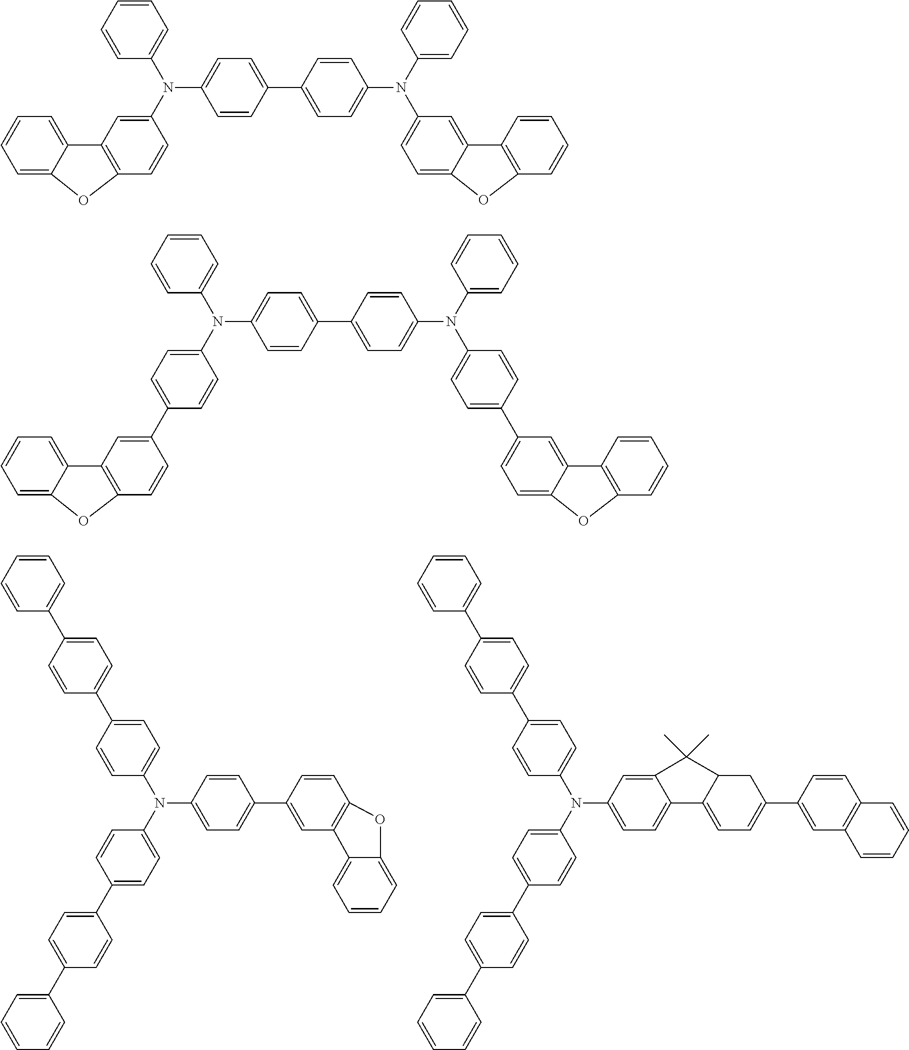

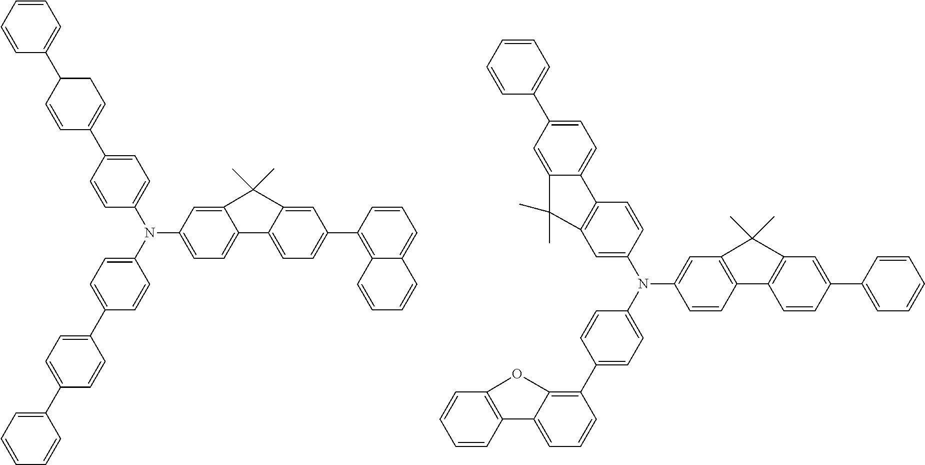 [SMILES: CC1(C)c2cc(-c3cccc4ccccc34)ccc2-c2ccc(N(c3ccc(C4=CCC(c5ccccc5)C=C4)cc3)c3ccc(-c4ccc(-c5ccccc5)cc4)cc3)cc21 ; CC1(C)c2cc(-c3ccccc3)ccc2-c2ccc(N(c3ccc(-c4cccc5c4oc4ccccc45)cc3)c3ccc4c(c3)C(C)(C)c3cc(-c5ccccc5)ccc3-4)cc21]

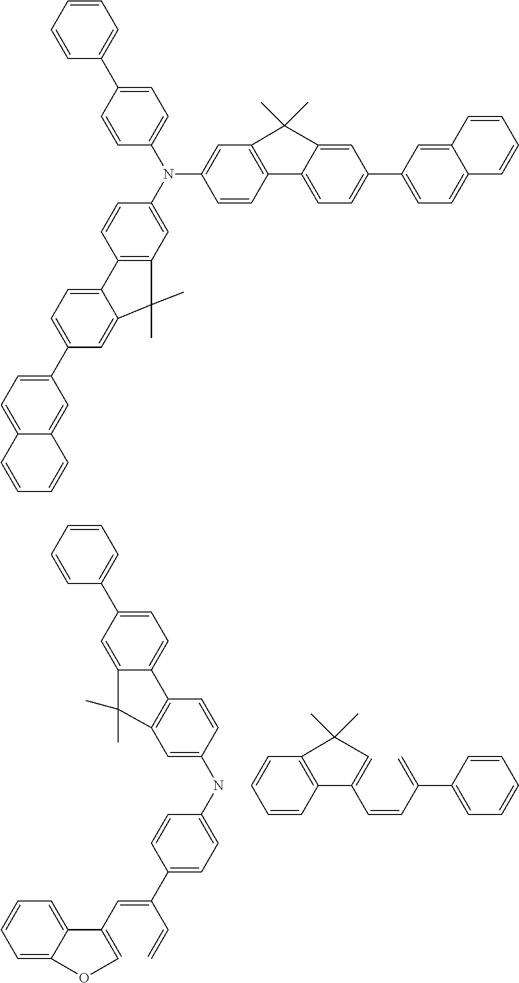

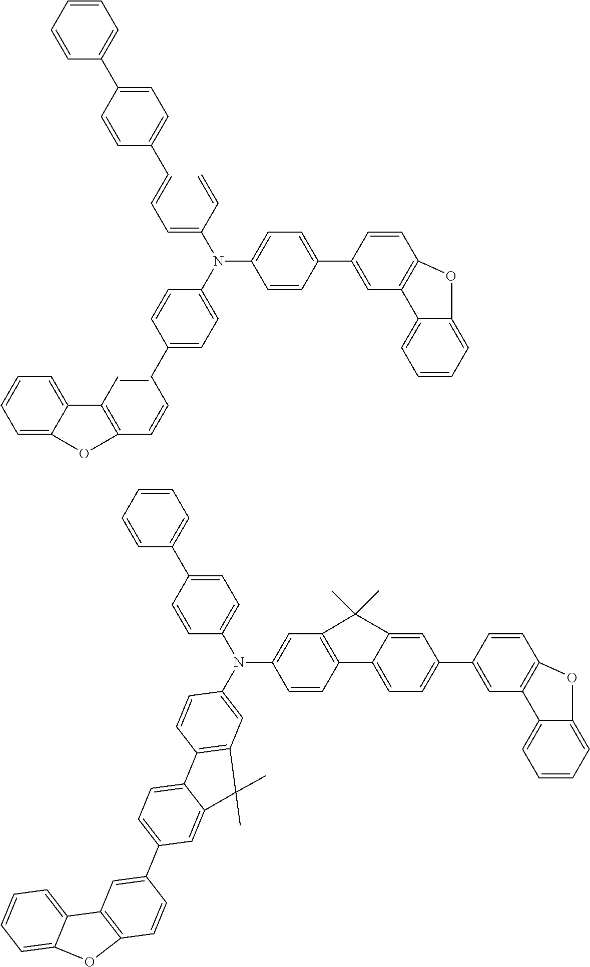 [SMILES: C=C/C(=C\C=C\c1ccc(-c2ccccc2)cc1)N(c1ccc(C/C=C\c2oc3ccccc3c2C)cc1)c1ccc(-c2ccc3oc4ccccc4c3c2)cc1 ; CC1(C)c2cc(-c3ccc4oc5ccccc5c4c3)ccc2-c2ccc(N(c3ccc(-c4ccccc4)cc3)c3ccc4c(c3)C(C)(C)c3cc(-c5ccc6oc7ccccc7c6c5)ccc3-4)cc21]

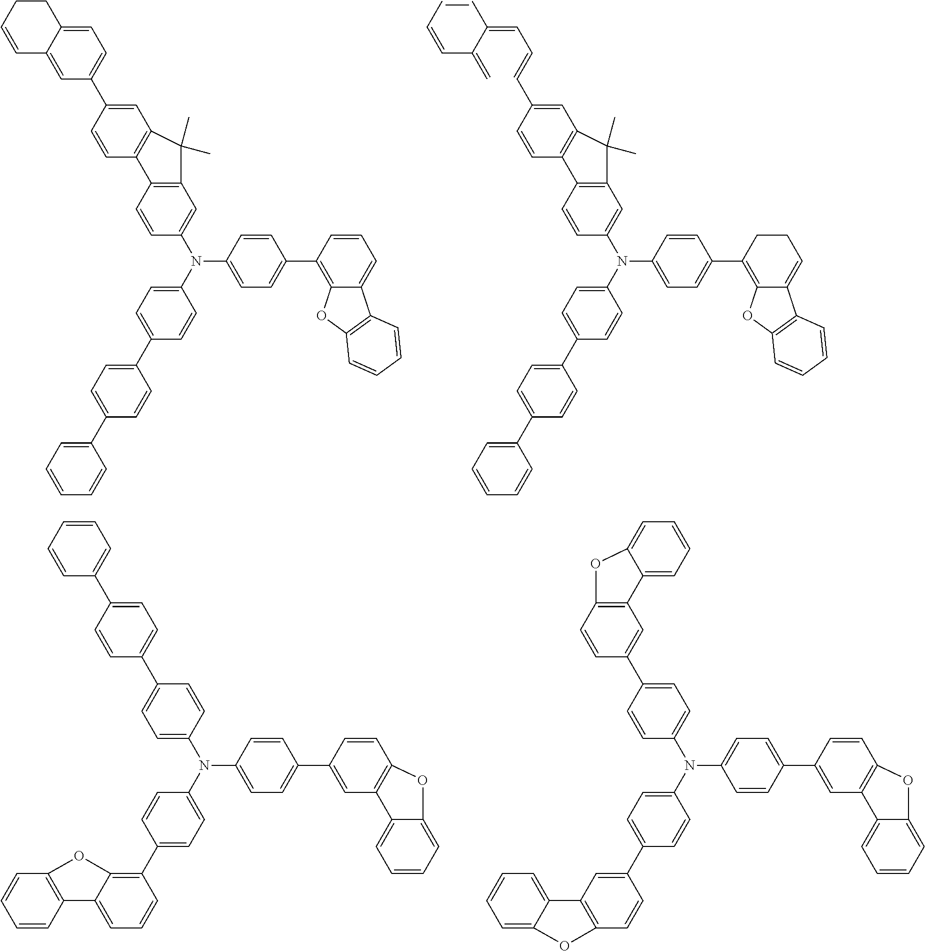

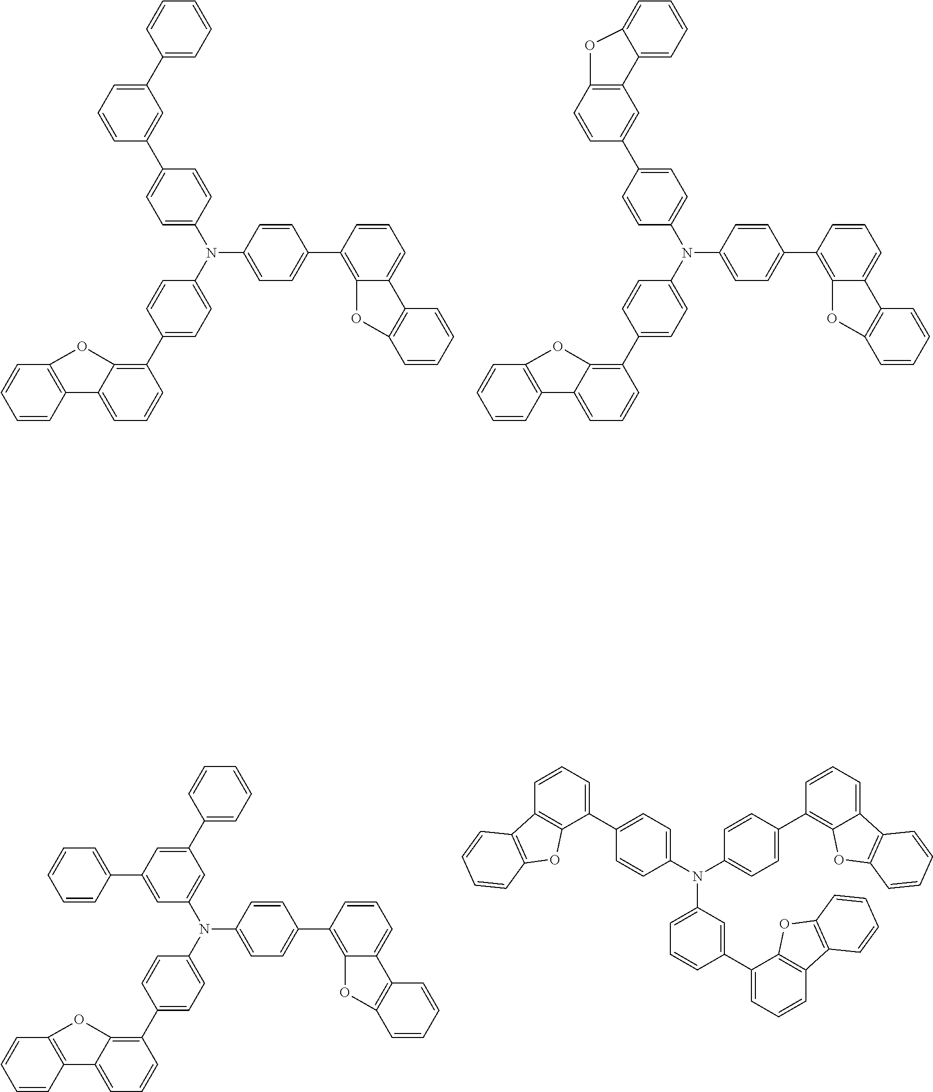 [SMILES: c1ccc(-c2cccc(-c3ccc(N(c4ccc(-c5cccc6c5oc5ccccc56)cc4)c4ccc(-c5cccc6c5oc5ccccc56)cc4)cc3)c2)cc1 ; c1ccc2c(c1)oc1ccc(-c3ccc(N(c4ccc(-c5cccc6c5oc5ccccc56)cc4)c4ccc(-c5cccc6c5oc5ccccc56)cc4)cc3)cc12 ; c1ccc(-c2cc(-c3ccccc3)cc(N(c3ccc(-c4cccc5c4oc4ccccc45)cc3)c3ccc(-c4cccc5c4oc4ccccc45)cc3)c2)cc1 ; c1cc(-c2cccc3c2oc2ccccc23)cc(N(c2ccc(-c3cccc4c3oc3ccccc34)cc2)c2ccc(-c3cccc4c3oc3ccccc34)cc2)c1]

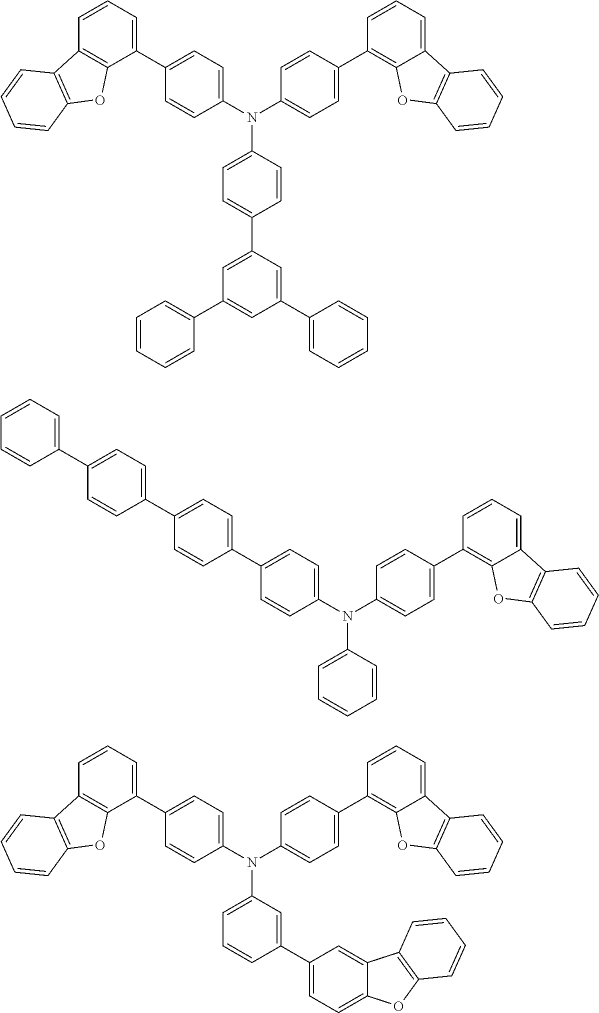 [SMILES: c1ccc(-c2cc(-c3ccccc3)cc(-c3ccc(N(c4ccc(-c5cccc6c5oc5ccccc56)cc4)c4ccc(-c5cccc6c5oc5ccccc56)cc4)cc3)c2)cc1 ; c1ccc(-c2ccc(-c3ccc(-c4ccc(N(c5ccccc5)c5ccc(-c6cccc7c6oc6ccccc67)cc5)cc4)cc3)cc2)cc1 ; c1cc(-c2ccc3oc4ccccc4c3c2)cc(N(c2ccc(-c3cccc4c3oc3ccccc34)cc2)c2ccc(-c3cccc4c3oc3ccccc34)cc2)c1]

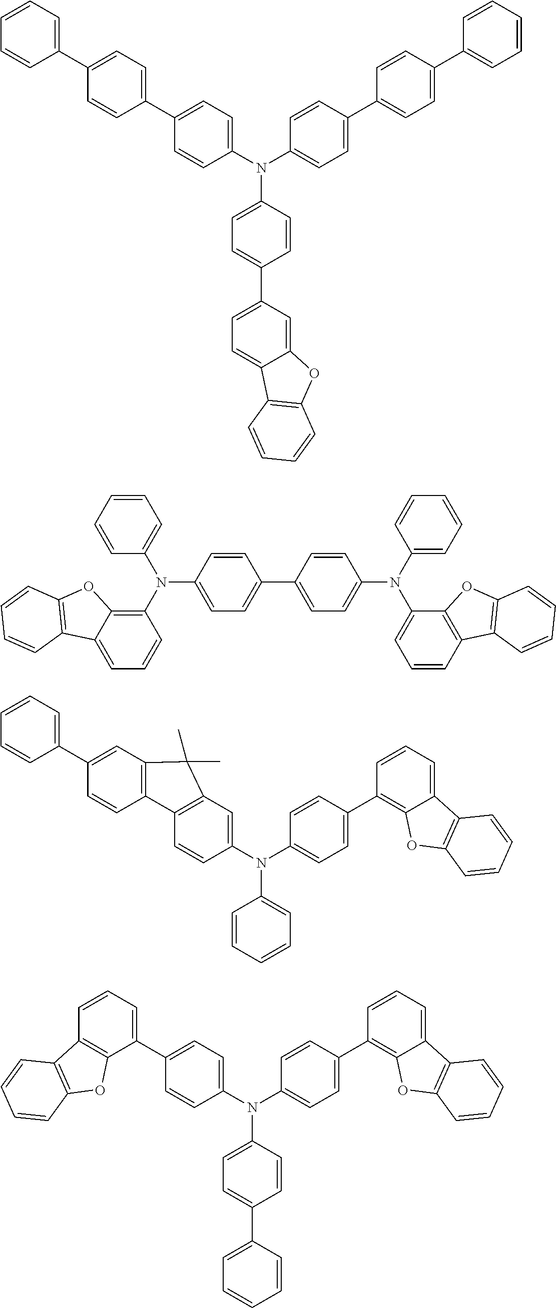

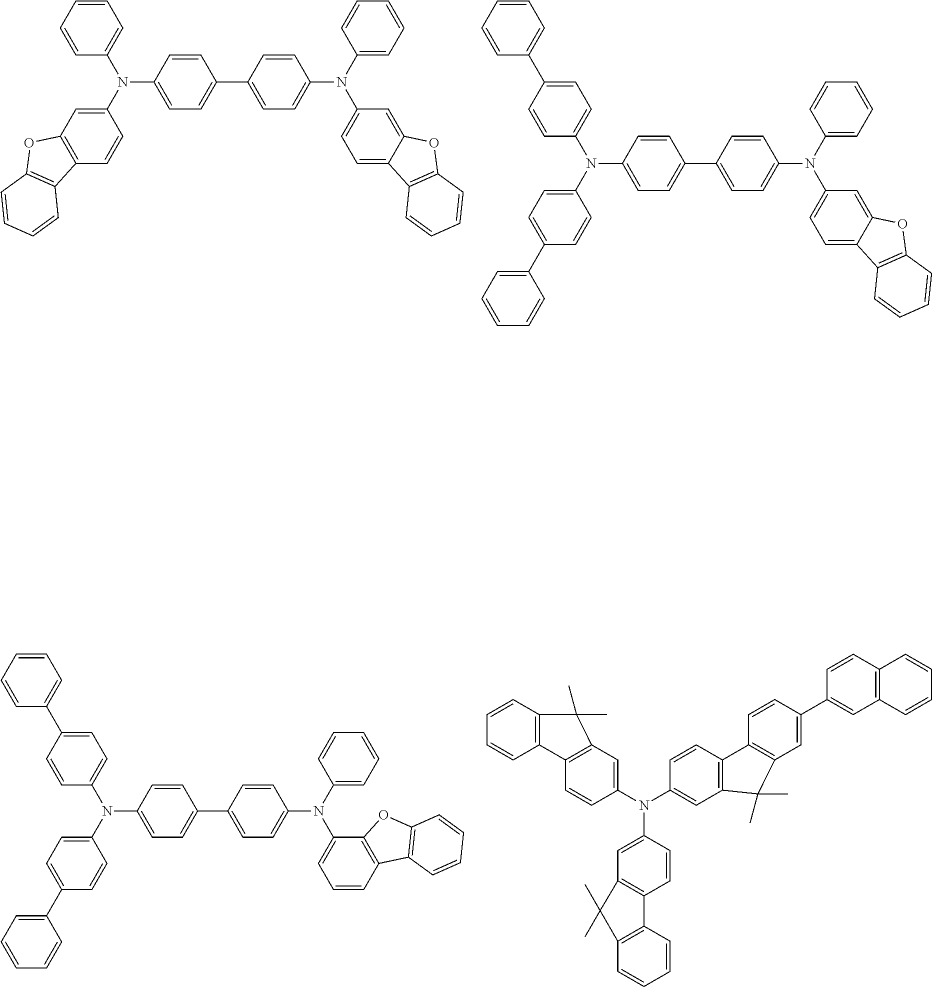 [SMILES: c1ccc(N(c2ccc(-c3ccc(N(c4ccccc4)c4ccc5c(c4)oc4ccccc45)cc3)cc2)c2ccc3c(c2)oc2ccccc23)cc1 ; c1ccc(-c2ccc(N(c3ccc(-c4ccccc4)cc3)c3ccc(-c4ccc(N(c5ccccc5)c5ccc6c(c5)oc5ccccc56)cc4)cc3)cc2)cc1 ; c1ccc(-c2ccc(N(c3ccc(-c4ccccc4)cc3)c3ccc(-c4ccc(N(c5ccccc5)c5cccc6c5oc5ccccc56)cc4)cc3)cc2)cc1 ; CC1(C)c2ccccc2-c2ccc(N(c3ccc4c(c3)C(C)(C)c3ccccc3-4)c3ccc4c(c3)C(C)(C)c3cc(-c5ccc6ccccc6c5)ccc3-4)cc21]

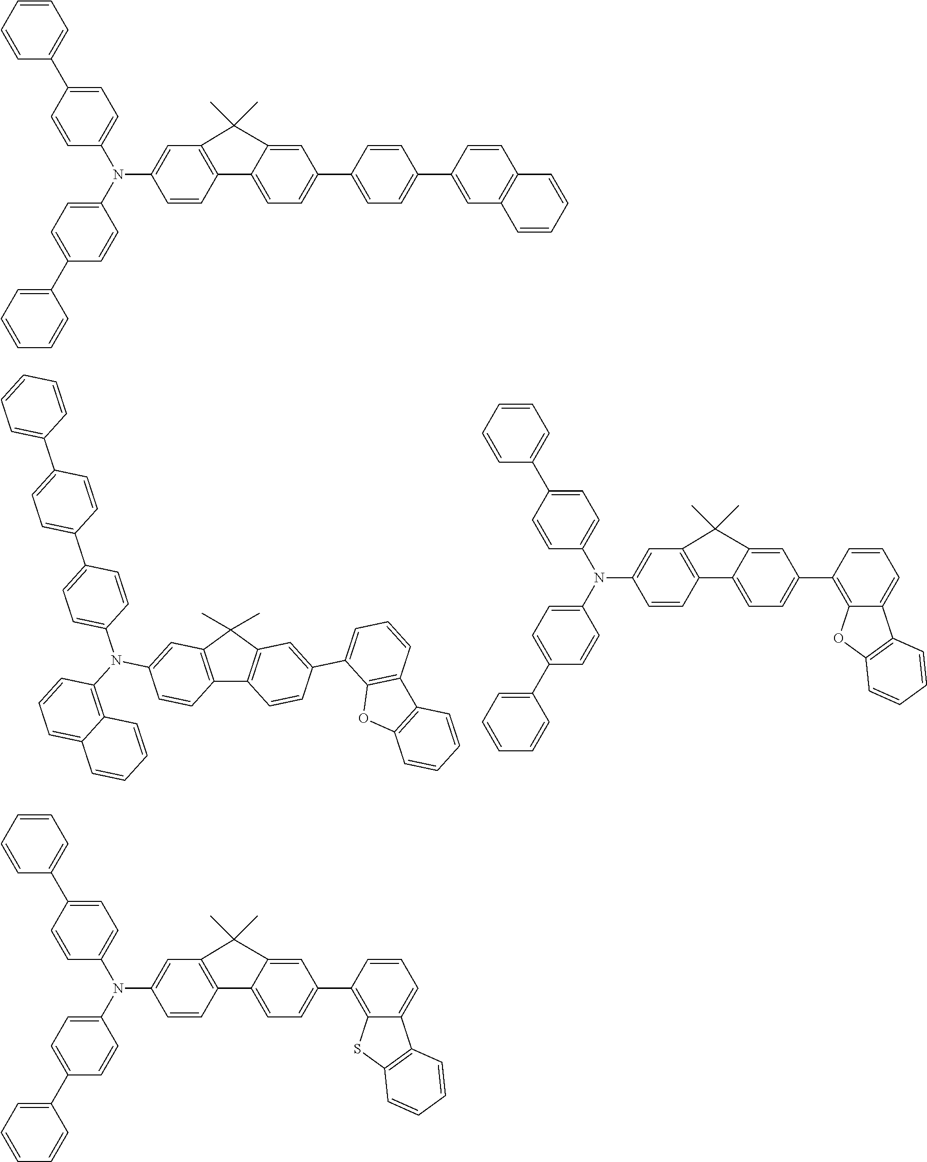

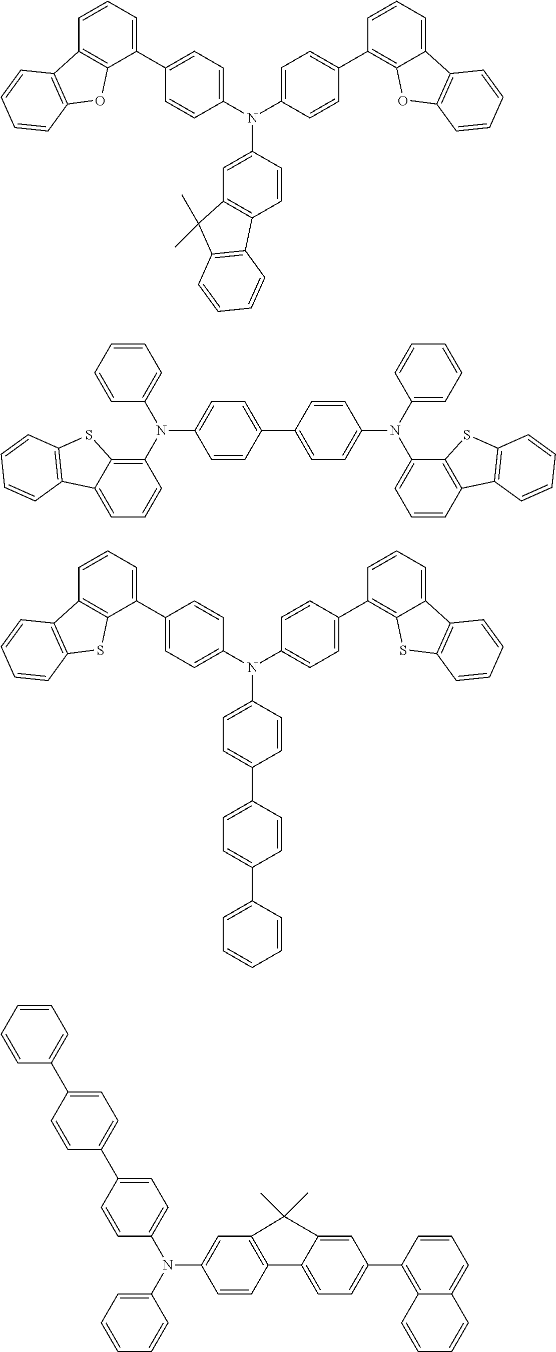

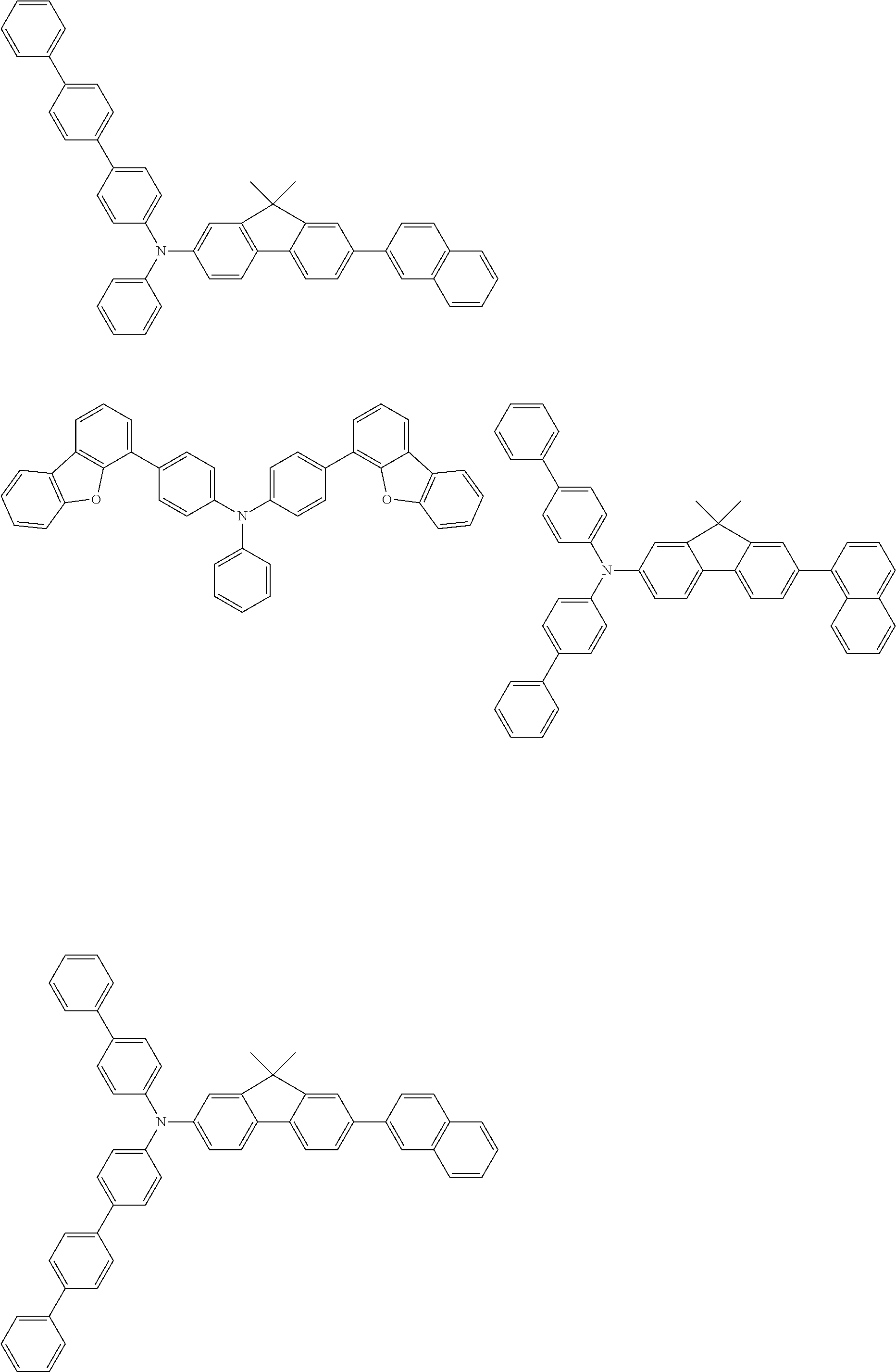 [SMILES: CC1(C)c2cc(-c3ccc4ccccc4c3)ccc2-c2ccc(N(c3ccccc3)c3ccc(-c4ccc(-c5ccccc5)cc4)cc3)cc21 ; c1ccc(N(c2ccc(-c3cccc4c3oc3ccccc34)cc2)c2ccc(-c3cccc4c3oc3ccccc34)cc2)cc1 ; CC1(C)c2cc(-c3cccc4ccccc34)ccc2-c2ccc(N(c3ccc(-c4ccccc4)cc3)c3ccc(-c4ccccc4)cc3)cc21 ; CC1(C)c2cc(-c3ccc4ccccc4c3)ccc2-c2ccc(N(c3ccc(-c4ccccc4)cc3)c3ccc(-c4ccc(-c5ccccc5)cc4)cc3)cc21]

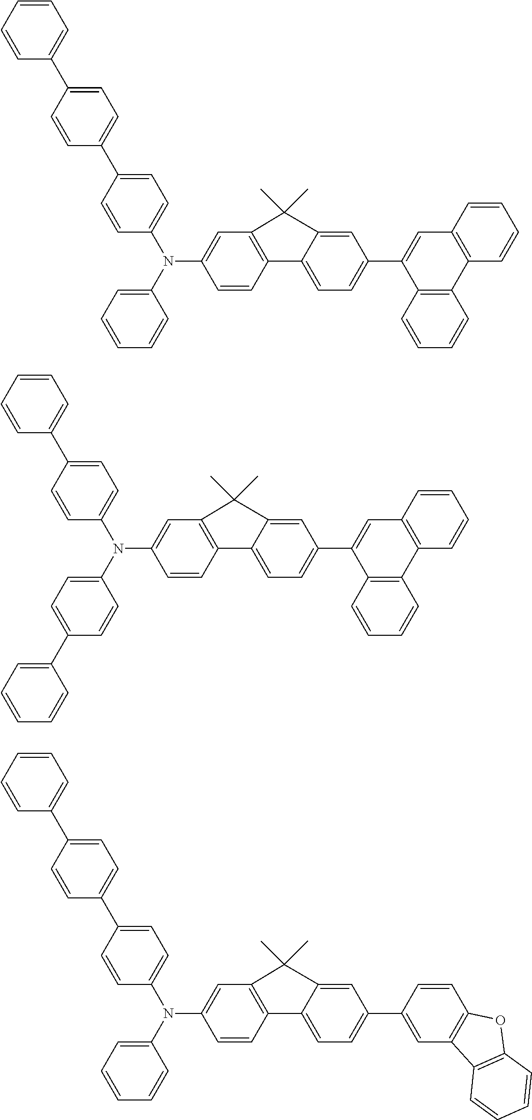

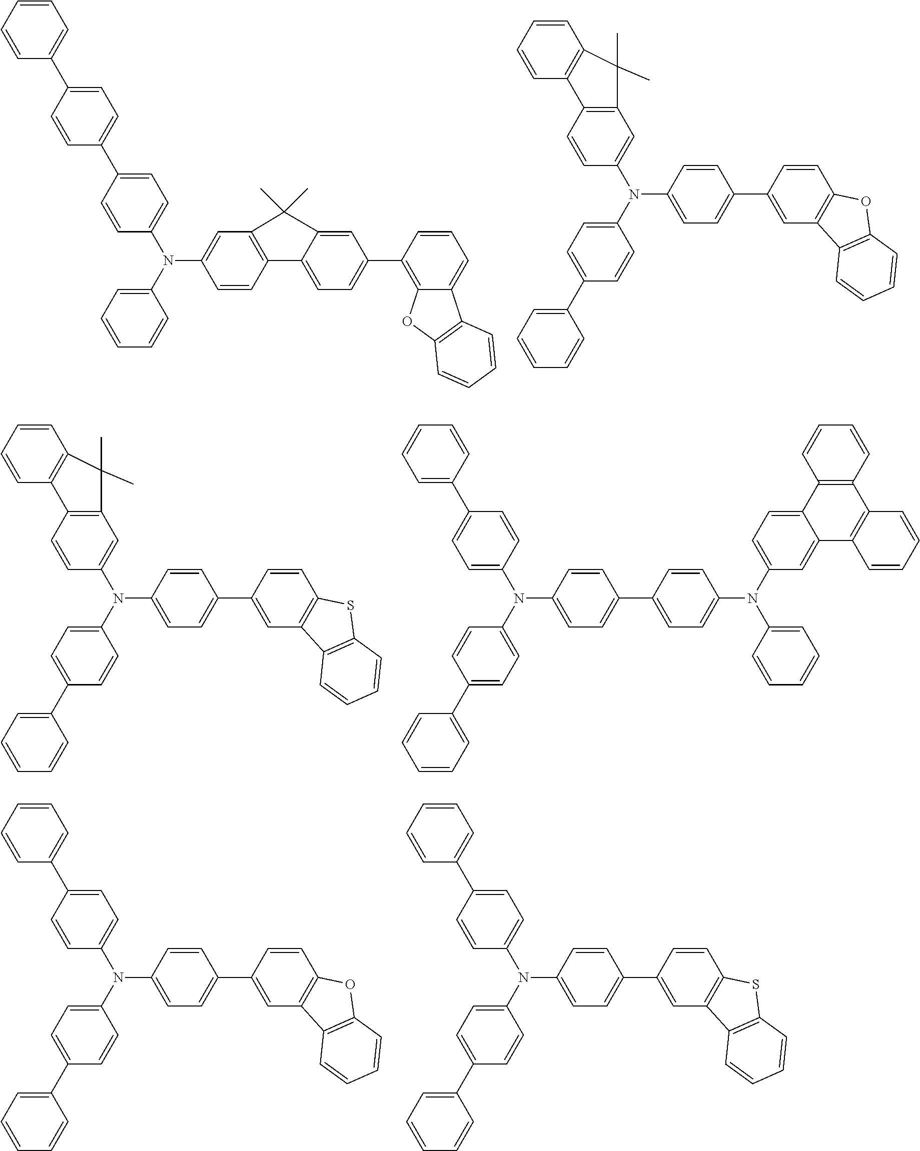

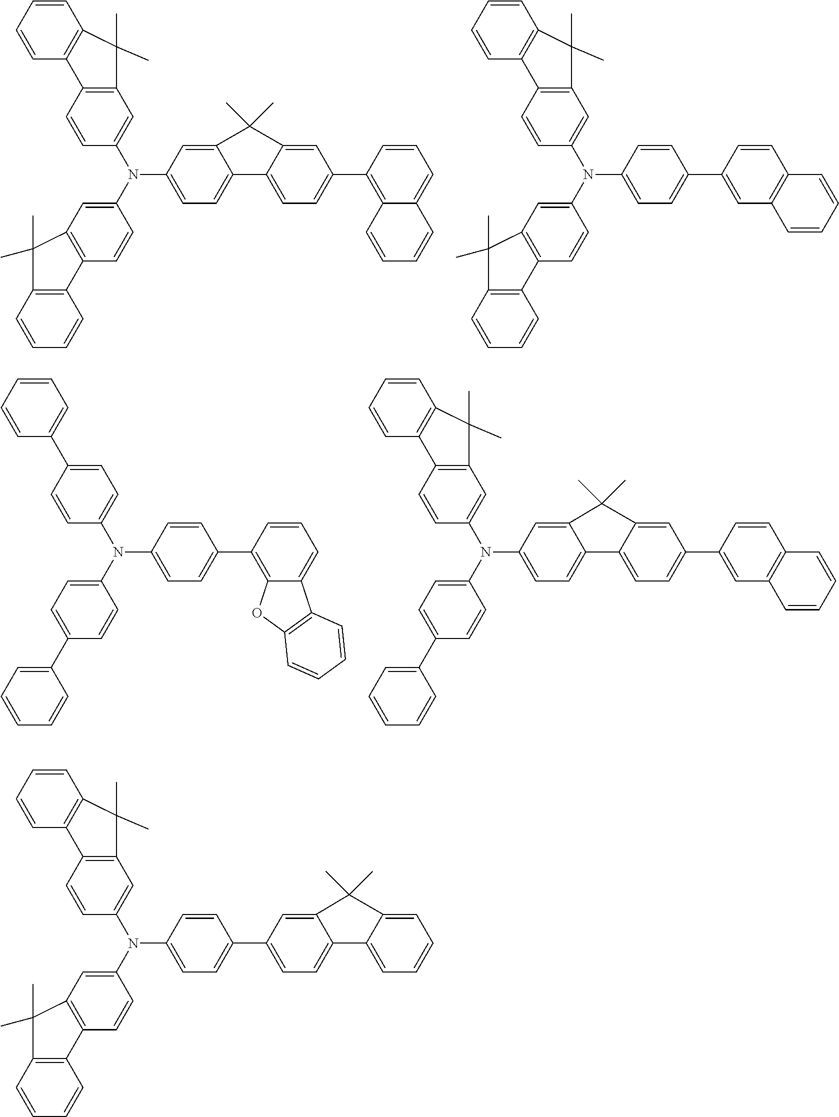

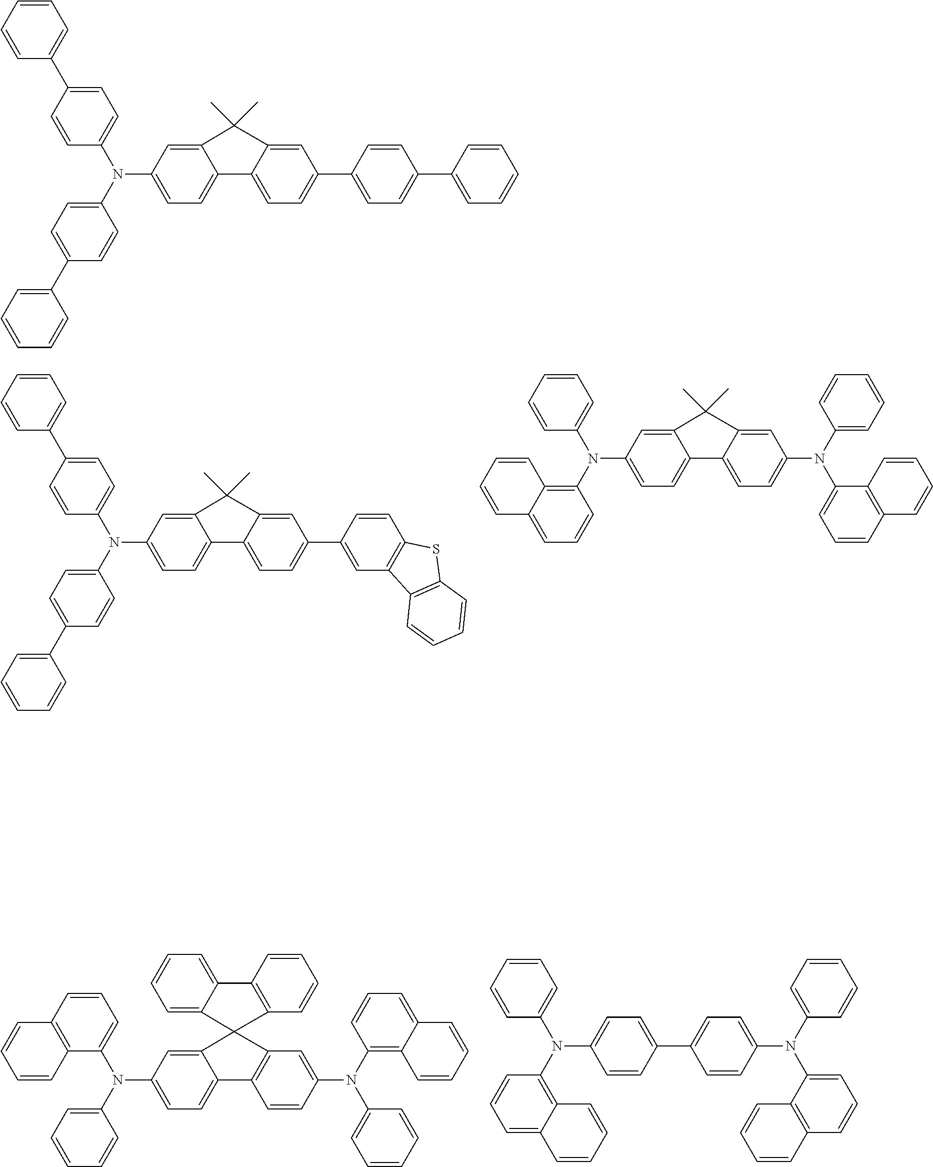

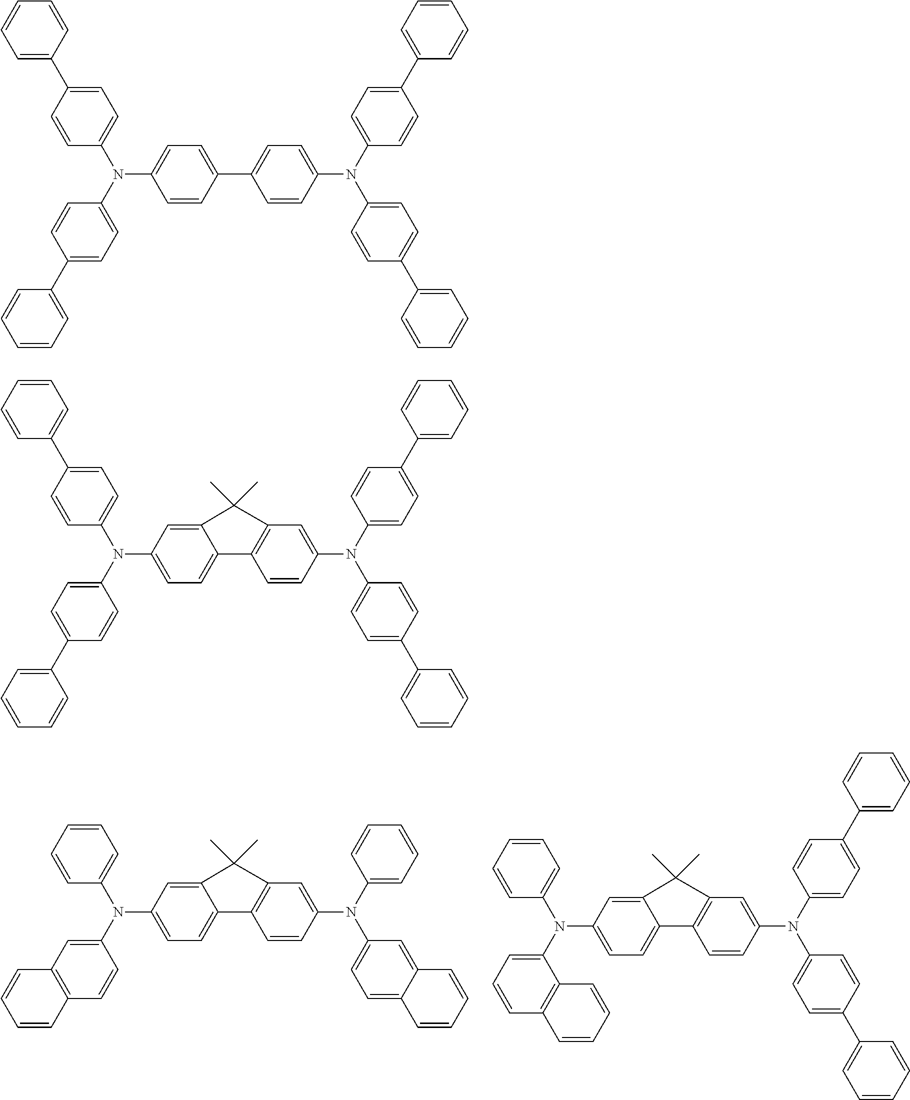

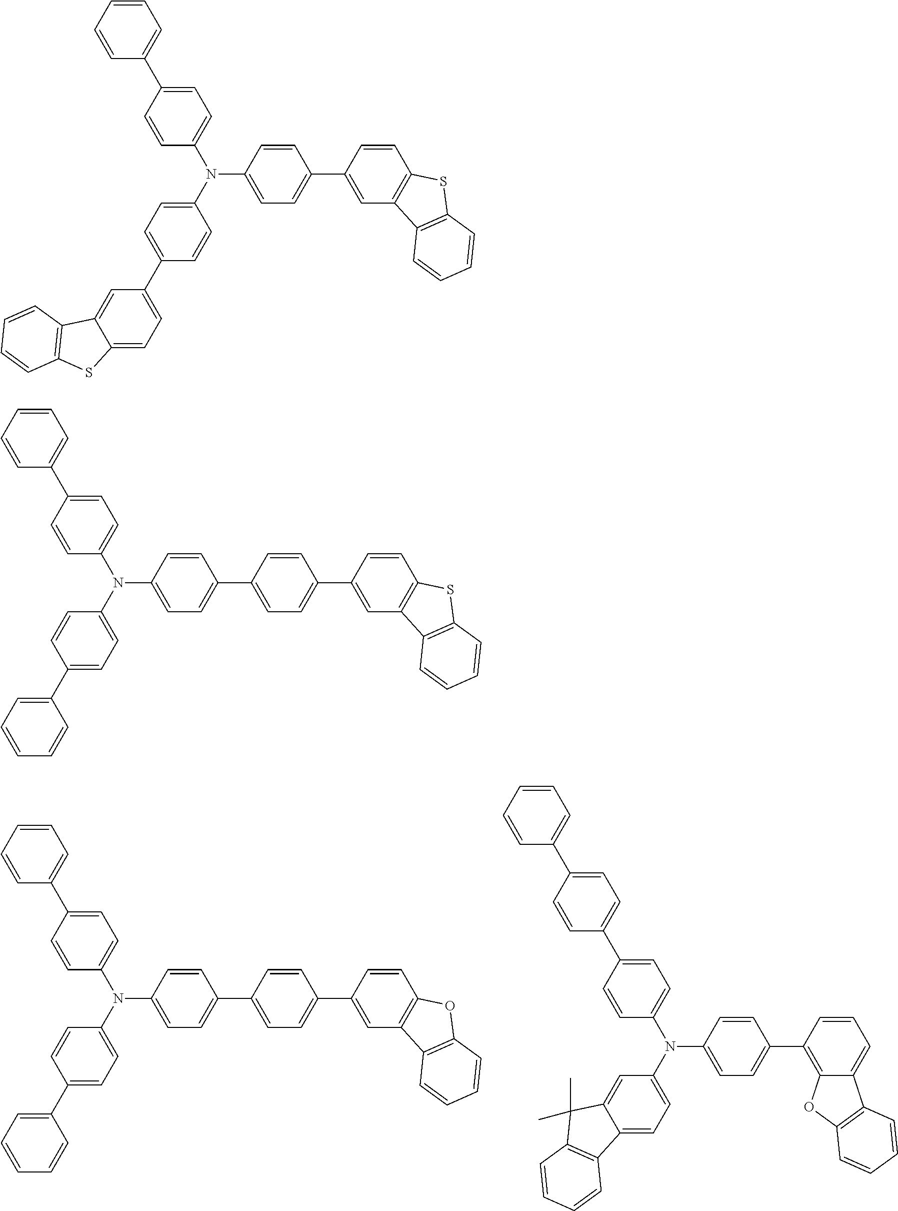

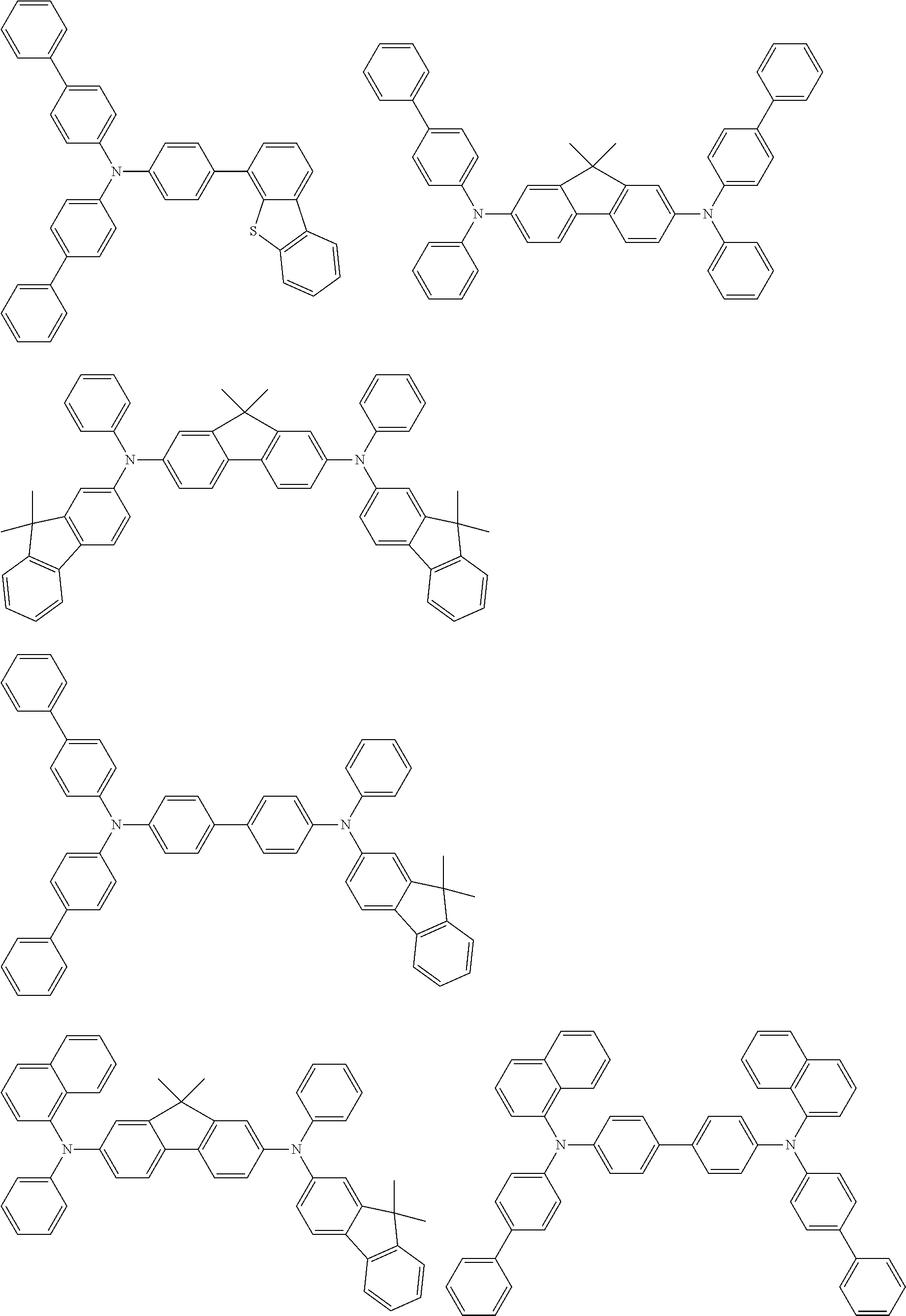

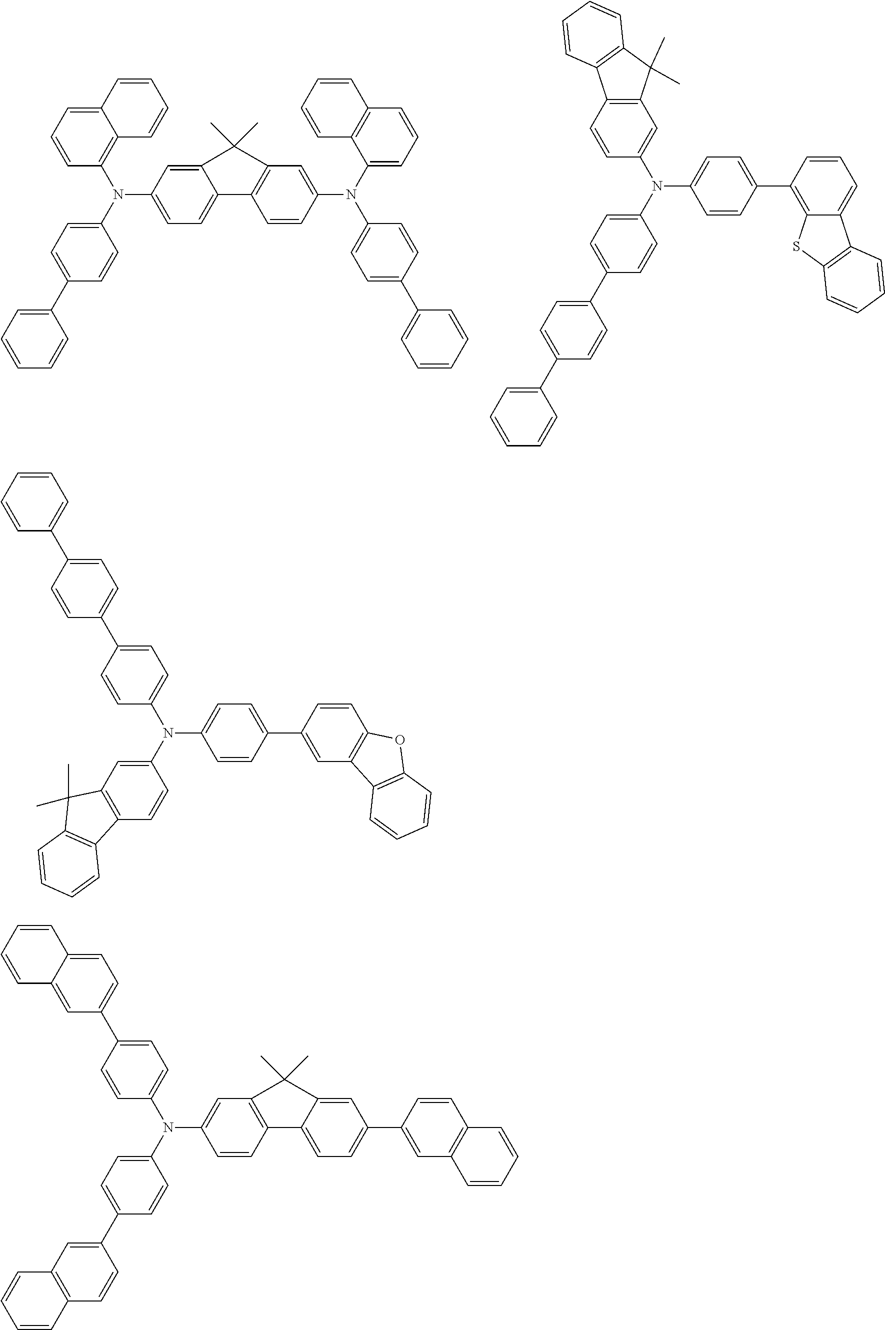

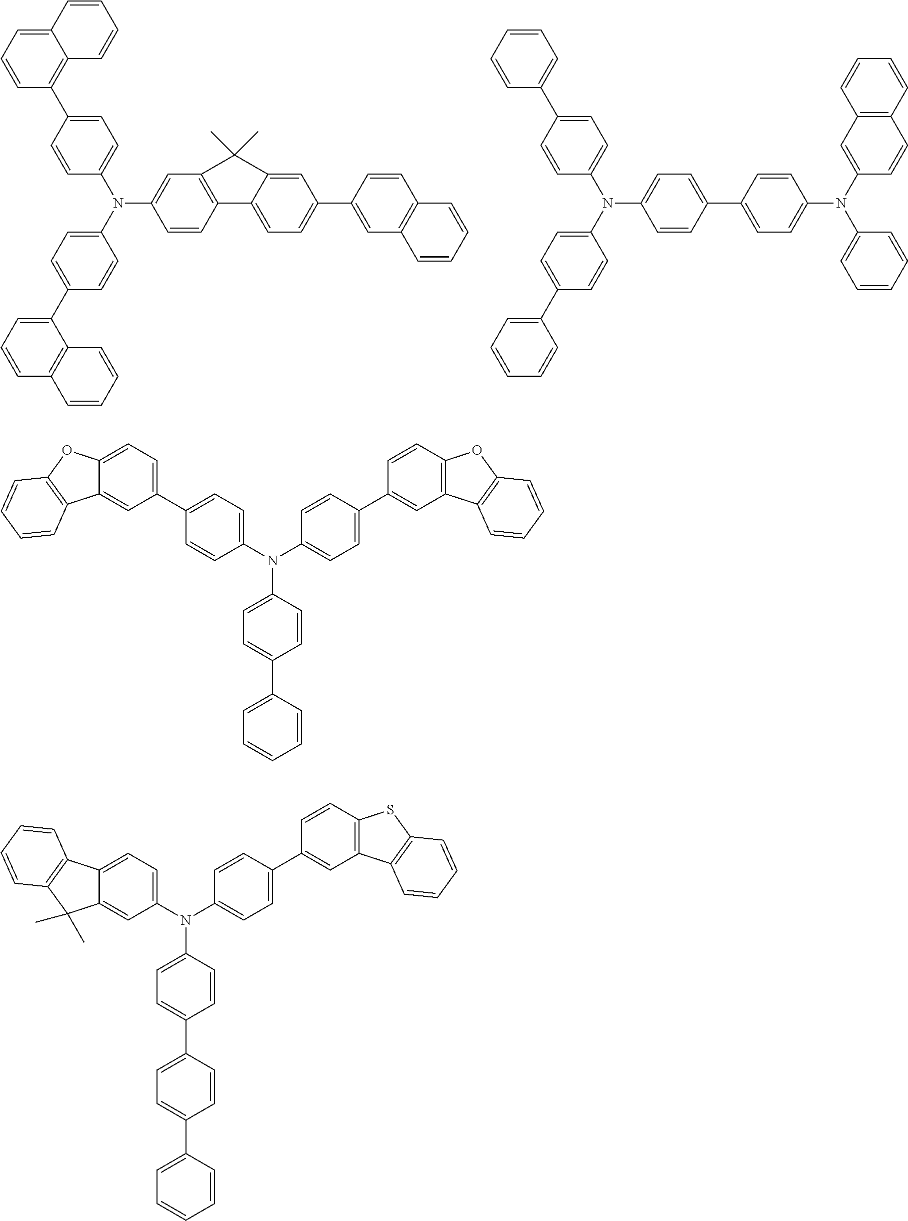

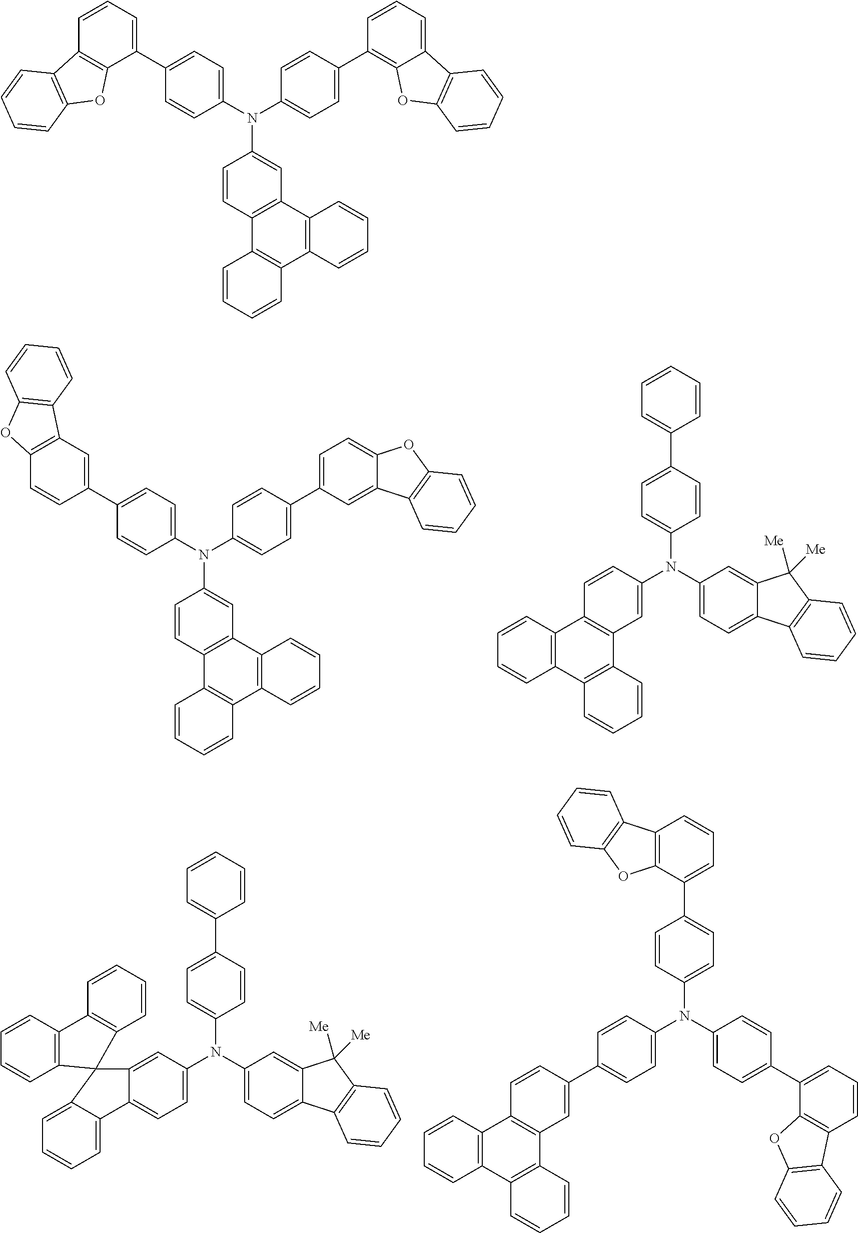

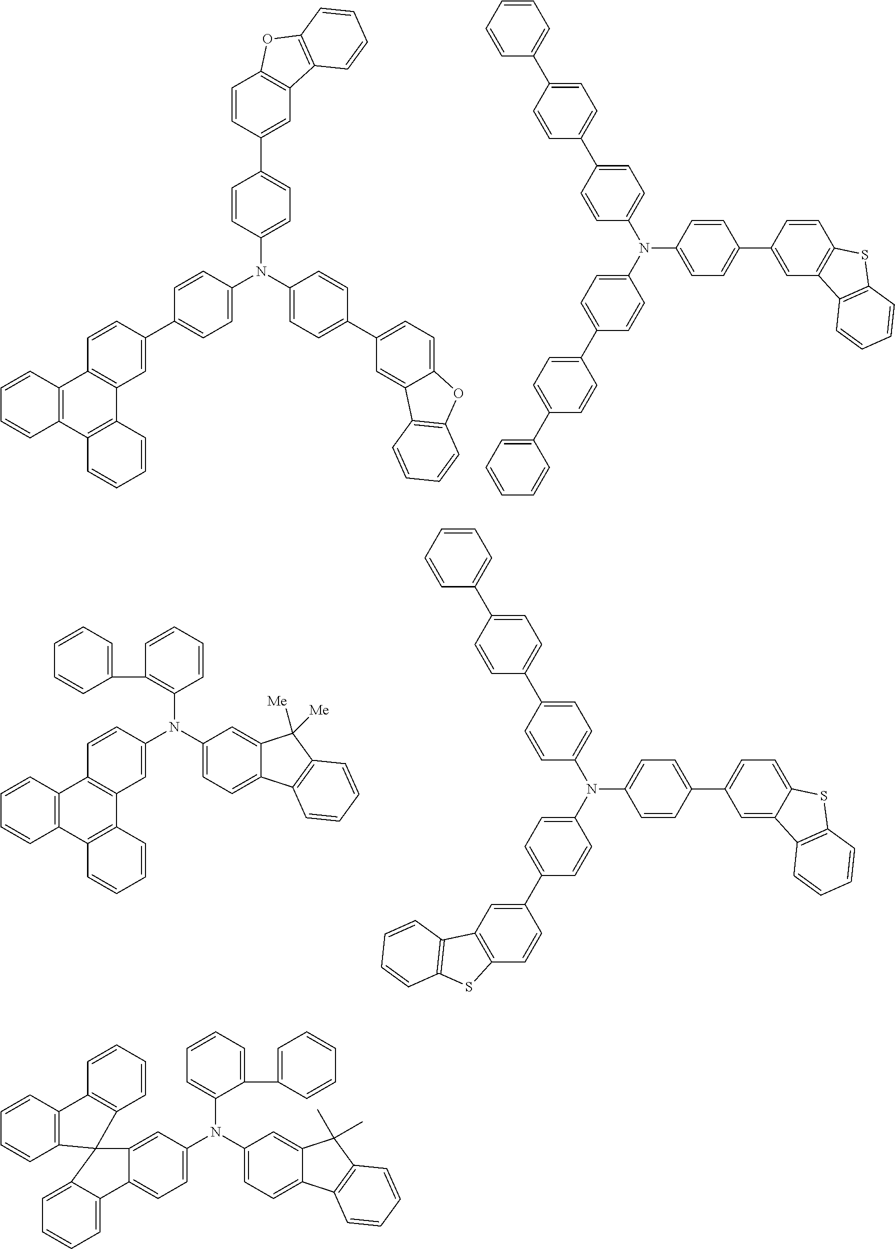

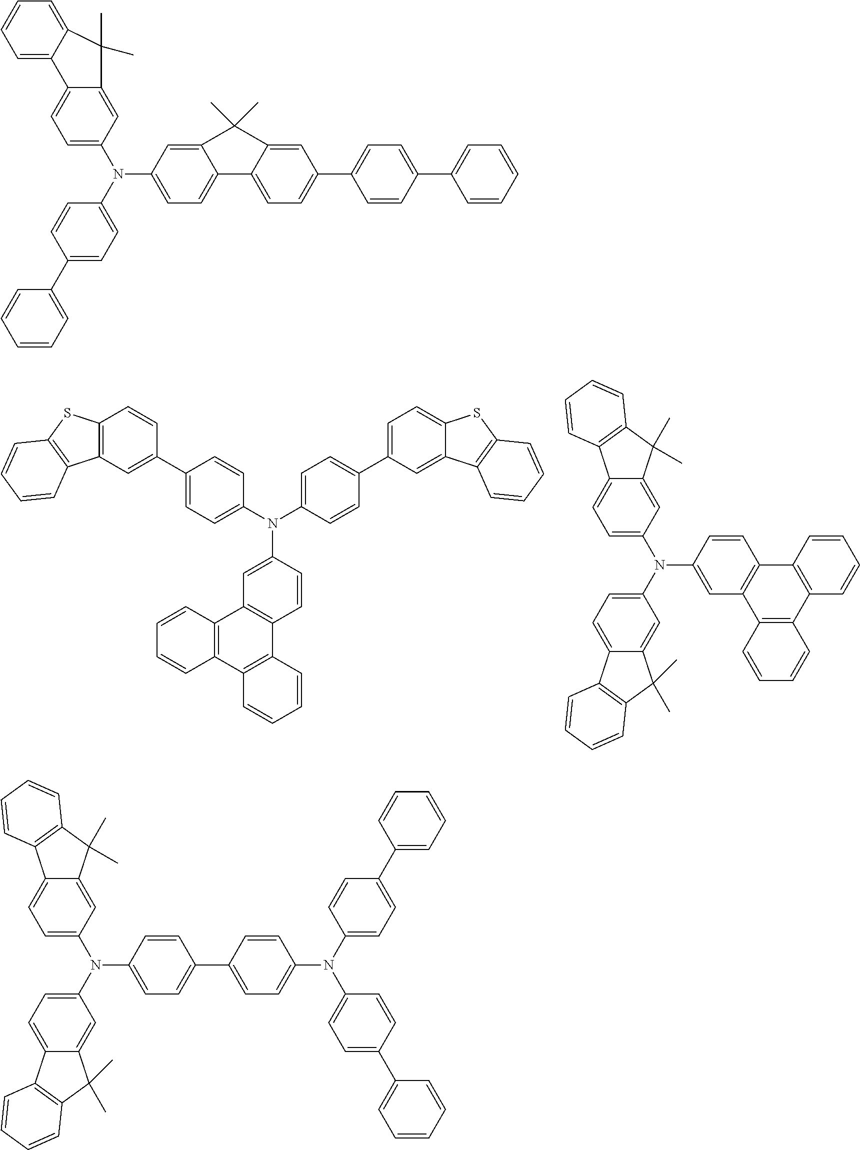

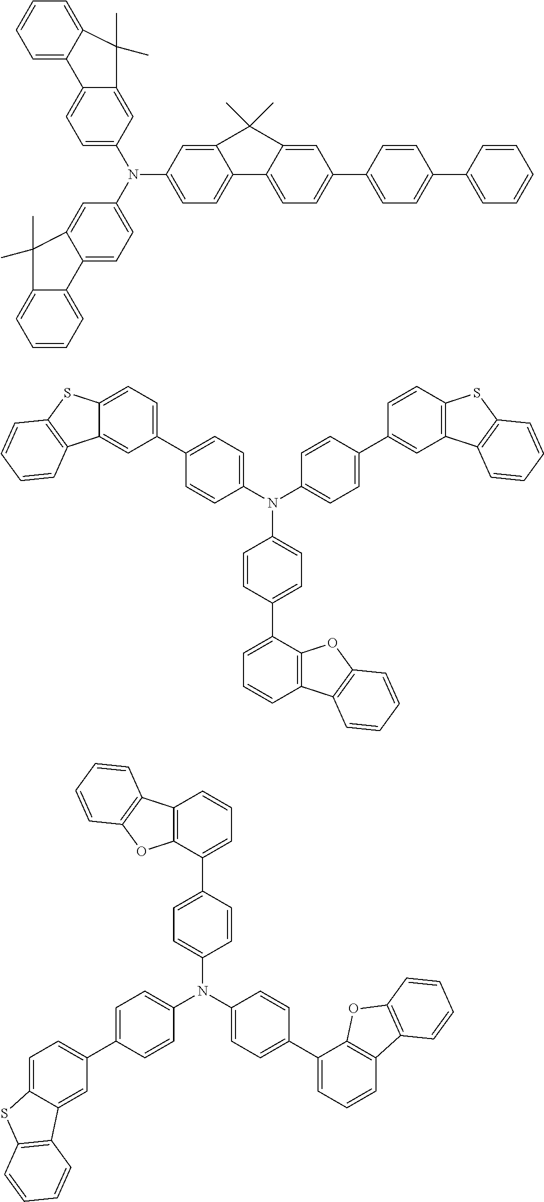

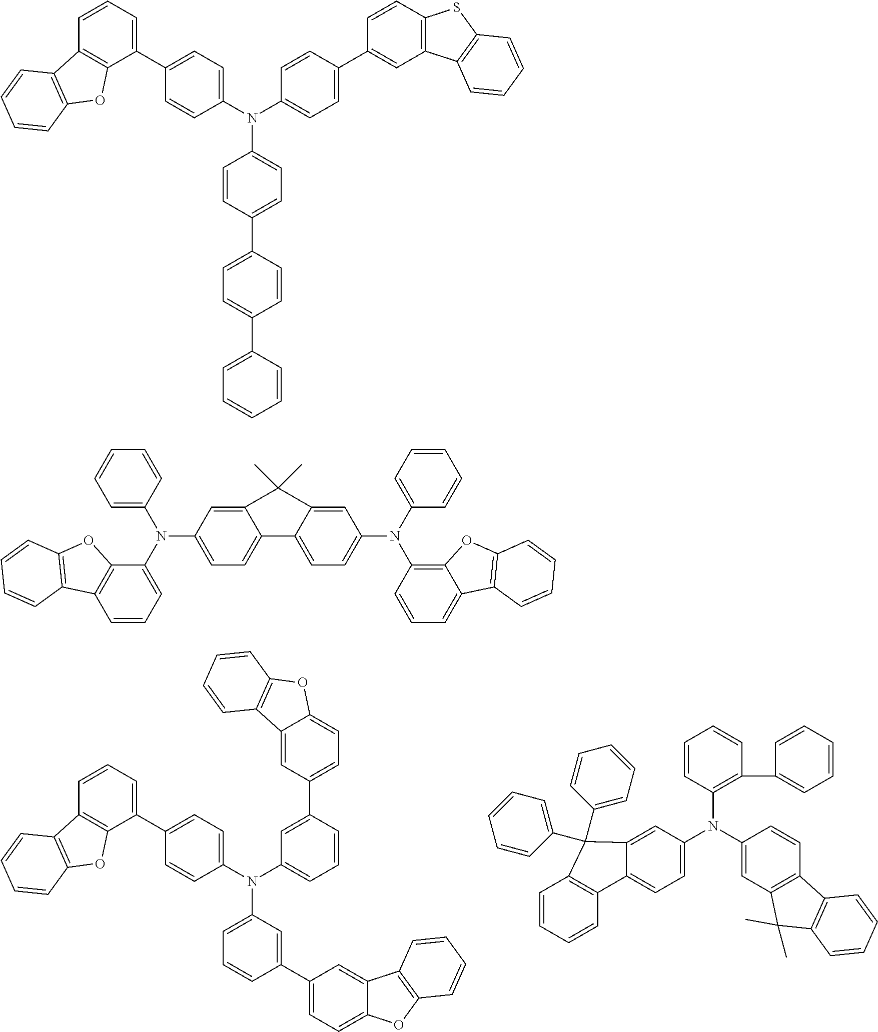 [SMILES: c1ccc(-c2ccc(-c3ccc(N(c4ccc(-c5ccc6sc7ccccc7c6c5)cc4)c4ccc(-c5cccc6c5oc5ccccc56)cc4)cc3)cc2)cc1 ; CC1(C)c2cc(N(c3ccccc3)c3cccc4c3oc3ccccc34)ccc2-c2ccc(N(c3ccccc3)c3cccc4c3oc3ccccc34)cc21 ; c1cc(-c2ccc3oc4ccccc4c3c2)cc(N(c2ccc(-c3cccc4c3oc3ccccc34)cc2)c2cccc(-c3ccc4oc5ccccc5c4c3)c2)c1 ; CC1(C)c2ccccc2-c2ccc(N(c3ccc4c(c3)C(c3ccccc3)(c3ccccc3)c3ccccc3-4)c3ccccc3-c3ccccc3)cc21]

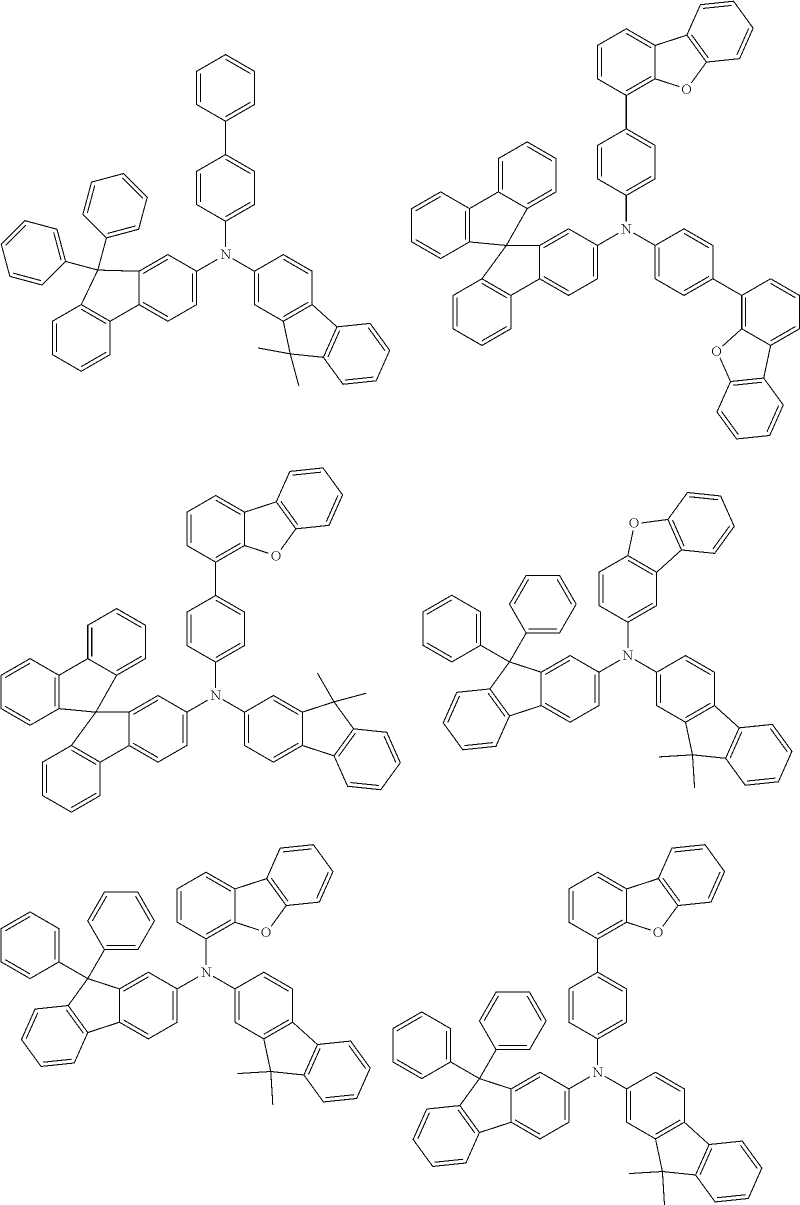

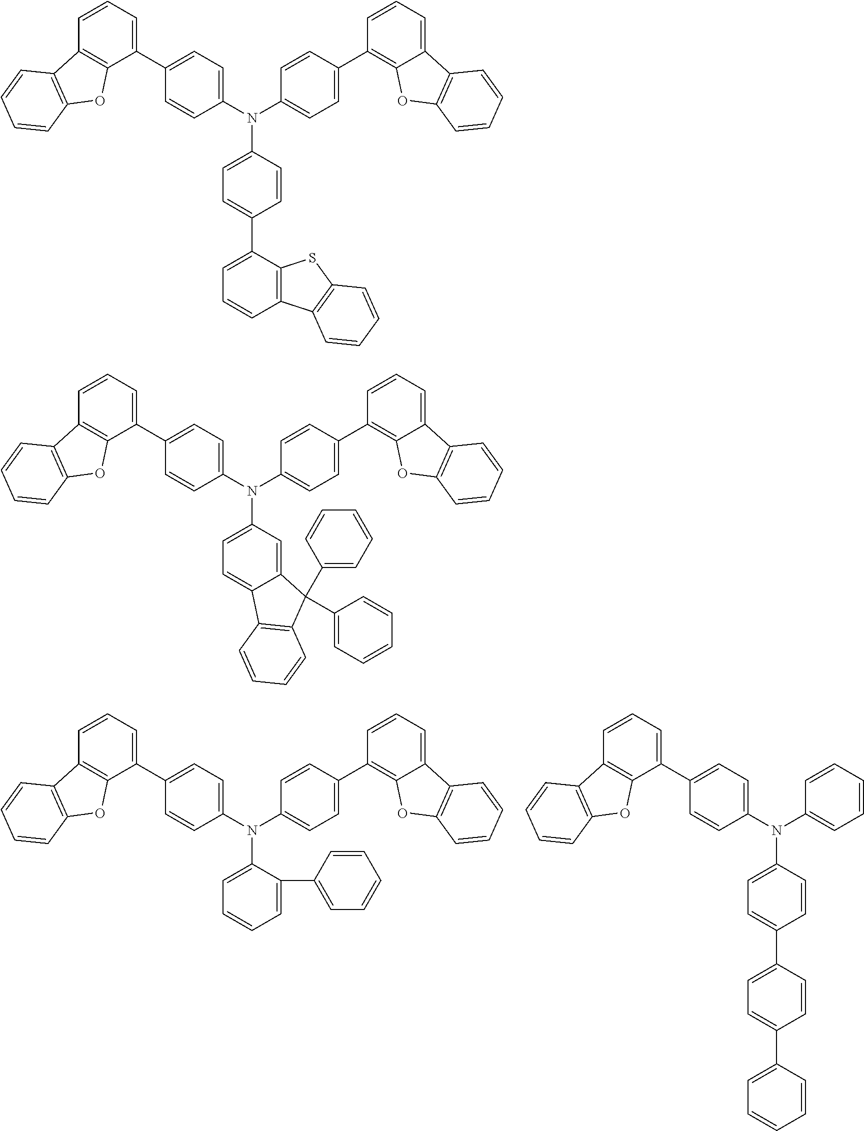

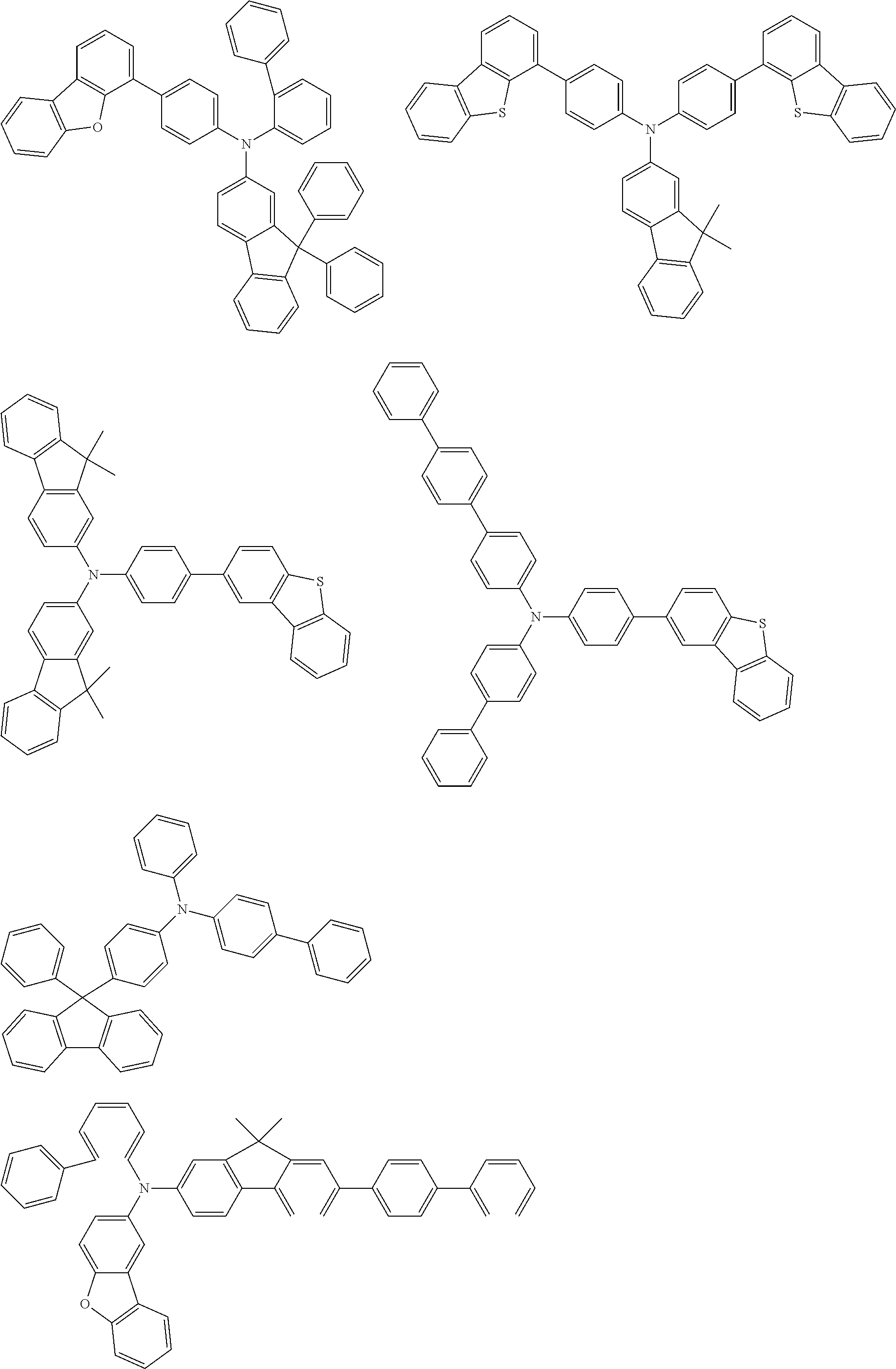

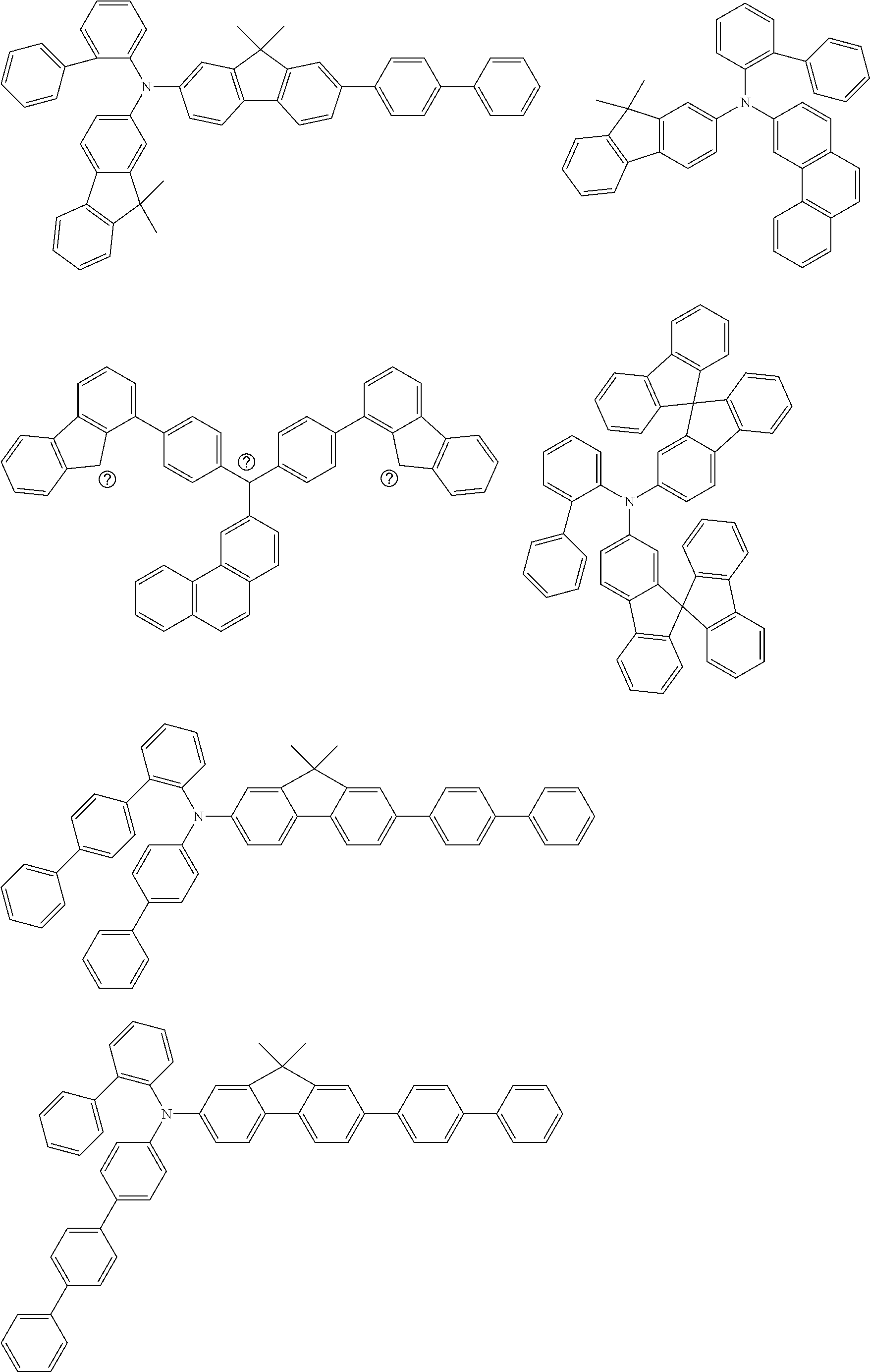

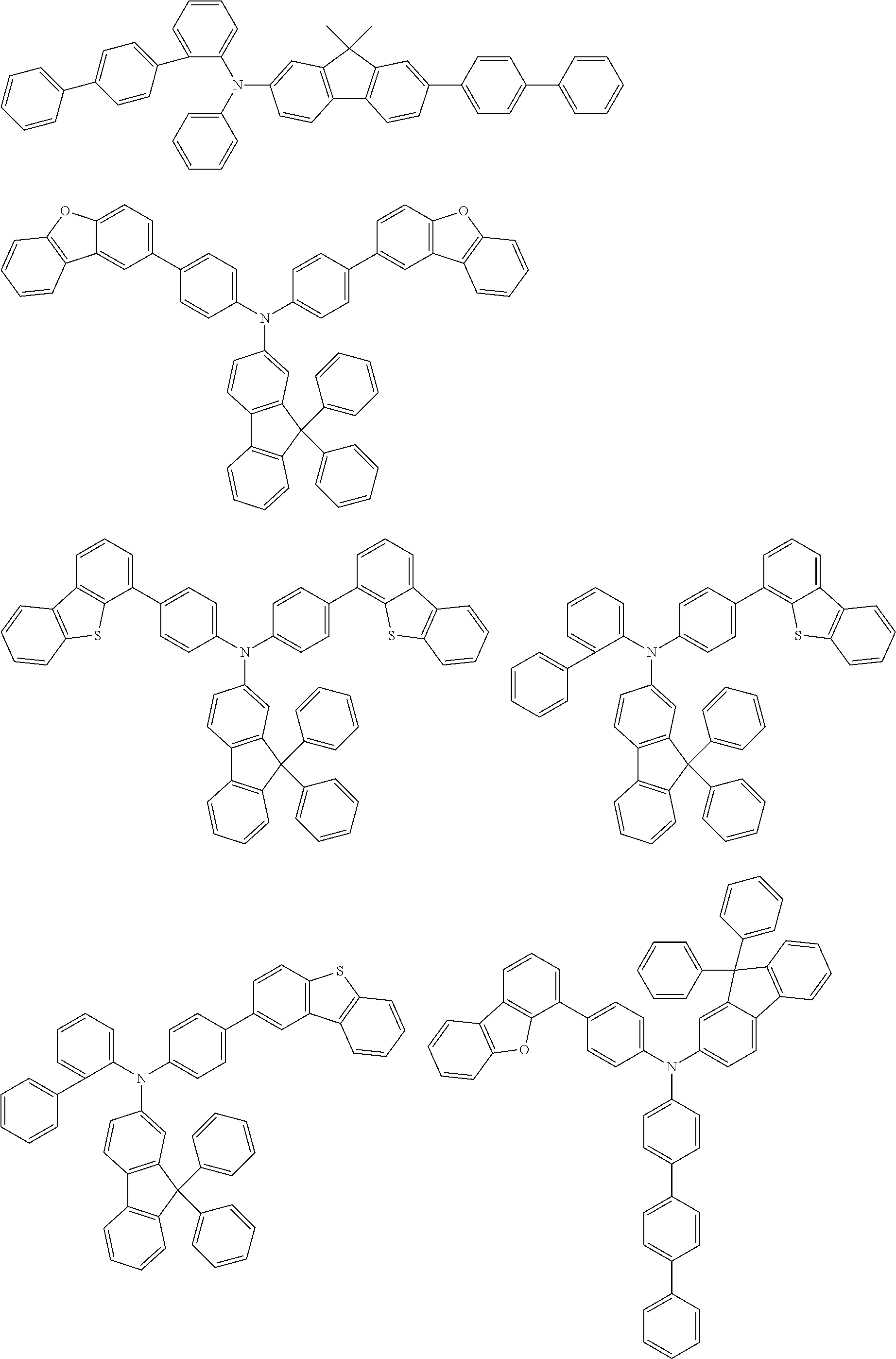

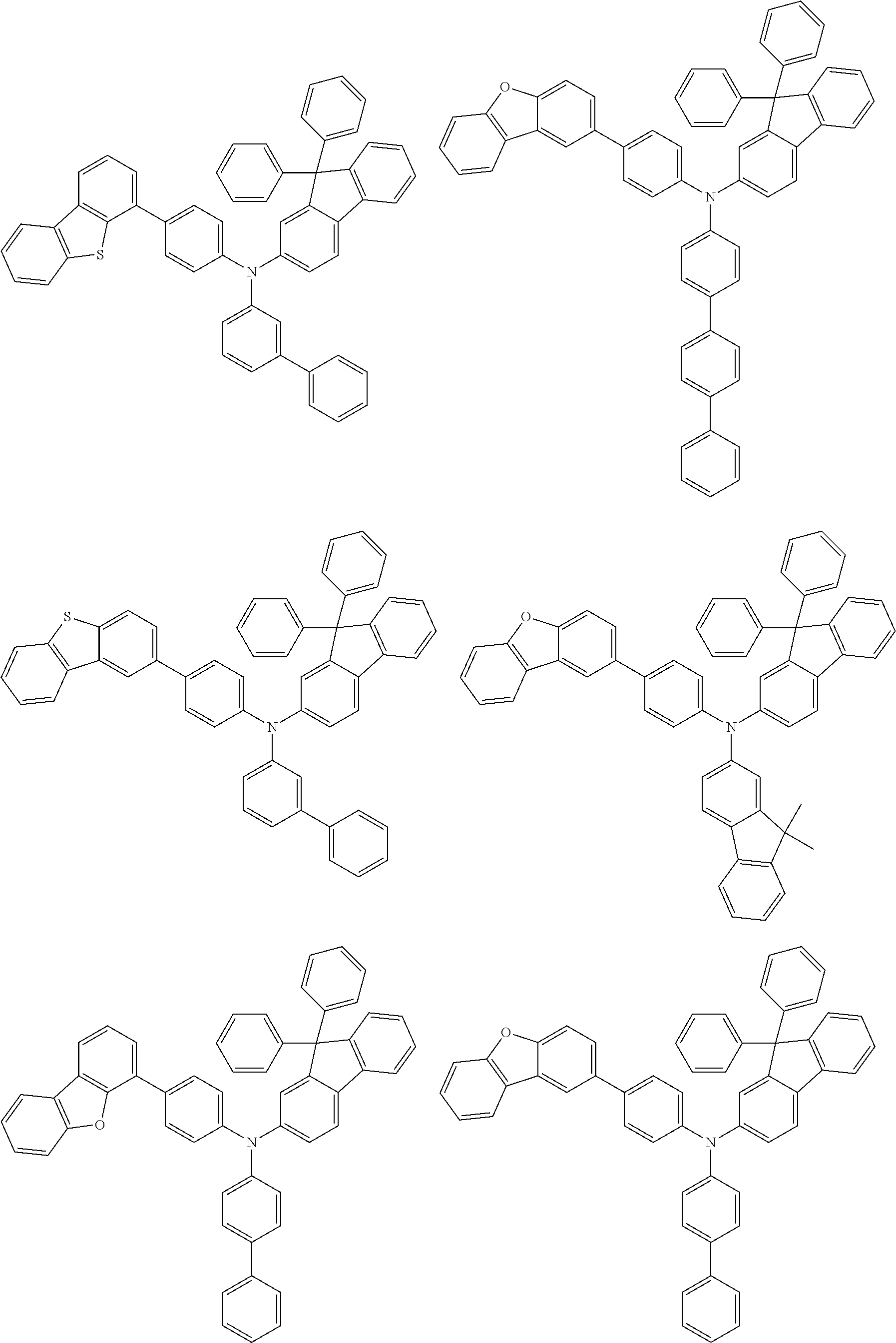

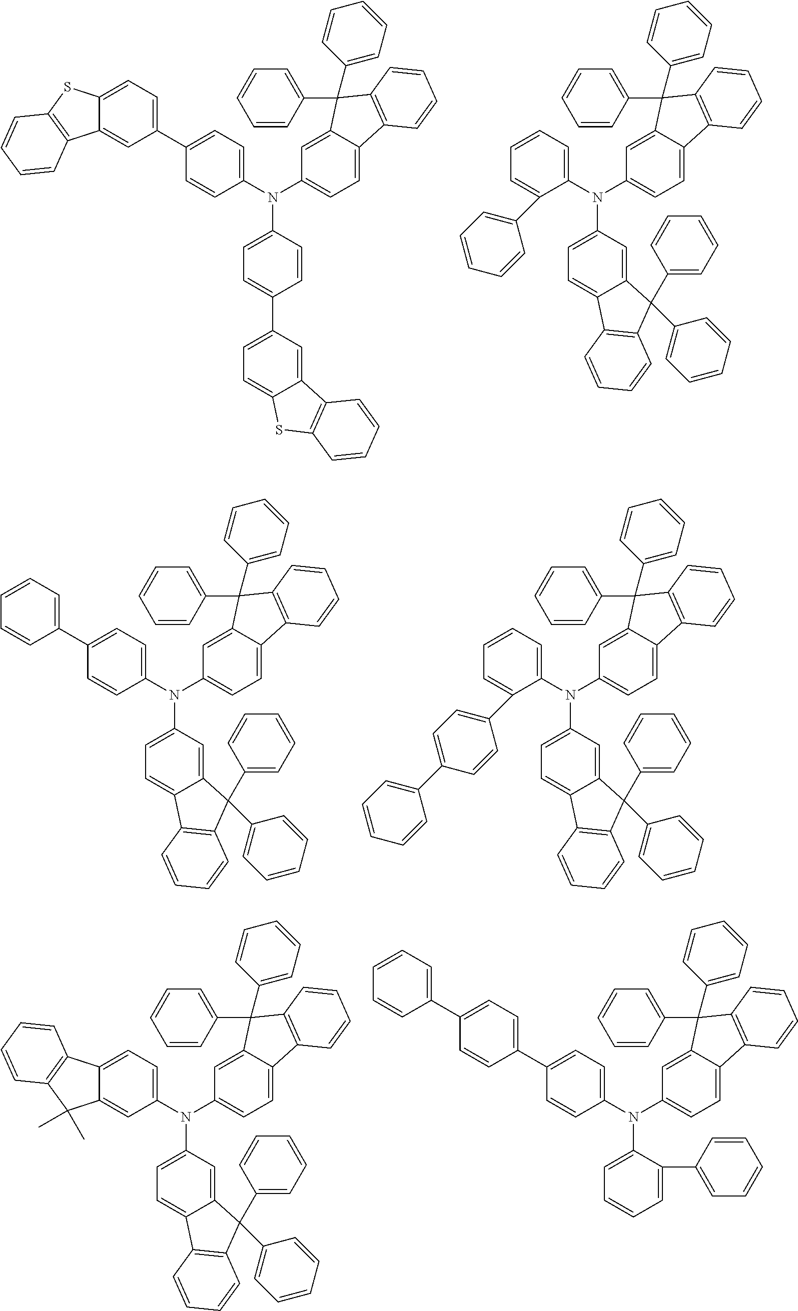

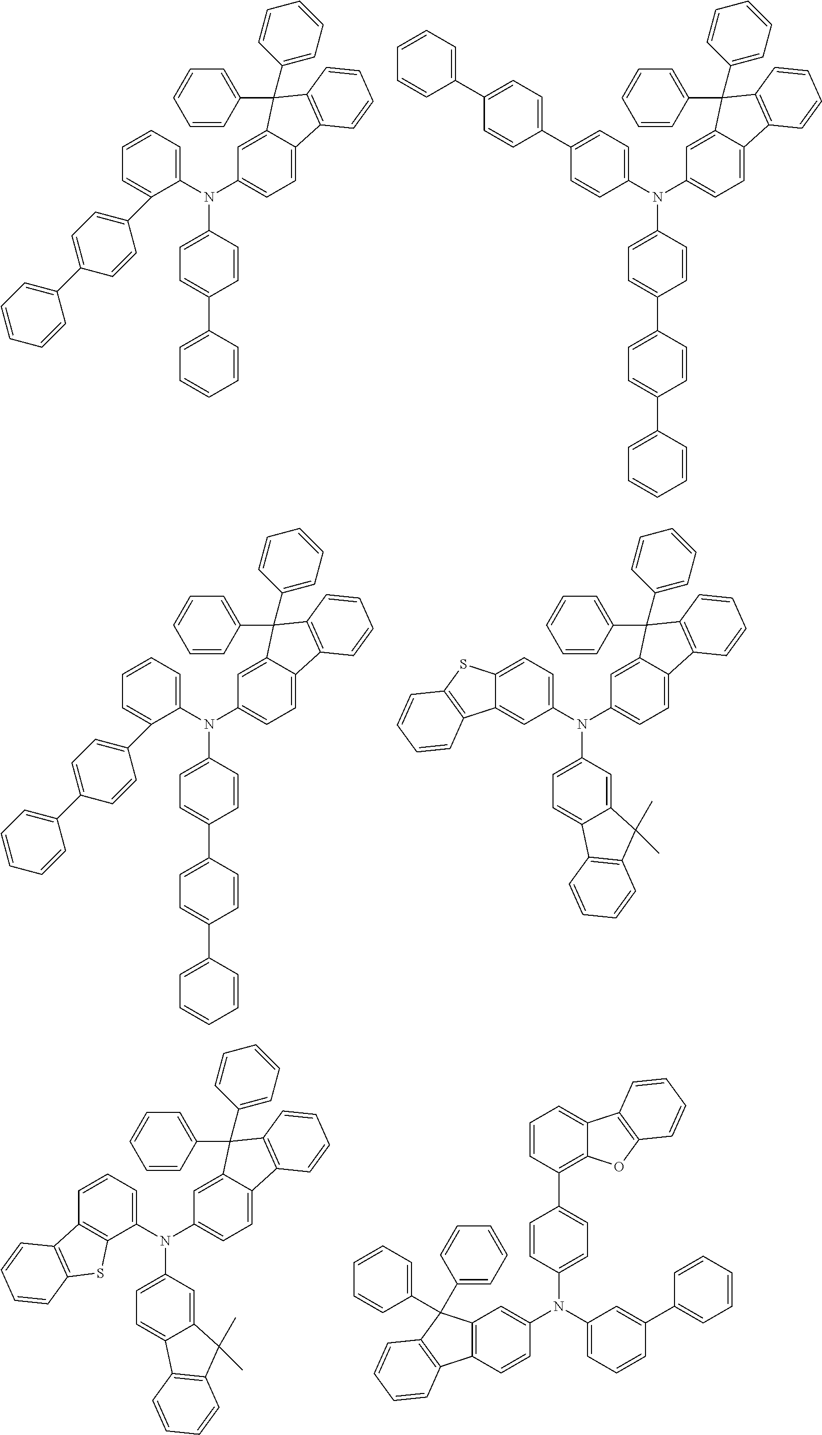

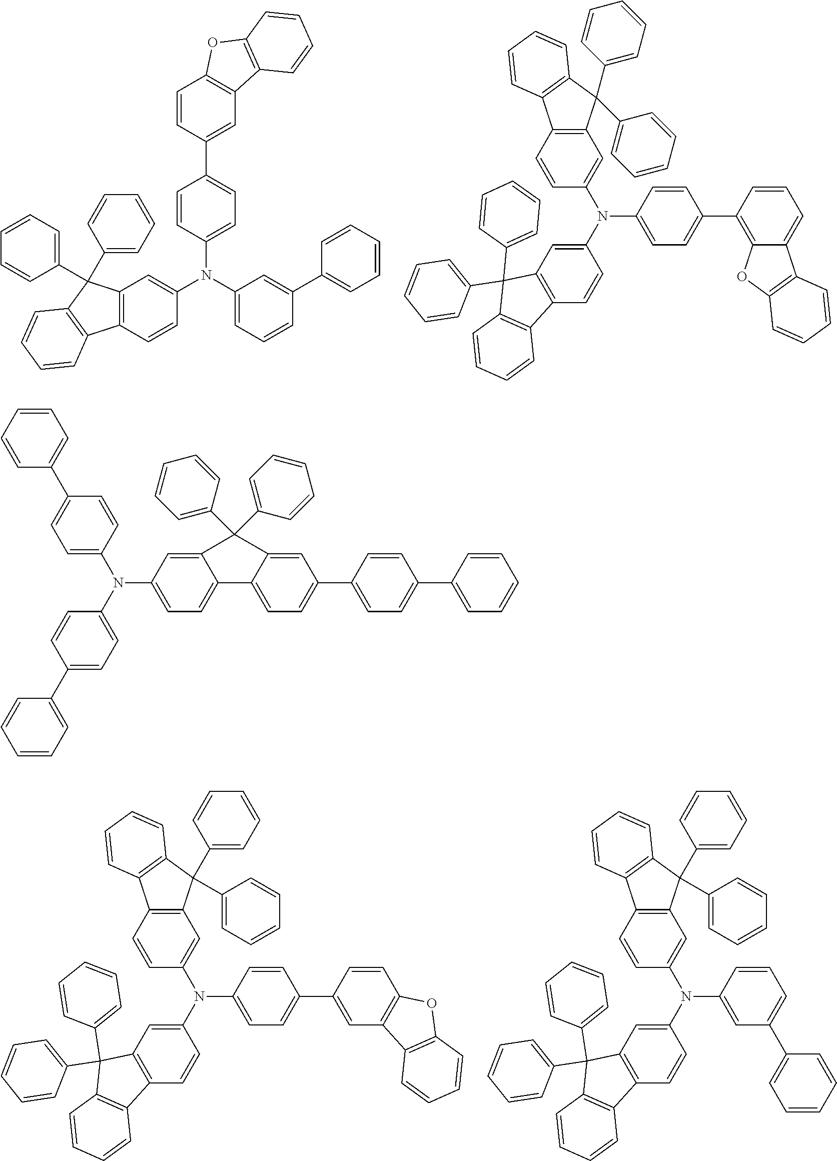

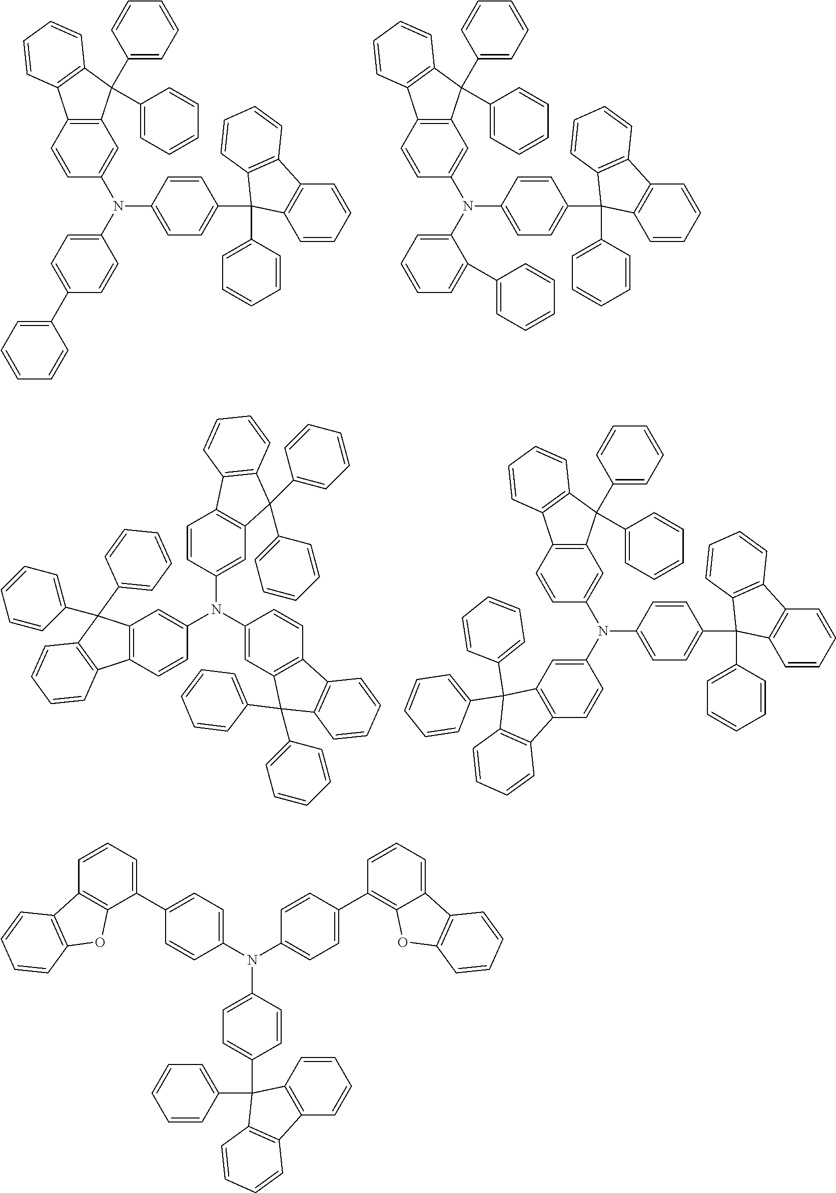

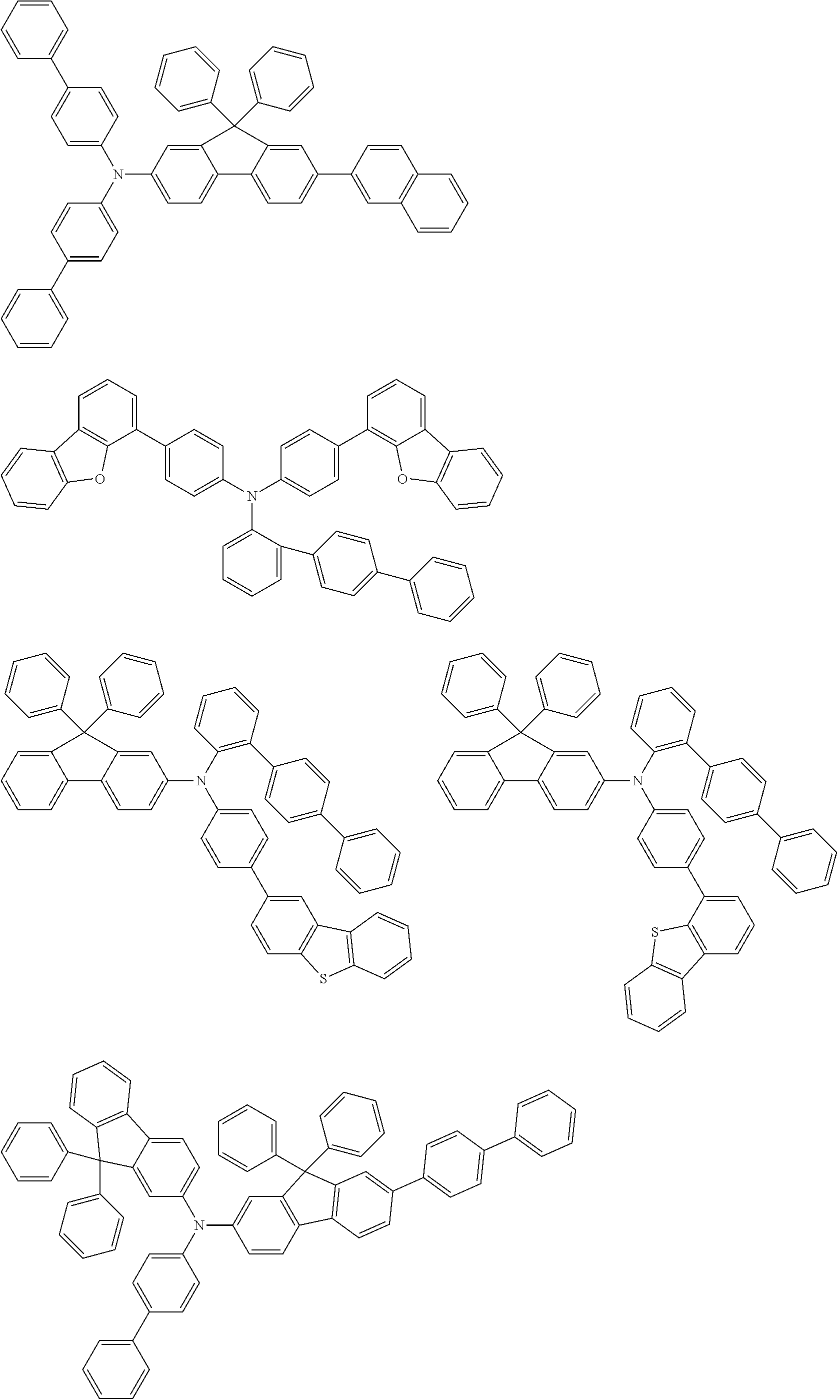

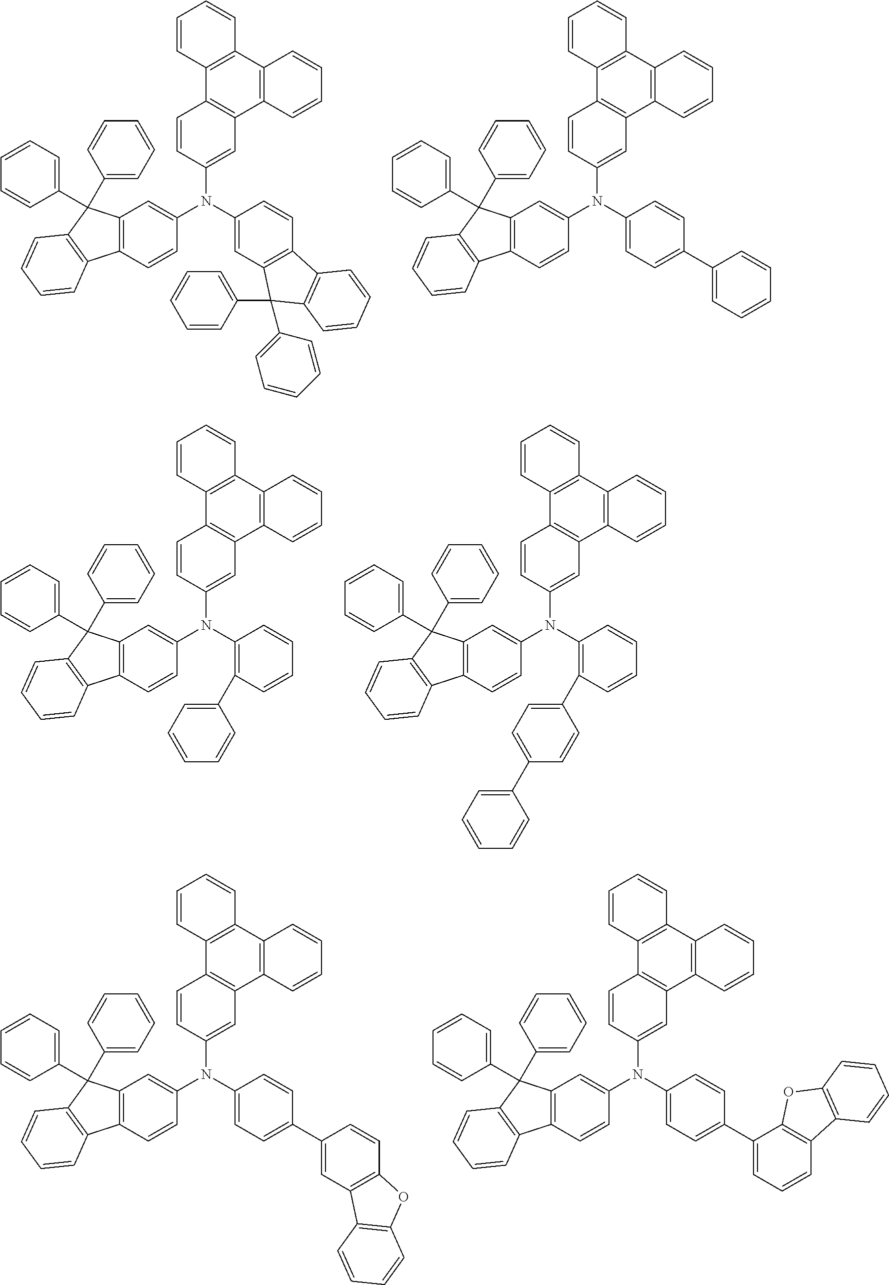

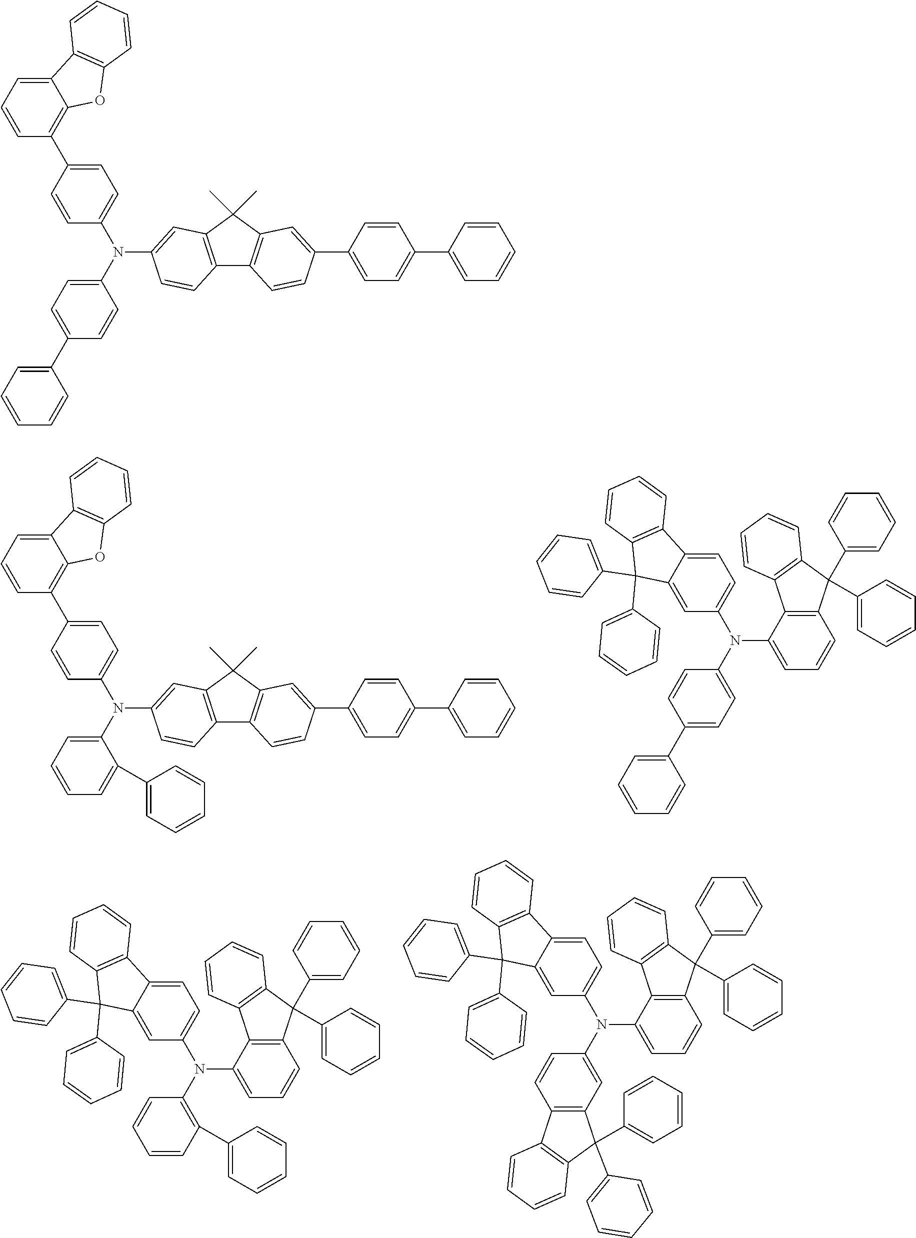

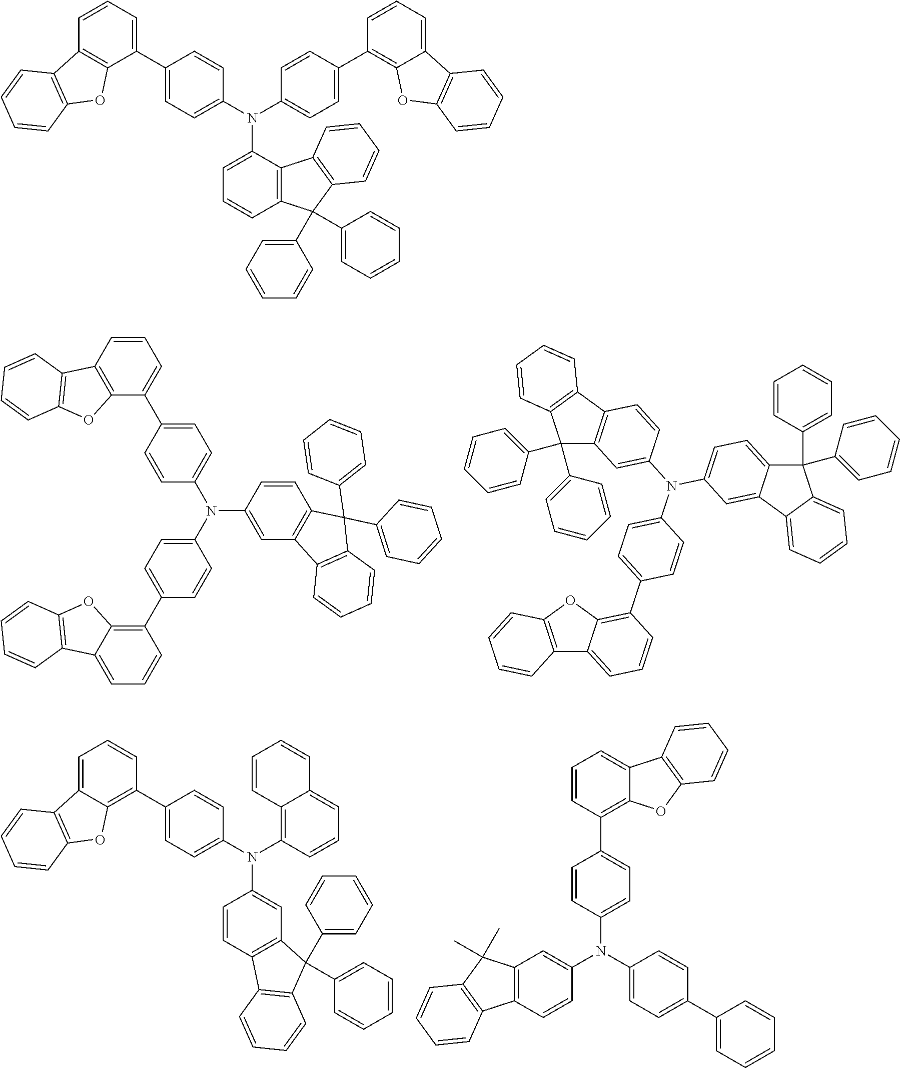

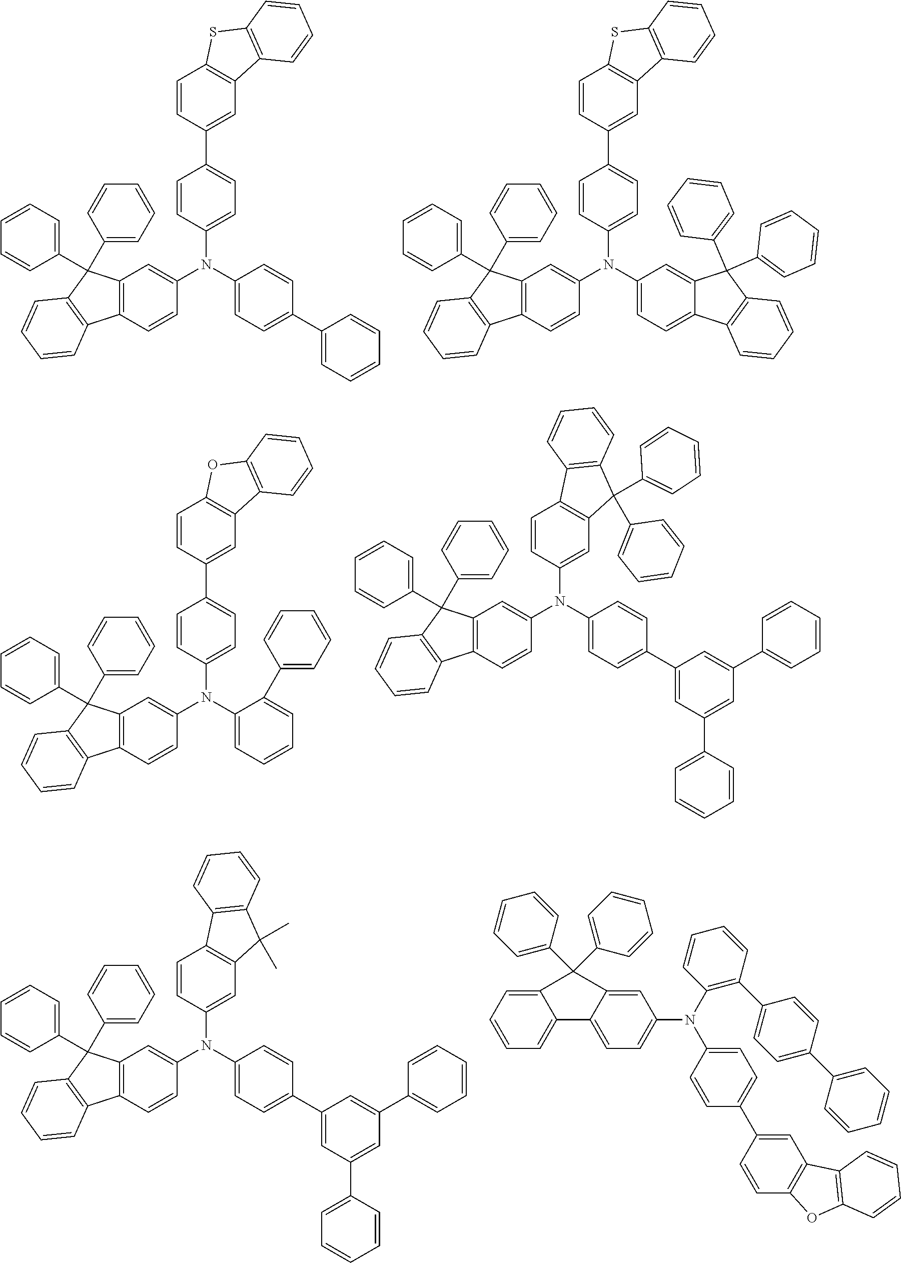

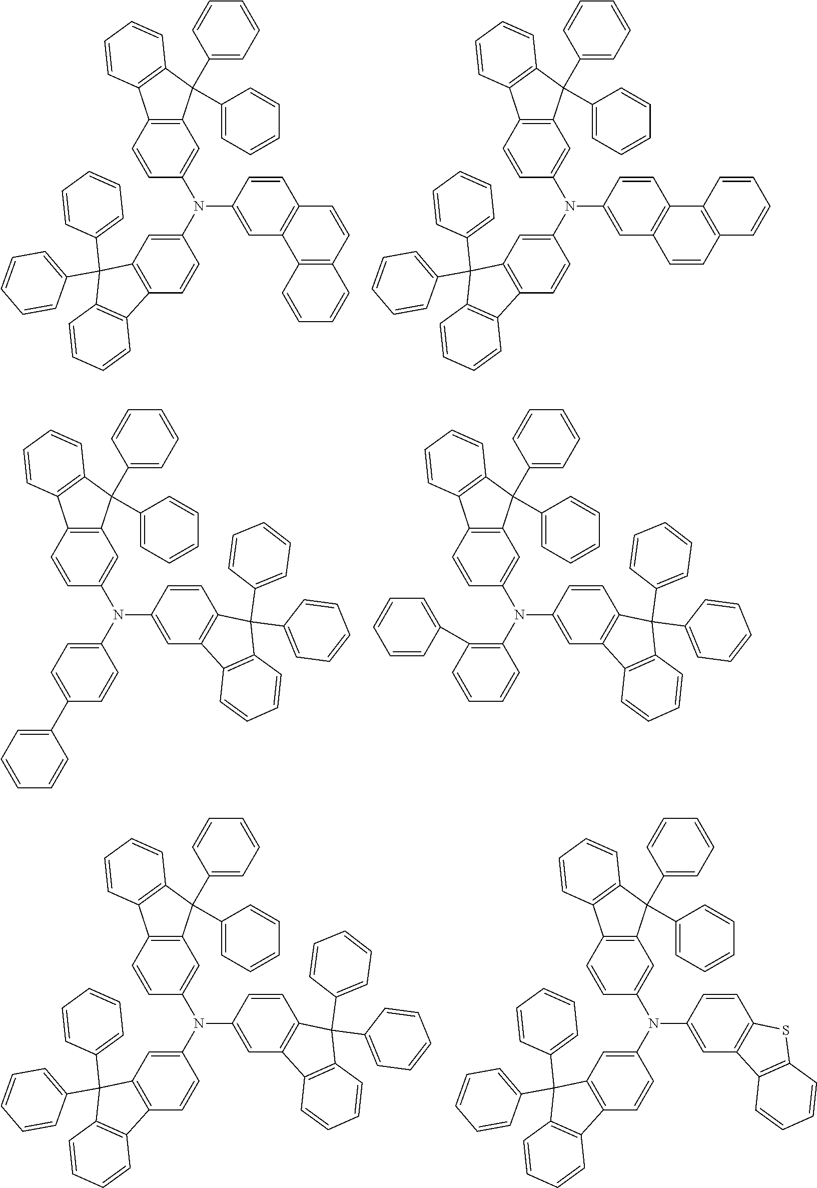

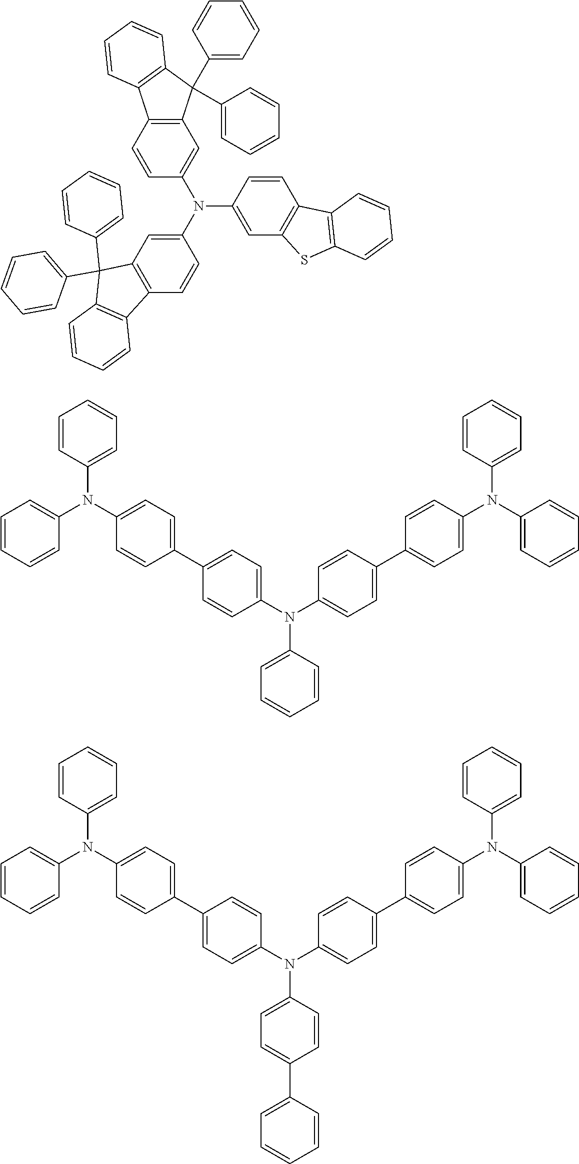

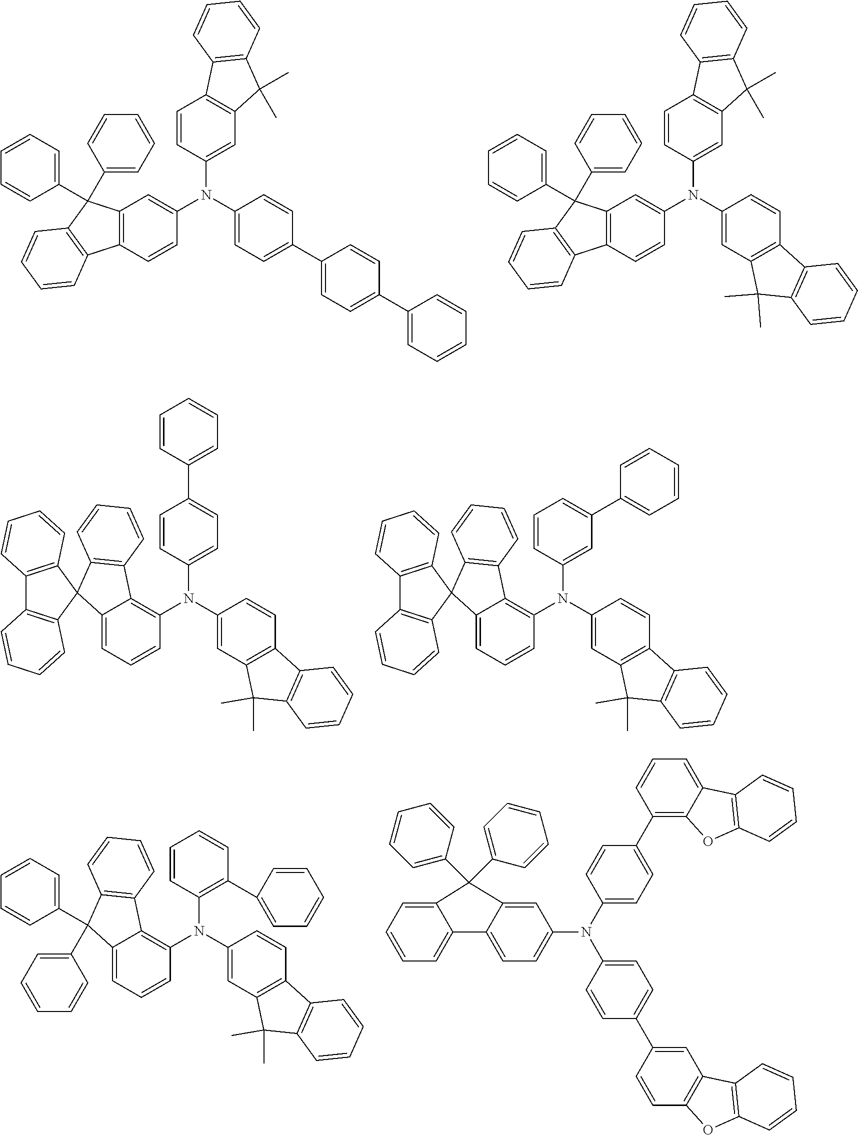

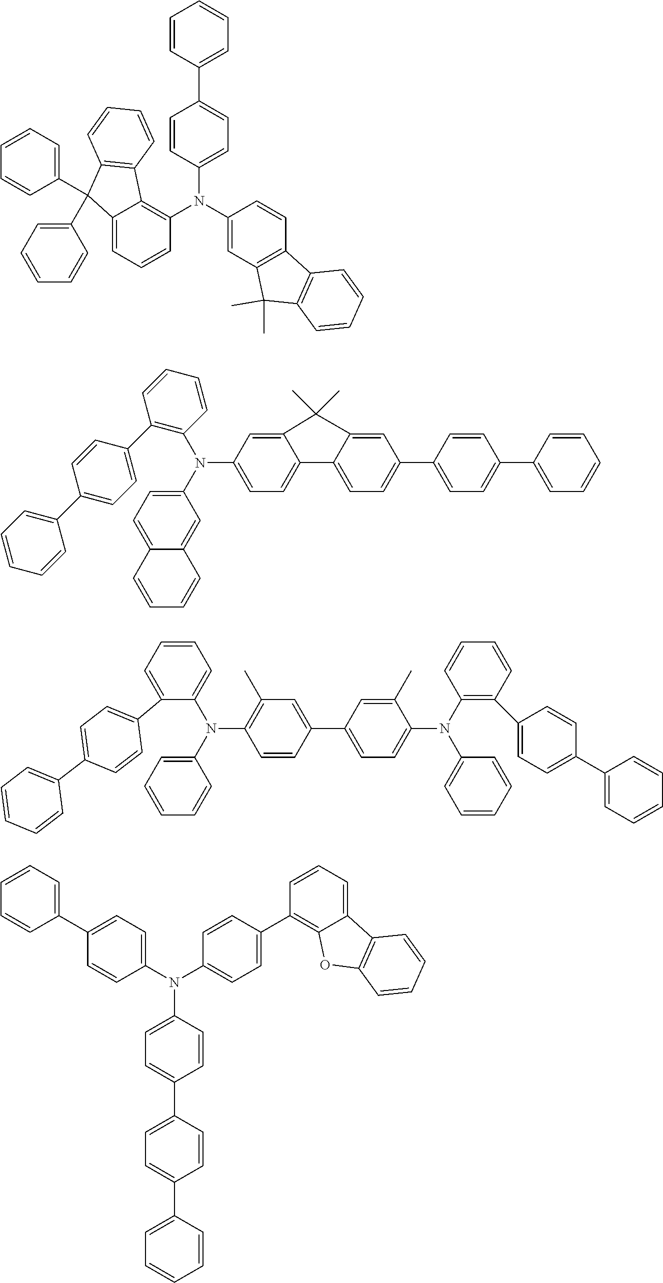

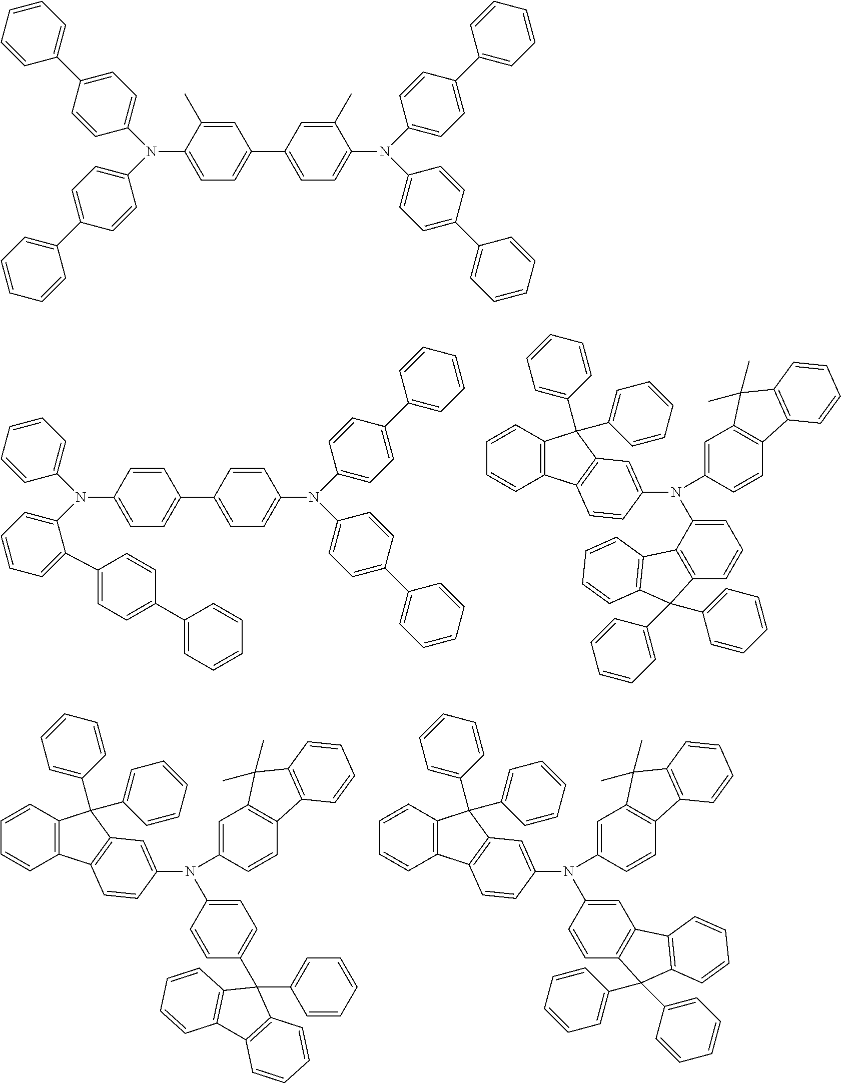

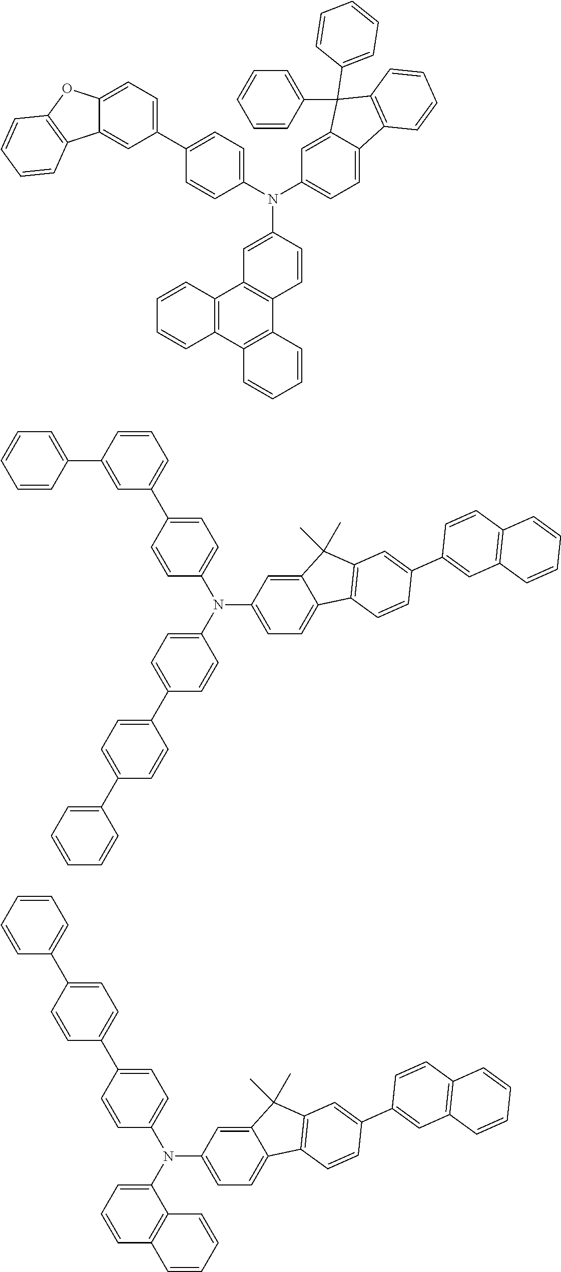

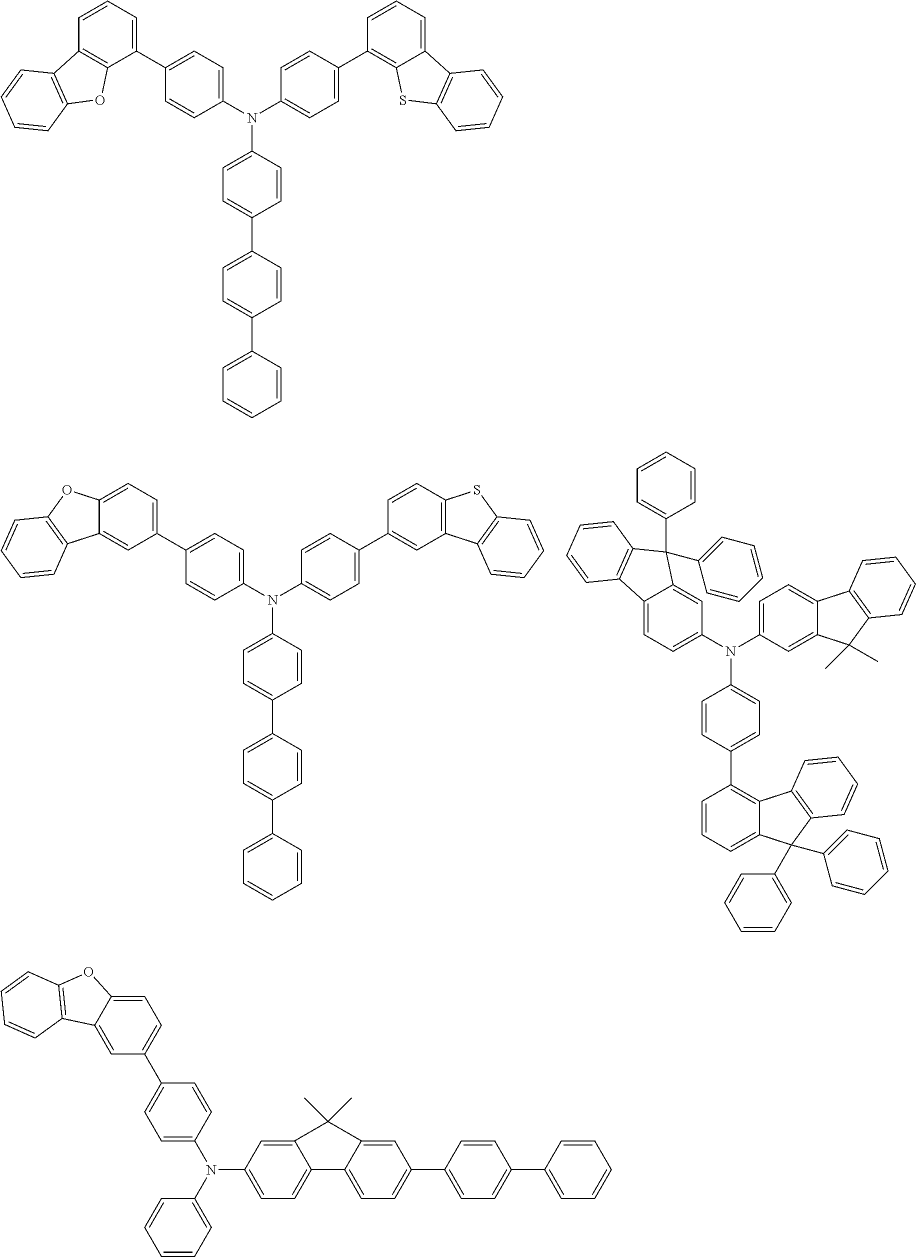

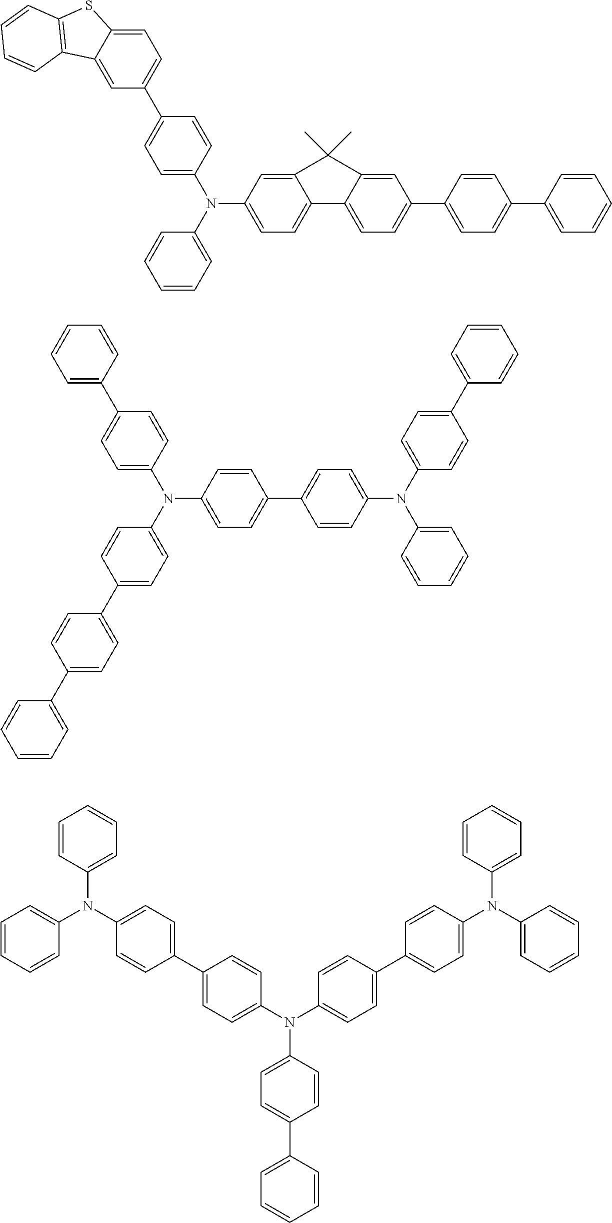 [SMILES: CC1(C)c2cc(-c3ccc(-c4ccccc4)cc3)ccc2-c2ccc(N(c3ccccc3)c3ccc(-c4ccc5sc6ccccc6c5c4)cc3)cc21 ; c1ccc(-c2ccc(-c3ccc(N(c4ccc(-c5ccccc5)cc4)c4ccc(-c5ccc(N(c6ccccc6)c6ccc(-c7ccccc7)cc6)cc5)cc4)cc3)cc2)cc1 ; c1ccc(-c2ccc(N(c3ccc(-c4ccc(N(c5ccccc5)c5ccccc5)cc4)cc3)c3ccc(-c4ccc(N(c5ccccc5)c5ccccc5)cc4)cc3)cc2)cc1]

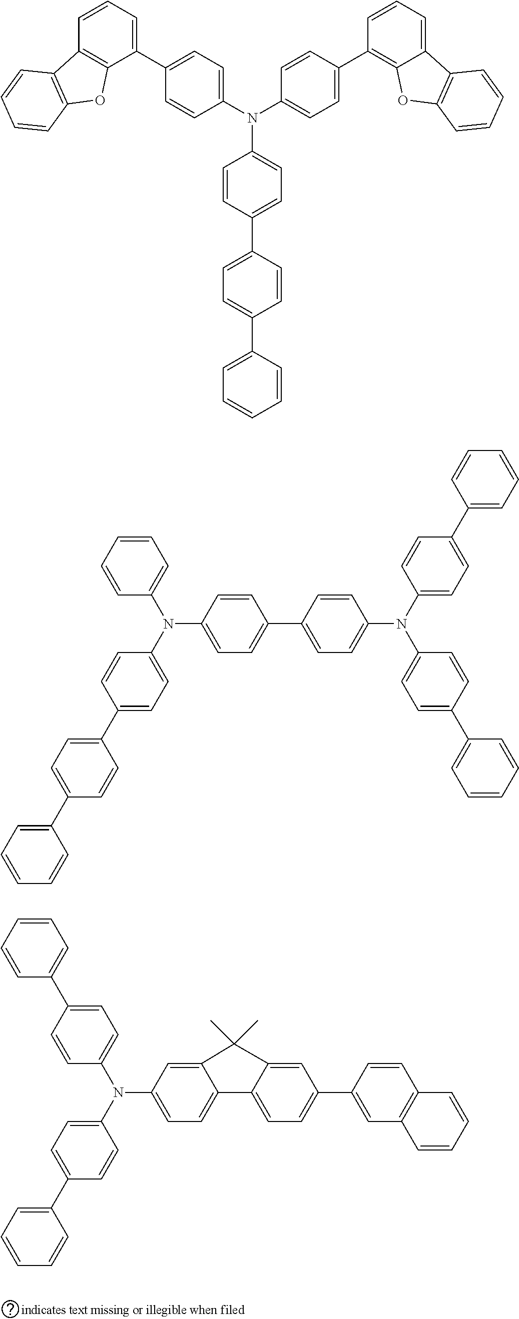

i-Pr is an isopropyl group.

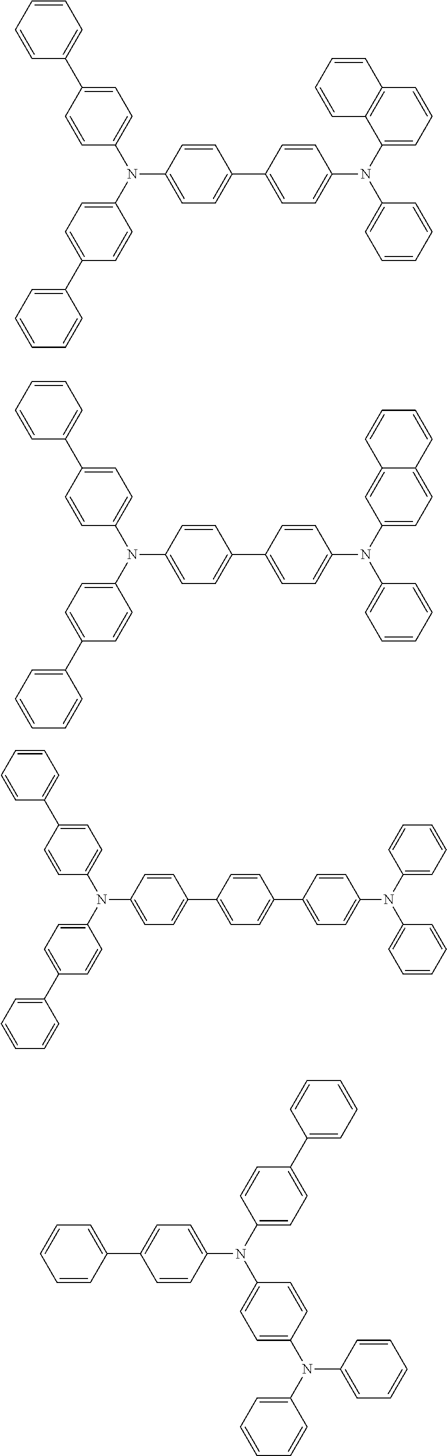

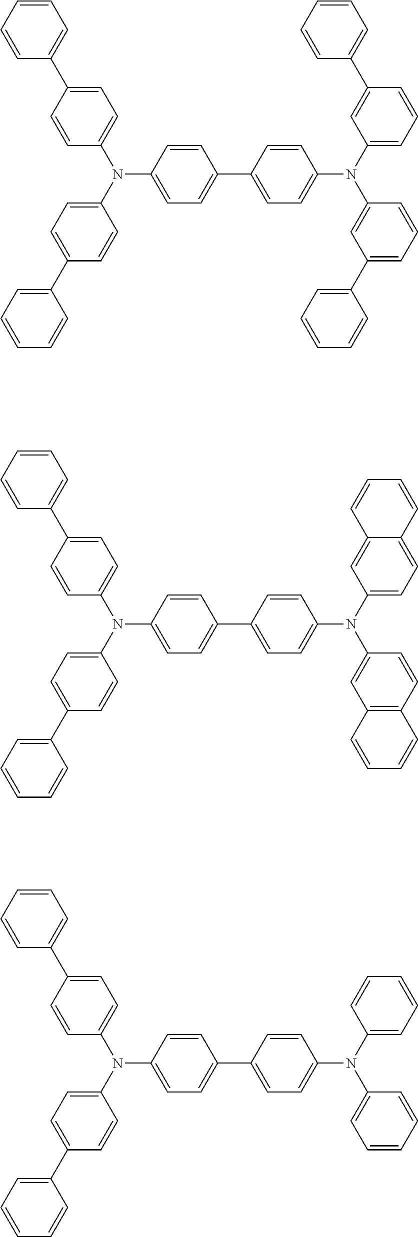 [SMILES: c1ccc(-c2ccc(N(c3ccc(-c4ccccc4)cc3)c3ccc(-c4ccc(N(c5cccc(-c6ccccc6)c5)c5cccc(-c6ccccc6)c5)cc4)cc3)cc2)cc1 ; c1ccc(-c2ccc(N(c3ccc(-c4ccccc4)cc3)c3ccc(-c4ccc(N(c5ccc6ccccc6c5)c5ccc6ccccc6c5)cc4)cc3)cc2)cc1 ; c1ccc(-c2ccc(N(c3ccc(-c4ccccc4)cc3)c3ccc(-c4ccc(N(c5ccccc5)c5ccccc5)cc4)cc3)cc2)cc1]

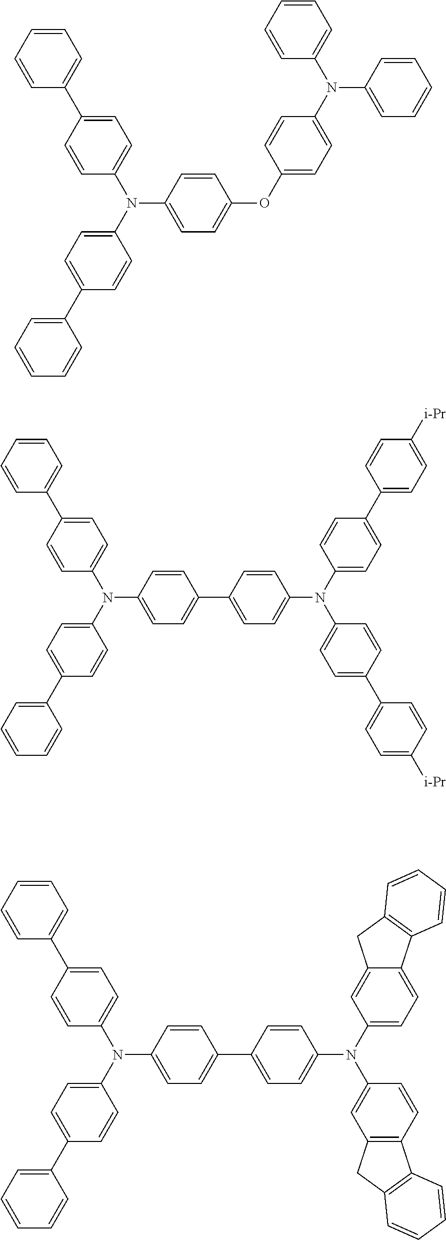 [SMILES: c1ccc(-c2ccc(N(c3ccc(Oc4ccc(N(c5ccccc5)c5ccccc5)cc4)cc3)c3ccc(-c4ccccc4)cc3)cc2)cc1 ; CC(C)c1ccc(-c2ccc(N(c3ccc(-c4ccc(C(C)C)cc4)cc3)c3ccc(-c4ccc(N(c5ccc(-c6ccccc6)cc5)c5ccc(-c6ccccc6)cc5)cc4)cc3)cc2)cc1 ; c1ccc(-c2ccc(N(c3ccc(-c4ccccc4)cc3)c3ccc(-c4ccc(N(c5ccc6c(c5)Cc5ccccc5-6)c5ccc6c(c5)Cc5ccccc5-6)cc4)cc3)cc2)cc1]

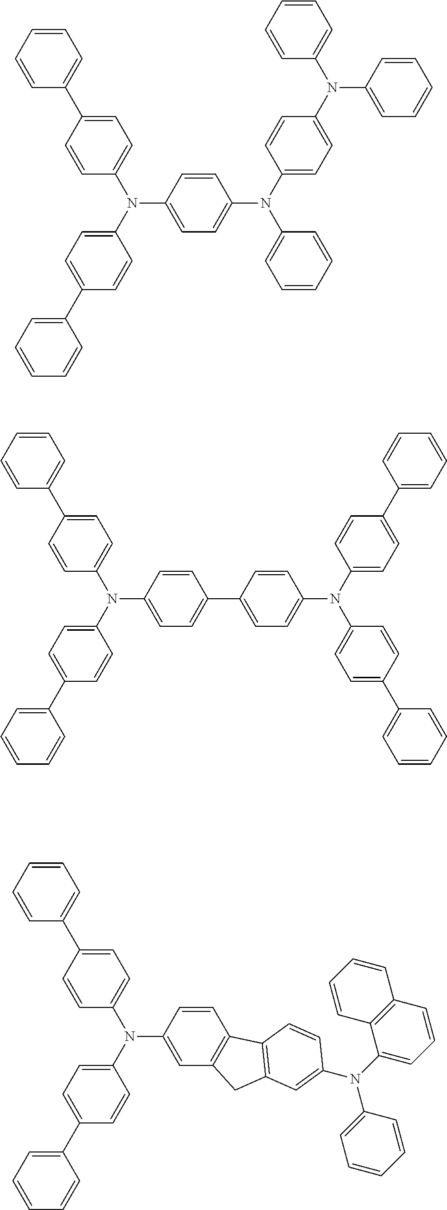 [SMILES: c1ccc(-c2ccc(N(c3ccc(-c4ccccc4)cc3)c3ccc(N(c4ccccc4)c4ccc(N(c5ccccc5)c5ccccc5)cc4)cc3)cc2)cc1 ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(-c2ccc(N(c3ccc(-c4ccccc4)cc3)c3ccc4c(c3)Cc3cc(N(c5ccccc5)c5cccc6ccccc56)ccc3-4)cc2)cc1]

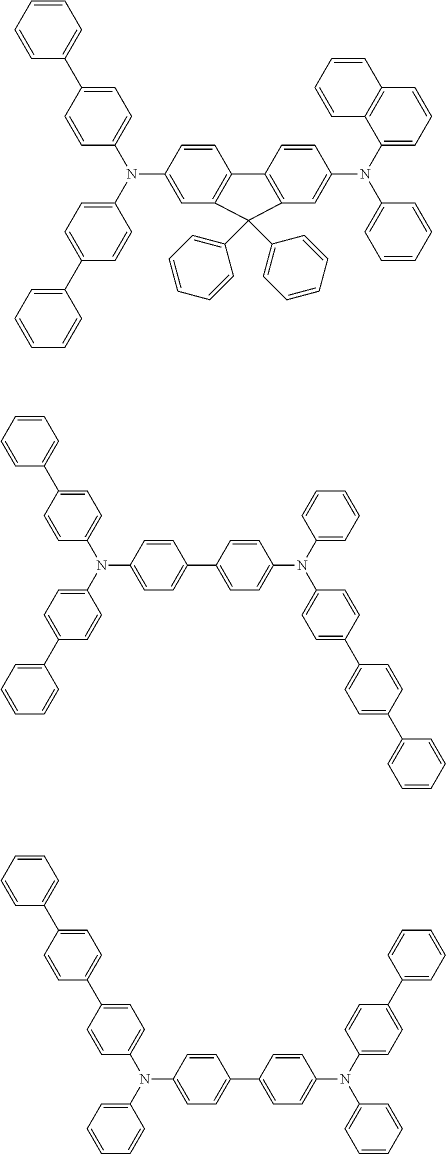 [SMILES: c1ccc(-c2ccc(N(c3ccc(-c4ccccc4)cc3)c3ccc4c(c3)C(c3ccccc3)(c3ccccc3)c3cc(N(c5ccccc5)c5cccc6ccccc56)ccc3-4)cc2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(-c2ccc(-c3ccc(N(c4ccccc4)c4ccc(-c5ccc(N(c6ccccc6)c6ccc(-c7ccccc7)cc6)cc5)cc4)cc3)cc2)cc1]

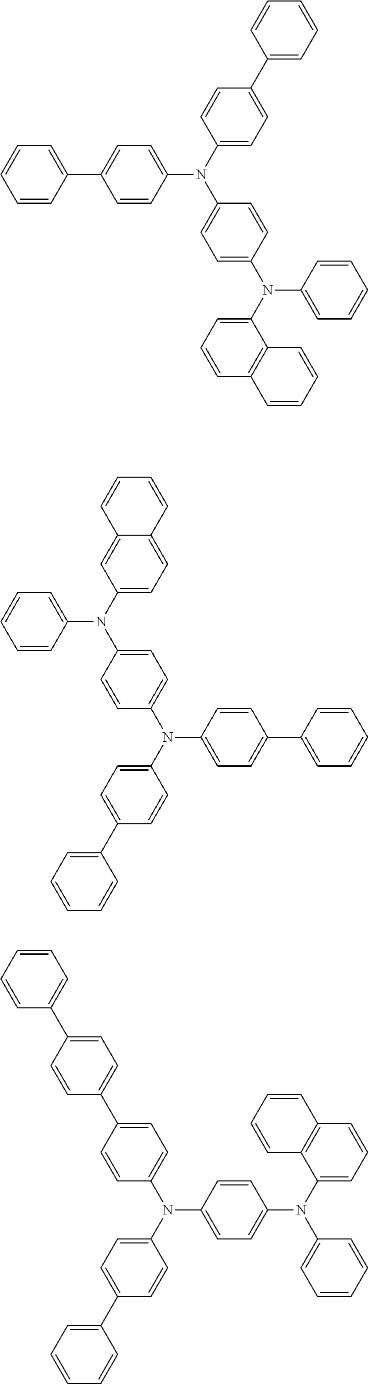

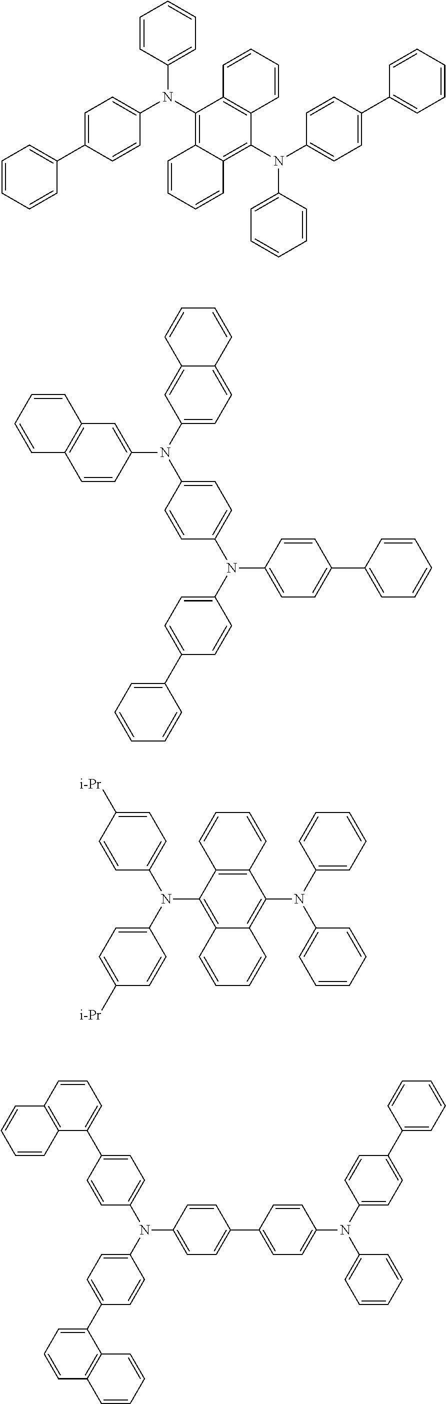 [SMILES: c1ccc(-c2ccc(N(c3ccccc3)c3c4ccccc4c(N(c4ccccc4)c4ccc(-c5ccccc5)cc4)c4ccccc34)cc2)cc1 ; c1ccc(-c2ccc(N(c3ccc(-c4ccccc4)cc3)c3ccc(N(c4ccc5ccccc5c4)c4ccc5ccccc5c4)cc3)cc2)cc1 ; CC(C)c1ccc(N(c2ccc(C(C)C)cc2)c2c3ccccc3c(N(c3ccccc3)c3ccccc3)c3ccccc23)cc1 ; c1ccc(-c2ccc(N(c3ccccc3)c3ccc(-c4ccc(N(c5ccc(-c6cccc7ccccc67)cc5)c5ccc(-c6cccc7ccccc67)cc5)cc4)cc3)cc2)cc1]

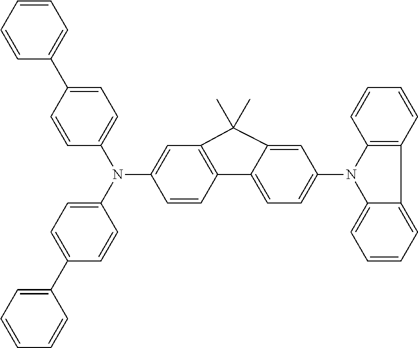 [SMILES: CC1(C)c2cc(N(c3ccc(-c4ccccc4)cc3)c3ccc(-c4ccccc4)cc3)ccc2-c2ccc(-n3c4ccccc4c4ccccc43)cc21]

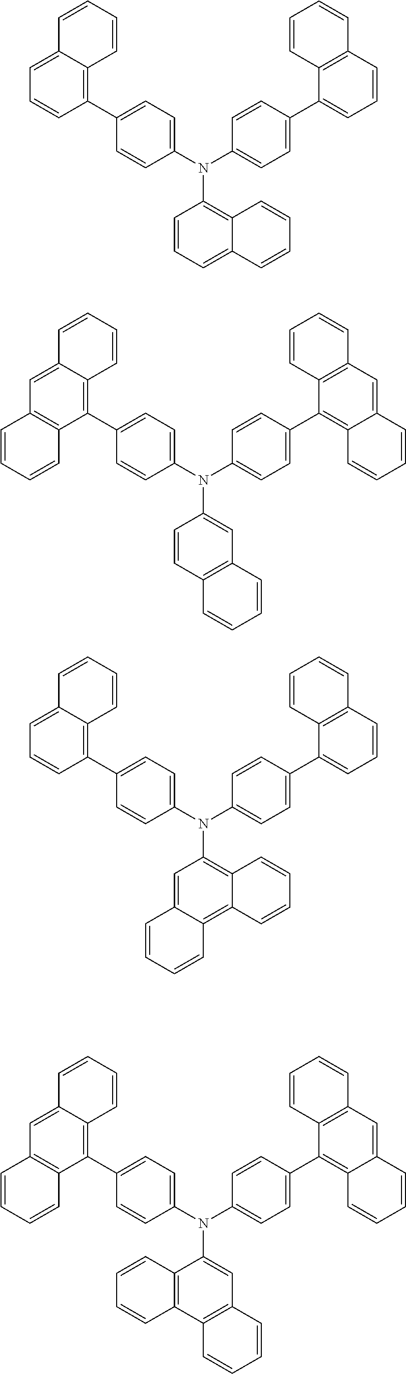 [SMILES: c1ccc2c(-c3ccc(N(c4ccc(-c5cccc6ccccc56)cc4)c4cccc5ccccc45)cc3)cccc2c1 ; c1ccc2cc(N(c3ccc(-c4c5ccccc5cc5ccccc45)cc3)c3ccc(-c4c5ccccc5cc5ccccc45)cc3)ccc2c1 ; c1ccc2c(-c3ccc(N(c4ccc(-c5cccc6ccccc56)cc4)c4cc5ccccc5c5ccccc45)cc3)cccc2c1 ; c1ccc2c(-c3ccc(N(c4ccc(-c5c6ccccc6cc6ccccc56)cc4)c4cc5ccccc5c5ccccc45)cc3)c3ccccc3cc2c1]

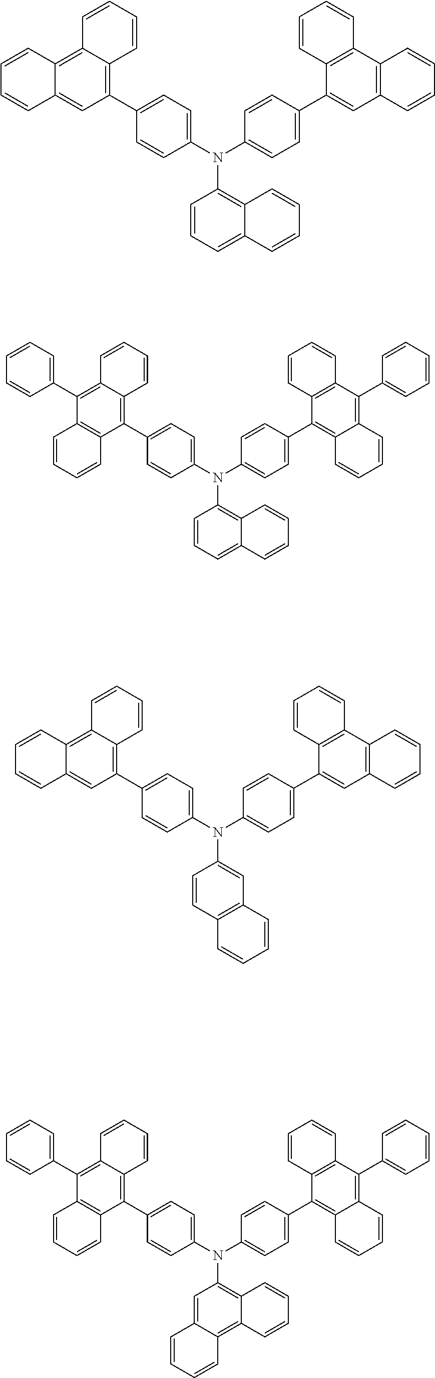 [SMILES: c1ccc2c(N(c3ccc(-c4cc5ccccc5c5ccccc45)cc3)c3ccc(-c4cc5ccccc5c5ccccc45)cc3)cccc2c1 ; c1ccc(-c2c3ccccc3c(-c3ccc(N(c4ccc(-c5c6ccccc6c(-c6ccccc6)c6ccccc56)cc4)c4cccc5ccccc45)cc3)c3ccccc23)cc1 ; c1ccc2cc(N(c3ccc(-c4cc5ccccc5c5ccccc45)cc3)c3ccc(-c4cc5ccccc5c5ccccc45)cc3)ccc2c1 ; c1ccc(-c2c3ccccc3c(-c3ccc(N(c4ccc(-c5c6ccccc6c(-c6ccccc6)c6ccccc56)cc4)c4cc5ccccc5c5ccccc45)cc3)c3ccccc23)cc1]

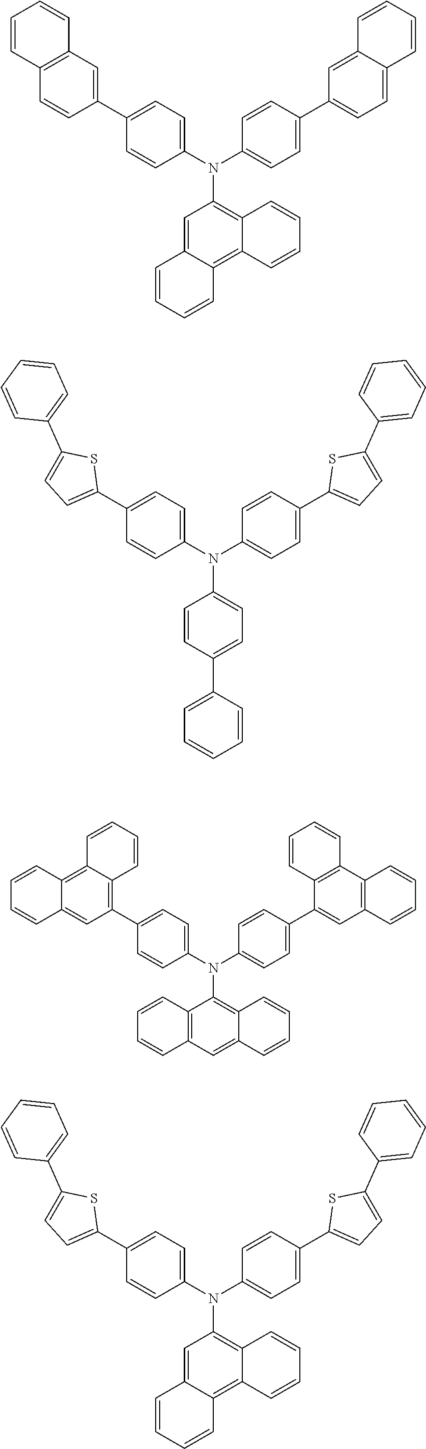 [SMILES: c1ccc2cc(-c3ccc(N(c4ccc(-c5ccc6ccccc6c5)cc4)c4cc5ccccc5c5ccccc45)cc3)ccc2c1 ; c1ccc(-c2ccc(N(c3ccc(-c4ccc(-c5ccccc5)s4)cc3)c3ccc(-c4ccc(-c5ccccc5)s4)cc3)cc2)cc1 ; c1ccc2c(N(c3ccc(-c4cc5ccccc5c5ccccc45)cc3)c3ccc(-c4cc5ccccc5c5ccccc45)cc3)c3ccccc3cc2c1 ; c1ccc(-c2ccc(-c3ccc(N(c4ccc(-c5ccc(-c6ccccc6)s5)cc4)c4cc5ccccc5c5ccccc45)cc3)s2)cc1]

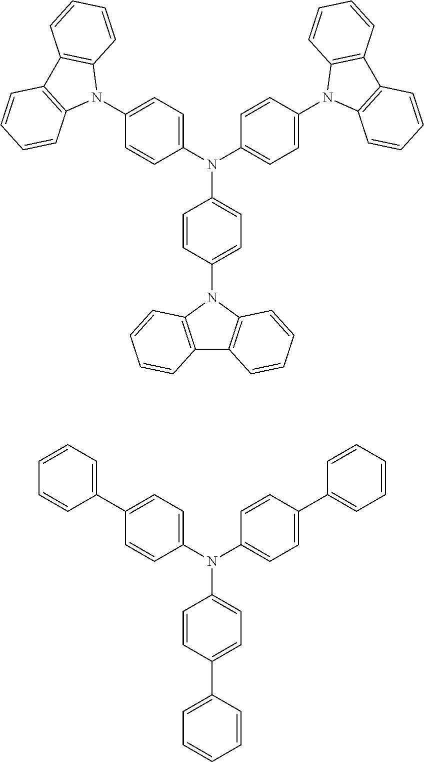 [SMILES: c1ccc2c(c1)c1ccccc1n2-c1ccc(N(c2ccc(-n3c4ccccc4c4ccccc43)cc2)c2ccc(-n3c4ccccc4c4ccccc43)cc2)cc1 ; c1ccc(-c2ccc(N(c3ccc(-c4ccccc4)cc3)c3ccc(-c4ccccc4)cc3)cc2)cc1]

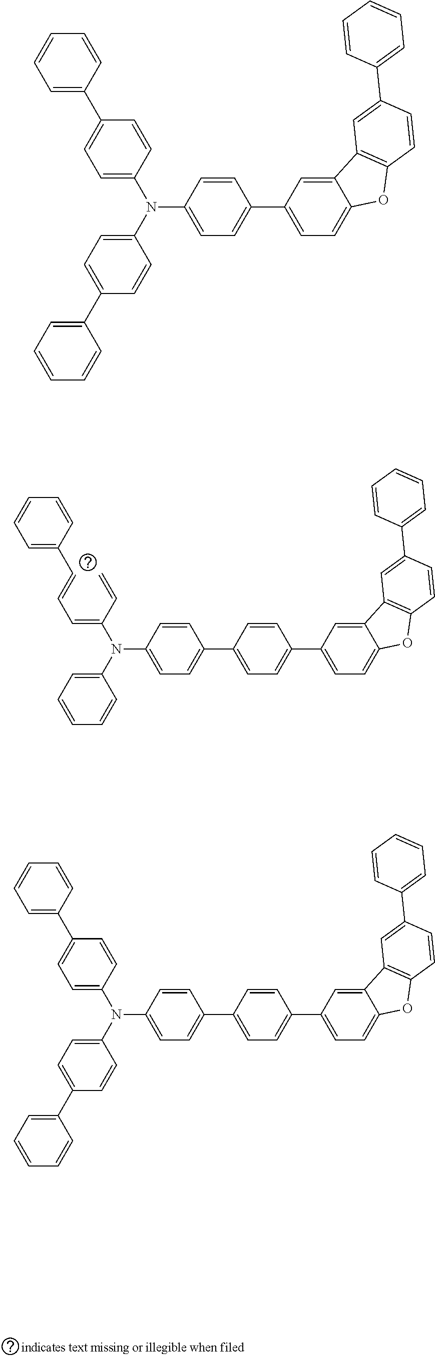 [SMILES: c1ccc(-c2ccc(N(c3ccc(-c4ccccc4)cc3)c3ccc(-c4ccc5oc6ccc(-c7ccccc7)cc6c5c4)cc3)cc2)cc1 ; C=C/C(=C\C=C\c1ccccc1)N(c1ccccc1)c1ccc(-c2ccc(-c3ccc4oc5ccc(-c6ccccc6)cc5c4c3)cc2)cc1 ; c1ccc(-c2ccc(N(c3ccc(-c4ccccc4)cc3)c3ccc(-c4ccc(-c5ccc6oc7ccc(-c8ccccc8)cc7c6c5)cc4)cc3)cc2)cc1]

In the compounds represented by the formulas (11) to (14), examples of the substituent, in the case of “a substituted or unsubstituted” includes a substituted or unsubstituted aryl group, a substituted or unsubstituted dibenzofuranyl group, a substituted or unsubstituted dibenzothiophenyl group, a substituted or unsubstituted alkyl group, a substituted or unsubstituted alkoxy group, a substituted or unsubstituted aralkyl group, a substituted or unsubstituted aryloxy group, a substituted or unsubstituted arylthio group, a substituted or unsubstituted alkoxycarbonyl group, an amino group substituted with a substituted or unsubstituted aryl group, an amino group substituted with a substituted or unsubstituted dibenzofuranyl group or a substituted or unsubstituted dibenzothiophenyl group, a halogen atom, a cyano group, a nitro group, a hydroxyl group, or a carboxyl group, or the like, as described in Ar₁₁ to Ar₁₇ and R₁ to R₃. Specific examples of each of these substituents are the same as those described above as Ar₁₁ to Ar₁₃ and R₁ to R₃.

Among the substituents mentioned above, an unsubstituted aryl group, a substituted or unsubstituted dibenzofuranyl group, and a substituted or unsubstituted dibenzothiophenyl group are preferable, and further, preferable specific substituents in the explanation of each substituent are preferable.

These substituents may be further substituted with the above substituents.

An ionization potential of the compound represented by the formula (11) is preferably smaller than an ionization potential of the compound represented by the formula (1).

The value of “(the ionization potential of the compound represented by the formula (1))−(the ionization potential of the compound represented by the formula (11))” is preferably more than 0 eV and 0.25 eV or less, more preferably 0.10 eV or more and 0.25 eV or less, and particularly preferably 0.20 eV or more and 0.25 eV or less.

The ionization potentials can be measured under atmospheric conditions using a photoelectron spectrometer AC-3 (manufactured by Riken Keiki Co., Ltd.). Specifically, it is measured by irradiating light to a compound to be measured, and measuring the amount of electrons generated by charge separation at that time.

The light-emitting layer may further comprise a complex of a metal.

The complex of a metal preferably comprises a metal atom selected from Ir, Pt, Tb, Eu, Os, Au, Cu, Re and Ru, and a ligand. The ligand preferably comprises an ortho-metalated bond.

An ortho-metallated complex of a metal atom selected from Ir, Tb and Eu is more preferable in that the quantum yield is high and the external quantum efficiency of the light-emitting device can be further improved.

Specific examples of preferable metal complexes are shown below.

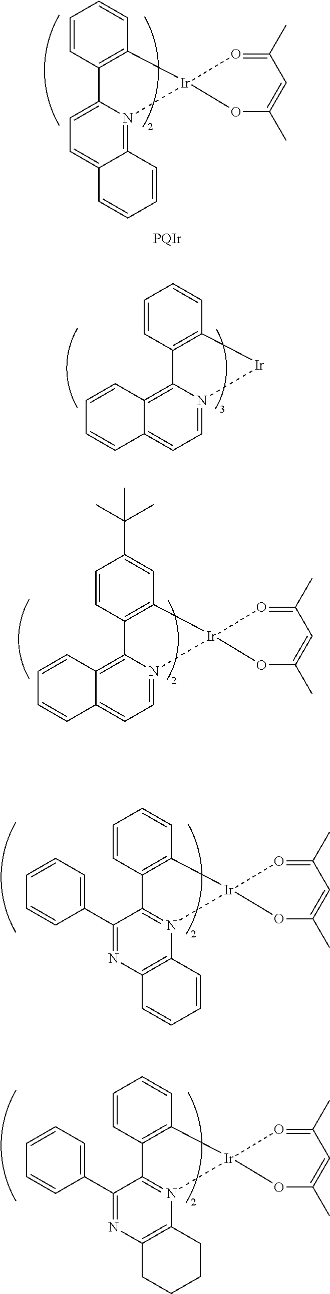

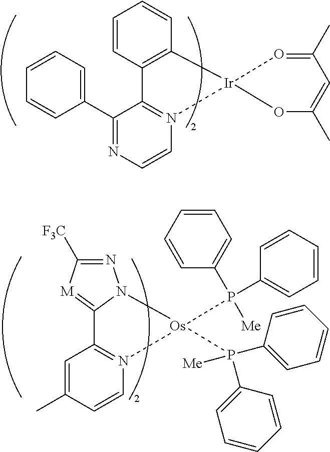

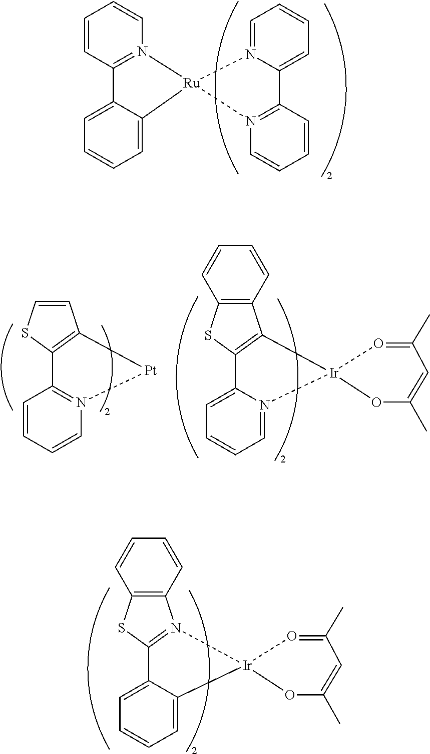

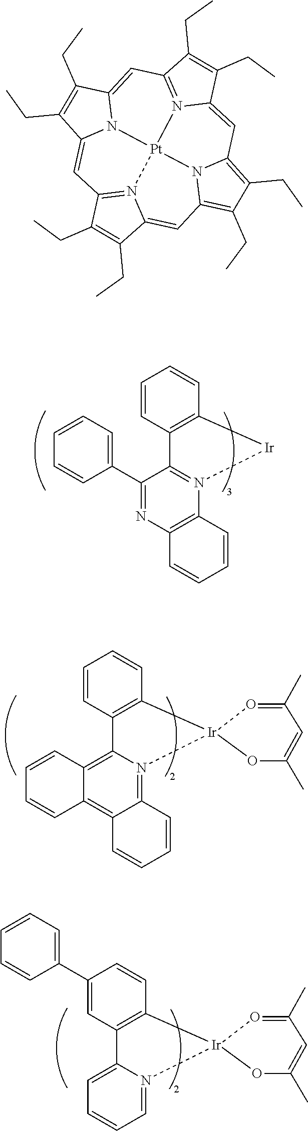

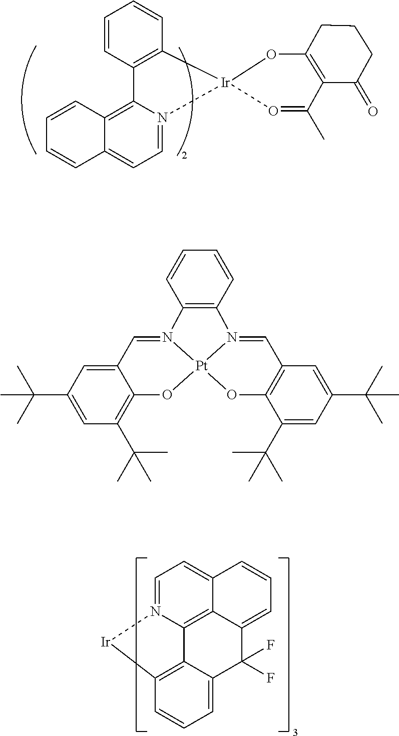

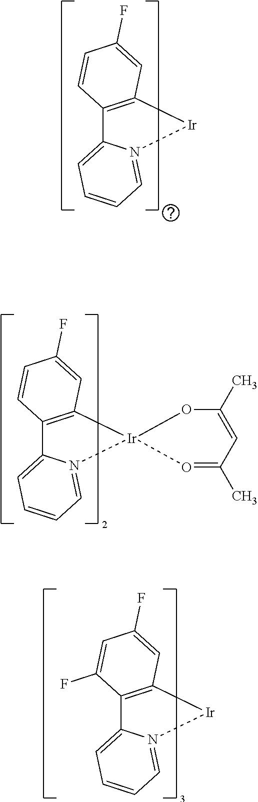

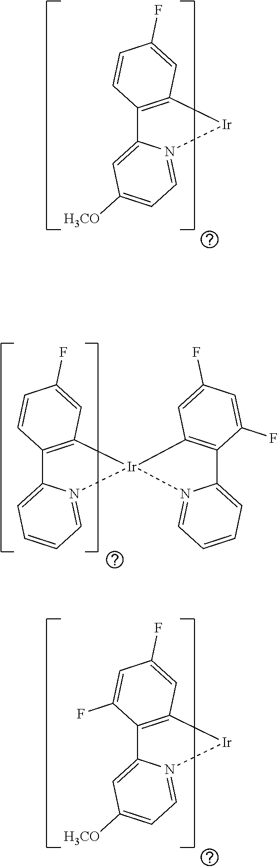

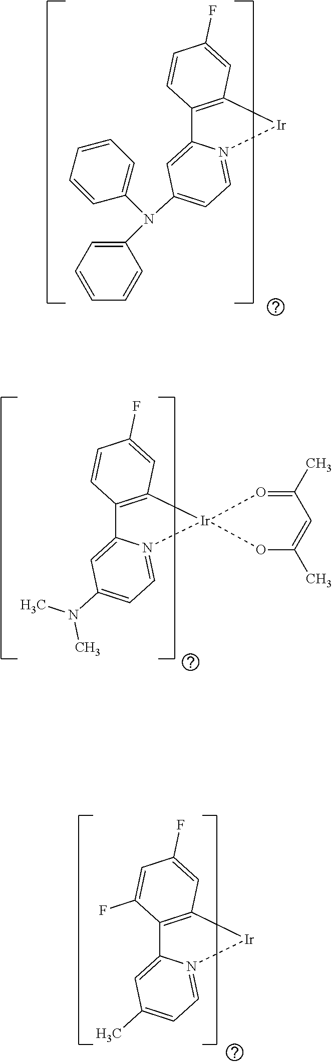

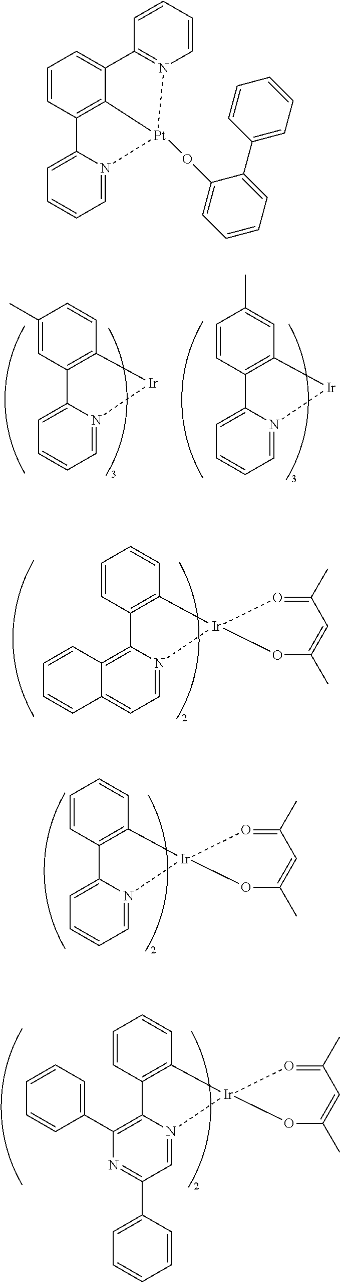

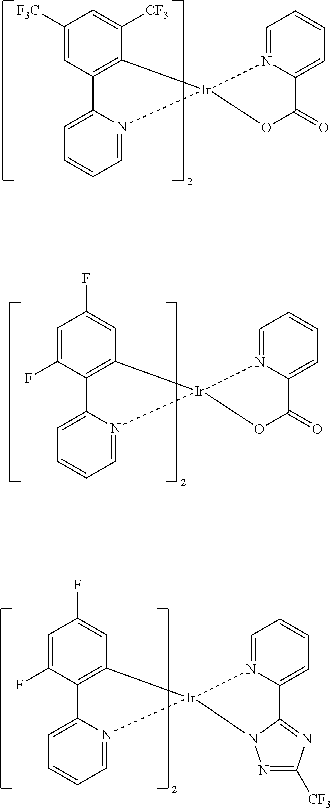

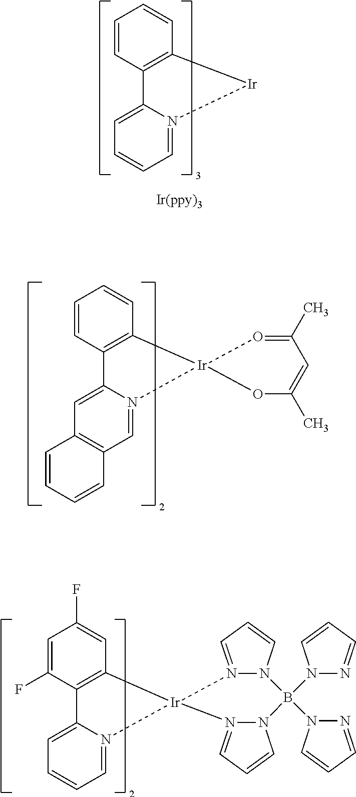

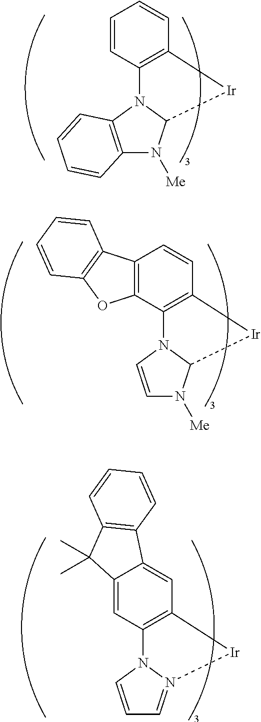

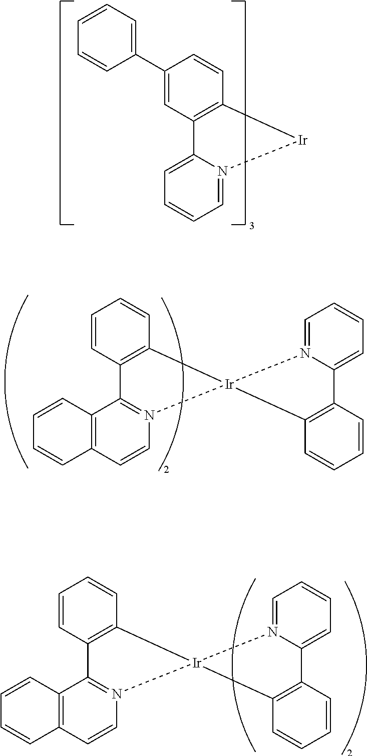

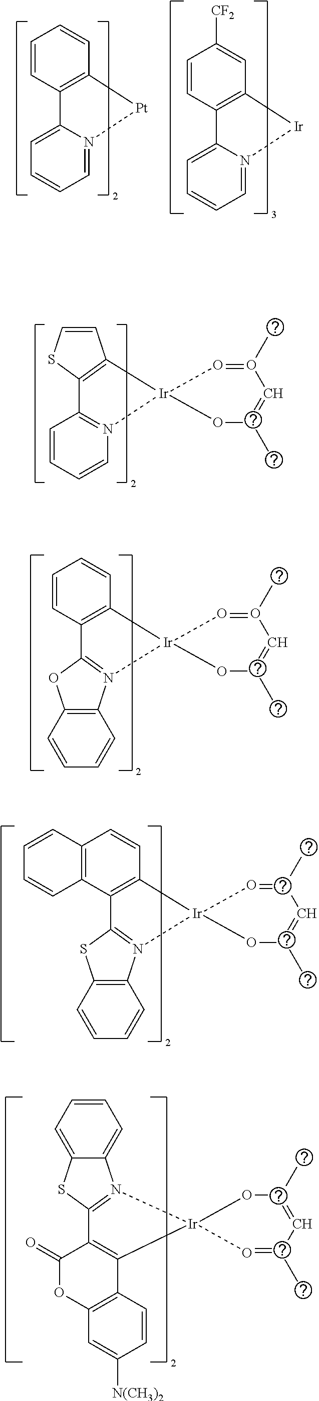

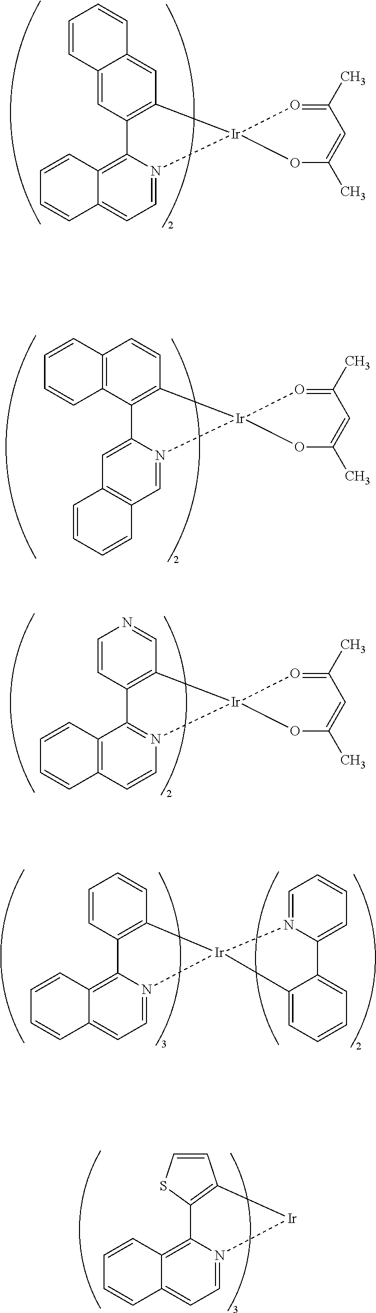

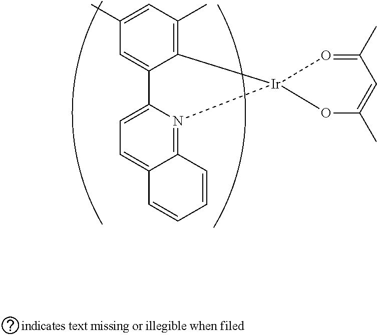

Two or more metal complexes may be combined, and it is preferable that at least one of the metal complexes has a maximum value of the emission wavelength of 450 nm or more and 750 nm or less. As a preferred example, the maximum value is 450 nm or more and 495 nm or less, 495 nm or more and 590 nm or less, or 590 nm or more and 750 nm or less.

By forming a light-emitting layer by doping the metal complex (dopant) having such emission wavelength to the compound represented by the formula (1) and the compound represented by the formula (11) (host material), a highly efficient organic EL device can be obtained.

In addition, in order to disperse substances with high light-emitting properties, the light-emitting layer may comprise 1) a metal complex such as an aluminum complex, a beryllium complex, or a zinc complex, 2) a heterocyclic compound such as an oxadiazole derivative, a benzimidazole derivative or a phenanthroline derivative, 3) a fused aromatic compound such as an anthracene derivative, a phenanthrene derivative, a pyrene derivative or a chrysene derivative, 4) an aromatic amine compound such as a triarylamine derivative or a fused polycyclic aromatic amine derivative, and the like.

Examples of the representative device configuration of the organic EL device of the present invention include,

(1) anode/light-emitting layer/cathode
(2) anode/hole injection layer/light-emitting layer/cathode
(3) anode/light-emitting layer/electron injection transporting layer/cathode
(4) anode/hole injection layer/light-emitting layer/electron injection transporting layer/cathode
(5) anode/organic semiconductor layer/light-emitting layer/cathode
(6) anode/organic semiconductor layer/electron barrier layer/light-emitting layer/cathode
(7) anode/organic semiconductor layer/light-emitting layer/adhesion improving layer/cathode
(8) anode/hole injection transporting layer/light-emitting layer/electron injection⋅transporting layer/cathode
(9) anode/insulating layer/light-emitting layer/insulating layer/cathode
(10) anode/inorganic semiconductor layer/insulating layer/light-emitting layer/insulating layer/cathode
(11) anode/organic semiconductor layer/insulating layer/light-emitting layer/insulating layer/cathode
(12) anode/insulating layer/hole injecting⋅transporting layer/light-emitting layer/insulating layer/cathode
(13) anode/insulating layer/hole injection⋅transporting layer/light-emitting layer/electron injection transporting layer/cathode, and the like.

Among the above, the constitution of (8) is preferably used, but it is not limited thereto.

In addition, the light-emitting layer may be either a phosphorescent light-emitting layer or a fluorescent light-emitting layer, or a plurality of emitting layers may be used. When there are plural light-emitting layers, a space layer may be provided between each of light-emitting layers for the purpose of preventing excitons generated in the phosphorescent light-emitting layer from diffusing into the fluorescent light-emitting layer.

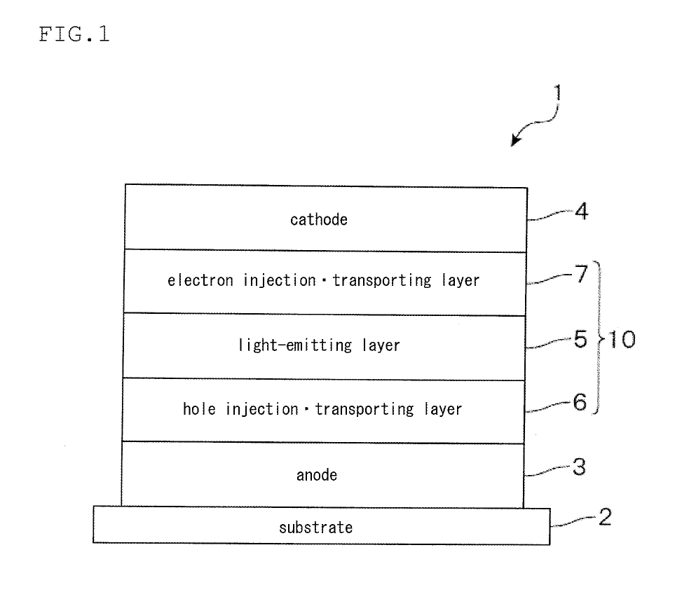

FIG. 1 shows a schematic configuration of an example of an organic EL device according to an embodiment of the present invention.

The organic EL device 1 comprises a transparent substrate 2, an anode 3, a cathode 4, and an organic thin film layer 10 disposed between the anode 3 and the cathode 4.

The organic thin film layer 10 comprises the above-mentioned light-emitting layer 5, and may comprise a hole injecting⋅transporting layer 6 and the like between the light-emitting layer 5 and the anode 3, and an electron injecting⋅transporting layer 7 and the like between the light-emitting layer 5 and the cathode 4.

Further, an electron barrier layer may be provided on the anode 3 side of the light-emitting layer 5 and a hole barrier layer may be provided on the cathode 4 side of the light-emitting layer 5.

Thus, it is possible to confine electrons and holes in the light-emitting layer 5 and increase the probability of generation of excitons in the light-emitting layer 5.

In addition, one aspect of the organic EL device of the present invention may be a fluorescent or phosphorescent monochromatic light-emitting device, a fluorescent/phosphorescent hybrid white light-emitting device, a simple type having a single light-emitting unit or a tandem type having a plurality of light light-emitting units. Here, the “light-emitting unit” refers to the smallest unit capable of light-emitting by recombination of injected holes and electrons, which comprises one or more organic layers, and one of which is a light-emitting layer. Representative layer configurations of the light-emitting unit are shown below.

(a) hole transporting layer/light-emitting layer (/electron transporting layer)
(b) hole transporting layer/first phosphorescent light-emitting layer/second phosphorescent light-emitting layer (/electron transporting layer)
(c) hole transporting layer/phosphorescent light-emitting layer/space layer/fluorescent light-emitting layer (/electron transporting layer)
(d) hole transporting layer/first phosphorescent light-emitting layer/second phosphorescent light-emitting layer/space layer/fluorescent light-emitting layer (/electron transporting layer)
(e) hole transporting layer/first phosphorescent light-emitting layer/space layer/second phosphorescent light-emitting layer/space layer/fluorescent light-emitting layer (/electron transporting layer)
(f) hole transporting layer/phosphorescent light-emitting layer/space layer/first fluorescent light-emitting layer/second fluorescent light-emitting layer (/electron transporting layer)

Examples of typical device configurations of the tandem type organic EL device includes the following element configuration.

Anode/first light-emitting unit/intermediate layer/second light-emitting unit/cathode

Here, as the first light-emitting unit and the second light-emitting unit, for example, the same as the light-emitting unit described above can independently be selected.

Generally, the intermediate layer is also called as an intermediate electrode, an intermediate conductive layer, a charge generation layer, an electron extracting layer, a connecting layer, or an intermediate insulating layer, and a known material composition which injects electrons into the first light-emitting unit and injects holes into the second light-emitting unit can be used.

A host material of the light-emitting layer includes a fluorescent host and a phosphorescent host. When combined with a fluorescent dopant, it is referred to as a fluorescent host, and when combined with a phosphorescent dopant, it is referred to as a phosphorescent host. They are not distinguished to the fluorescent host and the phosphorescent host only based on their molecular structure.

In other words, the fluorescent host means a material constituting a fluorescent light-emitting layer containing a fluorescent dopant, and it is not only usable as a host for a fluorescent light-emitting material.

Likewise, the phosphorescent host means a material constituting a phosphorescent light-emitting layer containing a phosphorescent dopant and it is not only usable as a host for a phosphorescent light-emitting material.

Further, in the present specification, the “hole injection⋅transporting layer” means “at least one of a hole injection layer and a hole transporting layer”, the “electron injection⋅transport layer” means “at least one of an electron injection layer and an electron transporting layer”.

The substrate is used as a support of the light-emitting device. As the substrate, for example, glass, quartz, plastic, and the like can be used. Further, a flexible substrate may be used. The term “flexible substrate” refers to a substrate that can be bent, for example, a plastic substrate made of polycarbonate, polyvinyl chloride, or the like.

For the anode formed on the substrate, it is preferable to use a metal, an alloy, an electrically conductive compound, a mixture thereof and the like having a large work function (specifically, 4.0 eV or more). Specifically, for example, indium oxide-tin oxide (ITO: Indium Tin Oxide), indium oxide-tin oxide containing silicon or silicon oxide, indium oxide-zinc oxide, tungsten oxide, indium oxide containing zinc oxide, graphene and the like can be used. In addition to these, gold (Au), platinum (Pt), nitrides of metal materials (for example, titanium nitride), and the like can be used.

The hole injecting layer is a layer containing a substance having a high hole injecting property. As a substance having a high hole injecting property, a substance selected from molybdenum oxide, titanium oxide, vanadium oxide, rhenium oxide, ruthenium oxide, chromium oxide, zirconium oxide, hafnium oxide, tantalum oxide, silver oxide, tungsten oxide, manganese oxide, aromatic amine compound, ladder compound such as fluorene derivative, polymer compound (oligomer, dendrimer, polymer, etc.), and the like can be used.

The hole transporting layer is a layer containing a substance having a high hole transporting property. For the hole transporting layer, aromatic amine compounds, carbazole derivatives, anthracene derivatives and the like can be used. Polymer compounds such as poly (N-vinylcarbazole) (abbreviation: PVK) and poly (4-vinyltriphenylamine) (abbreviation: PVTPA) can also be used. However, any substance other than these may be used as long as it has a hole transporting property higher than an electron transporting property. Note that the layer containing a substance having a high hole transporting property is not limited to a single layer, but may be a stack of two or more layers comprises the above substances.

For the hole transporting layer, it is preferable to use the same compound as the compound represented by the above formula (11).

The electron transporting layer is a layer containing a substance having a high electron transporting property. For the electron transporting layer, 1) a metal complex such as a lithium complex, an aluminum complex, a beryllium complex, or a zinc complex, 2) a heteroaromatic compound such as an imidazole derivative, a benzimidazole derivative, an azine derivative, a carbazole derivative, or a phenanthroline derivative, and 3) a polymer compound can be used.

The electron injection layer is a layer containing a substance having a high electron injection property. For the electron injection layer, an alkali metal, alkaline earth metal, or an alloy thereof such as a lithium (Li), a lithium complex, a lithium fluoride (LiF), a cesium fluoride (CsF), a calcium fluoride (CaF₂), or a lithium oxide (LiOₓ) can be used.

It is preferable to use a metal, an alloy, an electrically conductive compound, a mixture thereof, and the like having a small work function (specifically, 3.8 eV or less) for the cathode. Specific examples of such a cathode material include elements belong to Group 1 or Group 2 of the periodic table of the elements, that is, alkali metals such as lithium (Li) and cesium (Cs), and alkaline earths such as magnesium (Mg), alloys containing those (e.g. MgAg, AlLi) and an alloy containing those and the like.

In one aspect of the organic EL device of the present invention, a method for forming each layer is not particularly limited. A conventionally known forming method such as a vacuum deposition method and a spin coating method can be used. Each layer such as a light-emitting layer can be formed by a known coating method such as a vacuum deposition method, a molecular beam evaporation method (MBE method), or a coating method of solution dissolved in solvent, such as a dipping method, a spin coating method, a casting method, a bar coating method, or a roll coating method.

In the formation of the light-emitting layer, the compound represented by the formula (1) and the compound represented by the formula (11) may be formed by co-deposition.

Alternatively, the compound represented by the formula (1) and the compound represented by the formula (11) may be premixed to prepare a material for an organic EL device, and the light-emitting layer may be formed by deposition of the material for an organic EL device.

It is preferable that the premix be conducted by mixing the powder of the compound represented by the formula (1) and the powder of the compound represented by the formula (11).

In one aspect of the organic EL device of the present invention, the film thickness of each layer is not particularly limited. But generally, in order to suppress defects such as pinholes, to suppress the applied voltage level and to improve the efficiency, it is usually preferable in a range from several nm to 1 μm.

The organic EL device of the present invention can be used for a lighting device, a display device, and the like.

### EXAMPLES

### Synthesis Example

Host 1 and Host 1D as mentioned below were synthesized by the method described in WO 2012/108388.

Host 1A as mentioned below was synthesized in the same manner as in paragraphs 0174 to 0175 of WO 2012/108388, except that 8-bromofluoranthene was used instead of 3-bromofluoranthene.

Host 1B as mentioned below was synthesized in the same manner as in paragraphs 0174 to 0175 of WO 2012/108388, except that 2-bromo-3-azafluoranthene was used instead of 3-bromofluoranthene.

Host 1C as mentioned below was synthesized in the same manner as in paragraphs 0174 to 0175 of WO 2012/108388, except that 3-(9-naphthylcarbazol-3-yl) carbazole was used instead of intermediate 1-1.

### Example 1

A glass substrate of 25 mm×75 mm×1.1 mm thick with an ITO transparent electrode (GEOMATEC CO., LTD., ITO film thickness: 130 nm) was subjected to ultrasonic cleaning in isopropyl alcohol for 5 minutes, and cleaning with UV (ultraviolet)/ozone for 30 minutes.

The cleaned glass substrate with transparent electrode line was then mounted on the substrate holder of a vacuum deposition device. First, compound HA as mentioned below was deposited on the surface of the substrate on which the transparent electrode line was formed so as to cover the transparent electrode to form an HA film (hole injecting layer) having a thickness of 5 nm. Compound HT as mentioned below was deposited on the HA film as a hole transporting material to form a hole transporting layer having a thickness of 210 nm.

Compounds Host 1, Host 2 and Dopant as mentioned below were co-deposited on the hole transporting layer such that the weight ratio of Host 1:Host 2:Dopant became 78:20:2 to form a light-emitting layer having a thickness of 40 nm.

Subsequently to the formation of the light-emitting layer, compound ET and Liq as mentioned below were co-deposited on the light-emitting layer such that the weight ratio of ET:Liq became 50:50, to form an electron transporting layer having a thickness of 30 nm.

Next, Liq was deposited at a deposition rate of 0.1 angstrom/min to form an electron injecting electrode (cathode) having a film thickness of 1 nm. Metal Al was deposited on the Liq film to form a metal cathode with a film thickness of 80 nm. An organic EL device was fabricated in this manner.

The initial characteristics of the obtained organic EL device was measured at room temperature under driving with DC (direct current) constant current of 10 mA/cm². The results of the voltage, luminescent chromaticity and L/J luminous efficiency are shown in Table 1. Further, the voltage was applied to the organic EL device such that the current density was 50 mA/cm², and life time LT 90 was measured which is the time until the luminance was decrease to 90% based on the initial luminance. The results are shown in Table 1.

The luminescent chromaticity x and y were measured with a spectroradiometer (CS-1000, manufactured by Minolta).

The ionization potentials of the compounds HT, Host 1 and Host 2 as mentioned below were measured under atmospheric conditions using a photoelectron spectrometer AC-3 (manufactured by Riken Keiki Co., Ltd.). Specifically, it was determined by measuring the amount of electrons generated by charge separation when light is irradiated to a compound to be measured. The results are shown in Table 2.

### Examples 2 and 3, and Comparative Examples 1 to 6

Organic EL devices were prepared and evaluated in the same manner as in Example 1, except that the compound deposited to form the light-emitting layer of Example 1 was changed to the compounds shown in Table 1. The results are shown in Table 1.

The ionization potential of compounds Host 3 to 7 as mentioned below were measured in the same manner as in Example 1. The results are shown in Table 2.

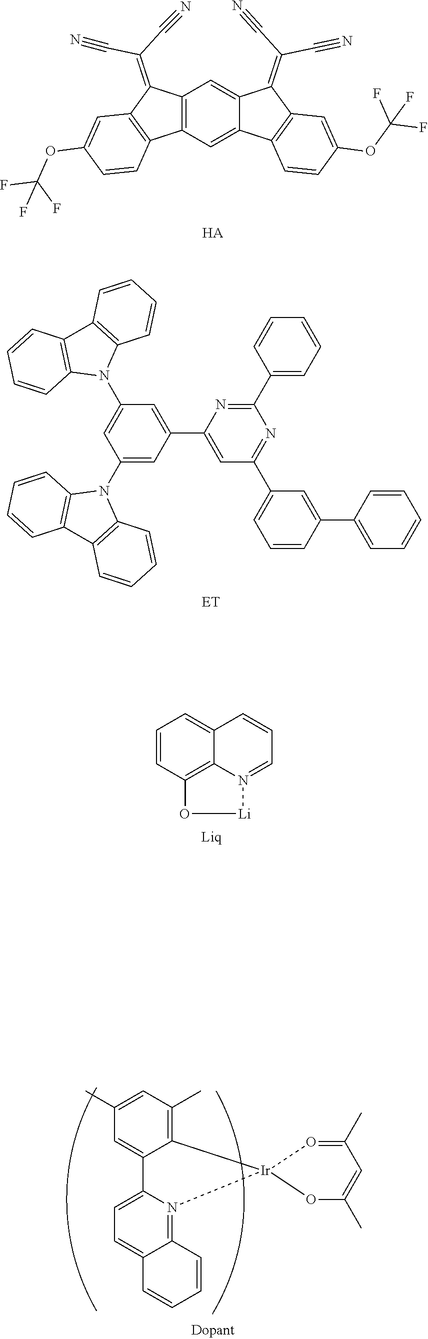

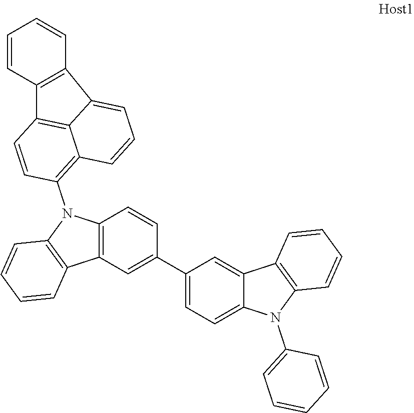 [SMILES: c1ccc(-n2c3ccccc3c3cc(-c4ccc5c(c4)c4ccccc4n5-c4ccc5c6c(cccc46)-c4ccccc4-5)ccc32)cc1]

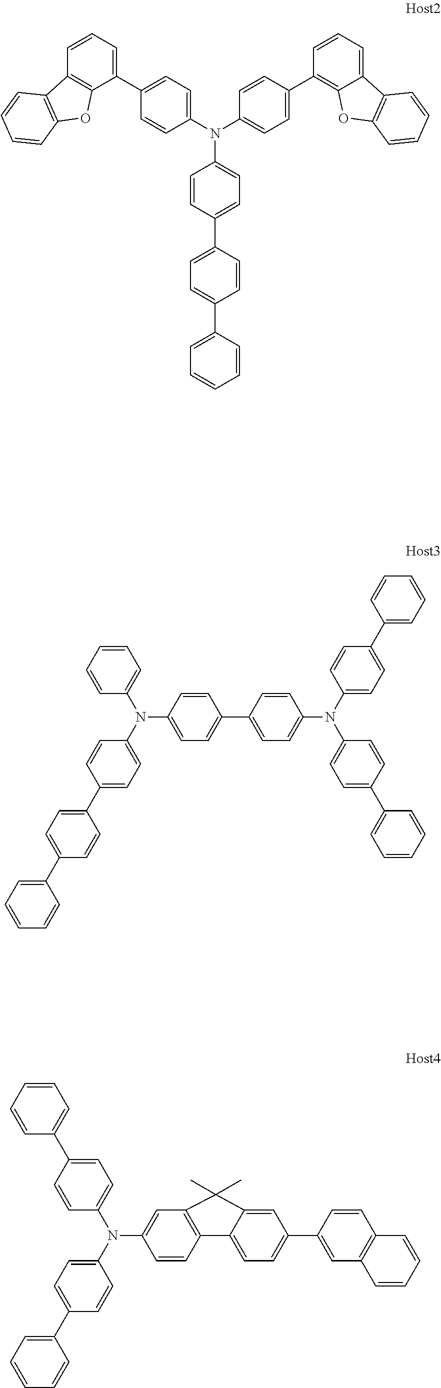 [SMILES: c1ccc(-c2ccc(-c3ccc(N(c4ccc(-c5cccc6c5oc5ccccc56)cc4)c4ccc(-c5cccc6c5oc5ccccc56)cc4)cc3)cc2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; CC1(C)c2cc(-c3ccc4ccccc4c3)ccc2-c2ccc(N(c3ccc(-c4ccccc4)cc3)c3ccc(-c4ccccc4)cc3)cc21]

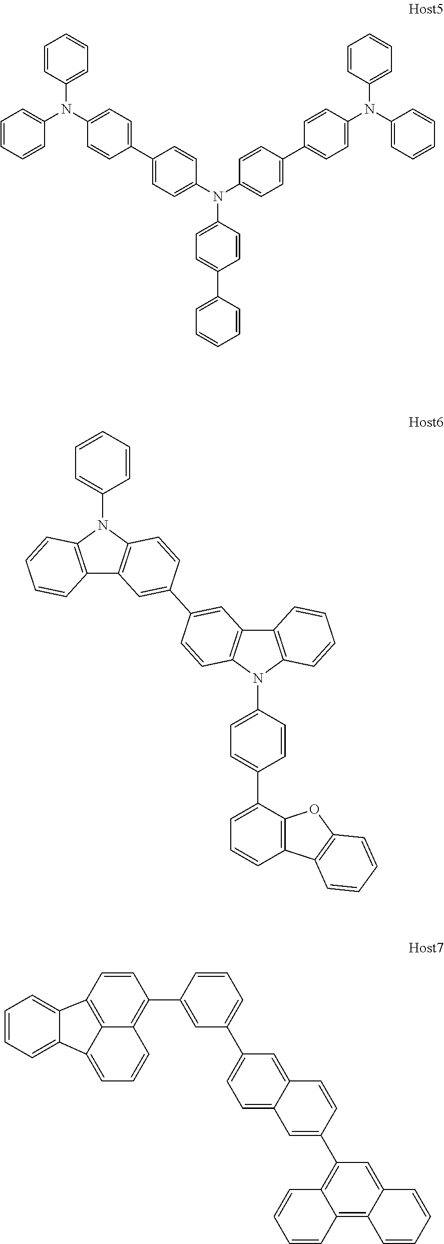

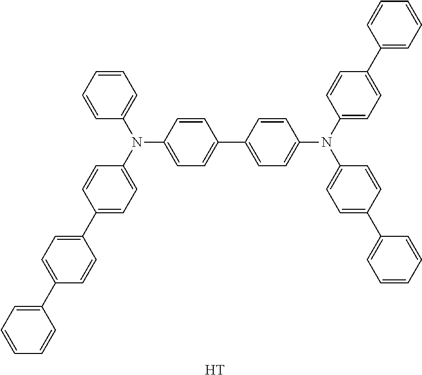

TABLE 1

| | | | | | | | L/J luminous | |
|---|---|---|---|---|---|---|---|---|
| | | | | Voltage | Chromaticity | Chromaticity | efficiency | LT90 |
| | Light-emitting layer | | | (V) | x | y | (cd/A) | (hr) |
| Example 1 | Host1 | Host2 | Dopant | 4.25 | 0.66 | 0.33 | 21.9 | 240 |
| Example 2 | Host1 | Host3 | Dopant | 4.22 | 0.66 | 0.34 | 22.5 | 319 |
| Example 3 | Host1 | Host4 | Dopant | 4.13 | 0.66 | 0.33 | 22.5 | 284 |
| Example 4 | Host1 | Host5 | Dopant | 4.02 | 0.661 | 0.336 | 22.5 | 309 |
| Comparative | Host1 | none | Dopant | 4.28 | 0.66 | 0.34 | 21.6 | 176 |
| Example 1 | | | | | | | | |
| Comparative | none | Host2 | Dopant | 4.64 | 0.647 | 0.343 | 6.2 | 15 |
| Example 2 | | | | | | | | |
| Comparative | none | Host3 | Dopant | 3.89 | 0.649 | 0.344 | 6.2 | 9 |
| Example 3 | | | | | | | | |
| Comparative | none | Host4 | Dopant | 3.91 | 0.633 | 0.350 | 5.1 | 8 |
| Example 4 | | | | | | | | |
| Comparative | Host1 | Host6 | Dopant | 4.29 | 0.663 | 0.334 | 20.8 | 190 |
| Example 5 | | | | | | | | |
| Comparative | Host7 | Host2 | Dopant | 4.75 | 0.656 | 0.334 | 20.3 | 110 |
| Example 6 | | | | | | | | |

In Comparative Example 1, the weight ratio of Host 1:Dopant was 98:2, in Comparative Example 2, the weight ratio of Host 2:Dopant was 98:2, in Comparative Example 3, the weight ratio of Host 3:Dopant was 98:2, and in Comparative Example 4, the weight ratio of Host 4:Dopant was 98:2.

Examples 1 to 4 exhibited excellent LT90 as compared to that of Comparative Examples 1 to 6. In particular, Examples 2 and 4 showed more excellent LT90.

Examples 1 to 4 exhibited excellent luminous efficiency as compared to that of Comparative Examples 1 to 6.

Examples 1 to 4 exhibited reduced voltage as compared to that of Comparative Examples 1, 2, 5 and 6.

In Comparative Examples 3 and 4, although the voltage was lower than that in Examples, the luminous efficiency and LT90 were significantly inferior to those in Examples.

TABLE 2

| | Ionization potential (eV) |
|---|---|
| HT | 5.51 |
| Host1 | 5.71 |
| Host2 | 5.65 |
| Host3 | 5.51 |
| Host4 | 5.54 |
| Host5 | 5.48 |
| Host6 | 5.60 |
| Host7 | 6.19 |

Further, with respect to the compounds used in the light-emitting layers of Examples 1 to 4, the calculation results of values of “(the ionization potential of the compound represented by the formula (1))−(the ionization potential of the compound represented by the formula (11))” are shown in Table 3.

TABLE 3

| | Compound | Compound | Difference in |
|---|---|---|---|
| | represented by | represented by | ionization |
| | the formula (1) | the formula (11) | potential (eV) |
| Example 1 | Host 1 | Host 2 | 0.06 |
| Example 2 | Host 1 | Host 3 | 0.20 |
| Example 3 | Host 1 | Host 4 | 0.17 |
| Example 4 | Host 1 | Host 5 | 0.23 |

### Example 5 and Comparative Example 7

In Example 5 and Comparative Example 7, organic EL devices were prepared and evaluated in the same manner as in Example 1, except that the compound used in the light-emitting layer and weight ratio were changed to the compound and weight ratio shown in Table 4. The results are shown in Table 4. The structure of the compound Host 1D is shown below.

The ionization potential of the compound Host 1D was measured in the same manner as in Example 1. The result is shown in Table 5.

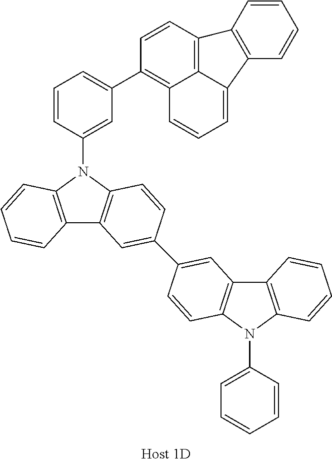 [SMILES: c1ccc(-n2c3ccccc3c3cc(-c4ccc5c(c4)c4ccccc4n5-c4cccc(-c5ccc6c7c(cccc57)-c5ccccc5-6)c4)ccc32)cc1]

TABLE 4

| | | | | | | | L/J luminous | |
|---|---|---|---|---|---|---|---|---|
| | | | | Voltage | Chromaticity | Chromaticity | efficiency | LT90 |
| | Light-emitting layer | | | (V) | x | y | (cd/A) | (hr) |
| Example 5 | Host 1 D | Host 3 | Dopant | 4.72 | 0.66 | 0.34 | 21.7 | 250 |
| | (88 wt %) | (10 wt %) | (2 wt %) | | | | | |
| Comparative | Host 1 D | none | Dopant | 4.75 | 0.66 | 0.34 | 21.2 | 215 |
| Example 7 | (98 wt %) | | (2 wt %) | | | | | |

Example 5 exhibited excellent LT90 as compared to that of Comparative Example 7.

Example 5 exhibited excellent luminous efficiency as compared to that of Comparative Example 7.

Example 5 exhibited reduced voltage as compared to that of Comparative Example 7.

TABLE 5

| | Ionization potential (eV) |
|---|---|
| Host 1 D | 5.71 |

Further, with respect to the compound used in the light-emitting layer of Example 5, the calculation results of values of “(the ionization potential of the compound represented by the formula (1))−(the ionization potential of the compound represented by the formula (11))” are shown in Table 6.

TABLE 6

| | Compound | Compound | Difference in |
|---|---|---|---|
| | represented by | represented by | ionization |
| | the formula (1) | the formula (11) | potential (eV) |
| Example 5 | Host 1 D | Host 3 | 0.20 |

### Example 6 and Comparative Example 8

In Example 6 and Comparative Example 8, organic EL devices were formed in the same manner as in Example 1 except the following condition: the compound HT of the hole transporting material of Example 1 was changed to the following compound HT1; the compound for the light-emitting layer deposition and the weight ratio of Example 1 were respectively changed to the compound and the weight ratio shown in Table 7; the following compound ET1 was deposited as a first electron transporting layer (hole blocking layer) to form a film (10 nm), and the following compound ET2 was subsequently deposited as a second electron transporting layer to form a film (15 nm) instead of the electron transporting layer of Example 1; and an electron injecting electrode (cathode) was deposited by using LiF instead of Liq in Example 1 at a deposition rate of 0.1 angstrom/s to formed a film (1 nm).

The voltage, luminescent chromaticity and L/J luminous efficiency of the obtained devices were evaluated in the same manner as in Example 1. Further, a voltage was applied to the organic EL devices such that the current density was 50 mA/cm², and life time LT95 was measured which is the time until the luminance was decrease to 95% based on the initial luminance. The results are shown in Table 7. The structure of the compound Host 1A is shown below.

The ionization potential of the following compound Host 1A was measured in the same manner as in Example 1. The results are shown in Table 8.

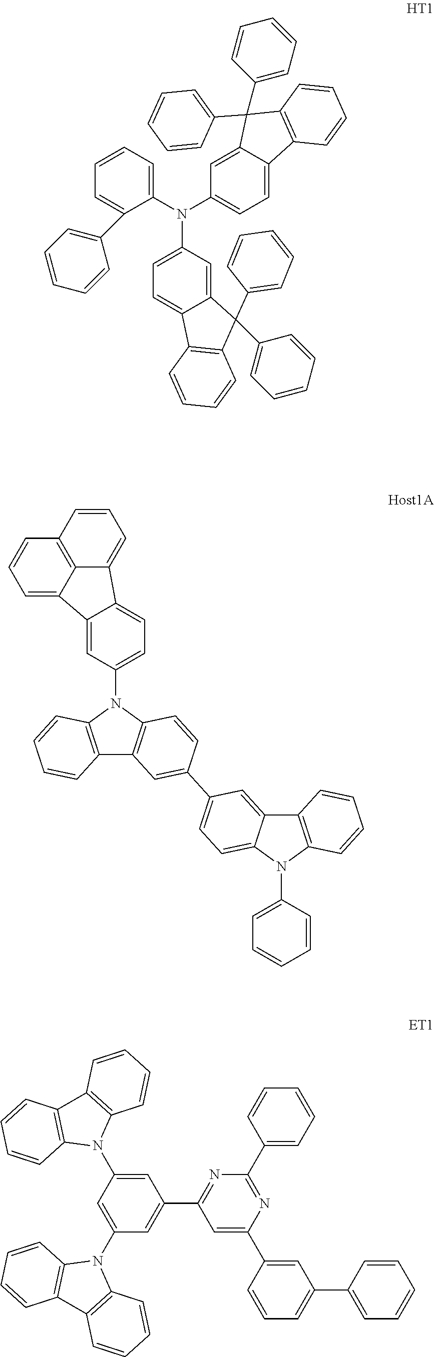 [SMILES: c1ccc(-c2ccccc2N(c2ccc3c(c2)C(c2ccccc2)(c2ccccc2)c2ccccc2-3)c2ccc3c(c2)C(c2ccccc2)(c2ccccc2)c2ccccc2-3)cc1 ; c1ccc(-n2c3ccccc3c3cc(-c4ccc5c(c4)c4ccccc4n5-c4ccc5c(c4)-c4cccc6cccc-5c46)ccc32)cc1 ; c1ccc(-c2cccc(-c3cc(-c4cc(-n5c6ccccc6c6ccccc65)cc(-n5c6ccccc6c6ccccc65)c4)nc(-c4ccccc4)n3)c2)cc1]

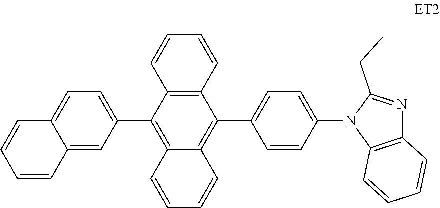 [SMILES: CCc1nc2ccccc2n1-c1ccc(-c2c3ccccc3c(-c3ccc4ccccc4c3)c3ccccc23)cc1]

TABLE 7

| | | | | | | | L/J luminous | |
|---|---|---|---|---|---|---|---|---|
| | | | | Voltage | Chromaticity | Chromaticity | efficiency | LT95 |
| | Light-emitting layer | | | (V) | x | y | (cd/A) | (hr) |
| Example 6 | Host 1 A | Host 2 | Dopant | 3.26 | 0.66 | 0.34 | 23.1 | 103 |
| | (88 wt %) | (10 wt %) | (2 wt %) | | | | | |
| Comparative | Host 1 A | none | Dopant | 3.23 | 0.66 | 0.34 | 22.5 | 77 |
| Example 8 | (98 wt %) | | (2 wt %) | | | | | |

Example 6 exhibited excellent LT95 as compared to that of Comparative Example 8.

Example 6 exhibited excellent luminous efficiency as compared to that of Comparative Example 8.

TABLE 8

| | Ionization potential (eV) |
|---|---|
| Host1 A | 5.73 |

Further, with respect to the compounds used in the light-emitting layer of Example 6, the calculation results of values of “(the ionization potential of the compound represented by the formula (1))−(the ionization potential of the compound represented by the formula (11))” are shown in Table 9.

TABLE 9

| | Compound | Compound | Difference in |
|---|---|---|---|
| | represented by | represented by | ionization |
| | the formula (1) | the formula (11) | potential (eV) |
| Example 6 | Host 1 A | Host 2 | 0.08 |

### Example 7 and Comparative Example 9

In Example 7 and Comparative Example 9, organic EL devices were formed in the same manner as in Example 1 except the following condition: the compound HT of the hole transporting material of Example 1 was changed to the following compound HT2; the compound for the light-emitting layer deposition and the weight ratio of Example 1 were respectively changed to the compound and the weight ratio shown in Table 10; the following compound ET1 was deposited as a first electron transporting layer (hole blocking layer) to form a film (10 nm), and the following compound ET2 was subsequently deposited as a second electron transporting layer to form a film (15 nm) instead of the electron transporting layer of Example 1; and an electron injecting electrode (cathode) was deposited by using LiF instead of Liq in Example 1 at a deposition rate of 0.1 angstrom/s to formed a film (1 nm).

The voltage, luminescent chromaticity and L/J luminous efficiency of the obtained devices were evaluated in the same manner as in Example 1. Further, a voltage was applied to the organic EL devices such that the current density was 50 mA/cm², and life time LT95 was measured which is the time until the luminance was decrease to 95% based on the initial luminance. The results are shown in Table 10. The structure of the compound Host 1B is shown below.

The ionization potential of the compound Host 1B was measured in the same manner as in Example 1. The result is shown in Table 11.

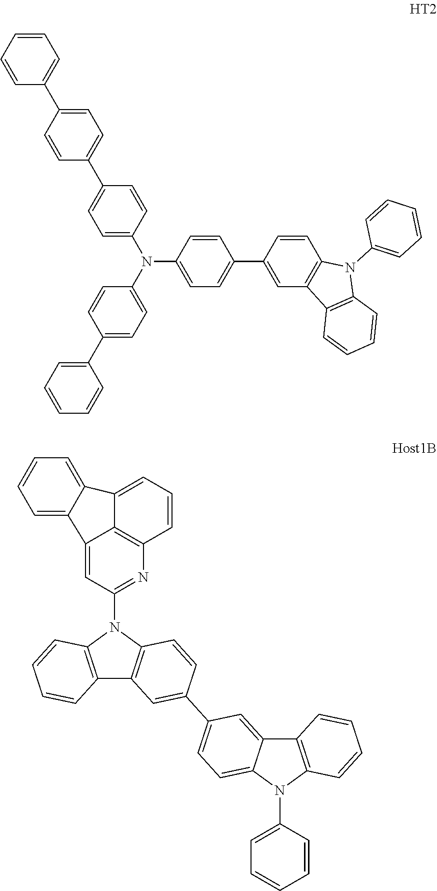 [SMILES: c1ccc(-c2ccc(-c3ccc(N(c4ccc(-c5ccccc5)cc4)c4ccc(-c5ccc6c(c5)c5ccccc5n6-c5ccccc5)cc4)cc3)cc2)cc1 ; c1ccc(-n2c3ccccc3c3cc(-c4ccc5c(c4)c4ccccc4n5-c4cc5c6c(cccc6n4)-c4ccccc4-5)ccc32)cc1]

TABLE 10

| | | | | | | | L/J luminous | |
|---|---|---|---|---|---|---|---|---|
| | | | | Voltage | Chromatcity | Chromaticity | efficiency | LT95 |
| | Light-emitting layer | | | (V) | x | y | (cd/A) | (hr) |
| Example 7 | Host 1 B | Host 2 | Dopant | 4.10 | 0.66 | 0.34 | 20.3 | 117 |
| | (88 wt %) | (10 wt %) | (2 wt %) | | | | | |
| Comparative | Host 1 B | none | Dopant | 4.10 | 0.66 | 0.34 | 19.8 | 88 |
| Example 9 | (98 wt %) | | (2 wt %) | | | | | |

Example 7 exhibited excellent LT95 as compared to that of Comparative Example 9.

Example 7 exhibited excellent luminous efficiency as compared to that of Comparative Example 9.

TABLE 11

| | Ionization potential (eV) |
|---|---|
| Host1 B | 5.69 |

Further, with respect to the compounds used in the light-emitting layer of Example 7, the calculation results of values of “(the ionization potential of the compound represented by the formula (1))−(the ionization potential of the compound represented by the formula (11))” are shown in Table 12.

TABLE 12

| | Compound | Compound | Difference in |
|---|---|---|---|
| | represented by | represented by | ionization |
| | the formula (1) | the formula (11) | potential (eV) |
| Example 7 | Host 1 B | Host 2 | 0.04 |

### Example 8 and Comparative Example 10

In Example 8 and Comparative Example 10, organic EL devices were formed in the same manner as in Example 1 except the following condition: the compound HT of the hole transporting material of Example 1 was changed to the above compound HT1; the compound for the light-emitting layer deposition and the weight ratio of Example 1 were respectively changed to the compound and the weight ratio shown in Table 13; the above compound ET2 was deposited as an electron transporting layer instead of the electron transporting layer of Example 1 to form a film (25 nm); and an electron injecting electrode (cathode) was deposited with LiF instead of Liq in Example 1 at a deposition rate of 0.1 angstrom/s to formed a film (1 nm). The organic EL devices were evaluated in the same manner as in Example 1. The results are shown in Table 13. The structure of the compound Host 1C is shown below.

The ionization potential of the following compound Host 1C was measured in the same manner as in Example 1. The result is shown in Table 14.

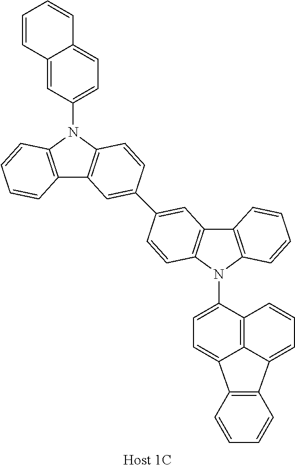 [SMILES: c1ccc2c(c1)-c1cccc3c(-n4c5ccccc5c5cc(-c6ccc7c(c6)c6ccccc6n7-c6ccc7ccccc7c6)ccc54)ccc-2c13]

TABLE 13

| | | | | | | | L/J luminous | |
|---|---|---|---|---|---|---|---|---|
| | | | | Voltage | Chromaticity | Chromaticity | efficiency | LT90 |
| | Light-emitting layer | | | (V) | x | y | (cd/A) | (hr) |
| Example 8 | Host 1C | Host 2 | Dopant | 3.10 | 0.66 | 0.34 | 18.9 | 142 |
| | (88 wt %) | (10 wt %) | (2 wt %) | | | | | |
| Comparative | Host 1C | none | Dopant | 3.07 | 0.66 | 0.34 | 17.1 | 98 |
| Example 10 | (98 wt %) | | (2 wt %) | | | | | |

Example 8 exhibited excellent LT95 as compared to that of Comparative Example 10.

Example 8 exhibited excellent luminous efficiency as compared to that of Comparative Example 10.

TABLE 14

| | Ionization potential (eV) |
|---|---|
| Host1 C | 5.72 |

Further, with respect to the compounds used in the light-emitting layer of Example 8, the calculation results of values of “(the ionization potential of the compound represented by the formula (1))−(the ionization potential of the compound represented by the formula (11))” are shown in Table 15.

TABLE 15

| | Compound | Compound | Difference in |
|---|---|---|---|
| | represented by | represented by | ionization |
| | the formula (1) | the formula (11) | potential (eV) |
| Example 8 | Host 1 C | Host 2 | 0.07 |

### Example 9 and Comparative Example 11

In Example 9 and Comparative Example 11, the organic EL devices were formed in the same manner as in Example 1 except the following condition: the compound HT of the hole transporting material of Example 1 was changed to the above compound HT2; the compound for the light-emitting layer deposition and the weight ratio of Example 1 were respectively changed to the compound and the weight ratio shown in Table 16; the above compound ET2 was deposited as an electron transporting layer instead of the electron transporting layer of Example 1 to form a film (25 nm); and an electron injecting electrode (cathode) was deposited with LiF instead of Liq in Example 1 at a deposition rate of 0.1 angstrom/s to formed a film (1 nm). The organic EL devices were evaluated in the same manner as in Example 1. The results are shown in Table 16.

TABLE 16

| | | | | | | | L/J luminous | |
|---|---|---|---|---|---|---|---|---|
| | | | | Voltage | Chromaticity | Chromaticity | efficiency | LT90 |
| | Light-emitting layer | | | (V) | x | y | (cd/A) | (hr) |
| Example 9 | Host 1 C | Host 2 | Dopant | 3.74 | 0.66 | 0.34 | 18.5 | 113 |
| | (88 wt %) | (10 wt %) | (2 wt %) | | | | | |
| Comparative | Host 1 C | none | Dopant | 3.71 | 0.66 | 0.33 | 16.8 | 74 |
| Example 11 | (98 wt %) | | (2 wt %) | | | | | |

Example 9 exhibited excellent LT95 as compared to that of Comparative Example 11.

Example 9 exhibited excellent luminous efficiency as compared to that of Comparative Example 11.

Although only some exemplary embodiments and/or examples of the invention have been described in detail above, those skilled in the art will readily appreciate that many modifications are possible in the exemplary embodiments and/or examples without materially departing from the novel teachings and advantages of the invention. Accordingly, all such modifications are intended to be included within the scope of the invention.

The specification of the Japanese patent application to which the present application claims priority under the Paris Convention is incorporated herein by reference in its entirety.

1. An organic electroluminescence device comprising a cathode, an anode, and a light-emitting layer that is provided between the cathode and the anode, and the light-emitting layer comprising a compound represented by the following formula (1) and a compound represented by the following formula (11), 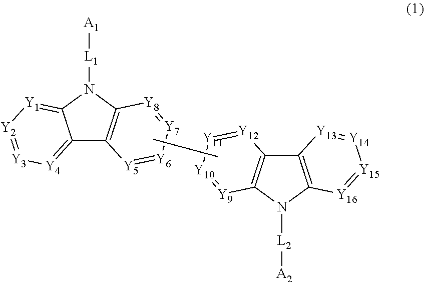
wherein A₁ is a substituted or unsubstituted aryl group including 6 to 18 ring carbon atoms,
A₂ is a substituted or unsubstituted fluoranthenyl group or a substituted or unsubstituted azafluoranthenyl group,
Y₁ to Y₁₆ are independently C(R),
R is independently a hydrogen atom, a substituted or unsubstituted aryl group including 5 to 30 ring carbon atoms, a substituted or unsubstituted alkyl group including 1 to 10 carbon atoms, a substituted or unsubstituted alkoxy group including 1 to 10 carbon atoms, a substituted or unsubstituted aralkyl group including 6 to 32 carbon atoms, a substituted or unsubstituted aryloxy group including 5 to 30 ring carbon atoms, a substituted or unsubstituted arylthio group including 5 to 30 ring carbon atoms, or a substituted or unsubstituted alkoxycarbonyl group including 2 to 11 carbon atoms, provided that R in one of Y₅ to Y₉ and R in one of Y₉ to Y₁₂ are a single bond which bonds with each other,
adjacent Rs may be bonded with each other to form a saturated or unsaturated, 5-membered or 6-membered ring structure which may have a substituent, and
L₁ and L₂ are independently a single bond, a substituted or unsubstituted arylene group including 6 to 18 ring carbon atoms or a substituted or unsubstituted heteroarylene group including 3 to 20 ring atoms, 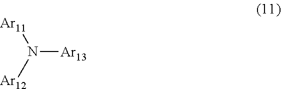
wherein Ar₁₁ to Ar₁₃ are independently a substituted or unsubstituted aryl group including 5 to 50 ring carbon atoms, a substituted or unsubstituted dibenzofuranyl group, a substituted or unsubstituted dibenzothiophenyl group, a substituted or unsubstituted alkyl group including 1 to 50 carbon atoms, a substituted or unsubstituted alkoxy group including 1 to 50 carbon atoms, a substituted or unsubstituted aralkyl group including 6 to 50 carbon atoms, a substituted or unsubstituted aryloxy group including 5 to 50 ring carbon atoms, a substituted or unsubstituted arylthio group including 5 to 50 ring carbon atoms, a substituted or unsubstituted alkoxycarbonyl group including 2 to 50 carbon atoms, an amino group substituted with a substituted or unsubstituted aryl group including 5 to 50 ring carbon atoms, an amino group substituted with a substituted or unsubstituted dibenzofuranyl group or a substituted or unsubstituted dibenzothiophenyl group, a halogen atom, a cyano group, a nitro group, a hydroxyl group, or a carboxyl group.

2. The organic electroluminescence device according to claim 1, wherein the compound represented by the formula (1) is a compound represented by the following formula (2), (3) or (4), 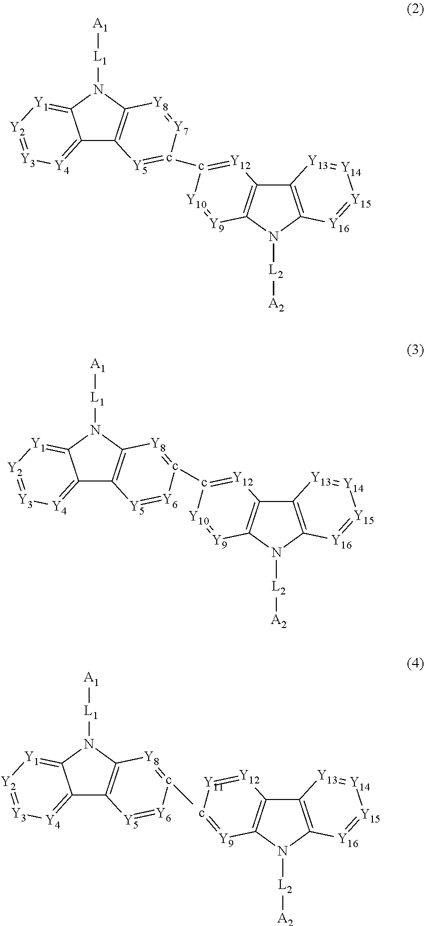
wherein A₁, A₂, Y₁ to Y₁₆, L₁ and L₂ are the same as defined for the formula (1).

3. The organic electroluminescence device according to claim 1, wherein R is independently a hydrogen atom, a substituted or unsubstituted alkyl group including 1 to 10 carbon atoms, a substituted or unsubstituted alkoxy group including 1 to 10 carbon atoms, a substituted or unsubstituted alkoxycarbonyl group including 2 to 11 carbon atoms or a single bond which bonds to a carbazole skeleton.

4. The organic electroluminescence device according to claim 1, wherein at least one of L₁ and L₂ is a single bond.

5. The organic electroluminescence device according to claim 1, wherein L₁ and L₂ are a single bond.

6. The organic electroluminescence device according to claim 1, wherein L₂ is a phenylene group.

7. The organic electroluminescence device according to claim 1, wherein A₁ is a phenyl group or a naphthyl group.

8. The organic electroluminescence device according to claim 1, wherein R is a phenyl group or a naphthyl group.

9. The organic electroluminescence device according to claim 1, wherein A₂ is a group represented by the following formula (7), 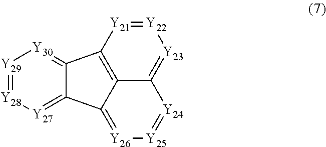
wherein Y₂₁ to Y₃₀ are independently C(R₄) or N,
R₄ is independently a hydrogen atom, a substituted or unsubstituted aryl group including 6 to 30 ring carbon atoms, a substituted or unsubstituted alkyl group including 1 to 10 carbon atoms, a substituted or unsubstituted alkoxy group including 1 to 10 carbon atoms, a substituted or unsubstituted aralkyl group including 7 to 32 carbon atoms, a substituted or unsubstituted aryloxy group including 6 to 30 ring carbon atoms, a substituted or unsubstituted arylthio group including 6 to 30 ring carbon atoms, or a substituted or unsubstituted alkoxycarbonyl group including 2 to 11 carbon atoms, provided that one of R₄ is a single bond which bonds to L₂ and at least one of Y₂₁ to Y₃₀ is N.

10. The organic electroluminescence device according to claim 9, wherein Y₂₃ is N.

11. The organic electroluminescence device according to claim 9, wherein Y₂₂ is C(R₄) and R₄ of Y₂₂ is a single bond which bonds to L₂.

12. The organic electroluminescence device according to claim 9, wherein Y₂₈ is C(R₄) and R₄ of Y₂₈ is a single bond which bonds to L₂.

13. The organic electroluminescence device according to claim 9, wherein the compound represented by the formula (1) is a compound represented by the following formula (8), 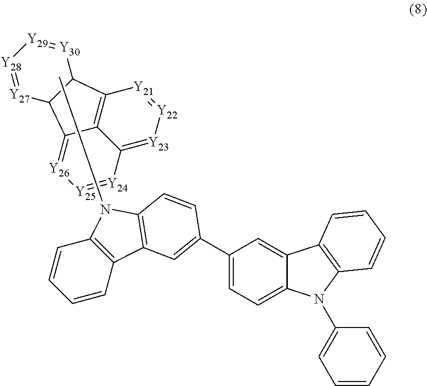
wherein Y₂₁ to Y₃₀ are the same as defined for the formula (7).

14. The organic electroluminescence device according to claim 1, wherein A₂ is a group represented by the following formula (5), 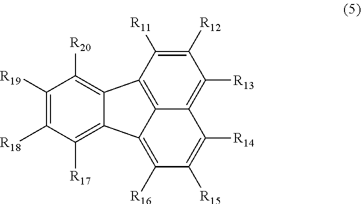
wherein R₁₁ to R₂₀ are independently a hydrogen atom, a substituted or unsubstituted aryl group including 5 to 30 ring carbon atoms, a substituted or unsubstituted alkyl group including 1 to 10 carbon atoms, a substituted or unsubstituted alkoxy group including 1 to 10 carbon atoms, a substituted or unsubstituted aralkyl group including 6 to 32 carbon atoms, a substituted or unsubstituted aryloxy group including 5 to 30 ring carbon atoms, a substituted or unsubstituted arylthio group including 5 to 30 ring carbon atoms, or a substituted or unsubstituted alkoxycarbonyl group including 2 to 11 carbon atoms, provided that one of R₁₁ to R₂₀ is a single bond which bonds to L₂.

15. The organic electroluminescence device according to claim 14, wherein R₁₄ is a single bond which bonds to L₂.

16. The organic electroluminescence device according to claim 14, wherein R₁₈ is a single bond which bonds to L₂.

17. The organic electroluminescence device according to claim 14, wherein the compound represented by the formula (1) is a compound represented by the following formula (6).

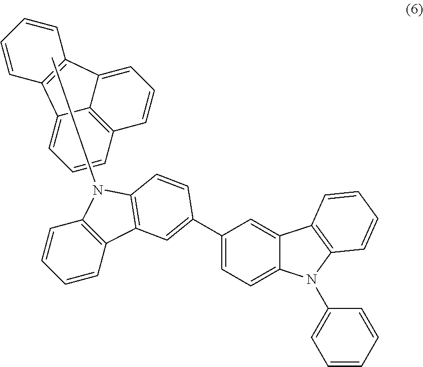

18. The organic electroluminescence device according to claim 1, wherein Ar₁₁ to Ar₁₃ are independently a substituted or unsubstituted aryl group including 5 to 50 ring carbon atoms.

19. The organic electroluminescence device according to claim 1, wherein at least one of Ar₁₁ to Ar₁₃ is selected from the group consisting of a substituted or unsubstituted phenyl group, a substituted or unsubstituted naphthyl group, a substituted or unsubstituted biphenyl group, a substituted or unsubstituted terphenyl group, a substituted or unsubstituted phenanthryl group, and a substituted or unsubstituted fluorenyl group.

20. The organic electroluminescence device according to claim 1, wherein at least one of Ar₁₁ to Ar₁₃ is a substituted aryl group including 5 to 50 ring carbon atoms, and at least one of the remaining groups is an aryl group including 5 to 50 ring carbon atoms substituted with a substituted or unsubstituted dibenzofuranyl group or a substituted or unsubstituted dibenzothiophenyl group.

21. The organic electroluminescence device according to claim 1, wherein the compound represented by the formula (11) is a compound represented by the following formula (12) or (13), 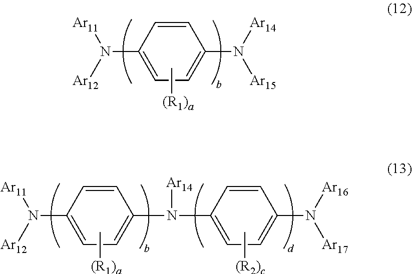
wherein Ar₁₁, Ar₁₂ and Ar₁₄ to Ar₁₇ are independently a substituted or unsubstituted aryl group including 5 to 50 ring carbon atoms, a substituted or unsubstituted dibenzofuranyl group, a substituted or unsubstituted dibenzothiophenyl group, a substituted or unsubstituted alkyl group including 1 to 50 carbon atoms, a substituted or unsubstituted alkoxy group including 1 to 50 carbon atoms, a substituted or unsubstituted aralkyl group including 6 to 50 carbon atoms, a substituted or unsubstituted aryloxy group including 5 to 50 ring carbon atoms, a substituted or unsubstituted arylthio group including 5 to 50 ring carbon atoms, a substituted or unsubstituted alkoxycarbonyl group including 2 to 50 carbon atoms, an amino group substituted with a substituted or unsubstituted aryl group including 5 to 50 ring carbon atoms, an amino group substituted with a substituted or unsubstituted dibenzofuranyl group or a substituted or unsubstituted dibenzothiophenyl group, a halogen atom, a cyano group, a nitro group, a hydroxyl group, or a carboxyl group,
R₁ and R₂ are independently a hydrogen atom, a substituted or unsubstituted aryl group including 5 to 50 ring carbon atoms, a substituted or unsubstituted dibenzofuranyl group, a substituted or unsubstituted dibenzothiophenyl group, a substituted or unsubstituted alkyl group including 1 to 50 carbon atoms, a substituted or unsubstituted alkoxy group including 1 to 50 carbon atoms, a substituted or unsubstituted aralkyl group including 6 to 50 carbon atoms, a substituted or unsubstituted aryloxy group including 5 to 50 ring carbon atoms, a substituted or unsubstituted arylthio group including 5 to 50 ring carbon atoms, a substituted or unsubstituted alkoxycarbonyl group including 2 to 50 carbon atoms, an amino group substituted with a substituted or unsubstituted aryl group including 5 to 50 ring carbon atoms, an amino group substituted with a substituted or unsubstituted dibenzofuranyl group or a substituted or unsubstituted dibenzothiophenyl group, a halogen atom, a cyano group, a nitro group, a hydroxyl group, or a carboxyl group,
a and c are independently an integer of 0 to 4, and b and d are independently an integer of 1 to 3,
plural R₁s may be bonded with each other to form a saturated or unsaturated, 5-membered or 6-membered ring structure which may have a substituent, and
plural R₂s may be bonded with each other to form a saturated or unsaturated, 5-membered or 6-membered ring structure which may have a substituent.

22. The organic electroluminescence device according to claim 21, wherein the compound represented by the formula (11) is a compound represented by the formula (12), wherein at least one of Ar₁₁, Ar₁₂, Ar₁₄ and Ar₁₅ is selected from the group consisting of a substituted or unsubstituted phenyl group, a substituted or unsubstituted naphthyl group, a substituted or unsubstituted biphenyl group, a substituted or unsubstituted terphenyl group, a substituted or unsubstituted phenanthryl group, and a substituted or unsubstituted fluorenyl group.

23. The organic electroluminescence device according to claim 21, wherein the compound represented by the formula (11) is a group represented by the formula (13), wherein at least one of Ar₁₁, Ar₁₂, Ar₁₄, Ar₁₆ and Ar₁₇ is selected from the group consisting of a substituted or unsubstituted phenyl group, a substituted or unsubstituted naphthyl group, a substituted or unsubstituted biphenyl group, a substituted or unsubstituted terphenyl group, a substituted or unsubstituted phenanthryl group, and a substituted or unsubstituted fluorenyl group.

24. The organic electroluminescence device according to claim 1, wherein the compound represented by the formula (11) is a compound represented by the following formula (14), 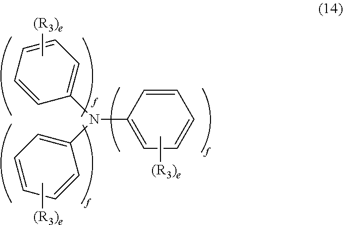
wherein R₃ is independently a hydrogen atom, a substituted or unsubstituted aryl group including 5 to 50 ring carbon atoms, a substituted or unsubstituted dibenzofuranyl group, a substituted or unsubstituted dibenzothiophenyl group, a substituted or unsubstituted alkyl group including 1 to 50 carbon atoms, a substituted or unsubstituted alkoxy group including 1 to 50 carbon atoms, a substituted or unsubstituted aralkyl group including 6 to 50 carbon atoms, a substituted or unsubstituted aryloxy group including 5 to 50 ring carbon atoms, a substituted or unsubstituted arylthio group including 5 to 50 ring carbon atoms, a substituted or unsubstituted alkoxycarbonyl group including 2 to 50 carbon atoms, an amino group substituted with a substituted or unsubstituted aryl group including 5 to 50 ring carbon atoms, an amino group substituted with a substituted or unsubstituted dibenzofuranyl group or a substituted or unsubstituted dibenzothiophenyl group, a halogen atom, a cyano group, a nitro group, a hydroxyl group, or a carboxyl group,
e is independently an integer of 0 to 4, and f is independently an integer of 1 to 3, and
adjacent Rₐs may be bonded with each other to form a saturated or unsaturated, 5-membered or 6-membered ring structure which may have a substituent.

25. The organic electroluminescence device according to claim 1, wherein R is independently a hydrogen atom, a substituted or unsubstituted aryl group including 5 to 10 ring carbon atoms, a substituted or unsubstituted alkyl group including 1 to 10 carbon atoms, a substituted or unsubstituted alkoxy group including 1 to 10 carbon atoms, a substituted or unsubstituted aralkyl group including 6 to 12 carbon atoms, a substituted or unsubstituted aryloxy group including 5 to 10 ring carbon atoms, a substituted or unsubstituted arylthio group including 5 to 10 ring carbon atoms, or a substituted or unsubstituted alkoxycarbonyl group including 2 to 11 carbon atoms,
L₁ and L₂ are independently a single bond, a substituted or unsubstituted arylene group including 6 to 18 ring carbon atoms or a substituted or unsubstituted heteroarylene group including 3 to 15 ring atoms.

26. The organic electroluminescence device according to claim 14, wherein R₁₁ to R₂₀ are independently a hydrogen atom, a substituted or unsubstituted aryl group including 5 to 10 ring carbon atoms, a substituted or unsubstituted alkyl group including 1 to 10 carbon atoms, a substituted or unsubstituted alkoxy group including 1 to 10 carbon atoms, a substituted or unsubstituted aralkyl group including 6 to 12 carbon atoms, a substituted or unsubstituted aryloxy group including 5 to 10 ring carbon atoms, a substituted or unsubstituted arylthio group including 5 to 10 ring carbon atoms, a substituted or unsubstituted alkoxycarbonyl group including 2 to 11 carbon atoms, and at least one of R₁₁ to R₂₀ is a single bond which bonds to L₂.

27. The organic electroluminescence device according to claim 14, wherein R is independently a hydrogen atom, a substituted or unsubstituted aryl group including 5 to 10 ring carbon atoms, a substituted or unsubstituted alkyl group including 1 to 10 carbon atoms, a substituted or unsubstituted alkoxy group including 1 to 10 carbon atoms, a substituted or unsubstituted aralkyl group including 6 to 12 carbon atoms, a substituted or unsubstituted aryloxy group including 5 to 10 ring carbon atoms, a substituted or unsubstituted arylthio group including 5 to 10 ring carbon atoms, or a substituted or unsubstituted alkoxycarbonyl group including 2 to 11 carbon atoms,
L₁ and L₂ are independently a single bond, a substituted or unsubstituted arylene group including 6 to 18 ring carbon atoms or a substituted or unsubstituted heteroarylene group including 3 to 15 ring atoms,
R₁₁ to R₂₀ are independently a hydrogen atom, a substituted or unsubstituted aryl group including 5 to 10 ring carbon atoms, a substituted or unsubstituted alkyl group including 1 to 10 carbon atoms, a substituted or unsubstituted alkoxy group including 1 to 10 carbon atoms, a substituted or unsubstituted aralkyl group including 6 to 12 carbon atoms, a substituted or unsubstituted aryloxy group including 5 to 10 ring carbon atoms, a substituted or unsubstituted arylthio group including 5 to 10 ring carbon atoms, or a substituted or unsubstituted alkoxycarbonyl group including 2 to 11 carbon atoms, and at least one of R₁₁ to R₂₀ is a single bond which bonds to L₂.

28. The organic electroluminescence device according to claim 1, wherein an ionization potential of the compound represented by the formula (11) is smaller than an ionization potential of the compound represented by the formula (1).

29. The organic electroluminescence device according to claim 1, wherein a value of “(an ionization potential of the compound represented by the formula (1))−(an ionization potential of the compound represented by the formula (11))” is more than 0 eV and 0.25 eV or less.

30. The organic electroluminescence device according to claim 1, wherein the light-emitting layer further comprises an ortho-metalated complex of a metal atom selected from Ir, Tb and Eu.

31. The organic electroluminescence device according to claim 1, wherein a hole transporting layer is provided between the anode and the light-emitting layer, and wherein the hole transporting layer comprises the compound represented by the formula (11).

32. The organic electroluminescence device according to claim 31, wherein the compound represented by the formula (11) is the compound represented by the formula (12) or (13).

33. The organic electroluminescence device according to claim 31, wherein the compound represented by the formula (11) is the compound represented by the formula (14).

34. The organic electroluminescence device according to claim 1, wherein an electron transporting layer is provided between the cathode and the light-emitting layer, and wherein the electron transporting layer comprises a carbazole derivative.

35. A lighting apparatus which is provided with the organic electroluminescence device according to claim 1.

36. A displaying apparatus which is provided with the organic electroluminescence device according to claim 1.

37. A mixed material comprising a compound represented by the following formula (1) and a compound represented by the following formula (11), 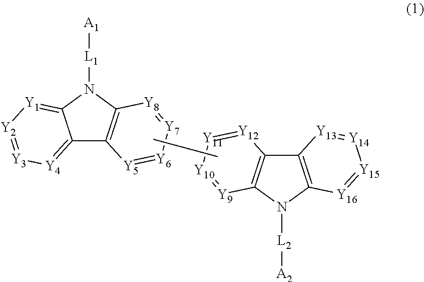
wherein A₁ is a substituted or unsubstituted aryl group including 6 to 18 ring carbon atoms,
A₂ is a substituted or unsubstituted fluoranthenyl group or a substituted or unsubstituted azafluoranthenyl group,
Y₁ to Y₁₆ are independently C(R), R is independently a hydrogen atom, a substituted or unsubstituted aryl group including 5 to 30 ring carbon atoms, a substituted or unsubstituted alkyl group including 1 to 10 carbon atoms, a substituted or unsubstituted alkoxy group including 1 to 10 carbon atoms, a substituted or unsubstituted aralkyl group including 6 to 32 carbon atoms, a substituted or unsubstituted aryloxy group including 5 to 30 ring carbon atoms, a substituted or unsubstituted arylthio group including 5 to 30 ring carbon atoms, or a substituted or unsubstituted alkoxycarbonyl group including 2 to 11 carbon atoms, provided that R in one of Y₅ to Y₈ and R in one of Y₉ to Y₁₂ are a single bond which bonds with each other,
adjacent Rs may be bonded with each other to form a saturated or unsaturated, 5-membered or 6-membered ring structure which may have a substituent, and
L₁ and L₂ are independently a single bond, a substituted or unsubstituted arylene group including 6 to 18 ring carbon atoms or a substituted or unsubstituted heteroarylene group including 3 to 20 ring atoms, 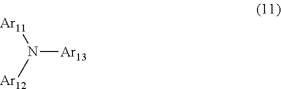
wherein Ar₁₁ to Ar₁₃ are independently a substituted or unsubstituted aryl group including 5 to 50 ring carbon atoms, a substituted or unsubstituted dibenzofuranyl group, a substituted or unsubstituted dibenzothiophenyl group, a substituted or unsubstituted alkyl group including 1 to 50 carbon atoms, a substituted or unsubstituted alkoxy group including 1 to 50 carbon atoms, a substituted or unsubstituted aralkyl group including 6 to 50 carbon atoms, a substituted or unsubstituted aryloxy group including 5 to 50 ring carbon atoms, a substituted or unsubstituted arylthio group including 5 to 50 ring carbon atoms, a substituted or unsubstituted alkoxycarbonyl group including 2 to 50 carbon atoms, an amino group substituted with a substituted or unsubstituted aryl group including 5 to 50 ring carbon atoms, an amino group substituted with a substituted or unsubstituted dibenzofuranyl group or a substituted or unsubstituted dibenzothiophenyl group, a halogen atom, a cyano group, a nitro group, a hydroxyl group, or a carboxyl group.

38. The mixed material according to claim 37, wherein R is independently a hydrogen atom, a substituted or unsubstituted aryl group including 5 to 10 ring carbon atoms, a substituted or unsubstituted alkyl group including 1 to 10 carbon atoms, a substituted or unsubstituted alkoxy group including 1 to 10 carbon atoms, a substituted or unsubstituted aralkyl group including 6 to 12 carbon atoms, a substituted or unsubstituted aryloxy group including 5 to 10 ring carbon atoms, a substituted or unsubstituted arylthio group including 5 to 10 ring carbon atoms, a substituted or unsubstituted alkoxycarbonyl group including 2 to 11 carbon atoms,
L₁ and L₂ are independently a single bond, a substituted or unsubstituted arylene group including 6 to 18 ring carbon atoms or a substituted or unsubstituted heteroarylene group including 3 to 15 ring atoms.

39. The mixed material according to claim 37, wherein an ionization potential of the compound represented by the formula (11) is smaller than an ionization potential of the compound represented by the formula (1).

40. The mixed material according to claim 37, wherein a value of “(an ionization potential of the compound represented by the formula (1))−(an ionization potential of the compound represented by the formula (11))” is more than 0 eV and 0.25 eV or less.

41. The mixed material according to claim 37 which is a material used for an organic electroluminescence device.
